(19) United States
(12) Patent Application Publication
(10) Pub. No.: US 2022/0372483 A1
(43) Pub. Date: Nov. 24, 2022

(54) Modified Guide RNAs for Gene Editing

(71) Applicant: Intellia Therapeutics, Inc., Cambridge, MA (US)

(72) Inventors: Seth C. Alexander, Medford, MA (US); Sabin Mulepati, Somerville, MA (US); Matthew Roy, Arlington, MA (US)

(73) Assignee: Intellia Therapeutics, Inc., Cambridge, MA (US)

(21) Appl. No.: 17/836,265

(22) Filed: Jun. 9, 2022

Related U.S. Application Data
(63) Continuation of application No. PCT/US2020/064250, filed on Dec. 10, 2020.
(60) Provisional application No. 62/946,905, filed on Dec. 11, 2019.

(51) Int. Cl. C12N 15/113; C12N 9/22

(52) U.S. Cl. CPC C12N 15/113; C12N 9/22; C12N 2310/20; C12N 2310/315

(57) ABSTRACT

This disclosure relates to modified guide RNAs having improved in vitro and in vivo activity in gene editing methods.

This patent application is a Continuation application of International Application No. PCT/US2020/064250, filed on Dec. 10, 2020, which claims the benefit of priority to U.S. Provisional Application No. 62/946,905, filed Dec. 11, 2019, the contents of each of which are incorporated herein by reference in its entirety for all purposes.

The instant application contains a Sequence Listing which has been submitted electronically in ASCII format and is hereby incorporated by reference in its entirety. Said ASCII copy, created on Jun. 9, 2022, is named 01155-0032-00US_ST25.txt and is 614,545 bytes in size.

This disclosure relates to the field of gene editing using CRISPR/Cas systems, a part of the prokaryotic immune system that recognizes and cuts exogenous genetic elements. The CRISPR/Cas system relies on a single nuclease, termed CRISPR-associated protein 9 (Cas9), which induces site-specific breaks in DNA. Cas9 is guided to specific DNA sequences by small RNA molecules termed guide RNA (gRNA). A complete guide RNA comprises tracrRNA (trRNA) and crisprRNA (crRNA). A crRNA comprising a guide region may also be referred to as a gRNA, with the understanding that to form a complete gRNA it should be or become associated covalently or noncovalently with a trRNA. The trRNA and crRNA may be contained within a single guide RNA (sgRNA) or in two separate RNA molecules of a dual guide RNA (dgRNA). Cas9 in combination with trRNA and crRNA or an sgRNA is termed the Cas9 ribonucleoprotein complex (RNP).

Oligonucleotides, and in particular RNA, are sometimes degraded in cells and in serum by non-enzymatic, endonuclease or exonuclease cleavage. Oligonucleotides can be synthesized with modifications at various positions to reduce or prevent such degradation. Given the cyclic nature and imperfect yield of oligonucleotide synthesis, shortening the gRNA while retaining or even improving its activity would be desirable, e.g., so that the gRNA can be obtained in greater yield, and/or compositions comprising the gRNA have greater homogeneity and/or fewer incomplete or erroneous products. Additionally, improved methods and compositions for preventing such degradation, improving stability of gRNAs and enhancing gene editing efficiency is desired, especially for therapeutic applications.

### SUMMARY

In some embodiments, genome editing tools are provided comprising guide RNA (gRNA) with one or more shortened regions and/or substitutions as described herein. The shortened regions or substitutions described herein may facilitate synthesis of the gRNA with greater yield and/or homogeneity, and/or may improve the stability of the gRNA and the gRNA/Cas9 complex and improve the activity of Cas9 (e.g., SaCas9, SpyCas9, and equivalents) to cleave target DNA.

In some embodiments, crisprRNA (crRNA) and/or tracrRNA (trRNA) with one or more shortened regions and/or substitutions as described herein are provided. In some embodiments, the modified crRNA and/or modified trRNA comprise a dual guide RNA (dgRNA). In some embodiments, the modified crRNA and/or modified trRNA comprise a single guide RNA (sgRNA). The shortened regions and/or substitutions described herein may facilitate synthesis of the gRNA with greater yield and/or homogeneity and/or may improve the stability of the gRNA and the gRNA/Cas9 complex and improve the activity of Cas9 (e.g., SauCas9, SpyCas9, and equivalents) to cleave target DNA. Compared to 100mer sgRNAs or other short guide RNAs, synthesis of the presently disclosed guide RNAs may increase crude yield of a guide RNA. Similarly, gRNA sample purity as measured by the proportion of full length product, e.g. crude purity, can be increased. gRNA can be obtained in greater yield, and/or compositions comprising the gRNA can have greater homogeneity and/or fewer incomplete or erroneous products. Guide RNA purity may be assessed using ion-pair reversed-phase high performance liquid chromatography (IP-RP-HPLC) and ion exchange HPLC methods, e.g. as in Kanavarioti et al, Sci Rep 9, 1019 (2019) (doi:10.1038/s41598-018-37642-z). Using UV spectroscopy at a wavelength of 260 nm, crude purity and final purity can be determined by the ratio of absorbance of the main peak to the cumulative absorbance of all peaks in the chromatogram. Synthetic yield is determined as the ratio of the absorbance at 260 nm of the final sample compared to the theoretical absorbance of input materials.

The following embodiments are encompassed.

Embodiment 1 is a guide RNA (gRNA) comprising a 5′ end modification or a 3′ end modification and a conserved portion of an gRNA comprising one or more of:

(a) a shortened hairpin 1 region or a substituted and optionally shortened hairpin 1 region, wherein (i) the shortened hairpin 1 region lacks 6-8 nucleotides; and (A) one or more of positions H1-1, H1-2, or H1-3 is deleted or substituted relative to SEQ ID NO: 400 and/or (B) one or more of positions H1-6 through H1-10 is substituted relative to SEQ ID NO: 400; or (ii) the shortened hairpin 1 region lacks 9-10 nucleotides including H1-1 and/or H1-12; or (iii) the shortened hairpin 1 region lacks 5-10 nucleotides and one or more of positions N18, H1-12, or N is substituted relative to SEQ ID NO: 400; or (iv) at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9, and the hairpin 1 region optionally lacks (aa) any one or two of H1-5 through H1-8, (bb) one, two, or three of the following pairs of nucleotides: H1-1 and H1-12, H1-2 and H1-11, H1-3 and H1-10 and/or H1-4 and H1-9, and/or (cc) 1-8 nucleotides of the hairpin 1 region; and/or

(b) a shortened upper stem region, wherein the shortened upper stem region lacks 1-6 nucleotides; and/or

(c) a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine.

Embodiment 1.01 is the gRNA of embodiment 1, wherein the hairpin 1 region is a substituted harpin 1 region and lacks 1 nucleotide.

Embodiment 1.02 is the gRNA of embodiment 1, wherein the hairpin 1 region is a substituted harpin 1 region and lacks 2 nucleotides.

Embodiment 1.03 is the gRNA of embodiment 1, wherein the hairpin 1 region is a substituted harpin 1 region and lacks 3 nucleotides.

Embodiment 1.04 is the gRNA of embodiment 1, wherein the hairpin 1 region is a substituted harpin 1 region and lacks 4 nucleotides.

Embodiment 1.05 is the gRNA of embodiment 1, wherein the hairpin 1 region is a substituted harpin 1 region and lacks 5 nucleotides.

Embodiment 1.06 is the gRNA of embodiment 1, wherein the hairpin 1 region is a substituted harpin 1 region and lacks 6 nucleotides.

Embodiment 1.07 is the gRNA of embodiment 1, wherein the hairpin 1 region is a substituted harpin 1 region and lacks 7 nucleotides.

Embodiment 1.08 is the gRNA of embodiment 1, wherein the hairpin 1 region is a substituted harpin 1 region and lacks 8 nucleotides.

Embodiment 1.09 is the gRNA of embodiment 1, wherein the gRNA comprises a substituted and optionally shortened hairpin 1 in which H1-1 and H1-12 are substituted with Watson-Crick pairing nucleotides.

Embodiment 1.10 is the gRNA of any one of the preceding embodiments, wherein the gRNA comprises a substituted and optionally shortened hairpin 1 in which H1-2 and H1-11 are substituted with Watson-Crick pairing nucleotides.

Embodiment 1.11 is the gRNA of any one of the preceding embodiments, wherein the gRNA comprises a substituted and optionally shortened hairpin 1 in which H1-3 and H1-10 are substituted with Watson-Crick pairing nucleotides.

Embodiment 1.12 is the gRNA of any one of the preceding embodiments, wherein the gRNA comprises a substituted and optionally shortened hairpin 1 in which H1-4 and H1-9 are substituted with Watson-Crick pairing nucleotides.

Embodiment 1.13 is the gRNA of any one of the preceding embodiments, wherein position H1-5 is deleted.

Embodiment 1.14 is the gRNA of any one of the preceding embodiments, wherein position H1-6 is deleted.

Embodiment 1.15 is the gRNA of any one of the preceding embodiments, wherein position H1-7 is deleted.

Embodiment 1.16 is the gRNA of any one of the preceding embodiments, wherein position H1-8 is deleted.

Embodiment 1.17 is the gRNA of any one of the preceding embodiments, wherein two of H1-5, H1-6, H1-7, and H1-8 are deleted.

Embodiment 1.18 is the gRNA of any one of the preceding embodiments, wherein positions H1-1 and H1-12 are deleted.

Embodiment 1.19 is the gRNA of any one of the preceding embodiments, wherein positions H1-2 and H1-11 are deleted.

Embodiment 1.20 is the gRNA of any one of the preceding embodiments, wherein positions H1-3 and H1-10 are deleted.

Embodiment 1.21 is the gRNA of any one of the preceding embodiments, wherein positions H1-4 and H1-9 are deleted.

Embodiment 1.22 is the gRNA of any one of the preceding embodiments, wherein two pairs of positions H1-1 and H1-12, positions H1-2 and H1-11, positions H1-3 and H1-10 and positions H1-4 and H1-9 are deleted.

Embodiment 1.23 is the gRNA of any one of the preceding embodiments, wherein three pairs of positions H1-1 and H1-12, positions H1-2 and H1-11, positions H1-3 and H1-10 and positions H1-4 and H1-9 are deleted.

Embodiment 2 is the gRNA of any one of the preceding embodiments, wherein position H1-1 is deleted.

Embodiment 3 is the gRNA of any one of embodiments 1-1.23, wherein position H1-1 is substituted.

Embodiment 4 is the gRNA of any one of the preceding embodiments, wherein position H1-2 is deleted.

Embodiment 5 is the gRNA of any one of embodiments 1-3, wherein position H1-2 is substituted.

Embodiment 6 is the gRNA of any one of the preceding embodiments, wherein position H1-3 is deleted.

Embodiment 7 is the gRNA of any one of embodiments 1-5, wherein position H1-3 is substituted.

Embodiment 8 is the gRNA of any one of the preceding embodiments, wherein position H1-4 is deleted.

Embodiment 9 is the gRNA of any one of embodiments 1-7, wherein position H1-5 is deleted.

Embodiment 10 is the gRNA of any one of the preceding embodiments, wherein position H1-6 is deleted.

Embodiment 11 is the gRNA of any one of embodiments 1-9, wherein position H1-6 is substituted.

Embodiment 12 is the gRNA of any one of the preceding embodiments, wherein position H1-7 is deleted.

Embodiment 13 is the gRNA of any one of embodiments 1-11, wherein position H1-7 is substituted.

Embodiment 14 is the gRNA of any one of the preceding embodiments, wherein position H1-8 is deleted.

Embodiment 15 is the gRNA of any one of embodiments 1-13, wherein position H1-8 is substituted.

Embodiment 16 is the gRNA of any one of the preceding embodiments, wherein position H1-9 is deleted.

Embodiment 17 is the gRNA of any one of embodiments 1-15, wherein position H1-9 is substituted.

Embodiment 18 is the gRNA of any one of the preceding embodiments, wherein position H1-10 is deleted.

Embodiment 19 is the gRNA of any one of embodiments 1-17, wherein position H1-10 is substituted.

Embodiment 20 is the gRNA of any one of the preceding embodiments, wherein position H1-11 is deleted.

Embodiment 21 is the gRNA of any one of the preceding embodiments, wherein position H1-12 is deleted.

Embodiment 22 is the gRNA of any one of embodiments 1-7, comprising a shortened hairpin 1 region that lacks 6-8 nucleotides.

Embodiment 23 is the gRNA of any one of the preceding embodiments, wherein the shortened hairpin 1 region has a length of 4 nucleotides.

Embodiment 24 is the gRNA of any one of embodiments 1-22, wherein the shortened hairpin 1 region has a length of 5 nucleotides.

Embodiment 25 is the gRNA of any one of embodiments 1-22, wherein the shortened hairpin 1 region has a length of 6 nucleotides.

Embodiment 26 is the gRNA of any one of embodiments 23-25, wherein the 4, 5, or 6 nucleotides of the shortened hairpin 1 region include less than or equal to 2 substitutions.

Embodiment 27 is the gRNA of embodiment 26, wherein the 4, 5, or 6 nucleotides of the shortened hairpin 1 region include one substitution.

Embodiment 28 is the gRNA of embodiment 26, wherein the 4, 5, or 6 nucleotides of the shortened hairpin 1 region are unsubstituted.

Embodiment 29 is the gRNA of any one of the preceding embodiments, wherein positions H1-2 through H1-4 are deleted.

Embodiment 30 is the gRNA of any one of the preceding embodiments, wherein positions H1-2 through H1-5 are deleted.

Embodiment 31 is the gRNA of any one of the preceding embodiments, wherein positions H1-9 through H1-11 are deleted.

Embodiment 32 is the gRNA of any one of the preceding embodiments, wherein positions H1-8 through H1-11 are deleted.

Embodiment 33 is the gRNA of any one of the preceding embodiments, wherein positions H1-2 through H1-4 and H1-9 through H1-11 are deleted.

Embodiment 34 is the gRNA of embodiment 33, wherein the shortened hairpin 1 region comprises:

(a) the sequence AGAAAU;
(b) a sequence having less than or equal to 2 mismatches to the sequence of (a); or
(c) a sequence having less than or equal to 1 mismatch to the sequence of (a).

Embodiment 35 is the gRNA of any one of embodiments 1-32, wherein positions H1-2 through H1-5 and H1-9 through H1-11 are deleted.

Embodiment 36 is the gRNA of embodiment 35, wherein each position of the upper stem region is modified, optionally wherein each position of the upper stem region is modified by 2′-O-methylation.

Embodiment 37 is the gRNA of any one of embodiments 35 or 36, wherein the shortened hairpin 1 region comprises:

(a) the sequence AAAAU;
(b) a sequence having less than or equal to 2 mismatches to the sequence of (a); or
(c) a sequence having less than or equal to 1 mismatch to the sequence of (a).

Embodiment 38 is the gRNA of any one of embodiments 1-32, wherein positions H1-2 through H1-5 and H1-8 through H1-11 are deleted.

Embodiment 39 is the gRNA of embodiment 38, wherein each position of the upper stem region is modified, optionally wherein each position of the upper stem region is modified by 2′-O-methylation.

Embodiment 40 is the gRNA of any one of embodiments 38 or 39, wherein the shortened hairpin 1 region comprises:

(a) the sequence AAAU;
(b) a sequence having less than or equal to 2 mismatches to the sequence of (a); or
(c) a sequence having less than or equal to 1 mismatch to the sequence of (a).

Embodiment 41 is the gRNA of any one of embodiments 1-32, wherein positions H1-1, H1-3 through H1-8, and H1-12 are deleted.

Embodiment 42 is the gRNA of embodiment 41, wherein each position of the upper stem region is modified, optionally wherein each position of the upper stem region is modified by 2′-O-methylation.

Embodiment 43 is the gRNA of any one of embodiments 41 or 42, wherein the shortened hairpin 1 region comprises:

(a) the sequence CAAG;
(b) a sequence having less than or equal to 2 mismatches to the sequence of (a); or
(c) a sequence having less than or equal to 1 mismatch to the sequence of (a).

Embodiment 44 is the gRNA of any one of the preceding embodiments, wherein positions H1-1 through H1-8 are deleted.

Embodiment 45 is the gRNA of any one of the preceding embodiments, wherein positions H1-11 through H1-12 are deleted.

Embodiment 46 is the gRNA of any one of embodiments 1-32, wherein positions H1-2 through H1-8 are deleted.

Embodiment 47 is the gRNA of embodiment 46, wherein the shortened hairpin 1 region comprises:

(a) the sequence AAAGU;
(b) a sequence having less than or equal to 2 mismatches to the sequence of (a); or
(c) a sequence having less than or equal to 1 mismatch to the sequence of (a).

Embodiment 48 is the gRNA of any one of embodiments 1-32, wherein positions H1-3 through H1-9 are deleted.

Embodiment 49 is the gRNA of embodiment 48, wherein the shortened hairpin 1 region comprises:

(a) the sequence ACAGU;
(b) a sequence having less than or equal to 2 mismatches to the sequence of (a); or
(c) a sequence having less than or equal to 1 mismatch to the sequence of (a).

Embodiment 50 is the gRNA of any one of the preceding embodiments, wherein position H1-7 is substituted with a G.

Embodiment 51 is the gRNA of any one of the preceding embodiments, wherein position H1-8 is substituted with a C.

Embodiment 52 is the gRNA of any one of the preceding embodiments, wherein positions H1-7 and H1-8 are substituted.

Embodiment 53 is the gRNA of any one of the preceding embodiments, wherein positions H1-7 and H1-8 are substituted with a G and a C, respectively.

Embodiment 54 is the gRNA of any one of the preceding embodiments, wherein positions H1-7 and H1-8 are substituted with a G and a C, respectively, and positions H1-2 through H1-4 and H1-9 through H1-11 are deleted.

Embodiment 55 is the gRNA of embodiment 54, wherein the shortened hairpin 1 region comprises:

(a) the sequence AGAGCU;
(b) a sequence having less than or equal to 2 mismatches to the sequence of (a); or
(c) a sequence having less than or equal to 1 mismatch to the sequence of (a).

Embodiment 56 is the gRNA of any one of the preceding embodiments, wherein position H1-6 is substituted with a C.

Embodiment 57 is the gRNA of any one of the preceding embodiments, wherein position H1-7 is substituted with a U.

Embodiment 58 is the gRNA of any one of the preceding embodiments, wherein positions H1-6 and H1-7 are substituted.

Embodiment 59 is the gRNA of any one of the preceding embodiments, wherein positions H1-6 and H1-7 are substituted with a C and a U, respectively.

Embodiment 60 is the gRNA of any one of the preceding embodiments, wherein positions H1-6 and H1-7 are substituted with a C and a U, respectively, and positions H1-2 through H1-4 and H1-9 through H1-11 are deleted.

Embodiment 61 is the gRNA of embodiment 60, wherein the shortened hairpin 1 region comprises:

(a) the sequence AGCUAU;
(b) a sequence having less than or equal to 2 mismatches to the sequence of (a); or
(c) a sequence having less than or equal to 1 mismatch to the sequence of (a).

Embodiment 62 is the gRNA of any one of the preceding embodiments, wherein position H1-1 is substituted with a C.

Embodiment 63 is the gRNA of any one of the preceding embodiments, wherein position H1-12 is substituted with a G.

Embodiment 64 is the gRNA of any one of the preceding embodiments, wherein positions H1-1 and H1-12 are substituted.

Embodiment 65 is the gRNA of any one of the preceding embodiments, wherein positions H1-1 and H1-12 are substituted with a C and a G, respectively.

Embodiment 66 is the gRNA of any one of the preceding embodiments, wherein positions H1-1 and H1-12 are substituted with a C and a G, respectively, and positions H1-2 through H1-4 and H1-9 through H1-11 are deleted.

Embodiment 67 is the gRNA of embodiment 66, wherein each position of the upper stem region is modified, optionally wherein each position of the upper stem region is modified by 2′-O-methylation.

Embodiment 68 is the gRNA of any one of embodiments 66 or 67, wherein the shortened hairpin 1 region comprises:

(a) the sequence CGAAAG;
(b) a sequence having less than or equal to 2 mismatches to the sequence of (a); or
(c) a sequence having less than or equal to 1 mismatch to the sequence of (a).

Embodiment 69 is the gRNA of any one of embodiments 1-22, comprising a shortened hairpin 1 region that lacks 9-10 nucleotides.

Embodiment 70 is the gRNA of embodiment 69, wherein the shortened hairpin 1 region has a length of 2 nucleotides.

Embodiment 71 is the gRNA of embodiment 69, wherein the shortened hairpin 1 region has a length of 3 nucleotides.

Embodiment 72 is the gRNA of embodiment 70 or 71, wherein the 2 or 3 nucleotides of the shortened hairpin 1 region are unsubstituted.

Embodiment 73 is the gRNA of any one of the preceding embodiments, wherein positions H1-11 through H1-12 are deleted.

Embodiment 74 is the gRNA of embodiment 73, wherein positions H1-1 through H1-8 and H1-11 through H1-12 are deleted.

Embodiment 75 is the gRNA of embodiment 74, wherein the shortened hairpin 1 region comprises:

(a) the sequence AA; or
(b) a sequence having less than or equal to 1 mismatch to the sequence of (a).

Embodiment 76 is the gRNA of any one of embodiments 1-21 or 69-72, wherein positions H1-1 through H1-9 and H1-12 are deleted.

Embodiment 77 is the gRNA of embodiment 76, wherein the shortened hairpin 1 region comprises:

(a) the sequence AG;
(b) a sequence having less than or equal to 1 mismatch to the sequence of (a).

Embodiment 78 is the gRNA of any one of embodiments 1-21, comprising a shortened hairpin 1 region that lacks 5-10 nucleotides.

Embodiment 79 is the gRNA of embodiment 78, wherein the shortened hairpin 1 region has a length of 7 nucleotides.

Embodiment 80 is the gRNA of any one of embodiments 78 or 79, wherein positions H1-4 through H1-11 are deleted.

Embodiment 81 is the gRNA of any one of the preceding embodiments, wherein position N18 is substituted.

Embodiment 82 is the gRNA of embodiment 81, wherein position N18 is substituted with a C.

Embodiment 83 is the gRNA of embodiment 82, wherein position N18 is substituted with a C and positions H1-4 through H1-11 are deleted.

Embodiment 84 is the gRNA of embodiment 83, wherein the gRNA comprises a segment containing position N18, the shortened hairpin 1 region, and position N, and the segment comprises:

(a) the sequence CACUUG;
(b) a sequence having less than or equal to 2 mismatches to the sequence of (a); or
(c) a sequence having less than or equal to 1 mismatch to the sequence of (a).

Embodiment 85 is the gRNA of any one of the preceding embodiments, wherein position H1-12 is substituted.

Embodiment 86 is the gRNA of embodiment 85, wherein position H1-12 is substituted with a C.

Embodiment 87 is the gRNA of embodiment 86, wherein position H1-12 is substituted with an A.

Embodiment 88 is the gRNA of any one of the preceding embodiments, wherein position N is substituted.

Embodiment 89 is the gRNA of embodiment 88, wherein position N is substituted with an A.

Embodiment 90 is the gRNA of embodiment 89, wherein position H1-12 is substituted with a C and position N is substituted with an A.

Embodiment 91 is the gRNA of embodiment 90, wherein position H1-12 is substituted with a C, position N is substituted with an A, and positions H1-4 through H1-11 are deleted.

Embodiment 92 is the gRNA of embodiment 91, wherein the gRNA comprises a segment containing position N18, the shortened hairpin 1 region, and position N, and the segment comprises:

(a) the sequence AACUCA;
(b) a sequence having less than or equal to 2 mismatches to the sequence of (a); or
(c) a sequence having less than or equal to 1 mismatch to the sequence of (a).

Embodiment 93 is the gRNA of embodiment 85, wherein position H1-12 is substituted with an A and position N is substituted with an A.

Embodiment 94 is the gRNA of embodiment 93, wherein position H1-12 is substituted with an A, position N is substituted with an A, and positions H1-4 through H1-11 are deleted.

Embodiment 95 is the gRNA of embodiment 94, wherein the gRNA comprises a segment containing position N18, the shortened hairpin 1 region, and position N, and the segment comprises:

(a) the sequence AACUAA;
(b) a sequence having less than or equal to 2 mismatches to the sequence of (a); or
(c) a sequence having less than or equal to 1 mismatch to the sequence of (a).

Embodiment 96 is the gRNA of any one of the preceding embodiments, comprising a shortened upper stem region.

Embodiment 97 is the gRNA of embodiment 96, wherein the shortened upper stem region lacks 1-6 nucleotides.

Embodiment 98 is the gRNA of embodiment 97, wherein the shortened upper stem region has a length of 6 nucleotides.

Embodiment 99 is the gRNA of embodiment 97, wherein the shortened upper stem region has a length of 7 nucleotides.

Embodiment 100 is the gRNA of embodiment 97, wherein the shortened upper stem region has a length of 8 nucleotides.

Embodiment 101 is the gRNA of embodiment 97, wherein the shortened upper stem region has a length of 9 nucleotides.

Embodiment 102 is the gRNA of embodiment 97, wherein the shortened upper stem region has a length of 10 nucleotides.

Embodiment 103 is the gRNA of embodiment 97, wherein the shortened upper stem region has a length of 11 nucleotides.

Embodiment 104 is the gRNA of any one of embodiments 98-103, wherein the 6, 7, 8, 9, 10, or 11 nucleotides of the shortened upper stem region include less than or equal to 4 substitutions.

Embodiment 105 is the gRNA of any one of embodiments 98-103, wherein the 6, 7, 8, 9, 10, or 11 nucleotides of the shortened upper stem region include less than or equal to 2 substitutions.

Embodiment 106 is the gRNA of any one of embodiments 98-103, wherein the 6, 7, 8, 9, 10, or 11 nucleotides of the shortened upper stem region include one substitution.

Embodiment 107 is the gRNA of any one of embodiments 98-103, wherein the 6, 7, 8, 9, 10, or 11 nucleotides of the shortened upper stem region are unsubstituted.

Embodiment 108 is the gRNA of any one of the preceding embodiments, wherein position US3 is deleted.

Embodiment 109 is the gRNA of any one of the preceding embodiments, wherein position US4 is deleted.

Embodiment 110 is the gRNA of any one of the preceding embodiments, wherein position US5 is deleted.

Embodiment 111 is the gRNA of any one of the preceding embodiments, wherein position US8 is deleted.

Embodiment 112 is the gRNA of any one of the preceding embodiments, wherein position US9 is deleted.

Embodiment 113 is the gRNA of any one of the preceding embodiments, wherein position US10 is deleted.

Embodiment 114 is the gRNA of any one of the preceding embodiments, wherein positions US4 and US9 are deleted.

Embodiment 115 is the gRNA of embodiment 114, wherein positions H1-2 through H1-5 and H1-8 through H1-11 are deleted.

Embodiment 116 is the gRNA of any one of embodiments 114 or 115, wherein the shortened upper stem region comprises:

(a) the sequence GCUGAAAGGC (SEQ ID NO: 1004);
(b) a sequence having less than or equal to 2 mismatches to the sequence of (a); or
(c) a sequence having less than or equal to 1 mismatch to the sequence of (a).

Embodiment 117 is the gRNA of any one of the preceding embodiments, wherein positions US3 and US4 are deleted.

Embodiment 118 is the gRNA of any one of the preceding embodiments, wherein positions US9 and US10 are deleted.

Embodiment 119 is the gRNA of embodiment 118, wherein positions US3, US4, US9, and US10 are deleted.

Embodiment 120 is the gRNA of embodiment 119, wherein positions H1-2 through H1-5 and H1-8 through H1-11 are deleted.

Embodiment 121 is the gRNA of any one of embodiments 119 or 120, wherein the shortened upper stem region comprises:

(a) the sequence GCGAAAGC;
(b) a sequence having less than or equal to 2 mismatches to the sequence of (a); or
(c) a sequence having less than or equal to 1 mismatch to the sequence of (a).

Embodiment 122 is the gRNA of embodiment 119 or 121, wherein positions H1-1 and H1-4 through H1-12 are deleted.

Embodiment 123 is the gRNA of embodiment 119, wherein positions US3, US4, US8, US9, and US10 are deleted.

Embodiment 124 is the gRNA of embodiment 123, wherein the shortened upper stem region comprises:

(a) the sequence GCGAAGC;
(b) a sequence having less than or equal to 2 mismatches to the sequence of (a); or
(c) a sequence having less than or equal to 1 mismatch to the sequence of (a).

Embodiment 125 is the gRNA of embodiment 119, wherein positions US3, US4, US5, US9, and US10 are deleted.

Embodiment 126 is the gRNA of embodiment 125, wherein the shortened upper stem region comprises:

(a) the sequence GCAAAGC (SEQ ID NO: 1005);
(b) a sequence having less than or equal to 2 mismatches to the sequence of (a); or
(c) a sequence having less than or equal to 1 mismatch to the sequence of (a).

Embodiment 127 is the gRNA of any one of embodiments 96-107, wherein position US3 is substituted, optionally with a G.

Embodiment 128 is the gRNA of any one of embodiments 96-107 or 127, wherein position US4 is substituted, optionally with a C.

Embodiment 129 is the gRNA of any one of embodiments 96-107 or 127-128, wherein position US9 is substituted, optionally with a G.

Embodiment 130 is the gRNA of any one of embodiments 96-107 or 127-129, wherein position US10 is substituted, optionally with a C.

Embodiment 131 is the gRNA of any one of embodiments 96-107 or 127-130, wherein positions US3 and US10 are substituted, optionally with a G and a C, respectively.

Embodiment 132 is the gRNA of any one of embodiments 96-107 or 127-131, wherein positions US4 and US9 are substituted, optionally with a C and a G, respectively.

Embodiment 133 is the gRNA of embodiment 132, wherein positions US3 and US10 are substituted with a G and a C, respectively, and positions US4 and US9 are substituted with a C and a G, respectively.

Embodiment 134 is the gRNA of embodiment 133, wherein position US5 is deleted.

Embodiment 135 is the gRNA of embodiment 133 or 134, wherein positions US3 and US10 are substituted with a G and a C, respectively, and positions US4 and US9 are substituted with a C and a G, respectively, and position US8 is deleted.

Embodiment 136 is the gRNA of embodiment 135, wherein positions H1-2 through H1-5 and H1-8 through H1-11 are deleted.

Embodiment 137 is the gRNA of embodiment 135 or 136, wherein the shortened upper stem region comprises:

(a) the sequence GCGCGAAGCGC (SEQ ID NO: 1008);
(b) a sequence having less than or equal to 2 mismatches to the sequence of (a); or
(c) a sequence having less than or equal to 1 mismatch to the sequence of (a).

Embodiment 138 is the gRNA of any one of embodiments 96-107 or 127-131, wherein positions US3 and US10 are substituted with a C and a G, respectively.

Embodiment 139 is the gRNA of embodiment 138, wherein positions US3 and US10 are substituted with a C and a G, respectively, and positions US4 and US9 are deleted.

Embodiment 140 is the gRNA of embodiment 139, wherein the shortened upper stem region comprises:

(a) the sequence GCGGAAACGC (SEQ ID NO: 1006);
(b) a sequence having less than or equal to 2 mismatches to the sequence of (a); or
(c) a sequence having less than or equal to 1 mismatch to the sequence of (a).

Embodiment 141 is the gRNA of any one of embodiments 96-107 or 127-131, wherein positions US3 and US10 are substituted with a G and a C, respectively, and positions US4 and US9 are deleted.

Embodiment 142 is the gRNA of embodiment 141, wherein the shortened upper stem region comprises:

(a) the sequence GCCGAAAGGC (SEQ ID NO: 1007);
(b) a sequence having less than or equal to 2 mismatches to the sequence of (a); or
(c) a sequence having less than or equal to 1 mismatch to the sequence of (a).

Embodiment 143 is the gRNA of any one of the preceding embodiments, wherein position LS6 is substituted.

Embodiment 144 is the gRNA of any one of the preceding embodiments, wherein position LS7 is substituted.

Embodiment 145 is the gRNA of any one of the preceding embodiments, wherein position US3 is substituted.

Embodiment 146 is the gRNA of any one of the preceding embodiments, wherein position US10 is substituted.

Embodiment 147 is the gRNA of any one of the preceding embodiments, wherein position B3 is substituted.

Embodiment 148 is the gRNA of embodiment 147, wherein position B3 is substituted with a G.

Embodiment 149 is the gRNA of any one of the preceding embodiments, wherein position N7 is substituted.

Embodiment 150 is the gRNA of embodiment 149, wherein position N7 is substituted with a C.

Embodiment 151 is the gRNA of embodiment 149, wherein position N7 is substituted with a U.

Embodiment 152 is the gRNA of any one of the preceding embodiments, wherein position N15 is substituted.

Embodiment 153 is the gRNA of embodiment 152, wherein position N15 is substituted with a C.

Embodiment 154 is the gRNA of embodiment 152, wherein position N15 is substituted with a U.

Embodiment 155 is the gRNA of any one of the preceding embodiments, wherein position N17 is substituted.

Embodiment 156 is the gRNA of embodiment 155, wherein position N17 is substituted with a G.

Embodiment 157 is the gRNA of any one of the preceding embodiments, wherein position H2-2 is substituted.

Embodiment 158 is the gRNA of any one of the preceding embodiments, wherein position H-14 is substituted.

Embodiment 159 is the gRNA of any one of the preceding embodiments, wherein positions LS 6 and LS7 are substituted.

Embodiment 160 is the gRNA of embodiment 159, wherein positions LS 6 and LS7 are substituted with a U and an A, respectively.

Embodiment 161 is the gRNA of any one of the preceding embodiments, wherein positions US3 and US10 are substituted.

Embodiment 162 is the gRNA of embodiment 161, wherein positions US3 and US10 are substituted with a G and a C, respectively.

Embodiment 163 is the gRNA of any one of the preceding embodiments, wherein positions H2-2 and H2-14 are substituted.

Embodiment 164 is the gRNA of embodiment 163, wherein positions H2-2 and H2-14 are substituted with an A and a U, respectively.

Embodiment 165 is the gRNA of embodiment 164, wherein positions H2-2 and H2-14 are substituted with a G and a C, respectively.

Embodiment 166 is the gRNA of any one of the preceding embodiments, wherein at least 2, 3, 4, 5, 6, 7, or 8 of positions US3, US10, LS6, LS7, B3, N15, N17, H2-2, and H2-14 are substituted.

Embodiment 167 is the gRNA of embodiment 166, wherein positions US3, US10, LS6, LS7, B3, N15, N17, H2-2, and H2-14 are substituted.

Embodiment 168 is the gRNA of any one of the preceding embodiments, wherein at least 2, 3, 4, or 5 of the following are true:

(a) positions US3 and US10 are substituted with a G and a C, respectively;
(b) positions LS 6 and LS7 are substituted with a U and an A, respectively;
(c) position B3 is substituted with a G;
(d) position N15 is substituted with a C;
(e) position N17 is substituted with a G; and/or
(F) positions H2-2 and H2-14 are substituted with an A and a U, respectively.

Embodiment 169 is the gRNA of embodiment, wherein positions US3 and US10 are substituted with a G and a C, respectively; positions LS 6 and LS7 are substituted with a U and an A, respectively; position B3 is substituted with a G; position N15 is substituted with a C; position N17 is substituted with a G; and positions H2-2 and H2-14 are substituted with an A and a U, respectively.

Embodiment 170 is the gRNA of any one of the preceding embodiments, wherein positions H1-4 through H1-11 are deleted.

Embodiment 171 is the gRNA of embodiment 170, wherein the shortened hairpin 1 region comprises:

(a) the sequence ACUU;
(b) a sequence having less than or equal to 2 mismatches to the sequence of (a); or
(c) a sequence having less than or equal to 1 mismatch to the sequence of (a).

Embodiment 172 is the gRNA of any one of the preceding embodiments, wherein position N2 is substituted with a C, optionally wherein positions H1-4 through H1-11 are deleted.

Embodiment 173 is the gRNA of any one of the preceding embodiments, wherein positions US1-US4 and US9-US12 are deleted, optionally wherein positions H1-4 through H1-11 are deleted.

Embodiment 174 is the gRNA of embodiment 173, wherein positions H1-2 to H1-11 are deleted.

Embodiment 175 is the gRNA of any one of the preceding embodiments, wherein positions H1-1 through H1-12 are deleted.

Embodiment 176 is the gRNA of any one of the preceding embodiments, wherein positions US2-US4 and US9-US11 are deleted.

Embodiment 177 is the gRNA of embodiment 176, wherein positions H1-2 to H1-11 are deleted.

Embodiment 178 is the gRNA of embodiment 176, wherein positions H1-1 and H1-4 through H1-12 are deleted.

Embodiment 179 is the gRNA of any one of embodiments 1-175, wherein positions US3-US5 and US8-US10 are deleted.

Embodiment 180 is the gRNA of any one of embodiments 1-175, wherein positions US3-US4 and US7-US10 are deleted.

Embodiment 181 is the gRNA of any one of embodiments 1-175, wherein positions US3-US10 are deleted.

Embodiment 182 is the gRNA of any one of embodiments 1-175, wherein positions US2-US5 and US8-US11 are deleted.

Embodiment 183 is the gRNA of any one of embodiments 1-175, wherein positions US2-US6 and US8-US11 are deleted.

Embodiment 184 is the gRNA of any one of embodiments 1-175, wherein positions US2-US11 are deleted.

Embodiment 185 is the gRNA of any one of embodiments 1-175, wherein positions US1-US5 and US8-US12 are deleted.

Embodiment 186 is the gRNA of any one of embodiments 1-175, wherein positions US1-US5 and US7-US12 are deleted.

Embodiment 187 is the gRNA of any one of the preceding embodiments, wherein position H2-15 is deleted.

Embodiment 188 is the gRNA of embodiment 187, wherein positions H2-14 and H2-15 are deleted.

Embodiment 189 is the gRNA of any one of the preceding embodiments, wherein position N6 is deleted, optionally wherein positions H1-4 through H1-11 are deleted.

Embodiment 190 is the gRNA of any one of the preceding embodiments, wherein position LS6 is substituted, optionally with a C.

Embodiment 191 is the gRNA of any one of the preceding embodiments, wherein position B3 is substituted, optionally with a C.

Embodiment 192 is the gRNA of any one of the preceding embodiments, wherein position N1 is substituted, optionally with a C.

Embodiment 193 is the gRNA of any one of the preceding embodiments, wherein position N7 is substituted, optionally with a G.

Embodiment 194 is the gRNA of any one of the preceding embodiments, wherein position N15 is substituted, optionally with a G.

Embodiment 195 is the gRNA of any one of the preceding embodiments, wherein position N17 is substituted with a non-pyrimidine, optionally with a G.

Embodiment 196 is the gRNA of any one of the preceding embodiments, wherein the gRNA is an sgRNA.

Embodiment 197 is the gRNA of any one of embodiments 1-195, which the gRNA is a crRNA or dgRNA.

Embodiment 198 is the gRNA of any one of the preceding embodiments, wherein the gRNA comprises a 5′ end modification.

Embodiment 199 is the gRNA of any one of the preceding embodiments, wherein the gRNA comprises a 3′ end modification.

Embodiment 200 is the gRNA of any one of the preceding embodiments, wherein the gRNA comprises a 5′ end modification and a 3′ end modification.

Embodiment 201 is the gRNA of any one of the preceding embodiments, wherein the gRNA comprises a 3′ tail.

Embodiment 202 is the gRNA of embodiment 201, wherein the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

Embodiment 203 is the gRNA of embodiment 202, wherein the 3′ tail comprises 1, 2, 3, 4, 5, 6, 7, 8, 9, or 10 nucleotides.

Embodiment 204 is the gRNA of any one of the preceding embodiments, wherein the gRNA does not comprise a 3′ tail.

Embodiment 205 is the gRNA of any one of the preceding embodiments, comprising a modification in the hairpin region.

Embodiment 206 is the gRNA of any one of the preceding embodiments, comprising a 3′ end modification, and a modification in the hairpin region.

Embodiment 207 is the gRNA of any one of the preceding embodiments, comprising a 3′ end modification, a modification in the hairpin region, and a 5′ end modification.

Embodiment 208 is the gRNA of any one of the preceding embodiments, comprising a 5′ end modification, and a modification in the hairpin region.

Embodiment 209 is the gRNA of any one of the preceding embodiments, further comprising a guide region.

Embodiment 210 is the gRNA of any one of the preceding embodiments, wherein the 3′ and/or 5′ end modification comprises a protective end modification, such as a modified nucleotide selected from 2′-O-methyl (2′-OMe) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

Embodiment 211 is the gRNA of any one of the preceding embodiments, wherein the modification in the hairpin region comprises a modified nucleotide selected from 2′-O-methyl (2′-Ome) modified nucleotide, a 2′-fluoro (2′-F) modified nucleotide, a phosphorothioate (PS) linkage between nucleotides, or combinations thereof.

Embodiment 212 is the gRNA of any one of the preceding embodiments, wherein the 3′ and/or 5′ end modification comprises or further comprises a 2′-O-methyl (2′-Ome) modified nucleotide.

Embodiment 213 is the gRNA of any one of the preceding embodiments, wherein the 3′ and/or 5′ end modification comprises or further comprises a 2′-fluoro (2′-F) modified nucleotide.

Embodiment 214 is the gRNA of any one of the preceding embodiments, wherein the 3′ and/or 5′ end modification comprises or further comprises a phosphorothioate (PS) linkage between nucleotides.

Embodiment 215 is the gRNA of any one of the preceding embodiments, wherein the 3′ and/or 5′ end modification comprises or further comprises an inverted abasic modified nucleotide.

Embodiment 216 is the gRNA of any one of the preceding embodiments, wherein the modification in the hairpin region comprises or further comprises a 2′-O-methyl (2′-Ome) modified nucleotide.

Embodiment 217 is the gRNA of any one of the preceding embodiments, wherein the modification in the hairpin region comprises or further comprises a 2′-fluoro (2′-F) modified nucleotide.

Embodiment 218 is the gRNA of any one of the preceding embodiments, wherein the 3′ end modification comprises any of:

i. a modification of any one or more of the last 7, 6, 5, 4, 3, 2, or 1 nucleotides;

ii. one modified nucleotide;

iii. two modified nucleotides;

iv. three modified nucleotides;

v. four modified nucleotides;

vi. five modified nucleotides;

vii. six modified nucleotides; and

viii. seven modified nucleotides.

Embodiment 219 is the gRNA of any one of the preceding embodiments, wherein the 3′ end modification comprises one or more of:

i. a phosphorothioate (PS) linkage between nucleotides;

ii. a 2′-Ome modified nucleotide;

iii. a 2′-O-moe modified nucleotide;

iv. a 2′-F modified nucleotide;

v. an inverted abasic modified nucleotide; and

vi. a combination of one or more of (i.)-(v.).

Embodiment 220 is the gRNA of any one of the preceding embodiments, wherein the gRNA comprises a 3′ tail comprising one or more of:

i. a phosphorothioate (PS) linkage between nucleotides;

ii. a 2′-Ome modified nucleotide;

iii. a 2′-O-moe modified nucleotide;

iv. a 2′-F modified nucleotide;

v. an inverted abasic modified nucleotide; and

vi. a combination of one or more of (i.)-(v.).

Embodiment 221 is the gRNA any one of the preceding embodiments, wherein the gRNA comprises one or more of:

i. 1, 2, 3, 4, 5, 6, 7, 8, 9, or 10 PS linkages between nucleotides;

ii. 1, 2, 3, 4, 5, 6, 7, 8, 9, 10, 12, 14, 16, or 18 PS linkages between nucleotides;

iii. about 1-3, 1-5, 1-6, 1-7, 1-8, 1-9, or 1-10 PS linkages between nucleotides;

iv. about 1-3, 1-5, 1-6, 1-7, 1-8, 1-9, 1-10, 1-12, 1-14, 1-16, 1-18, or 1-20 PS linkages between nucleotides; and

v. PS linkages between each nucleotide.

Embodiment 222 is the gRNA of any one of the preceding embodiments, wherein the 3′ end modification comprises at least one PS linkage, and wherein one or more of:

i. there is one PS linkage, and the linkage is between the last and second to last nucleotide;

ii. there are two PS linkages between the last three nucleotides;

iii. there are PS linkages between any one or more of the last four nucleotides;

iv. there are PS linkages between any one or more of the last five nucleotides; and

v. there are PS linkages between any one or more of the last 2, 3, 4, 5, 6, 7, 8, 9, or 10 nucleotides.

Embodiment 223 is the gRNA of embodiment 222, wherein the 3′ end modification further comprises at least one 2′-OMe, 2′-O-moe, inverted abasic, or 2′-F modified nucleotide.

Embodiment 224 is the gRNA of any one of the preceding embodiments, wherein the 3′ end modification comprises:

i. a modification of one or more of the last 1-7 nucleotides, wherein the modification is a PS linkage, inverted abasic nucleotide, 2′-Ome, 2′-O-moe, 2′-F, or combinations thereof;

ii. a modification to the last nucleotide with 2′-Ome, 2′-O-moe, 2′-F, or combinations thereof, and an optional one or two PS linkages to the next nucleotide and/or the first nucleotide of the 3′ tail;

iii. a modification to the last and/or second to last nucleotide with 2′-Ome, 2′-O-moe, 2′-F, or combinations thereof, and optionally one or more PS linkages;

iv. a modification to the last, second to last, and/or third to last nucleotides with 2′-Ome, 2′-O-moe, 2′-F, or combinations thereof, and optionally one or more PS linkages;

v. a modification to the last, second to last, third to last, and/or fourth to last nucleotides with 2′-Ome, 2′-O-moe, 2′-F, or combinations thereof, and optionally one or more PS linkages; or

vi. a modification to the last, second to last, third to last, fourth to last, and/or fifth to last nucleotides with 2′-Ome, 2′-O-moe, 2′-F, or combinations thereof, and optionally one or more PS linkages.

Embodiment 225 is the gRNA of any one of the preceding embodiments, wherein the sgRNA comprise a 3′ tail, wherein the 3′ tail comprises a modification of any one or more of the nucleotides present in the 3′ tail.

Embodiment 226 is the gRNA of embodiment 225, wherein the 3′ tail is fully modified.

Embodiment 227 is the gRNA of embodiment 225, wherein the gRNA comprises a shortened hairpin 1 region and the gRNA comprises modifications at least H2-1 to H2-12.

Embodiment 228 is the gRNA of any one of the preceding embodiments, wherein the gRNA comprises any one or more of:

i. the 3′ end modification as shown in any one of SEQ ID Nos: 101-190, 301-394, or 795-798;

ii. (i) a 2′-OMe modified nucleotide at the last nucleotide of the conserved region of the gRNA, (ii) three consecutive 2′O-moe modified nucleotides immediately 5′ to the 2′-OMe modified nucleotide, and (iii) three consecutive PS linkages between the last three nucleotides of the conserved region of the gRNA;

iii. (i) five consecutive 2′-OMe modified nucleotides from the 3′ end of the 3′ terminus, and (ii) three PS linkages between the last three nucleotides of the conserved region of the gRNA;

iv. an inverted abasic modified nucleotide at the last nucleotide of the conserved region of the gRNA;

v. (i) an inverted abasic modified nucleotide at the last nucleotide of the conserved region of the gRNA, and (ii) three consecutive 2′-OMe modified nucleotides at the last three nucleotides of the conserved region of the gRNA;

vi. (i) 15 consecutive 2′-OMe modified nucleotides from the 3′ end of the 3′ terminus, (ii) five consecutive 2′-F modified nucleotides immediately 5′ to the 2′-OMe modified nucleotides, and (iii) three PS linkages between the last three nucleotides of the conserved region of the gRNA;

vii. (i) alternating 2′-OMe modified nucleotides and 2′-F modified nucleotides at the last 20 nucleotides of the conserved region of the gRNA, and (ii) three PS linkages between the last three nucleotides of the conserved region of the gRNA;

viii. (i) two or three consecutive 2′-OMe modified nucleotides, and (ii) three PS linkages between the last three nucleotides of the conserved region of the gRNA;

ix. one PS linkage between the last and next to last nucleotides of the conserved region of the gRNA; and

x. 15 or 20 consecutive 2′-OMe modified nucleotides, and (ii) three PS linkages between the last three nucleotides of the conserved region of the gRNA.

Embodiment 229 is the gRNA of any one of the preceding embodiments, wherein the 5′ end modification comprises any one or more of:

i. a modification of any one or more of nucleotides 1-7 of the guide region;

ii. one modified nucleotide;

iii. two modified nucleotides;

iv. three modified nucleotides;

v. four modified nucleotides;

vi. five modified nucleotides;

vii. six modified nucleotides; and

viii. seven modified nucleotides.

Embodiment 230 is the gRNA of any one of the preceding embodiments, wherein the 5′ end modification comprises a modification of between 1 and 7, between 1 and 5, between 1 and 4, between 1 and 3, or between 1 and 2 nucleotides.

Embodiment 231 is the gRNA of any one of the preceding embodiments, wherein the 5′ end modification comprises one or more of:

i. a phosphorothioate (PS) linkage between nucleotides;

ii. a 2′-OMe modified nucleotide;

iii. a 2′-O-moe modified nucleotide;

iv. a 2′-F modified nucleotide;

v. an inverted abasic modified nucleotide;

vi. a deoxyribonucleotide;

vii. an inosine; and

viii. combinations of one or more of (i.)-(vii.).

Embodiment 232 is the gRNA any one of the preceding embodiments, wherein the 5′ end modification comprises:

i. 1, 2, 3, 4, 5, 6, and/or 7 PS linkages between nucleotides; or

ii. about 1-2, 1-3, 1-4, 1-5, 1-6, or 1-7 PS linkages between nucleotides.

Embodiment 233 is the gRNA of any one of the preceding embodiments, wherein gRNA is an sgRNA and the 5′ end modification comprises at least one PS linkage, and wherein:

i. there is one PS linkage, and the linkage is between nucleotides 1 and 2 of the guide region;

ii. there are two PS linkages, and the linkages are between nucleotides 1 and 2, and 2 and 3 of the guide region;

iii. there are PS linkages between any one or more of nucleotides 1 and 2, 2 and 3, and 3 and 4 of the guide region;

iv. there are PS linkages between any one or more of nucleotides 1 and 2, 2 and 3, 3 and 4, and 4 and 5 of the guide region;

v. there are PS linkages between any one or more of nucleotides 1 and 2, 2 and 3, 3 and 4, 4 and 5, and 5 and 6 of the guide region;

vi. there are PS linkages between any one or more of nucleotides 1 and 2, 2 and 3, 3 and 4, 4 and 5, 5 and 6, and 6 and 7 of the guide region; or vii. there are PS linkages between any one or more of nucleotides 1 and 2, 2 and 3, 3 and 4, 4 and 5, 5 and 6, 6 and 7, and 7 and 8 of the guide region.

Embodiment 234 is the gRNA of embodiment 233, wherein the 5′ end modification further comprises at least one 2′-OMe, 2′-O-moe, inverted abasic, or 2′-F modified nucleotide.

Embodiment 235 is the gRNA of any one of the preceding embodiments, wherein the gRNA is an sgRNA comprising:

i. a modification of one or more of nucleotides 1-7 of the variable region, wherein the modification is a PS linkage, inverted abasic nucleotide, 2′-OMe, 2′-O-moe, 2′-F, 2′-H (a deoxyribonucleotide), an inosine, and/or combinations thereof;

ii. a modification to the first nucleotide of the guide region with 2′-OMe, 2′-O-moe, 2′-F, 2′-H, an inosine, or combinations thereof, and an optional PS linkage to the next nucleotide;

iii. a modification to the first and/or second nucleotide of the variable region with 2′-OMe, 2′-O-moe, 2′-F, 2′-H, an inosine, or combinations thereof, and optionally one or more PS linkages;

iv. a modification to the first, second, and/or third nucleotides of the variable region with 2′-OMe, 2′-O-moe, 2′-F, 2′-H, an inosine, or combinations thereof, and optionally one or more PS linkages;

v. a modification to the first, second, third, and/or fourth nucleotides of the variable region with 2′-OMe, 2′-O-moe, 2′-F, 2′-H, an inosine, or combinations thereof, and optionally one or more PS linkages; or

vi. a modification to the first, second, third, fourth, and/or fifth nucleotides of the variable region with 2′-OMe, 2′-O-moe, 2′-F, 2′-H, an inosine, or combinations thereof, and optionally one or more PS linkages.

Embodiment 236 is the gRNA of any one of the preceding embodiments, wherein the gRNA is an sgRNA comprising any one or more of:

i. a 5′ end modification as shown in any one of SEQ ID Nos: 101-190 or 795-798;

ii. 2′-OMe modified nucleotides at nucleotides 1, 2, and 3 of the guide region;

iii. 2′-OMe modified nucleotides at nucleotides 1, 2, and 3 of the guide region and PS linkages between nucleotides 1 and 2, 2 and 3, and 3 and 4 of the guide region;

iv. 2′-OMe modified nucleotides at nucleotides 1, 2, 3, 4, and 5 of the guide region;

v. 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region;

vi. 2′O-moe modified nucleotides at nucleotides 1, 2, and 3 of the guide region;

vii. 2′O-moe modified nucleotides at nucleotides 1, 2, and 3 of the guide region and PS linkages between nucleotides 1 and 2, 2 and 3, and 3 and 4 of the guide region;

viii. an inverted abasic modified nucleotide at nucleotide 1 of the guide region;

ix. an inverted abasic modified nucleotide at nucleotide 1 of the guide region and 2′-OMe modified nucleotides at nucleotides 1, 2, and 3 of the guide region; and

x. an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the variable region.

Embodiment 237 is the gRNA of any one of the preceding embodiments, wherein the upper stem region comprises at least one modification.

Embodiment 238 is the gRNA of any one of the preceding embodiments, wherein the upper stem modification comprises any one or more of:

i. a modification to any one or more of US1-US12 in the upper stem region;

ii. a modification of at least 1, 2, 3, 4, 5, 6, 7, 8, 9, 10, 11, or all 12 nucleotides in the upper stem region; and

iii. a modification of about 1-2, 1-3, 1-4, 1-5, 1-6, 1-7, 1-8, 1-9, 1-10, or 1-12 nucleotides in the upper stem region.

Embodiment 239 is the gRNA of embodiment 238, wherein the upper stem modification comprises one or more of:

i. a 2′-OMe modified nucleotide;

ii. a 2′-O-moe modified nucleotide;

iii. a 2′-F modified nucleotide; and

iv. combinations of one or more of (i.)-(iii.).

Embodiment 240 is the gRNA of any one of the preceding embodiments, wherein the 5′ end modification comprises any one or more of:

i. a 5′ end modification as shown in any one of SEQ ID Nos: 101-190 or 795-798;

ii. 2′-OMe modified nucleotides at nucleotides 1, 2, and 3 of the variable region;

iii. 2′-OMe modified nucleotides at nucleotides 1, 2, and 3 of the variable region and PS linkages between nucleotides 1 and 2, 2 and 3, and 3 and 4 of the variable region;

iv. 2′-OMe modified nucleotides at nucleotides 1, 2, 3, 4, and 5 of the variable region;

v. 2′-OMe modified nucleotides at nucleotides 1, 2, 3, 4, and 5 of the variable region and PS linkages between nucleotides 1 and 2, 2 and 3, 3 and 4, 4 and 5, and 5 and 6 of the variable region;

vi. 2′O-moe modified nucleotides at nucleotides 1, 2, and 3 of the variable region;

vii. 2′O-moe modified nucleotides at nucleotides 1, 2, and 3 of the variable region and PS linkages between nucleotides 1 and 2, 2 and 3, and 3 and 4 of the variable region;

viii. an inverted abasic modified nucleotide at nucleotide 1 of the variable region;

ix. an inverted abasic modified nucleotide at nucleotide 1 of the variable region and 2′-OMe modified nucleotides at nucleotides 1, 2, and 3 of the variable region; and

x. an inverted abasic modified nucleotide at nucleotide 1 of the variable region, 2′-OMe modified nucleotides at nucleotides 1, 2, and 3 of the variable region, and PS linkages between nucleotides 1 and 2, 2 and 3, 3 and 4, 4 and 5, and 5 and 6 of the variable region.

Embodiment 241 is the gRNA of any one of the preceding embodiments, wherein the gRNA comprises any one or more of:

i. a 3′ end modification shown in any one of SEQ ID Nos: 101-190, 301-395, or 795-798;

ii. (i) a 2′-OMe modified nucleotide at the last nucleotide of the conserved region of an sgRNA or gRNA, (ii) three consecutive 2′O-moe modified nucleotides immediately 5′ to the 2′-OMe modified nucleotide, and (iii) three consecutive PS linkages between the last three nucleotides;

iii. (i) five consecutive 2′-OMe modified nucleotides, and (ii) three PS linkages between the last three nucleotides;

iv. an inverted abasic modified nucleotide at the last nucleotide of the conserved region of an sgRNA or gRNA;

v. (i) an inverted abasic modified nucleotide at the last nucleotide of the conserved region of an sgRNA or gRNA, and (ii) three consecutive 2′-OMe modified nucleotides at the last three nucleotides of the conserved region of an sgRNA or gRNA;

vi. (i) 15 consecutive 2′-OMe modified nucleotides, (ii) five consecutive 2′-F modified nucleotides immediately 5′ to the 2′-OMe modified nucleotides, and (iii) three PS linkages between the last three nucleotides;

vii. (i) alternating 2′-OMe modified nucleotides and 2′-F modified nucleotides at the last 20 nucleotides of the conserved region of an sgRNA or gRNA, and (ii) three PS linkages between the last three nucleotides;

viii. (i) two or three consecutive 2′-OMe modified nucleotides, and (ii) three PS linkages between the last three nucleotides;

ix. one PS linkage between the last and next to last nucleotides; and

x. 15 or 20 consecutive 2′-OMe modified nucleotides, and (ii) three PS linkages between the last three nucleotides.

Embodiment 242 is the gRNA of any one of the preceding embodiments, comprising a nucleotide sequence having at least 99, 98, 97, 96, 95, 94, 93, 92, 91, 90, 85, 80, 75, or 70% identity to the nucleotide sequence of any one of SEQ ID Nos: 1-90, 201-290, 401-490, or 601-690.

Embodiment 243 is the gRNA of any one of the preceding embodiments, comprising a nucleotide sequence having at least 99, 98, 97, 96, 95, 94, 93, 92, 91, 90, 85, 80, 75, or 70% identity to the nucleotide sequence of any one of SEQ ID Nos: 101-190, 301-394, 501-594, or 701-798, wherein the modification at each nucleotide of the gRNA that corresponds to a nucleotide of the reference sequence identifier in Table 1A is identical to or equivalent to the modification shown in the reference sequence identifier in Table 1A.

Embodiment 244 is a guide RNA comprising any of SEQ ID Nos: 1-90, 201-290, 401-490, or 601-690.

Embodiment 245 is a guide RNA comprising any of SEQ ID Nos: 101-190, 301-394, 501-594, or 701-798, including the modifications of Table 1A.

Embodiment 246 is the gRNA of any one of the preceding embodiments, comprising a YA modification at at least one guide region YA site.

Embodiment 247 is the gRNA of any one of the preceding embodiments, comprising a YA modification at at least one guide region YA site that is not a 5′ end modification.

Embodiment 248 is the gRNA of any one of the preceding embodiments, comprising a YA modification at one or more guide region YA sites, wherein the guide region YA site is at or after nucleotide 8 from the 5′ end of the 5′ terminus.

Embodiment 249 is the gRNA of any one of the preceding embodiments comprising a YA modification at one or more guide region YA sites, wherein the gRNA comprises one or more of:

i. a modification at one or more of H1-1 and H2-1;

ii. a YA modification at 1, 2, 3, 4, or 5 guide region YA sites;

iii. a YA modification at 1, 2, 3, 4, or 5 guide region YA sites, wherein the modification of at least one guide region YA site is different from any 5′ end modification of the sgRNA;

iv. a YA modification at one or more guide region YA sites, wherein the guide region YA site is at or after nucleotide 8 from the 5′ end of the 5′ terminus;

v. a YA modification at one or more guide region YA sites, wherein the guide region YA site is within nucleotides 5-end, 6-end, 7-end, 8-end, 9-end, or 10-end from the 5′ end of the 5′ terminus;

vi. a YA modification at one or more guide region YA sites, wherein the guide region YA site is within 17, 16, 15, 14, 13, 12, 11, 10, or 9 nucleotides of the 3′ terminal nucleotide of the guide region;

vii. a YA modification at a guide region YA site other than a 5′ end modification;

viii. a YA modification at two or more guide region YA sites, wherein the guide region YA sites are at or after nucleotide 8 from the 5′ end of the 5′ terminus;

ix. a YA modification at two or more guide region YA sites, wherein the two guide region YA sites are within nucleotides 5-end, 6-end, 7-end, 8-end, 9-end, or 10-end from the 5′ end of the 5′ terminus;

x. a YA modification at two or more guide region YA sites, wherein the guide region YA sites are within 17, 16, 15, 14, 13, 12, 11, 10, or 9 nucleotides of the 3′ terminal nucleotide of the guide region;

xi. a YA modification at two or more guide region YA sites other than a 5′ end modification; and

xii. a YA modification at two or more guide region YA sites, wherein the modifications of the guide region YA sites comprise a modification that at least one nucleotide located 5′ of the guide region YA site does not comprise.

Embodiment 250 is the gRNA of any one of the preceding embodiments, comprising a YA modification wherein the modification comprises 2′-fluoro, 2′-H, 2′-OMe, ENA, UNA, inosine, or PS.

Embodiment 251 is the gRNA of any one of the preceding embodiments, comprising a YA modification wherein the modification alters the structure of the dinucleotide motif to reduce RNA endonuclease activity.

Embodiment 252 is the gRNA of any one of the preceding embodiments, comprising a YA modification wherein the modification interferes with recognition or cleavage of a YA site by an RNase and/or stabilizes an RNA structure.

Embodiment 253 is the gRNA of any one of the preceding embodiments, comprising a YA modification wherein the modification comprises one or more of:

i. a ribose modification selected from 2′-O-alkyl, 2′-F, 2′-moe, 2′-F arabinose, and 2′-H (deoxyribose);

ii. a bicyclic ribose analog, such as LNA, BNA, and ENA;

iii. an unlocked nucleic acid modification;

iv. a base modification, such as inosine, pseudouridine, and 5′-methylcytosine; and

v. an internucleoside linkage modification such as phosphorothioate.

Embodiment 254 is the gRNA of any one of the preceding embodiments, comprising a YA modification at one or more conserved region YA sites.

Embodiment 255 is the gRNA of any one of the preceding embodiments, comprising a YA modification at one or more of conserved region YA sites 2, 3, 4, and 10.

Embodiment 256 is the gRNA of any one of the preceding embodiments, comprising a YA modification at one or more of conserved region YA sites 1 and 8.

Embodiment 257 is the gRNA of any one of the preceding embodiments, comprising a YA modification of conserved region YA site 1.

Embodiment 258 is the gRNA of any one of the preceding embodiments, comprising a YA modification of conserved region YA site 2.

Embodiment 259 is the gRNA of any one of the preceding embodiments, comprising a YA modification of conserved region YA site 3.

Embodiment 260 is the gRNA of any one of the preceding embodiments, comprising a YA modification of conserved region YA site 4.

Embodiment 261 is the gRNA of any one of the preceding embodiments, comprising a YA modification of conserved region YA site 5.

Embodiment 262 is the gRNA of any one of the preceding embodiments, comprising a YA modification of conserved region YA site 6.

Embodiment 263 is the gRNA of any one of the preceding embodiments, comprising a YA modification of conserved region YA site 7.

Embodiment 264 is the gRNA of any one of the preceding embodiments, comprising a YA modification of conserved region YA site 8.

Embodiment 265 is the gRNA of any one of the preceding embodiments, comprising a YA modification of conserved region YA site 9.

Embodiment 266 is the gRNA of any one of the preceding embodiments, comprising a YA modification of conserved region YA site 10.

Embodiment 267 is the gRNA of any one of the preceding embodiments, comprising one or more of:

i. YA modifications of conserved region YA sites 2, 3, 4, and 10;

ii. YA modifications of conserved region YA sites 2, 3, and 4;

iii. YA modifications of conserved region YA sites 2, 3, and 10;

iv. YA modifications of conserved region YA sites 2, 4, and 10;

v. YA modifications of conserved region YA sites 3, 4, and 10;

vi. YA modifications of conserved region YA sites 2 and 10;

vii. YA modifications of conserved region YA sites 2 and 4;

viii. YA modifications of conserved region YA sites 2 and 3;

ix. YA modifications of conserved region YA sites 3 and 4;

x. YA modifications of conserved region YA sites 3 and 10;

xi. YA modifications of conserved region YA sites 4 and 10

xii. YA modifications of conserved region YA sites 1 and 5;

xiii. YA modifications of conserved region YA sites 1 and 6;

xiv. YA modifications of conserved region YA sites 1 and 7;

xv. YA modifications of conserved region YA sites 1 and 8;

xvi. YA modifications of conserved region YA sites 1 and 9;

xvii. YA modifications of conserved region YA sites 8 and 5;

xviii. YA modifications of conserved region YA sites 8 and 6;

xix. YA modifications of conserved region YA sites 8 and 7; and

xx. YA modifications of conserved region YA sites 8 and 9;

xxi. optionally wherein the sgRNA further comprises YA modifications of conserved region YA sites 2, 3, 4, and/or 10.

Embodiment 268 is the gRNA of any one of the preceding embodiments, wherein at least one modified YA site comprises a 2′-OMe modification, optionally at the pyrimidine of the YA site.

Embodiment 269 is The gRNA of any one of the preceding embodiments, wherein at least one modified YA site comprises a 2′-fluoro modification, optionally at the pyrimidine of the YA site.

Embodiment 270 is the gRNA of any one of the preceding embodiments, wherein at least one modified YA site comprises a PS modification, optionally at the pyrimidine of the YA site.

Embodiment 271 is the gRNA of any one of the preceding embodiments, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.

Embodiment 272 is the gRNA of any one of the preceding embodiments, wherein the gRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, 2′-H, inosine, or phosphorothioate modifications.

Embodiment 273 is the gRNA of any one of embodiments 271-272, wherein 2′-OMe modifications are not present in the guide region at nucleotides 6-11 and 13-end.

Embodiment 274 is the gRNA of any one of embodiments 271-273, wherein 2′-fluoro modifications are not present in the guide region at nucleotides 1-7, 15, 16, and 19-end.

Embodiment 275 is the gRNA of any one of embodiments 271-274, wherein phosphorothioate modifications are not present in the guide region at nucleotides 4, 5, 11-14, 17, and 18.

Embodiment 276 is the gRNA of any one of embodiments 271-275, wherein the guide region comprises an unmodified nucleotide 20.

Embodiment 277 is the gRNA of any one of embodiments 271-276, wherein the guide region consists of 20 nucleotides.

Embodiment 278 is the gRNA of any one of embodiments 271-277, wherein the guide region comprises a YA site at nucleotides 5-6 and a modification at nucleotide 5.

Embodiment 279 is the gRNA of any one of embodiments 271-278, wherein the guide region comprises a YA site at nucleotides 12-13 and a modification at nucleotide 12.

Embodiment 280 is the gRNA of any one of embodiments 271-279, wherein the guide region comprises a YA site at nucleotides 15-16 and a modification at nucleotide 15.

Embodiment 281 is the gRNA of any one of embodiments 271-280, wherein the guide region comprises a YA site at nucleotides 16-17 and a modification at nucleotide 16.

Embodiment 282 is the gRNA of any one of embodiments 271-281, wherein the guide region comprises a YA site at nucleotides 19-20 and a modification at nucleotide 19.

Embodiment 283 is the gRNA of any one of embodiments 271-277 or 279-282, wherein the guide region does not comprise a YA site at nucleotides 5-6 and nucleotide 5 is unmodified.

Embodiment 284 is the gRNA of any one of embodiments 271-278 or 280-283, wherein the guide region does not comprise a YA site at nucleotides 12-13 and nucleotide 12 is unmodified.

Embodiment 285 is the gRNA of any one of embodiments 271-279 or 281-284, wherein the guide region does not comprise a YA site at nucleotides 15-16 and nucleotide 15 is unmodified.

Embodiment 286 is the gRNA of any one of embodiments 271-280 or 282-285, wherein the guide region does not comprise a YA site at nucleotides 16-17 and nucleotide 16 is unmodified.

Embodiment 287 is the gRNA of any one of embodiments 271-281 or 283-286, wherein the guide region does not comprise a YA site at nucleotides 19-20 and nucleotide 19 is unmodified.

Embodiment 288 is the gRNA of any one of embodiments 271-287, wherein the gRNA comprises a guide region that comprises one or more of the following:

i. 2′-OMe and phosphorothioate modifications at nucleotide 1;

ii. 2′-OMe and phosphorothioate modifications at nucleotide 2;

iii. 2′-OMe and phosphorothioate modifications at nucleotide 3;

iv. a 2′-OMe modification at nucleotide 4;

v. a phosphorothioate modification at nucleotide 6;

vi. a phosphorothioate modification at nucleotide 7;

vii. 2′-fluoro and phosphorothioate modifications at nucleotide 8;

viii. 2′-fluoro and phosphorothioate modifications at nucleotide 9;

ix. 2′-fluoro and phosphorothioate modifications at nucleotide 10;

x. a 2′-fluoro modification at nucleotide 11;

xi. a 2′-fluoro modifications at nucleotide 13;

xii. a 2′-fluoro modifications at nucleotide 14;

xiii. a 2′-fluoro modifications at nucleotide 17; and

xiv. a 2′-fluoro modifications at nucleotide 18.

Embodiment 289 is the gRNA of any one of embodiments 271-288, wherein the guide region comprises each of the modifications set forth in the preceding embodiment.

Embodiment 290 is the gRNA of any one of embodiments 271-289, wherein the guide region comprises at least 1, 2, 3, or 4 of the following:

i. a 2′-OMe modification at nucleotide 5 if nucleotides 5 and 6 form a YA site;

ii. a 2′-OMe modification at nucleotide 12 if nucleotides 12 and 13 form a YA site;

iii. a phosphorothioate modification at nucleotide 15 if nucleotides 15 and 16 form a YA site;

iv. a phosphorothioate modification at nucleotide 16 if nucleotides 16 and 17 form a YA site; and

v. a phosphorothioate or 2′-fluoro modification at nucleotide 19 if nucleotides 19 and 20 form a YA site.

Embodiment 291 is the gRNA of any one of embodiments 271-290, wherein the guide region comprises a YA site at nucleotides 5-6 and a 2′-OMe modification at nucleotide 5.

Embodiment 292 is the gRNA of any one of embodiments 271-291, wherein the guide region comprises a YA site at nucleotides 12-13 and a 2′-OMe modification at nucleotide 12.

Embodiment 293 is the gRNA of any one of embodiments 271-292, wherein the guide region comprises a YA site at nucleotides 15-16 and a phosphorothioate modification at nucleotide 15.

Embodiment 294 is the gRNA of any one of embodiments 271-293, wherein the guide region comprises a YA site at nucleotides 16-17 and a phosphorothioate modification at nucleotide 16.

Embodiment 295 is the gRNA of any one of embodiments 271-294, wherein the guide region comprises a YA site at nucleotides 19-20 and a phosphorothioate modification at nucleotide 19.

Embodiment 296 is the gRNA of any one of embodiments 271-295, wherein the guide region comprises a 2′-fluoro modification at nucleotide 19.

Embodiment 297 is the gRNA of any one of embodiments 271-296, wherein the guide region comprises an unmodified nucleotide 15 or only a phosphorothioate modification at nucleotide 15.

Embodiment 298 is the gRNA of any one of embodiments 271-297, wherein the guide region comprises an unmodified nucleotide 16 or only a phosphorothioate modification at nucleotide 16.

Embodiment 299 is the gRNA of any one of the preceding embodiments, comprising:

i. a YA modification at 1, 2, 3, 4, or 5 guide region YA sites;

ii. a YA modification at 1, 2, 3, 4, or 5 guide region YA sites, wherein the modification of at least one guide region YA site is different from any 5′ end modification of the sgRNA;

iii. a YA modification at one or more guide region YA sites that are at or after nucleotide 8 from the 5′ end of the 5′ terminus;

iv. a YA modification at one or more guide region YA sites that are is within nucleotides 5-end, 6-end, 7-end, 8-end, 9-end, or 10-end from the 5′ end of the 5′ terminus;

v. a YA modification at one or more guide region YA sites that are within 17, 16, 15, 14, 13, 12, 11, 10, or 9 nucleotides of the 3′ terminal nucleotide of the guide region;

vi. a YA modification at a guide region YA site other than a 5′ end modification; or

vii. a YA modification at a guide region YA site, wherein the modification of the guide region YA site comprises a modification at at least one nucleotide located 5′ of the guide region YA site does not comprise.

Embodiment 300 is the gRNA of embodiment 300, comprising:

i. a YA modification at two or more guide region YA sites that are at or after nucleotide 8 from the 5′ end of the 5′ terminus;

ii. a YA modification at two or more guide region YA sites that are within nucleotides 5-end, 6-end, 7-end, 8-end, 9-end, or 10-end from the 5′ end of the 5′ terminus;

iii. a YA modification at two or more guide region YA sites that are within 17, 16, 15, 14, 13, 12, 11, 10, or 9 nucleotides of the 3′ terminal nucleotide of the guide region;

iv. a YA modification at two or more guide region YA sites other than a 5′ end modification; or

v. a YA modification at a two or more guide region YA sites, wherein the modifications of the guide region YA sites comprise a modification at at least one nucleotide located 5′ of the guide region YA site does not comprise.

Embodiment 301 is the gRNA of embodiment 300, comprising:

i. a YA modification at three or more guide region YA sites that are at or after nucleotide 8 from the 5′ end of the 5′ terminus;

ii. a YA modification at three or more guide region YA sites that are within nucleotides 5-end, 6-end, 7-end, 8-end, 9-end, or 10-end from the 5′ end of the 5′ terminus;

iii. a YA modification at three or more guide region YA sites that are within 17, 16, 15, 14, 13, 12, 11, 10, or 9 nucleotides of the 3′ terminal nucleotide of the guide region;

iv. a YA modification at three or more guide region YA sites other than a 5′ end modification; or

v. a YA modification at a three or more guide region YA sites, wherein the modifications of the guide region YA sites comprise a modification at at least one nucleotide located 5′ of the guide region YA site does not comprise.

Embodiment 302 is the gRNA of any one of embodiments 299-301, wherein at least 1, 2, 3, 4, 5, 6, 7, or 8 of nucleotides 8-11, 13-14, and 17-18 from the 5′ end of the 5′ terminus comprise a YA modification.

Embodiment 303 is the gRNA of embodiment 302, wherein the modification of at least 1, 2, 3, 4, 5, 6, 7, or 8 of nucleotides 8-11, 13-14, and 17-18 from the 5′ end of the 5′ terminus comprises 2′-fluoro, 2′-H, 2′-OMe, or PS.

Embodiment 304 is the gRNA of embodiment 303, wherein the modification is 2′-fluoro.

Embodiment 305 is the gRNA of embodiment 303, wherein the modification is 2′-OMe or 2′-H.

Embodiment 306 is the gRNA of embodiment 303, wherein the modification is PS.

Embodiment 307 is the gRNA of any one of embodiments 299-306, wherein at least 1, 2, 3, 4, or 5 of nucleotides 6-10 from the 5′ end of the 5′ terminus comprise a YA modification, optionally wherein the modification comprises 2′-fluoro, 2′-H, 2′-OMe, inosine, or PS.

Embodiment 308 is the gRNA of embodiment 307, wherein the modification is PS.

Embodiment 309 is the gRNA of embodiment 307, wherein the modification is 2′-fluoro or 2′-H.

Embodiment 310 is the gRNA of embodiment 307, wherein the modification is 2′-OMe.

Embodiment 311 is the gRNA of any one of embodiments 299-310, comprising any one or more of the following:

i. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a modification other than 2′-fluoro at one or more of nucleotides 6-10 from the 5′ terminus;

ii. a YA modification other than PS at one or more of nucleotides 8-11, 13-14, and 17-18 from the 5′ end of the 5′ terminus, and 1, 2, 3, 4, or 5 YA modifications at nucleotides 6-10 from the 5′ end of the 5′ terminus, optionally wherein the modifications are PS modifications;

iii. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and modifications other than 2′-fluoro at nucleotides 6-10 from the 5′ end of the 5′ terminus;

iv. YA modifications other than PS at each of nucleotides 8-11, 13-14, and 17-18 from the 5′ end of the 5′ terminus, and 1, 2, 3, 4, or 5 YA modifications at nucleotides 6-10 from the 5′ end of the 5′ terminus, wherein the modifications are optionally PS modifications;

v. 1, 2, 3, 4, 5, 6, 7, or 8 YA modifications at nucleotides 8-11, 13-14, and 17-18 from the 5′ end of the 5′ terminus, wherein the YA modifications are optionally 2′-fluoro modifications, and one or more PS modification at any one of nucleotides 6-10 from the 5′ end of the 5′ terminus;

vi. at least one 2′-fluoro modification at any one of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications;

vii. 1, 2, 3, 4, 5, 6, 7, or 8 YA modifications of nucleotides 8-11, 13-14, and 17-18 from the 5′ end of the 5′ terminus, wherein the YA modifications are optionally 2′-fluoro modifications, and a PS modification at each of nucleotides 6-10 from the 5′ end of the 5′ terminus; or

viii. a 2′-fluoro modification at each of nucleotides 8-11, 13-14, and 17-18 from the 5′ end of the 5′ terminus, and 1, 2, 3, 4, or 5 YA modifications of nucleotides 6-10 from the 5′ end of the 5′ terminus, wherein the modifications are optionally PS modifications.

Embodiment 312 is the gRNA of any one of embodiments 299-311, wherein:

i. nucleotides 4-20 from the 5′ end of the 5′ terminus comprise at least 2, 3, or 4 modified YA sites including a first modified YA site comprising a 2′-OMe modification and a second modified YA site comprising a 2′-fluoro modification or a PS modification;

ii. nucleotides 4-20 from the 5′ end of the 5′ terminus comprise at least 2, 3, or 4 modified YA sites including a first modified YA site comprising a 2′-fluoro modification and a second modified YA site comprising a 2′-OMe modification or a PS modification;

iii. nucleotides 4-20 from the 5′ end of the 5′ terminus comprise at least 2, 3, or 4 modified YA sites including a first modified YA site comprising a PS modification and a second modified YA site comprising a 2′-OMe modification or a 2′-fluoro modification;

iv. nucleotides 4-20 from the 5′ end of the 5′ terminus comprise at least 2, 3, or 4 modified YA sites including a YA modification;

v. nucleotides 4-20 from the 5′ end of the 5′ terminus comprise at least 3 or 4 modified YA sites including a first modified YA site comprising a 2′-OMe modification, a second modified YA site comprising a 2′-fluoro modification, and a third modified YA site comprising a PS modification;

vi. nucleotides 4-20 from the 5′ end of the 5′ terminus comprise at least 3 or 4 modified YA sites including a first modified YA site comprising a 2′-OMe modification, a second modified YA site comprising a 2′-fluoro modification, a third modified YA site comprising a 2′-fluoro modification, and a fourth modified YA site comprising a PS modification;

vii. nucleotides 4-20 from the 5′ end of the 5′ terminus comprise at least 3 or 4 modified YA sites including a YA modification;

viii. nucleotides 4-20 from the 5′ end of the 5′ terminus comprise at least 4 modified YA sites including a first modified YA site comprising a 2′-OMe modification, a second modified YA site comprising a 2′-fluoro modification, a third modified YA site comprising a PS modification, and a fourth modified YA site comprising a PS modification; or

ix. nucleotides 4-40 from the 5′ end of the 5′ terminus comprise at least 4 modified YA sites including a YA modification.

Embodiment 313 is the gRNA of any one of embodiments 299-312, wherein nucleotides 4-20 from the 5′ end of the 5′ terminus comprise at least 5 modified YA sites.

Embodiment 314 is the gRNA of any one of embodiments 299-313, wherein the at least 5 modified YA sites include a fifth modified YA site comprising a PS modification, optionally wherein the third modified YA site comprises a 2′-fluoro modification.

Embodiment 315 is The gRNA of any one of embodiments 299-314, wherein the first, second, and (if applicable) third, fourth, and fifth of the at least 5 modified YA sites are arranged in the 5′ to 3′ direction.

Embodiment 316 is the gRNA of any one of embodiments 299-315, wherein the first, second, and (if applicable) third, fourth, and fifth of the at least 5 modified YA sites are not arranged in the 5′ to 3′ direction.

Embodiment 317 is the gRNA of any one of embodiments 299-316, wherein nucleotides 4-20 from the 5′ end of the 5′ terminus comprise at least 2, 3, 4, or 5 modified YA sites comprising a deoxyribonucleotide, optionally wherein the deoxyribonucleotide is the pyrimidine of the YA sites.

Embodiment 318 is the gRNA of any one of embodiments 299-317, wherein:

i. at least 1, 2, 3, or 4 of nucleotides 8-11 from the 5′ end of the 5′ terminus comprise a YA modification, which is optionally a 2′-fluoro modification;

ii. at least 1, 2, 3, 4, 5, 6, 7, or 8 of nucleotides 8-11, 13, 14, 17, and 18 from the 5′ end of the 5′ terminus comprise a YA modification, optionally wherein the YA modifications are 2′-OMe if present at nucleotides 8-11 and 2′-fluoro if present at nucleotides 13, 14, 17, or 18;

iii. at least one or both of nucleotides 17 and 18 from the 5′ end of the 5′ terminus comprise a YA modification, which is optionally a 2′-fluoro modification;

iv. at least one or both of nucleotides 17 and 18 from the 5′ end of the 5′ terminus comprise a YA modification, which is optionally a 2′-fluoro modification; or

v. at least 1, 2, 3, 4, 5, 6, 7, 8, 9, 10, 11, 12, or 13 of nucleotides 4-14, 17, and 18 from the 5′ end of the 5′ terminus comprise a YA modification, which is optionally a 2′-fluoro modification.

Embodiment 319 is the gRNA of any one of embodiments 299-318, wherein at least 1, 2, 3, 4, 5, or 6 of nucleotides 4-10 from the 5′ end of the 5′ terminus comprise a YA modification, which is optionally a 2′-OMe modification.

Embodiment 320 is the gRNA of any one of embodiments 299-319, wherein nucleotides 4-10 from the 5′ end of the 5′ terminus comprise a YA modification, which is optionally a 2′-OMe modification.

Embodiment 321 is the gRNA of any one of embodiments 299-320, wherein:

i. at least one of nucleotides 1-3 from the 5′ end of the 5′ terminus comprise a 5′ protective end modification, which is optionally a 2′-OMe modification;

ii. at least two of nucleotides 1-3 from the 5′ end of the 5′ terminus comprise a 5′ protective end modification, which is optionally a 2′-OMe modification; or

iii. each of nucleotides 1-3 from the 5′ end of the 5′ terminus comprise a 5′ protective end modification, which is optionally a 2′-OMe modification.

Embodiment 322 is the gRNA of any one of embodiments 299-321, wherein at least 1, 2, 3, 4, or 5 of nucleotides 11, 13, 14, 17, and 18 from the 5′ end of the 5′ terminus comprise a 5′ end modification, which is optionally a 2′-fluoro modification.

Embodiment 323 is the gRNA of any one of embodiments 299-322, wherein nucleotide 15 from the 5′ end of the 5′ terminus is unmodified or modified only with phosphorothioate.

Embodiment 324 is the gRNA of any one of embodiments 299-323, wherein nucleotide 16 from the 5′ terminus is unmodified or modified only with phosphorothioate.

Embodiment 325 is the gRNA of any one of 299-324, wherein nucleotide 3 from the 5′ end of the 5′ terminus is unmodified or modified only with phosphorothioate.

Embodiment 326 is the gRNA of any one of the preceding embodiments, comprising a YA modification or substitution of conserved region YA site 1.

Embodiment 327 is the gRNA of any one of the preceding embodiments, comprising a YA modification or substitution of conserved region YA site 2.

Embodiment 328 is the gRNA of any one of the preceding embodiments, comprising a YA modification or substitution of conserved region YA site 3.

Embodiment 329 is the gRNA of any one of the preceding embodiments, comprising a YA modification or substitution of conserved region YA site 4.

Embodiment 330 is the gRNA of any one of the preceding embodiments, comprising a YA modification or substitution of conserved region YA site 5.

Embodiment 331 is the gRNA of any one of the preceding embodiments, comprising a YA modification or substitution of conserved region YA site 6.

Embodiment 332 is the gRNA of any one of the preceding embodiments, comprising a YA modification or substitution of conserved region YA site 7.

Embodiment 333 is the gRNA of any one of the preceding embodiments, comprising a YA modification or substitution of conserved region YA site 8.

Embodiment 334 is the gRNA of any one of the preceding embodiments, comprising a YA modification or substitution of conserved region YA site 9.

Embodiment 335 is the gRNA of any one of the preceding embodiments, comprising a YA modification or substitution of conserved region YA site 10.

Embodiment 336 is the gRNA of any one of embodiments 326-335, comprising:

i. YA modifications of conserved region YA sites 2, 3, 4, and 10;

ii. YA modifications of conserved region YA sites 2, 3, and 4;

iii. YA modifications of conserved region YA sites 2, 3, and 10;

iv. YA modifications of conserved region YA sites 2, 4, and 10;

v. YA modifications of conserved region YA sites 3, 4, and 10;

vi. YA modifications of conserved region YA sites 2 and 10;

vii. YA modifications of conserved region YA sites 2 and 4;

viii. YA modifications of conserved region YA sites 2 and 3;

ix. YA modifications of conserved region YA sites 3 and 4;

x. YA modifications of conserved region YA sites 3 and 10; or

xi. YA modifications of conserved region YA sites 4 and 10.

Embodiment 337 is the gRNA of any one of embodiments 326-336, comprising:

i. YA modifications of conserved region YA sites 1 and 5;

ii. YA modifications of conserved region YA sites 1 and 6;

iii. YA modifications of conserved region YA sites 1 and 7;

iv. YA modifications of conserved region YA sites 1 and 8;

v. YA modifications of conserved region YA sites 1 and 9;

vi. YA modifications of conserved region YA sites 8 and 5;

vii. YA modifications of conserved region YA sites 8 and 6;

viii. YA modifications of conserved region YA sites 8 and 7; or

ix. YA modifications of conserved region YA sites 8 and 9;

optionally wherein the sgRNA further comprises YA modifications of conserved region YA sites 2, 3, 4, and 10.

Embodiment 338 is the gRNA of any one of embodiments 299-337, wherein at least one modified YA site comprises a 2′-OMe modification, optionally at the pyrimidine of the YA site.

Embodiment 339 is the gRNA of any one of embodiments 299-338, wherein at least one modified YA site comprises a 2′-fluoro modification, optionally at the pyrimidine of the YA site.

Embodiment 340 is the gRNA of any one of embodiments 299-339, wherein at least one modified YA site comprises a PS modification, optionally at the pyrimidine of the YA site.

Embodiment 341 is the gRNA of any one of embodiments 299-340, wherein at least 2, 3, 4, 5, 6, 7, 8, 9, or 10 modified YA sites comprise a 2′-OMe modification, optionally at the pyrimidines of the YA sites.

Embodiment 342 is the gRNA of any one of embodiments 299-341, wherein at least 2, 3, 4, 5, 6, 7, 8, 9, or 10 modified YA sites comprise a 2′-fluoro modification, optionally at the pyrimidines of the YA sites.

Embodiment 343 is the gRNA of any one of embodiments 299-342, wherein at least 2, 3, 4, 5, 6, 7, 8, 9, or 10 modified YA sites comprise a PS modification, optionally at the pyrimidines of the YA sites.

Embodiment 344 is the gRNA of any one of embodiments 299-343, wherein at least 2, 3, 4, 5, 6, 7, 8, 9, or 10 modified YA sites comprise a ribose modification at the 2′ position, optionally at the pyrimidines of the YA sites, and optionally chosen from a 2′-O-alkyl, 2′-H, and 2′-fluoro modification.

Embodiment 345 is the gRNA of any one of embodiments 299-344, wherein:

i. conserved region YA sites 1 and 8 comprise 2′-fluoro modifications, optionally at the pyrimidines of the YA sites;

ii. conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;

iii. conserved region YA site 1 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;

iv. conserved region YA site 8 comprises a 2′-fluoro modification and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;

v. conserved region YA site 1 comprises a 2′-fluoro modification at the pyrimidine of the YA sites and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;

vi. conserved region YA site 8 comprises a 2′-fluoro modification at the pyrimidine of the YA site and YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites;

vii. conserved region YA sites 1 and 8 comprise 2′-fluoro modifications and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites; or

viii. conserved region YA sites 1 and 8 comprise 2′-fluoro modifications at the pyrimidines of the YA sites and conserved region YA sites 5 and 6; 5 and 7; 5 and 9; 6 and 7; 6 and 9; 5, 6, and 7; 5, 6, and 9; 6, 7, and 9; or 5, 6, 7, and 9 comprise 2′-OMe modifications, optionally at the pyrimidines of the YA sites.

Embodiment 346 is the gRNA of any one of embodiments 299-345, wherein conserved region YA sites 7 and 9 comprise YA modifications, which are optionally 2′-OMe modifications.

Embodiment 347 is the gRNA of any one of embodiments 299-346, wherein conserved region YA sites 5, 6, 7, and 9 comprise YA modifications, which are optionally 2′-OMe modifications.

Embodiment 348 is the gRNA of any one of embodiments 299-347, wherein conserved region YA site 8 comprises a 2′-fluoro modification.

Embodiment 349 is the gRNA of any one of embodiments 299-348, wherein conserved region YA site 8 comprises a deoxyribonucleotide modification.

Embodiment 350 is the gRNA of any one of embodiments 299-349, wherein conserved region YA site 8 is abolished by a base substitution, optionally wherein the base substitution eliminates the uracil of YA site 8, further optionally wherein the base substitution is a uracil to guanine substitution.

Embodiment 351 is the gRNA of any one of embodiments 299-350, wherein conserved region YA site 1 comprises a 2′-fluoro modification.

Embodiment 352 is the gRNA of any one of embodiments 299-351, wherein conserved region YA site 1 comprises a PS modification.

Embodiment 353 is the gRNA of any one of embodiments 299-352, wherein 1, 2, 3, 4, 5, 6, or 7 of LS5, LS7, LS8, LS9, LS10, LS11, and LS12 comprise modifications, optionally wherein the modifications are 2′-fluoro and/or 2′-OMe modifications.

Embodiment 354 is the gRNA of any one of embodiments 299-353, wherein modifications at LS5, LS7, LS9, and LS11, if present, comprise 2′-fluoro modifications, optionally wherein each of LS5, LS7, LS9, and LS11 comprise 2′-fluoro modifications.

Embodiment 355 is the gRNA of any one of embodiments 299-354, wherein modifications at LS8, LS10, and LS12, if present, comprise 2′-OMe modifications, optionally wherein each of LS8, LS10, and LS12 comprise 2′-OMe modifications.

Embodiment 356 is the gRNA of any one of embodiments 299-355, wherein 1, 2, 3, 4, 5, 6, 7, 8, 9, or 10 of N2, N3, N4, N5, N6, N7, N10, N11, N16, and N17 comprise modifications, which are optionally 2′-OMe modifications.

Embodiment 357 is the gRNA of any one of embodiments 299-356, wherein H2-2 comprises a modification, optionally wherein H2 is otherwise unmodified.

Embodiment 358 is the gRNA of any one of embodiments 299-357, wherein H2-2 comprises a 2′-OMe modification.

Embodiment 359 is the gRNA of any one of embodiments 299-358, wherein US3, US9, and US12 comprise modifications, optionally wherein the US is otherwise unmodified.

Embodiment 360 is the gRNA of any one of embodiments 299-359, wherein US3, US9, and US12 comprise 2′-OMe modifications.

Embodiment 361 is the gRNA of any one of embodiments 299-360, wherein nucleotides 6-10 from the 5′ end of the 5′ terminus comprise a PS modification and nucleotides 8-11, 13, 14, 17, and 18 from the 5′ end of the 5′ terminus comprise a 2′-fluoro modification.

Embodiment 362 is the gRNA of any one of embodiments 299-361, wherein each guide region YA site comprises a 2′-fluoro modification, optionally excepting nucleotides 15 and/or 16 from the 5′ end of the 5′ terminus.

Embodiment 363 is the gRNA of any one of embodiments 299-362, wherein nucleotides 4, 8, and 11 from the 5′ end of the 5′ terminus comprise YA modifications, optionally wherein nucleotide 4 comprises a 2′-OMe modification and nucleotides 8 and 11 comprise a 2′-fluoro modification.

Embodiment 364 is the gRNA of any one of embodiments 299-363, wherein 1, 2, 3, 4, 5, 6, 7, 8, 9, 10, 11, 12, 13, 14, 15, or more modified YA sites comprise a YA modification at the pyrimidine position of the YA site.

Embodiment 365 is the gRNA of embodiment 364, wherein 1, 2, 3, 4, 5, 6, 7, 8, 9, or 10 modified conserved region YA sites comprise a YA modification at the pyrimidine position of the YA site.

Embodiment 366 is the gRNA of any one of embodiments 299-365, wherein 1, 2, 3, 4, 5, 6, 7, 8, 9, 10, 11, 12, 13, 14, 15, or more modified YA sites comprise a YA modification at the adenine position of the YA site.

Embodiment 367 is the gRNA of embodiment 366, wherein 1, 2, 3, 4, 5, 6, 7, 8, 9, or 10 modified conserved region YA sites comprise a YA site modification at the adenine position of the YA site.

Embodiment 368 is the gRNA of any one of embodiments 299-367, comprising:

i. a modification of H1-1;

ii. a modification of H2-1; or

iii. modifications of H1-1 and H2-1.

Embodiment 369 is the gRNA of embodiment 368, wherein H1-1 and/or H2-1 comprises a 2′-OMe modification.

Embodiment 370 is the gRNA of embodiment 369, wherein H1-1 and/or H2-1 comprises a 2′-fluoro modification.

Embodiment 371 is the gRNA of embodiment 370, wherein H1-1 and/or H2-1 comprises a PS modification.

Embodiment 372 is the gRNA of any one of embodiments 299-371, comprising a modification at B3, optionally wherein B6 does not comprise a 2′-OMe modification or comprises a modification other than 2′-OMe.

Embodiment 373 is the gRNA of any one of embodiments 299-372, comprising a modification at B4, optionally wherein B6 does not comprise a 2′-OMe modification or comprises a modification other than 2′-OMe.

Embodiment 374 is the gRNA of any one of embodiments 299-373, comprising a modification at B5, optionally wherein B6 does not comprise a 2′-OMe modification or comprises a modification other than 2′-OMe.

Embodiment 375 is the gRNA of any one of embodiments 299-374, comprising a modification at LS10, optionally wherein LS10 comprises a modification other than 2′-fluoro.

Embodiment 376 is the gRNA of any one of embodiments 299-375, comprising a modification at N2.

Embodiment 377 is the gRNA of any one of embodiments 299-376, comprising a modification at N3.

Embodiment 378 is the gRNA of any one of embodiments 299-377, comprising a modification at N4.

Embodiment 379 is the gRNA of any one of embodiments 299-378, comprising a modification at N5.

Embodiment 380 is the gRNA of any one of embodiments 299-379, comprising a modification at N6.

Embodiment 381 is the gRNA of any one of embodiments 299-380, comprising a modification at N7.

Embodiment 382 is the gRNA of any one of embodiments 299-381, comprising a modification at N10.

Embodiment 383 is the gRNA of any one of embodiments 299-382, comprising a modification at N11.

Embodiment 384 is the gRNA of any one of embodiments 299-383, wherein:

i. nucleotide 8 from the 5′ end of the 5′ terminus does not comprise a 2′-fluoro modification;

ii. nucleotide 9 from the 5′ end of the 5′ terminus does not comprise a 2′-fluoro modification;

iii. nucleotide 10 from the 5′ end of the 5′ terminus does not comprise a 2′-fluoro modification;

iv. nucleotide 11 from the 5′ end of the 5′ terminus does not comprise a 2′-fluoro modification;

v. nucleotide 13 from the 5′ end of the 5′ terminus does not comprise a 2′-fluoro modification;

vi. nucleotide 14 from the 5′ end of the 5′ terminus does not comprise a 2′-fluoro modification;

vii. nucleotide 17 from the 5′ end of the 5′ terminus does not comprise a 2′-fluoro modification; and/or

viii. nucleotide 18 from the 5′ end of the 5′ terminus does not comprise a 2′-fluoro modification.

Embodiment 385 is the gRNA of any one of embodiments 299-384, wherein:

i. nucleotide 6 from the 5′ end of the 5′ terminus does not comprise a 2′-fluoro modification;

ii. nucleotide 7 from the 5′ end of the 5′ terminus does not comprise a 2′-fluoro modification;

iii. nucleotide 8 from the 5′ end of the 5′ terminus does not comprise a 2′-fluoro modification;

iv. nucleotide 9 from the 5′ end of the 5′ terminus does not comprise a 2′-fluoro modification; and/or

v. nucleotide 10 from the 5′ end of the 5′ terminus does not comprise a 2′-fluoro modification.

Embodiment 386 is the gRNA of any one of embodiments 299-385, wherein:

i. nucleotide 6 from the 5′ end of the 5′ terminus does not comprise a phosphorothioate linkage;

ii. nucleotide 7 from the 5′ end of the 5′ terminus does not comprise a phosphorothioate linkage;

iii. nucleotide 8 from the 5′ end of the 5′ terminus does not comprise a phosphorothioate linkage;

iv. nucleotide 9 from the 5′ end of the 5′ terminus does not comprise a phosphorothioate linkage; and/or

v. nucleotide 10 from the 5′ end of the 5′ terminus does not comprise a phosphorothioate linkage.

Embodiment 387 is the gRNA of any one of embodiments 299-386, wherein:

i. nucleotide 7 from the 5′ end of the 5′ terminus does not comprise a 2′-OMe modification;

ii. nucleotide 8 from the 5′ end of the 5′ terminus does not comprise a 2′-OMe modification;

iii. nucleotide 9 from the 5′ end of the 5′ terminus does not comprise a 2′-OMe modification; and/or

iv. nucleotide 10 from the 5′ end of the 5′ terminus does not comprise a 2′-OMe modification.

Embodiment 388 is the gRNA of any one of embodiments 299-387, wherein nucleotide 20 from the 5′ end of the 5′ terminus does not comprise a 2′-OMe modification.

Embodiment 389 is the gRNA of any one of embodiments 299-388, wherein the guide RNA comprises a 2′-fluoro modification at any one or more of nucleotides 1-11 and 13-20 from the 5′ end of the 5′ terminus and nucleotide 12 from the 5′ end of the 5′ terminus does not comprise a 2′-fluoro modification.

Embodiment 390 is the gRNA of any one of embodiments 299-389, wherein the guide RNA comprises a 2′-fluoro modification at any one or more of nucleotides 1-20 from the 5′ end of the 5′ terminus and:

i. nucleotide 11 from the 5′ end of the 5′ terminus does not comprise a 2′-fluoro modification;

ii. nucleotide 12 from the 5′ end of the 5′ terminus does not comprise a 2′-fluoro modification;

iii. nucleotide 13 from the 5′ end of the 5′ terminus does not comprise a 2′-fluoro modification;

iv. nucleotide 14 from the 5′ end of the 5′ terminus does not comprise a 2′-fluoro modification;

v. nucleotide 17 from the 5′ end of the 5′ terminus does not comprise a 2′-fluoro modification; and/or

vi. nucleotide 18 from the 5′ end of the 5′ terminus does not comprise a 2′-fluoro modification.

Embodiment 391 is the gRNA of any one of embodiments 299-390, wherein:

i. B2 does not comprise a 2′-OMe modification;

ii. B3 does not comprise a 2′-OMe modification;

iii. B4 does not comprise a 2′-OMe modification; and/or

iv. B5 does not comprise a 2′-OMe modification.

Embodiment 392 is the gRNA of any one of embodiments 299-391, wherein:

i. LS1 does not comprise a 2′-OMe modification;

ii. LS8 does not comprise a 2′-OMe modification; and/or

iii. LS10 does not comprise a 2′-OMe modification.

Embodiment 393 is the gRNA of any one of embodiments 299-392, wherein:

i. N2 does not comprise a 2′-OMe modification;

ii. N3 does not comprise a 2′-OMe modification;

iii. N4 does not comprise a 2′-OMe modification;

iv. N5 does not comprise a 2′-OMe modification;

v. N6 does not comprise a 2′-OMe modification;

vi. N7 does not comprise a 2′-OMe modification;

vii. N10 does not comprise a 2′-OMe modification;

viii. N11 does not comprise a 2′-OMe modification;

ix. N16 does not comprise a 2′-OMe modification; and/or

x. N17 does not comprise a 2′-OMe modification.

Embodiment 394 is the gRNA of any one of embodiments 299-393, wherein:

i. H1-2 does not comprise a phosphorothioate linkage;

ii. H1-3 does not comprise a phosphorothioate linkage;

iii. H1-4 does not comprise a phosphorothioate linkage;

iv. H1-5 does not comprise a phosphorothioate linkage;

v. H1-6 does not comprise a phosphorothioate linkage;

vi. H1-7 does not comprise a phosphorothioate linkage;

vii. H1-8 does not comprise a phosphorothioate linkage;

viii. H1-9 does not comprise a phosphorothioate linkage;

ix. H1-10 does not comprise a phosphorothioate linkage;

x. H2-1 does not comprise a phosphorothioate linkage;

xi. H2-2 does not comprise a phosphorothioate linkage;

xii. H2-3 does not comprise a phosphorothioate linkage;

xiii. H2-4 does not comprise a phosphorothioate linkage;

xiv. H2-5 does not comprise a phosphorothioate linkage;

xv. H2-6 does not comprise a phosphorothioate linkage;

xvi. H2-7 does not comprise a phosphorothioate linkage;

xvii. H2-8 does not comprise a phosphorothioate linkage;

xviii. H2-9 does not comprise a phosphorothioate linkage;

xix. H2-10 does not comprise a phosphorothioate linkage;

xx. H2-11 does not comprise a phosphorothioate linkage;

xxi. H2-12 does not comprise a phosphorothioate linkage;

xxii. H2-13 does not comprise a phosphorothioate linkage;

xxiii. H2-14 does not comprise a phosphorothioate linkage; and/or

xxiv. H2-15 does not comprise a phosphorothioate linkage.

Embodiment 395 is the gRNA of any one of the embodiments 299-394, wherein conserved region YA sites 1, 5, 6, 7, and 9 comprise YA modifications, which are optionally 2′-OMe modifications; and conserved region YA site 8 comprises a modification, which is optionally a 2′-fluoro modification.

Embodiment 396 is the gRNA of any one of the preceding embodiments, wherein one or more of the following are true:

i. nucleotide 4 from the 5′ end of the 5′ terminus comprises a 2′-OMe modification;

ii. nucleotides 6-10 from the 5′ end of the 5′ terminus comprise PS modifications;

iii. nucleotides 8-11, 13, 14, 17, and 18 from the 5′ end of the 5′ terminus comprise 2′-fluoro modifications;

iv. LS5, LS7, LS9, and LS11 comprise 2′-fluoro modifications;

v. LS8, LS10, and LS12 comprise 2′-OMe modifications;

vi. N2, N3, N4, N5, N6, N7, N10, N11, N16, and N17 comprise 2′-OMe modifications; and

vii. N14 comprises a 2′-fluoro modification.

Embodiment 397 is the gRNA of any one of embodiments 299-396, wherein at least one YA modification comprises a modification of the pyrimidine position of the YA site.

Embodiment 398 is the gRNA of any one of embodiments 299-397, wherein at least one YA modification comprises a modification of the adenine position of the YA site.

Embodiment 399 is the gRNA of any one of embodiments 299-398, wherein at least 2, 3, 4, 5, 6, 7, 8, 9, or 10 YA sites comprise YA modifications at the pyrimidines positions of the YA sites.

Embodiment 400 is the gRNA of any one of embodiments 299-399, wherein at least 2, 3, 4, 5, 6, 7, 8, 9, or 10 YA sites comprise YA modifications at the adenine positions of the YA sites.

Embodiment 401 is the gRNA of any one of embodiments 299-400, wherein at least one YA modification comprises a 2′-OMe modification.

Embodiment 402 is the gRNA of any one of embodiments 299-401, wherein at least 2, 3, 4, 5, 6, 7, 8, 9, or 10 YA sites comprise a 2′-OMe modification.

Embodiment 403 is the gRNA of any one of embodiments 299-402, wherein each modified conserved region YA site comprises a modification at the pyrimidine position of the YA site.

Embodiment 404 is the gRNA of any one of embodiments 299-403, wherein each modified guide region YA site, or each modified conserved region and guide region YA site, comprises a modification at the pyrimidine position of the YA site.

Embodiment 405 is the gRNA of any one of embodiments 299-404, wherein each modified conserved region YA site comprises a modification at the adenine position of the YA site.

Embodiment 406 is the gRNA of any one of embodiments 299-405, wherein each modified guide region YA site, or each modified conserved region and guide region YA site, comprises a modification at the adenine position of the YA site.

Embodiment 407 is the gRNA of any one of embodiments 299-406, which is an sgRNA comprising a modification at LS5.

Embodiment 408 is the gRNA of any one of embodiments 299-407, which is an sgRNA comprising a modification at LS7.

Embodiment 409 is the gRNA of any one of embodiments 299-408, which is an sgRNA comprising a modification at LS9, optionally wherein if LS9 is modified and LS5, LS7, and LS12 are not, then the modification of LS9 is other than 2′-fluoro.

Embodiment 410 is the gRNA of any one of embodiments 299-409, which is an sgRNA comprising a modification at LS12, optionally wherein if LS12 is modified and LS9 is not, then the modification of LS12 is other than 2′-OMe.

Embodiment 411 is the gRNA of any one of embodiments 299-410, which is an sgRNA comprising at least one YA modification that stabilizes a secondary structure, optionally wherein the secondary structure is the lower stem.

Embodiment 412 is the gRNA of any one of embodiments 299-411, which is an sgRNA comprising at least one modification of LS8 and/or LS11, optionally wherein the modification of LS8 and/or LS11 stabilizes a secondary structure.

Embodiment 413 is the gRNA of any one of embodiments 299-412, comprising a YA modification that stabilizes a secondary structure chosen from:

i. ENA;

ii. LNA; or

iii. a bicyclic ribose modification.

Embodiment 414 is the gRNA of any one of embodiments 299-413, which is an sgRNA comprising a modification at N6.

Embodiment 415 is the gRNA of any one of embodiments 299-414, which is an sgRNA comprising a modification at N14.

Embodiment 416 is the gRNA of any one of embodiments 299-415, which is an sgRNA comprising a modification at N17, optionally wherein if N17 is modified and N6 and N14 are not, then the modification of N17 is other than 2′-fluoro and other than 2′-OMe.

Embodiment 417 is the gRNA of any one of embodiments 299-416, wherein at least 1, 2, or 3 of nucleotides 1-3 from the 5′ end of the 5′ terminus comprise deoxyribonucleotides, optionally wherein nucleotides 1-3 from the 5′ end of the 5′ terminus comprise PS modifications.

Embodiment 418 is the gRNA of any one of embodiments 299-417, wherein the gRNA is an sgRNA and at least 1, 2, or 3 of nucleotides 1-3 from the 3′ end of the 3′ terminus comprise deoxyribonucleotides, optionally wherein nucleotides 2-3 from the 3′ end of the 3′ terminus comprise PS modifications.

Embodiment 419 is the gRNA of any one of embodiments 299-418, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.

Embodiment 420 is the gRNA of any one of embodiments 299-419, wherein the gRNA is an sgRNA and hairpin 2 comprises deoxyribonucleotides, optionally wherein all or all but 1, 2, 3, 4, 5, 6, 7, 8, 9, or 10 nucleotides of hairpin 1 and hairpin 2 are deoxyribonucleotides.

Embodiment 421 is the gRNA of any one of embodiments 299-420, wherein the gRNA is an sgRNA and hairpin 1 and hairpin 2 comprise deoxyribonucleotides, optionally wherein all or all but 1, 2, 3, 4, 5, 6, 7, 8, 9, 10, 11, 12, or 13 nucleotides of hairpin 1 and hairpin 2 are deoxyribonucleotides.

Embodiment 422 is the gRNA of any one of embodiments 299-421, wherein the gRNA is an sgRNA and all or all but 1, 2, 3, 4, 5, 6, 7, 8, 9, 10, 11, 12, or 13 nucleotides from the beginning of hairpin 1 to the 3′ end of the sgRNA are deoxyribonucleotides, optionally wherein nucleotides 1-3 from the 3′ end of the 3′ terminus are deoxyribonucleotides.

Embodiment 423 is the gRNA of any one of embodiments 299-422, wherein the gRNA is an sgRNA and the upper stem comprises deoxyribonucleotides.

Embodiment 424 is the gRNA of any one of embodiments 299-423, wherein the gRNA is an sgRNA and all or all but 1, 2, 3, 4, 5, 6, 7, 8, 9, or 10 nucleotides of the upper stem are deoxyribonucleotides.

Embodiment 425 is the gRNA of any one of embodiments 299-424, wherein at least 1, 2, or 3 of nucleotides 1-3 from the 5′ end of the 5′ terminus comprise ENA, optionally wherein nucleotides 1-3 from the 5′ end of the 5′ terminus comprise PS modifications.

Embodiment 426 is the gRNA of any one of embodiments 299-425, wherein the gRNA is an sgRNA and at least 1, 2, or 3 of nucleotides 2-4 from the 3′ end of the 3′ terminus comprise ENA, optionally wherein nucleotides 2-3 from the 3′ end of the 3′ terminus comprise PS modifications.

Embodiment 427 is the gRNA of any one of embodiments 299-426, wherein at least 1, 2, or 3 of nucleotides 1-3 from the 5′ end of the 5′ terminus comprise UNA, optionally wherein nucleotides 1-3 from the 5′ end of the 5′ terminus comprise PS modifications.

Embodiment 428 is the gRNA of any one of embodiments 299-427, wherein the gRNA is an sgRNA and at least 1, 2, or 3 of nucleotides 2-4 from the 3′ end of the 3′ terminus comprise UNA, optionally wherein nucleotides 2-3 from the 3′ end of the 3′ terminus comprise PS modifications.

Embodiment 429 is the gRNA of any one of embodiments 299-428, wherein the gRNA is an sgRNA and nucleotide 4 from the 3′ end of the 3′ terminus comprises a PS modification, optionally wherein nucleotide 4 from the 3′ end of the 3′ terminus comprises a 2′-OMe modification.

Embodiment 430 is the gRNA of any one of embodiments 299-429, wherein the modification reduces gRNA degradation without significantly altering the ability of the guide to cleave a target nucleic acid.

Embodiment 431 is the gRNA of any one of embodiments 299-430, comprising a YA modification wherein the modification comprises 2′-fluoro, 2′-H, 2′-O-Me, ENA, UNA, or PS.

Embodiment 432 is the gRNA of any one of embodiments 299-431, comprising a YA modification wherein the modification alters the structure of the dinucleotide motif to reduce RNA endonuclease activity.

Embodiment 433 is the gRNA of any one of embodiments 299-432, comprising a YA modification wherein the modification interferes with recognition or cleavage of a YA site by an RNase and/or stabilizes an RNA structure.

Embodiment 434 is the gRNA of any one of embodiments 299-433, comprising a YA modification wherein the modification comprises one or more of:

i. a ribose modification selected from 2′-O-alkyl, 2′-F, 2′-moe, 2′-F arabinose, and 2′-H (deoxyribose);

ii. a bicyclic ribose analog, such as LNA, BNA, and ENA;

iii. an unlocked nucleic acid modification;

iv. a base modification, such as inosine, pseudouridine, and 5′-methylcytosine; and

v. an internucleoside linkage modification such as phosphorothioate.

Embodiment 435 is the gRNA of any one of the preceding embodiments, wherein the gRNA comprises a guide region that comprises a modification at nucleotide 5, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 436 is the gRNA of any one of the preceding embodiments, wherein the gRNA comprises a guide region that comprises a modification at nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 437 is the gRNA of any one of the preceding embodiments, wherein the gRNA comprises a guide region that comprises a 2′-OMe modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 438 is the gRNA of any one of the preceding embodiments, wherein the gRNA comprises a guide region that comprises a 2′-F modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 439 is the gRNA of any one of the preceding embodiments, wherein the gRNA comprises a guide region that comprises a 2′-H modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 440 is the gRNA of any one of the preceding embodiments, wherein the gRNA comprises a guide region that comprises a phosphorothioate modification at nucleotide 5 and/or nucleotide 12, optionally wherein the guide region comprises 2′-OMe modifications at nucleotides 1-4, phosphorothioate modifications at nucleotides 1-3 and 6-10, and/or 2′-F modifications at nucleotides 8-11, 13, 14, 17, and 18.

Embodiment 441 is the gRNA of any one of the preceding embodiments, wherein the gRNA comprises a guide region that comprises modifications at:

i. nucleotides 8-10;

ii. nucleotides 8 and 9;

iii. nucleotides 8 and 10; or

iv. nucleotides 9 and 10,

optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

Embodiment 442 is the gRNA of any one of the preceding embodiments, wherein the gRNA comprises a guide region that comprises 2′-F modifications at:

i. nucleotides 8-10;

ii. nucleotides 8 and 9;

iii. nucleotides 8 and 10;

iv. nucleotides 9 and 10; or

v. nucleotide 8;

optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

Embodiment 443 is the gRNA of any one of the preceding embodiments, wherein the gRNA comprises a guide region that comprises 2′-F modifications at:

i. nucleotides 8-10;

ii. nucleotides 8 and 9;

iii. nucleotides 8 and 10;

iv. nucleotides 9 and 10; or

v. nucleotide 8;

wherein nucleotides 8-10 do not comprise phosphorothioate modifications, and optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

Embodiment 444 is the gRNA of any one of the preceding embodiments, wherein the gRNA comprises a guide region that comprises 2′-F modifications at nucleotides 8-10 and:

i. phosphorothioate modifications at 1, 2, or 3 of nucleotides 8-10;

ii. a phosphorothioate modification at nucleotide 8;

iii. a phosphorothioate modification at nucleotide 9;

iv. a phosphorothioate modification at nucleotide 10;

v. a phosphorothioate modification at nucleotides 8 and 9;

vi. a phosphorothioate modification at nucleotides 8 and 10;

vii. a phosphorothioate modification at nucleotides 9 and 10; or

viii. a phosphorothioate modification at nucleotides 8-10

optionally wherein the guide region comprises 2′-OMe modifications at nucleotides 1-4, phosphorothioate modifications at nucleotides 1-3 and 6-7, and/or 2′-F modifications at nucleotides 11, 13, 14, 17, and 18.

Embodiment 445 is the gRNA of any one of the preceding embodiments, wherein the gRNA comprises a guide region that comprises:

i. a 2′-F or phosphorothioate modification at nucleotides 5 and 6;

ii. a 2′-F modification at nucleotides 5 and 6;

iii. a phosphorothioate modification at nucleotides 5 and 6;

iv. a 2′-F modification at nucleotide 5 and a phosphorothioate modification at nucleotide 6; or

v. a 2′-F modification at nucleotide 6 and a phosphorothioate modification at nucleotide 5;

optionally wherein the guide region comprises 2′-OMe modifications at nucleotides 1-4, phosphorothioate modifications at nucleotides 1-3 and 7-10, and/or 2′-F modifications at nucleotides 8-11, 13, 14, 17, and 18.

Embodiment 446 is the gRNA of any one of the preceding embodiments, wherein the gRNA comprises a guide region that comprises 2′-F modifications at at least 1, 2, 3, 4, 5, or 6 of nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.

Embodiment 447 is the gRNA of any one of the preceding embodiments, wherein the gRNA comprises a guide region that comprises 2′-F modifications at at least 1, 2, 3, 4, 5, 6, 7, 8, 9, or 10 of nucleotides 1˜4 and 6-11, optionally wherein the guide region comprises phosphorothioate modifications at nucleotides 1-3 and/or 2′-F modifications at nucleotides 13, 14, 17, and 18.

Embodiment 448 is the gRNA of any one of the preceding embodiments, wherein the gRNA comprises a guide region that comprises 2′-F modifications at nucleotides 6-11, optionally wherein the guide region comprises 2′-OMe modifications at nucleotides 1-4, phosphorothioate modifications at nucleotides 1-3, and/or 2′-F modifications at nucleotides 13, 14, 17, and 18.

Embodiment 449 is the gRNA of any one of the preceding embodiments, wherein the gRNA comprises a guide region that comprises 2′-F modifications at nucleotides 1-4, optionally wherein the guide region comprises phosphorothioate modifications at nucleotides 1-3 and 6-10, and/or 2′-F modifications at nucleotides 6-11, 13, 14, 17, and 18.

Embodiment 450 is the gRNA of any one of the preceding embodiments, wherein the gRNA comprises a guide region that comprises a 2′-F modification at nucleotide 9 and not a phosphorothioate modification at nucleotide 9, optionally wherein the guide region comprises 2′-OMe modifications at nucleotides 1-4, phosphorothioate modifications at nucleotides 1-3 and 6-8 and 10, and/or 2′-F modifications at nucleotides 8, 10, 11, 13, 14, 17, and 18.

Embodiment 451 is the gRNA of any one of the preceding embodiments, wherein the gRNA comprises a guide region that does not comprise 2′-F modifications at at least 1, 2, 3, 4, 5, 6, 7, or 8 of nucleotides 8-11, 13, 14, 17, and 18, optionally wherein the guide region comprises 2′-OMe modifications at nucleotides 1˜4 and/or phosphorothioate modifications at nucleotides 1-3 and 6-10.

Embodiment 452 is the gRNA of any one of the preceding embodiments, wherein the gRNA comprises a guide region that does not comprise 2′-F modifications at nucleotides 8-11, 13, 14, 17, and 18, optionally wherein the guide region comprises 2′-OMe modifications at nucleotides 1˜4 and/or phosphorothioate modifications at nucleotides 1-3 and 6-10.

Embodiment 453 is the gRNA of any one of the preceding embodiments, wherein the gRNA comprises a guide region that comprises 2′-OMe modifications at at least 1, 2, 3, or 4 of nucleotides 9, 11, 13, and 14, optionally wherein the guide region comprises 2′-OMe modifications at nucleotides 1˜4 and/or phosphorothioate modifications at nucleotides 1-3 and 6-10.

Embodiment 454 is the gRNA of any one of the preceding embodiments, wherein the gRNA comprises a guide region that comprises 2′-OMe modifications at nucleotides 9, 11, 13, and 14, optionally wherein the guide region comprises 2′-OMe modifications at nucleotides 1˜4 and/or phosphorothioate modifications at nucleotides 1-3 and 6-10.

Embodiment 455 is the gRNA of any one of the preceding embodiments, wherein the gRNA comprises a guide region that comprises phosphorothioate modifications at one or both of nucleotides 8 and 10, optionally wherein the guide region comprises 2′-OMe modifications at nucleotides 1-4, phosphorothioate modifications at nucleotides 1-3 and 6-7, and/or 2′-F modifications at nucleotides 8-11, 13, 14, 17, and 18.

Embodiment 456 is the gRNA of any one of the preceding embodiments, wherein the gRNA comprises a guide region that comprises modifications at at least 1, 2, 3, 4, 5, 6, 7, 8, 9, 10, 11, 12, 13, or all of the following nucleotides: 1, 2, 3, 4, 6, 7, 8, 9, 10, 11, 13, 14, 17, and 18, optionally wherein the modifications are 2′-OMe, 2′-fluoro, or phosphorothioate modifications.

Embodiment 457 is the gRNA of any one of the the preceding embodiments, wherein the gRNA comprises a guide region that comprises modifications at nucleotides 1, 2, 3, 4, 6, 7, 8, 9, 10, 11, 13, 14, 17, and 18, optionally wherein the modifications are 2′-OMe, 2′-fluoro, or phosphorothioate modifications.

Embodiment 458 is the gRNA of any one of the preceding embodiments, wherein 2′-OMe modifications are not present in the guide region at nucleotides 6-11 and 13-end.

Embodiment 459 is the gRNA of any one of the preceding embodiments, wherein 2′-fluoro modifications are not present in the guide region at nucleotides 1-7, 15, 16, and 19-end.

Embodiment 460 is the gRNA of any one of the preceding embodiments, wherein phosphorothioate modifications are not present in the guide region at nucleotides 4, 5, 11-14, 17, and 18.

Embodiment 461 is the gRNA of any one of the preceding embodiments, wherein the guide region comprises an unmodified nucleotide 20.

Embodiment 462 is the gRNA of any one of the preceding embodiments, wherein the guide region consists of 20 nucleotides.

Embodiment 463 is the gRNA of any one of the preceding embodiments, wherein the guide region comprises a YA site at nucleotides 5-6 and a modification at nucleotide 5.

Embodiment 464 is the gRNA of any one of the preceding embodiments, wherein the guide region comprises a YA site at nucleotides 12-13 and a modification at nucleotide 12.

Embodiment 465 is the gRNA of any one of the preceding embodiments, wherein the guide region comprises a YA site at nucleotides 15-16 and a modification at nucleotide 15.

Embodiment 466 is the gRNA of any one of the preceding embodiments, wherein the guide region comprises a YA site at nucleotides 16-17 and a modification at nucleotide 16.

Embodiment 467 is the gRNA of any one of the preceding embodiments, wherein the guide region comprises a YA site at nucleotides 19-20 and a modification at nucleotide 19.

Embodiment 468 is the gRNA of any one of the preceding embodiments, wherein the guide region does not comprise a YA site at nucleotides 5-6 and nucleotide 5 is unmodified.

Embodiment 469 is the gRNA of any one of the preceding embodiments, wherein the guide region does not comprise a YA site at nucleotides 12-13 and nucleotide 12 is unmodified.

Embodiment 470 is the gRNA of any one of the preceding embodiments, wherein the guide region does not comprise a YA site at nucleotides 15-16 and nucleotide 15 is unmodified.

Embodiment 471 is the gRNA of any one of the preceding embodiments, wherein the guide region does not comprise a YA site at nucleotides 16-17 and nucleotide 16 is unmodified.

Embodiment 472 is the gRNA of any one of the preceding embodiments, wherein the guide region does not comprise a YA site at nucleotides 19-20 and nucleotide 19 is unmodified.

Embodiment 473 is the gRNA of any one of the preceding embodiments, wherein the gRNA comprises a guide region that comprises at least 1, 2, 3, 4, 5, 6, 7, 8, 9, 10, 11, 12, 13, or all of the following:

i. 2′-OMe and phosphorothioate modifications at nucleotide 1;

ii. 2′-OMe and phosphorothioate modifications at nucleotide 2;

iii. 2′-OMe and phosphorothioate modifications at nucleotide 3;

iv. a 2′-OMe modification at nucleotide 4;

v. a phosphorothioate modification at nucleotide 6;

vi. a phosphorothioate modification at nucleotide 7;

vii. 2′-fluoro and phosphorothioate modifications at nucleotide 8;

viii. 2′-fluoro and phosphorothioate modifications at nucleotide 9;

ix. 2′-fluoro and phosphorothioate modifications at nucleotide 10;

x. a 2′-fluoro modification at nucleotide 11;

xi. a 2′-fluoro modifications at nucleotide 13;

xii. a 2′-fluoro modifications at nucleotide 14;

xiii. a 2′-fluoro modifications at nucleotide 17; and

xiv. a 2′-fluoro modifications at nucleotide 18.

Embodiment 474 is the gRNA of any one of the preceding embodiments, wherein the guide region comprises each of the modifications set forth in the preceding embodiment.

Embodiment 475 is the gRNA of any one of the preceding embodiments, wherein the guide region comprises at least 1, 2, 3, or 4 of the following:

i. a 2′-OMe modification at nucleotide 5 if nucleotides 5 and 6 form a YA site;

ii. a 2′-OMe modification at nucleotide 12 if nucleotides 12 and 13 form a YA site;

iii. a phosphorothioate or 2′-H modification at nucleotide 15 if nucleotides 15 and 16 form a YA site;

iv. a phosphorothioate modification at nucleotide 16 if nucleotides 16 and 17 form a YA site; and

v. a phosphorothioate or 2′-fluoro modification at nucleotide 19 if nucleotides 19 and 20 form a YA site.

Embodiment 476 is the gRNA of any one of the preceding embodiments, wherein the guide region comprises a YA site at nucleotides 5-6 and a a 2′-OMe modification at nucleotide 5.

Embodiment 477 is the gRNA of any one of the preceding embodiments, wherein the guide region comprises a YA site at nucleotides 12-13 and a 2′-OMe modification at nucleotide 12.

Embodiment 478 is the gRNA of any one of the preceding embodiments, wherein the guide region comprises a YA site at nucleotides 15-16 and a phosphorothioate modification at nucleotide 15.

Embodiment 479 is the gRNA of any one of the preceding embodiments, wherein the guide region comprises a YA site at nucleotides 16-17 and a phosphorothioate modification at nucleotide 16.

Embodiment 480 is the gRNA of any one of the preceding embodiments, wherein the guide region comprises a YA site at nucleotides 19-20 and a phosphorothioate modification at nucleotide 19.

Embodiment 481 is the gRNA of any one of the preceding embodiments, wherein the guide region comprises a 2′-fluoro modification at nucleotide 19.

Embodiment 482 is the gRNA of any one of the preceding embodiments, wherein the guide region comprises an unmodified nucleotide 15 or only a phosphorothioate modification at nucleotide 15.

Embodiment 483 is the gRNA of any one of the preceding embodiments, wherein the guide region comprises an unmodified nucleotide 16 or only a phosphorothioate modification at nucleotide 16.

Embodiment 484 is an LNP composition comprising a gRNA of any one of the preceding embodiments.

Embodiment 485 is a composition comprising a gRNA of any one of embodiments 1-483 associated with a lipid nanoparticle (LNP).

Embodiment 486 is a composition comprising the gRNA of any one of embodiments 1-483, or the composition of any one of embodiments 452-453, further comprising a nuclease or an mRNA which encodes the nuclease.

Embodiment 487 is the composition of embodiment 486, wherein the nuclease is a Cas protein.

Embodiment 488 is the composition of embodiment 487, wherein the Cas protein is a

Cas9.

Embodiment 489 is the composition of embodiment 488, wherein the Cas9 is an S. pyogenes Cas9 or an S. aureus Cas9.

Embodiment 490 is the composition of any one of embodiments 485-489, wherein the nuclease is a nickase or a dCas.

Embodiment 491 is the composition of any one of embodiments 485-490, wherein the nuclease is modified.

Embodiment 492 is the composition of embodiment 491, wherein the modified nuclease comprises a nuclear localization signal (NLS).

Embodiment 493 is the composition of any one of embodiments 484-492, comprising an mRNA which encodes the nuclease.

Embodiment 494 is the composition of embodiment 493, wherein the mRNA comprises the sequence of any one of SEQ ID NOs: 1099-1127 or 1129-1146.

Embodiment 495 is a pharmaceutical formulation comprising the gRNA of any one of embodiments 1-483 or the composition of any one of embodiments 484-494 and a pharmaceutically acceptable carrier.

Embodiment 496 is a method of modifying a target DNA comprising, delivering a Cas protein or a nucleic acid encoding a Cas protein, and any one or more of the following to a cell:

i. the gRNA of any one of embodiments 1-483;

ii. the composition of any one of embodiments 484-494; and

iii. the pharmaceutical formulation of embodiment 495.

Embodiment 497 is the method of embodiment 496, wherein the method results in an insertion or deletion in a gene.

Embodiment 498 is the method of embodiment 496 or embodiment 497, further comprising delivering to the cell a template, wherein at least a part of the template incorporates into a target DNA at or near a double strand break site induced by the Cas protein.

Embodiment 499 is the gRNA of any one of embodiments 1-483, the composition of embodiments 484-494, or the pharmaceutical formulation of embodiment 495 for use in preparing a medicament for treating a disease or disorder.

Embodiment 500 is a use of the gRNA of any one of embodiments 1-483, the composition of embodiments 484-494, or the pharmaceutical formulation of embodiment 495 in the manufacture of a medicament for treating a disease or disorder.

Embodiment A1. A guide RNA (gRNA) comprising a 5′ end modification or a 3′ end modification and a conserved portion of an gRNA comprising one or more of:

(a) a shortened hairpin 1 region or a substituted and optionally shortened hairpin 1 region, wherein
(i) at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9, and the hairpin 1 region optionally lacks
   (aa) any one or two of H1-5 through H1-8,
   (bb) one, two, or three of the following pairs of nucleotides: H1-1 and H1-12, H1-2 and H1-11, H1-3 and H1-10 and/or H1-4 and H1-9, and/or
   (cc) 1-8 nucleotides of the hairpin 1 region; or
(ii) the shortened hairpin 1 region lacks 6-8 nucleotides, preferably 6 nucleotides; and
   (A) one or more of positions H1-1, H1-2, or H1-3 is deleted or substituted relative to SEQ ID NO: 400 and/or
   (B) one or more of positions H1-6 through H1-10 is substituted relative to SEQ ID NO: 400; or
(iii) the shortened hairpin 1 region lacks 5-10 nucleotides, preferably 5-6 nucleotides, and one or more of positions N18, H1-12, or n is substituted relative to SEQ ID NO: 400; and/or

(b) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 400; and/or

(c) a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; and/or

(d) an upper stem region, wherein the upper stem modification comprises a modification to any one or more of US1-US12 in the upper stem region.

Embodiment A2. The gRNA of embodiment A1, wherein position H1-1 is deleted.

Embodiment A3. The gRNA of embodiment A1, wherein position H1-1 is substituted.

Embodiment A4. The gRNA of any one of embodiments A1-A3, wherein position H1-2 is deleted.

Embodiment A5. The gRNA of any one of embodiments A1-A3, wherein position H1-2 is substituted.

Embodiment A6. The gRNA of any one of embodiments A1-A5, wherein position H1-3 is deleted.

Embodiment A7. The gRNA of any one of embodiments A1-A5, wherein position H1-3 is substituted.

Embodiment A8. The gRNA of any one of embodiments A1-A7, wherein position H1-4 is deleted.

Embodiment A9. The gRNA of any one of embodiments A1-A7, wherein position H1-5 is deleted.

Embodiment A10. The gRNA of any one of embodiments A1-A9, wherein position H1-6 is deleted.

Embodiment A11. The gRNA of any one of embodiments A1-A9, wherein position H1-6 is substituted.

Embodiment A12. The gRNA of any one of embodiments A1-A11, wherein position H1-7 is deleted.

Embodiment A13. The gRNA of any one of embodiments A1-A11, wherein position H1-7 is substituted.

Embodiment A14. The gRNA of any one of embodiments A1-A13, wherein position H1-8 is deleted.

Embodiment A15. The gRNA of any one of embodiments A1-A13, wherein position H1-8 is substituted.

Embodiment A16. The gRNA of any one of embodiments A1-A15, wherein position H1-9 is deleted.

Embodiment A17. The gRNA of any one of embodiments A1-A15, wherein position H1-9 is substituted.

Embodiment A18. The gRNA of any one of embodiments A1-A17, wherein position H1-10 is deleted.

Embodiment A19. The gRNA of any one of embodiments A1-A17, wherein position H1-10 is substituted.

Embodiment A20. The gRNA of any one of embodiments A1-A19, wherein position H1-11 is deleted.

Embodiment A21. The gRNA of any one of embodiments A1-A20, wherein position H1-12 is deleted.

Embodiment A22. The gRNA of any one of embodiments A1-A21, wherein positions H1-11 through H1-12 are deleted.

Embodiment A23. The gRNA of any one of embodiments A1-A22, wherein positions H1-7 is substituted with a G and/or H1-8 is substituted with a C.

Embodiment A24. The gRNA of any one of embodiments A1-A23, wherein positions H1-6 and/or H1-7 are substituted.

Embodiment A25. The gRNA of any one of embodiments A1-A24, wherein position H1-6 is substituted with a C and/or position H1-7 is substituted with a U.

Embodiment A26. The gRNA of any one of embodiments A1-A25, wherein positions H1-1 and/or H1-12 are substituted.

Embodiment A27. The gRNA of any one of embodiments A1-A26, wherein position H1-1 is substituted with a C and/or position H1-12 is substituted with a G.

Embodiment A28. The gRNA of any one of embodiments A1-A27, wherein position N18 is substituted.

Embodiment A29. The gRNA of embodiment A28, wherein position N18 is substituted with a C.

Embodiment A30. The gRNA of any one of embodiments A1-A29, wherein position H1-12 is substituted.

Embodiment A31. The gRNA of embodiment A30, wherein position H1-12 is substituted with a C or an A.

Embodiment A32. The gRNA of any one of embodiments A1-A31, wherein position n is substituted.

Embodiment A33. The gRNA of embodiment A32, wherein position n is substituted with an A.

Embodiment A34. The gRNA of any one of embodiments A1-A33, comprising a shortened upper stem region, wherein the shortened upper stem region lacks 1-6 nucleotides.

Embodiment A35. The gRNA of any one of embodiments A1-A34, wherein the gRNA is an sgRNA.

Embodiment A36. The gRNA of any one of embodiments A1-A35, wherein the gRNA comprises a 5′ end modification.

Embodiment A37. The gRNA of any one of embodiments A1-A36, wherein the gRNA comprises a 3′ end modification.

Embodiment A38. The gRNA of any one of embodiments A1-A37, wherein the gRNA comprises a 5′ end modification and a 3′ end modification.

Embodiment A39. The gRNA of any one of embodiments A1-A38, wherein the gRNA comprises a 3′ tail.

Embodiment A40. The gRNA of embodiment A39, wherein the 3′ tail comprises 1-2, 1-3, 1-4, 1-5, 1-7, 1-10 nucleotides or 1, 2, 3, 4, 5, 6, 7, 8, 9, or 10 nucleotides.

Embodiment A41. The gRNA of any one of embodiments A1-A38, wherein the gRNA does not comprise a 3′ tail.

Embodiment A42. The gRNA of any one of embodiments A1-A41, comprising a modification in the hairpin region.

Embodiment A43. The gRNA of embodiment A42, further comprising a 3′ end modification.

Embodiment A44. The gRNA of embodiment A42, further comprising a 3′ end modification and a 5′ end modification.

Embodiment A45. The gRNA of embodiment A42, further comprising a 5′ end modification.

Embodiment A46. The gRNA of any one of embodiments A1-A45, further comprising a guide region.

Embodiment A47. The gRNA of embodiment A46, wherein the guide region is 17, 18, 19, or 20 nucleotides in length.

Embodiment A48. The gRNA of any one of embodiments A1-A47, wherein the 3′ and/or 5′ end modification comprises a protective end modification, optionally a modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof.

Embodiment A49. The gRNA of any one of embodiments A1-A48, wherein the modification in the hairpin region comprises a modified nucleotide selected from a 2′-O-methyl (2′-Ome) modified nucleotide, a 2′-fluoro (2′-F) modified nucleotide, a phosphorothioate (PS) linkage between nucleotides, or a combination thereof.

Embodiment A50. The gRNA of any one of embodiments A1-A49, wherein the 3′ and/or 5′ end modification comprises or further comprises a 2′-O-methyl (2′-Ome) modified nucleotide.

Embodiment A51. The gRNA of any one of embodiments A1-A50, wherein the 3′ and/or 5′ end modification comprises or further comprises a 2′-fluoro (2′-F) modified nucleotide.

Embodiment A52. The gRNA of any one of embodiments A1-A51, wherein the 3′ and/or 5′ end modification comprises or further comprises a phosphorothioate (PS) linkage between nucleotides.

Embodiment A53. The gRNA of any one of embodiments A1-A52, wherein the 3′ and/or 5′ end modification comprises or further comprises an inverted abasic modified nucleotide.

Embodiment A54. The gRNA of any one of embodiments A1-A53, wherein the modification in the hairpin region comprises or further comprises a 2′-O-methyl (2′-Ome) modified nucleotide.

Embodiment A55. The gRNA of any one of embodiments A1-A54, wherein the modification in the hairpin region comprises or further comprises a 2′-fluoro (2′-F) modified nucleotide.

Embodiment A56. The gRNA of any one of embodiments A1-A55, wherein the sgRNA comprise a 3′ tail, wherein the 3′ tail comprises a modification of any one or more of the nucleotides present in the 3′ tail.

Embodiment A57. The gRNA of embodiment A56, wherein the 3′ tail is fully modified.

Embodiment A58. The gRNA of any one of embodiments A1-A57, wherein the upper stem region comprises at least one modification.

Embodiment A59. The gRNA of embodiment A58, wherein the upper stem modification comprises any one or more of:
i. a modification of any one or more of US1-US12 in the upper stem region; and
ii. a modification of at least 1, 2, 3, 4, 5, 6, 7, 8, 9, 10, 11, or all 12 nucleotides in the upper stem region.

Embodiment A60. The gRNA of embodiment A59, wherein the upper stem modification comprises one or more of:
i. a 2′-OMe modified nucleotide;
ii. a 2′-O-moe modified nucleotide;
iii. a 2′-F modified nucleotide; and
iv. combinations of one or more of (i.)-(iii.).

Embodiment A61. The gRNA of any one of embodiments A1-A60, comprising a nucleotide sequence having at least 99, 98, 97, 96, 95, 94, 93, 92, 91, 90, 85, 80, 75, or 70% identity to the nucleotide sequence of any one of SEQ ID NOs: 1-98, 201-294, 401-494, 601-698, or 801-875.

Embodiment A62. The gRNA of any one of embodiments A1-A61, comprising a nucleotide sequence having at least 99, 98, 97, 96, 95, 94, 93, 92, 91, 90, 85, 80, 75, or 70% identity to the nucleotide sequence of any one of SEQ ID Nos: 101-198, 301-394, 501-594, 701-798, or 901-975, wherein the modification at each nucleotide of the gRNA that corresponds to a nucleotide of the reference sequence identifier in Table 1A is identical to or equivalent to the modification shown in the reference sequence identifier in Table 1A.

Embodiment A63. A guide RNA comprising any of SEQ ID NOs: 1-98, 201-294, 401-494, 601-698, or 801-875.

Embodiment A64. A guide RNA comprising any of SEQ ID NOs: 101-198, 301-394, 501-594, 701-798, or 901-975, including the modifications of Table 1A.

Embodiment A65. The gRNA of any one of embodiments A1-A64, comprising a YA modification of one or more guide region YA sites.

Embodiment A66. The gRNA of any one of embodiments A1-A65, comprising a YA modification wherein the modification comprises 2′-fluoro, 2′-H, 2′-OMe, ENA, UNA, inosine, or PS modification.

Embodiment A67. The gRNA of any one of embodiments A1-A66, comprising a YA modification of one or more conserved region YA sites.

Embodiment A68. The gRNA of any one of embodiments A1-A67, wherein at least one modified YA site comprises

(i) a 2′-OMe modification, optionally of the pyrimidine of the YA site;

(ii) a 2′-fluoro modification, optionally of the pyrimidine of the YA site; and/or

(iii) a PS modification, optionally of the pyrimidine of the YA site.

Embodiment A69. An LNP composition comprising a gRNA of any one of embodiments A1-A68.

Embodiment A70. A composition comprising a gRNA of any one of embodiments A1-A68 associated with a lipid nanoparticle (LNP).

Embodiment A71. A composition comprising the gRNA of any one of embodiments A1-A68, or the composition of embodiment A69 or A70, further comprising a nuclease or an mRNA which encodes the nuclease.

Embodiment A72. The composition of embodiment A71, wherein the nuclease is a Cas protein.

Embodiment A73. The composition of embodiment A72, wherein the Cas protein is a Cas9.

Embodiment A74. The composition of embodiment A73, wherein the Cas9 is an S. pyogenes Cas9 or an S. aureus Cas9.

Embodiment A75. The composition of any one of embodiments A71-A74, wherein the nuclease is a nickase or a dCas.

Embodiment A76. The composition of any one of embodiments A71-A75, wherein the nuclease is modified.

Embodiment A77. The composition of embodiment A76, wherein the modified nuclease comprises a nuclear localization signal (NLS).

Embodiment A78. The composition of any one of embodiments A71-A77, comprising an mRNA which encodes the nuclease.

Embodiment A79. The composition of embodiment A78, wherein the mRNA comprises the sequence of any one of SEQ ID NOs: 1099-1127 or 1129-1146.

Embodiment A80. A pharmaceutical formulation comprising the gRNA of any one of embodiments A1-A68 or the composition of any one of embodiments A69-A79 and a pharmaceutically acceptable carrier.

Embodiment A81. A method of modifying a target DNA comprising, delivering a Cas protein or a nucleic acid encoding a Cas protein, and any one or more of the following to a cell:
i. the gRNA of any one of embodiments A1-A68;
ii. the composition of any one of embodiments A69-A79; and
iii. the pharmaceutical formulation of embodiment A80.

Embodiment A82. The method of embodiment A81, wherein the method results in an insertion or deletion in a gene.

Embodiment A83. The method of embodiment A81 or A82, further comprising delivering to the cell a template, wherein at least a part of the template incorporates into a target DNA at or near a double strand break site induced by the Cas protein.

Embodiment A84. The gRNA of any one of embodiments A1-A68, the composition of embodiments A69-A79, or the pharmaceutical formulation of embodiment A80 for use in preparing a medicament for treating a disease or disorder.

Embodiment A85. Use of the gRNA of any one of embodiments A1-A68, the composition of embodiments A69-A79, or the pharmaceutical formulation of embodiment A80 in the manufacture of a medicament for treating a disease or disorder.

### FIGURE LEGENDS

FIG. 1A shows an exemplary sgRNA (SEQ ID NO: 801, methylation not shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), and the hairpin region which includes hairpin 1 and hairpin 2 regions. A nucleotide between hairpin 1 and hairpin 2 is labeled n. A guide region may be present on an sgRNA and is indicated in this figure as “(N)ₓ” preceding the conserved region of the sgRNA.

FIG. 1B shows an exemplary sgRNA constant region sequence (SEQ ID NO: 802) in a possible secondary structure and including a 20-nucleotide guide sequence represented as Neo.

FIG. 1C labels the 10 conserved region YA sites in an exemplary sgRNA sequence (SEQ ID NO: 801, methylation not shown) from 1 to 10. The numbers 25, 45, 50, 56, 64, 67, and 83 indicate the position of the pyrimidine of YA sites 1, 5, 6, 7, 8, 9, and 10 in an sgRNA with a guide region indicated as (N)ₓ, e.g., wherein x is optionally 20.

FIG. 2 shows the editing frequency of a deletion series for the indicated guides in Primary Cynomolgus Hepatocytes (PCH).

FIGS. 3A and 3B show dose response curves for % editing results from experiments in which short guides were delivered in vitro to Primary Cynomolgus Hepatocytes (PCH) using Lipofection.

FIGS. 4A and 4B show dose response curves for % editing results from experiments in which short guides were delivered in vitro to Primary Mouse Hepatocytes (PMH) using LNPs.

FIGS. 5A and 5B show dose response curves for % editing results from experiments in which short guides were delivered in vitro to Primary Cynomologus Hepatocytes (PCH) using LNPs.

FIGS. 5C and 5D show dose response curves for % editing results from experiments in which short guides were delivered in vitro to Primary Mouse Hepatocytes (PMH) using LNPs.

FIGS. 5E and 5F shows dose response curves for % editing results from experiments in which short guides were delivered in vitro to Primary Cynomologus Hepatocytes (PCH) using LNPs.

FIGS. 6A and 6B show in vivo % editing and serum TTR results, respectively, for the indicated guides.

FIGS. 7A and 7B show in vivo % editing and serum TTR results, respectively, for the indicated guides.

FIGS. 8A and 8B show in vivo % editing and serum TTR results, respectively, for the indicated guides.

FIGS. 9A and 9B show in vivo % editing and serum TTR results, respectively, for the indicated guides.

FIGS. 10A and 10B show in vivo % editing and serum TTR results, respectively, for the indicated guides.

FIGS. 11A and 11B show in vivo % editing and serum TTR results, respectively, for the indicated guides.

FIGS. 12A and 12B show in vivo % editing and serum TTR results, respectively, for the indicated guides.

### DETAILED DESCRIPTION

Provided herein are modified guide RNAs (gRNAs) for use in gene editing methods. Sequences of engineered and tested gRNAs are shown in Table 1A.

Certain of the gRNAs provided herein are modified dual guide RNAs (dgRNAs) for use in gene editing methods. Sequences of engineered and tested dgRNAs are shown in Table 1. Certain of the dgRNAs have certain modifications at YA sites in the dgRNA, including modifications in the crRNA and/or the trRNA.

Certain of the gRNAs provided herein are modified single guide RNAs (sgRNAs) for use in gene editing methods. Sequences of engineered and tested sgRNAs are shown in Table 1. Certain of the sgRNAs have certain modifications at YA sites in the sgRNA, including modifications in the crRNA portion of the sgRNA and/or the trRNA portion of the sgRNA.

This disclosure further provides uses of these gRNAs (e.g., sgRNA, dgRNA, or crRNA) to alter the genome of a target nucleic acid in vitro (e.g., cells cultured in vitro for use in ex vivo therapy or other uses of genetically edited cells) or in a cell in a subject such as a human (e.g., for use in in vivo therapy).

TABLE 1A
Table of gRNA Sequences

| | SEQ | | SEQ | |
|---|---|---|---|---|
| Guide | ID | | ID | |
| ID | NO. | sgRNA unmodified sequence | NO. | sgRNA modified sequence |
| G015631 | 1 | ACACAAAUACCAGUCCAGCGGUUUUAGAGCUAG | 101 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUAAGCACCGAGUCGGUGC | | AACUAAGCACCGAGUCGG*mU*mG*mC |
| G015632 | 2 | ACACAAAUACCAGUCCAGCGGUUUUAGAGCUAG | 102 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUCAGCACCGAGUCGGUGC | | AACUCAGCACCGAGUCGG*mU*mG*mC |
| G015633 | 3 | ACACAAAUACCAGUCCAGCGGUUUUAGAGCUAG | 103 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCCACUUGGCACCGAGUCGGUGC | | CACUUGGCACCGAGUCGG*mU*mG*mC |
| G015634 | 4 | ACACAAAUACCAGUCCAGCGGUUUUAGAGCUAG | 104 | mA*mC*mA*CAAAUACCAGUCCAGCGGUUUUAGAGCU |
| | | AAAUAGCAAGUUAAAAUACGGCUAGUCCGUUA | | AGAAAUAGCAAGUUAAAAUACGGCUAGUCCGUUAUC |
| | | UCAACUUGGCACCGAGUCGGUGC | | AACUUGGCACCGAGUCGG*mU*mG*mC |
| G015635 | 5 | ACACAAAUACCAGUCCAGCGGUUUUAGAGCUAG | 105 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAGAGCUGGCACCGAGUCGGUGC | | AAGAGCUGGCACCGAGUCGG*mU*mG*mC |
| G015636 | 6 | ACACAAAUACCAGUCCAGCGGUUUUAGAGCUAG | 106 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAGAAAUGGCACCGAGUCGGUGC | | AAGAAAUGGCACCGAGUCGG*mU*mG*mC |
| G015637 | 7 | ACACAAAUACCAGUCCAGCGGUUUUAGAGCUAG | 107 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCACGAAAGGGCACCGAGUCGGUGC | | ACGAAAGGGCACCGAGUCGG*mU*mG*mC |
| G015638 | 8 | ACACAAAUACCAGUCCAGCGGUUUUAGAGCUAG | 108 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAAAAUGGCACCGAGUCGGUGC | | AAAAAUGGCACCGAGUCGG*mU*mG*mC |
| G015639 | 9 | ACACAAAUACCAGUCCAGCGGUUUUAGAGCUAG | 109 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAAAGUGGCACCGAGUCGGUGC | | AAAAGUGGCACCGAGUCGG*mU*mG*mC |
| G015640 | 10 | ACACAAAUACCAGUCCAGCGGUUUUAGAGCUAG | 110 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACAGUGGCACCGAGUCGGUGC | | AACAGUGGCACCGAGUCGG*mU*mG*mC |
| G015641 | 11 | ACACAAAUACCAGUCCAGCGGUUUUAGAGCUAG | 111 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCACAAGGGCACCGAGUCGGUGC | | ACAAGGGCACCGAGUCGG*mU*mG*mC |
| G015642 | 12 | ACACAAAUACCAGUCCAGCGGUUUUAGAGCUAG | 112 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAAAUGGCACCGAGUCGGUGC | | AAAAUGGCACCGAGUCGG*mU*mG*mC |
| G015643 | 13 | ACACAAAUACCAGUCCAGCGGUUUUAGAGCUAG | 113 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAAGGCACCGAGUCGGUGC | | AAAGGCACCGAGUCGG*mU*mG*mC |
| G015644 | 14 | ACACAAAUACCAGUCCAGCGGUUUUAGAGCUAG | 114 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAAGGGCACCGAGUCGGUGC | | AAGGGCACCGAGUCGG*mU*mG*mC |
| G015645 | 15 | ACACAAAUACCAGUCCAGCGGUUUUAGAGCUAG | 115 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAGGCACCGAGUCGGUGC | | AGGCACCGAGUCGG*mU*mG*mC |
| G015646 | 16 | ACACAAAUACCAGUCCAGCGGUUUUAGAGCUAG | 116 | mA*mC*mA*CAAAUACCAGUCCAGCGGUUUUAGAGCU |
| | | AAUAGCAAGUUAAAAUAAGGCUAGUCCGUUAU | | AGAAUAGCAAGUUAAAAUAAGGCUAGUCCGUUAUCA |
| | | CAACUUGGCACCGAGUCGGUGC | | ACUUGGCACCGAGUCGG*mU*mG*mC |
| G015647 | 17 | ACACAAAUACCAGUCCAGCGGUUUUAGAGCGCA | 117 | mA*mC*mA*CAAAUACCAGUCCAGCGGUUUUAGAGCG |
| | | AAGCGCAAGUUAAAAUAAGGCUAGUCCGUUAU | | CAAAGCGCAAGUUAAAAUAAGGCUAGUCCGUUAUCA |
| | | CAACUUGGCACCGAGUCGGUGC | | ACUUGGCACCGAGUCGG*mU*mG*mC |
| G015648 | 18 | ACACAAAUACCAGUCCAGCGGUUUUAGAGCGCG | 118 | mA*mC*mA*CAAAUACCAGUCCAGCGGUUUUAGAGCG |
| | | AAGCGCAAGUUAAAAUAAGGCUAGUCCGUUAU | | CGAAGCGCAAGUUAAAAUAAGGCUAGUCCGUUAUCA |
| | | CAACUUGGCACCGAGUCGGUGC | | ACUUGGCACCGAGUCGG*mU*mG*mC |
| G015649 | 19 | ACACAAAUACCAGUCCAGCGGUUUUAGAGCGGA | 119 | mA*mC*mA*CAAAUACCAGUCCAGCGGUUUUAGAGCG |
| | | AACGCAAGUUAAAAUAAGGCUAGUCCGUUAUC | | GAAACGCAAGUUAAAAUAAGGCUAGUCCGUUAUCAA |
| | | AACUUGGCACCGAGUCGGUGCU | | CUUGGCACCGAGUCGGU*mG*mC*mU |
| G015650 | 20 | ACACAAAUACCAGUCCAGCGGUUUUAGAGCGGA | 120 | mA*mC*mA*CAAAUACCAGUCCAGCGGUUUUAGAGCG |
| | | AACGCAAGUUAAAAUAAGGCUAGUCCGUUAUC | | GAAACGCAAGUUAAAAUAAGGCUAGUCCGUUAUCAA |
| | | AACUUGGCACCGAGUCGGUGC | | CUUGGCACCGAGUCGG*mU*mG*mC |
| G015651 | 21 | ACACAAAUACCAGUCCAGCGGUUUUAGAGCCGA | 121 | mA*mC*mA*CAAAUACCAGUCCAGCGGUUUUAGAGCC |
| | | AAGGCAAGUUAAAAUAAGGCUAGUCCGUUAUC | | GAAAGGCAAGUUAAAAUAAGGCUAGUCCGUUAUCAA |
| | | AACUUGGCACCGAGUCGGUGC | | CUUGGCACCGAGUCGG*mU*mG*mC |
| G015652 | 22 | ACACAAAUACCAGUCCAGCGGUUUUAGAGCUGA | 122 | mA*mC*mA*CAAAUACCAGUCCAGCGGUUUUAGAGCU |
| | | AAAGCAAGUUAAAAUAAGGCUAGUCCGUUAUC | | GAAAAGCAAGUUAAAAUAAGGCUAGUCCGUUAUCAA |
| | | AACUUGGCACCGAGUCGGUGC | | CUUGGCACCGAGUCGG*mU*mG*mC |
| G015653 | 23 | ACACAAAUACCAGUCCAGCGGUUUUAGAGCGAA | 123 | mA*mC*mA*CAAAUACCAGUCCAGCGGUUUUAGAGCG |
| | | AGCAAGUUAAAAUAAGGCUAGUCCGUUAUCAA | | AAAGCAAGUUAAAAUAAGGCUAGUCCGUUAUCAACU |
| | | CUUGGCACCGAGUCGGUGC | | UGGCACCGAGUCGG*mU*mG*mC |
| G015654 | 24 | ACACAAAUACCAGUCCAGCGGUUUUAGAGCGAA | 124 | mA*mC*mA*CAAAUACCAGUCCAGCGGUUUUAGAGCG |
| | | GCAAGUUAAAAUAAGGCUAGUCCGUUAUCAAC | | AAGCAAGUUAAAAUAAGGCUAGUCCGUUAUCAACUU |
| | | UUGGCACCGAGUCGGUGC | | GGCACCGAGUCGG*mU*mG*mC |
| G015655 | 25 | ACACAAAUACCAGUCCAGCGGUUUUAGAGCAAA | 125 | mA*mC*mA*CAAAUACCAGUCCAGCGGUUUUAGAGCA |
| | | GCAAGUUAAAAUAAGGCUAGUCCGUUAUCAAC | | AAGCAAGUUAAAAUAAGGCUAGUCCGUUAUCAACUU |
| | | UUGGCACCGAGUCGGUGC | | GGCACCGAGUCGG*mU*mG*mC |
| G015656 | 26 | ACACAAAUACCAGUCCAGCGGUUUUAGAGGAAA | 126 | mA*mC*mA*CAAAUACCAGUCCAGCGGUUUUAGAGGA |
| | | CAAGUUAAAAUAAGGCUAGUCCGUUAUCAACU | | AACAAGUUAAAAUAAGGCUAGUCCGUUAUCAACUUG |
| | | UGGCACCGAGUCGGUGC | | GCACCGAGUCGG*mU*mG*mC |
| G015657 | 27 | ACACAAAUACCAGUCCAGCGGUUUUAGAGCAAG | 127 | mA*mC*mA*CAAAUACCAGUCCAGCGGUUUUAGAGCA |
| | | CAAGUUAAAAUAAGGCUAGUCCGUUAUCAACU | | AGCAAGUUAAAAUAAGGCUAGUCCGUUAUCAACUUG |
| | | UGGCACCGAGUCGGUGC | | GCACCGAGUCGG*mU*mG*mC |
| G015658 | 28 | ACACAAAUACCAGUCCAGCGGUUUUAGAGCGAG | 128 | mA*mC*mA*CAAAUACCAGUCCAGCGGUUUUAGAGCG |
| | | CAAGUUAAAAUAAGGCUAGUCCGUUAUCAACU | | AGCAAGUUAAAAUAAGGCUAGUCCGUUAUCAACUUG |
| | | UGGCACCGAGUCGGUGC | | GCACCGAGUCGG*mU*mG*mC |
| G015659 | 29 | ACACAAAUACCAGUCCAGCGGUUUUAGAGCGCA | 129 | mA*mC*mA*CAAAUACCAGUCCAGCGGUUUUAGAGCG |
| | | AGUUAAAAUAAGGCUAGUCCGUUAUCAACUUG | | CAAGUUAAAAUAAGGCUAGUCCGUUAUCAACUUGGC |
| | | GCACCGAGUCGGUGC | | ACCGAGUCGG*mU*mG*mC |
| G015660 | 30 | ACACAAAUACCAGUCCAGCGGUUUUAGAGAACA | 130 | mA*mC*mA*CAAAUACCAGUCCAGCGGUUUUAGAGAA |
| | | AGUUAAAAUAAGGCUAGUCCGUUAUCAACUUG | | CAAGUUAAAAUAAGGCUAGUCCGUUAUCAACUUGGC |
| | | GCACCGAGUCGGUGC | | ACCGAGUCGG*mU*mG*mC |
| G015661 | 31 | ACACAAAUACCAGUCCAGCGGUUUUAGAGACAA | 131 | mA*mC*mA*CAAAUACCAGUCCAGCGGUUUUAGAGAC |
| | | GUUAAAAUAAGGCUAGUCCGUUAUCAACUUGG | | AAGUUAAAAUAAGGCUAGUCCGUUAUCAACUUGGCA |
| | | CACCGAGUCGGUGC | | CCGAGUCGG*mU*mG*mC |
| G015662 | 32 | ACACAAAUACCAGUCCAGCGGUUUUAGAGCAAG | 132 | mA*mC*mA*CAAAUACCAGUCCAGCGGUUUUAGAGCA |
| | | UUAAAAUAAGGCUAGUCCGUUAUCAACUUGGC | | AGUUAAAAUAAGGCUAGUCCGUUAUCAACUUGGCAC |
| | | ACCGAGUCGGUGC | | CGAGUCGG*mU*mG*mC |
| G015663 | 33 | ACACAAAUACCAGUCCAGCGGUUUUAGAAAAAG | 133 | mA*mC*mA*CAAAUACCAGUCCAGCGGUUUUAGAAAA |
| | | UUAAAAUAAGGCUAGUCCGUUAUCAACUUGGC | | AGUUAAAAUAAGGCUAGUCCGUUAUCAACUUGGCAC |
| | | ACCGAGUCGGUGC | | CGAGUCGG*mU*mG*mC |
| G015664 | 34 | ACACAAAUACCAGUCCAGCGGUUUUAGAAAAGU | 134 | mA*mC*mA*CAAAUACCAGUCCAGCGGUUUUAGAAAA |
| | | UAAAAUAAGGCUAGUCCGUUAUCAACUUGGCAC | | GUUAAAAUAAGGCUAGUCCGUUAUCAACUUGGCACC |
| | | CGAGUCGGUGC | | GAGUCGG*mU*mG*mC |
| G015665 | 35 | ACACAAAUACCAGUCCAGCGGUUUUAGAGCUAG | 135 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUCGGUG | | AACUUGGCACCGAGUCG*mG*mU*mG |
| G015666 | 36 | ACACAAAUACCAGUCCAGCGGUUUUAGAGCUAG | 136 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUCGGU | | AACUUGGCACCGAGUC*mG*mG*mU |
| G015667 | 37 | ACACAAAUACCAGUCCAGCGGUUUUAGAGCUAG | 137 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUCGG | | AACUUGGCACCGAGU*mC*mG*mG |
| G015668 | 38 | ACACAAAUACCAGUCCAGCGGUUUUAGAGCUAG | 138 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUCG | | AACUUGGCACCGAG*mU*mC*mG |
| G015669 | 39 | ACACAAAUACCAGUCCAGCGGUUUUAGAGCUAG | 139 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUC | | AACUUGGCACCGA*mG*mU*mC |
| G015670 | 40 | ACACAAAUACCAGUCCAGCGGUUUUAGAGCUAG | 140 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGU | | AACUUGGCACCG*mA*mG*mU |
| G015671 | 41 | ACACAAAUACCAGUCCAGCGGUUUUAGAGCGAA | 141 | mA*mC*mA*CAAAUACCAGUCCAGCGGUUUUAGAGCG |
| | | AGCAAGUUAAAAUAAGGCUAGUCCGUUAUCAC | | AAAGCAAGUUAAAAUAAGGCUAGUCCGUUAUCACUG |
| | | UGGCACCGAGUCGGUGC | | GCACCGAGUCGG*mU*mG*mC |
| G015672 | 42 | ACACAAAUACCAGUCCAGCGGUUUUAGAGCGAA | 142 | mA*mC*mA*CAAAUACCAGUCCAGCGGUUUUAGAGCG |
| | | AGCAAGUUAAAAUAAGGCUAGUCCGUUAUCAG | | AAAGCAAGUUAAAAUAAGGCUAGUCCGUUAUCAGGC |
| | | GCACCGAGUCGGUGC | | ACCGAGUCGG*mU*mG*mC |
| G015673 | 43 | ACACAAAUACCAGUCCAGCGGUUUUAGAGAAAA | 143 | mA*mC*mA*CAAAUACCAGUCCAGCGGUUUUAGAGAA |
| | | AGUUAAAAUAAGGCUAGUCCGUUAUCAACUUG | | AAAGUUAAAAUAAGGCUAGUCCGUUAUCAACUUGGC |
| | | GCACCGAGUCGGUGC | | ACCGAGUCGG*mU*mG*mC |
| G015674 | 44 | ACACAAAUACCAGUCCAGCGGUUUUAGAGGAAA | 144 | mA*mC*mA*CAAAUACCAGUCCAGCGGUUUUAGAGGA |
| | | CAAGUUAAAAUAAGGCUAGUCCGUUAUCAAUG | | AACAAGUUAAAAUAAGGCUAGUCCGUUAUCAAUGGC |
| | | GCACCGAGUCGGUGC | | ACCGAGUCGG*mU*mG*mC |
| G015675 | 45 | ACACAAAUACCAGUCCAGCGGUUUUAGAGAAAA | 145 | mA*mC*mA*CAAAUACCAGUCCAGCGGUUUUAGAGAA |
| | | AGUUAAAAUAAGGCUAGUCCGUUAUCAAUGGC | | AAAGUUAAAAUAAGGCUAGUCCGUUAUCAAUGGCAC |
| | | ACCGAGUCGGUGC | | CGAGUCGG*mU*mG*mC |
| G015676 | 46 | ACACAAAUACCAGUCCAGCGGUUUUAGAGGAAA | 146 | mA*mC*mA*CAAAUACCAGUCCAGCGGUUUUAGAGGA |
| | | CAAGUUAAAAUAAGGCUAGUCCGUUAUCACUG | | AACAAGUUAAAAUAAGGCUAGUCCGUUAUCACUGGC |
| | | GCACCGAGUCGGUGC | | ACCGAGUCGG*mU*mG*mC |
| G015677 | 47 | ACACAAAUACCAGUCCAGCGGUUUUAGAGAAAA | 147 | mA*mC*mA*CAAAUACCAGUCCAGCGGUUUUAGAGAA |
| | | AGUUAAAAUAAGGCUAGUCCGUUAUCAGGCACC | | AAAGUUAAAAUAAGGCUAGUCCGUUAUCAGGCACCG |
| | | GAGUCGGUGC | | AGUCGG*mU*mG*mC |
| G015678 | 48 | ACACAAAUACCAGUCCAGCGGUUUUAGAGCGAA | 148 | mA*mC*mA*CAAAUACCAGUCCAGCGGUUUUAGAGCG |
| | | AGCAAGUUAAAAUAAGGCUAGUCCGUUAUCAA | | AAAGCAAGUUAAAAUAAGGCUAGUCCGUUAUCAAGC |
| | | GCUAUGGCACCGAGUCGGUGC | | UAUGGCACCGAGUCGG*mU*mG*mC |
| G015679 | 49 | ACACAAAUACCAGUCCAGCGGUUUUAGAGCUAG | 149 | mA*mC*mA*CAAAUACCAGUCCAGCGGUUUUAGAGCU |
| | | AAAUAGCAAGUUAAAAUAAGGCAGUCCGUUAU | | AGAAAUAGCAAGUUAAAAUAAGGCAGUCCGUUAUCA |
| | | CAACUUGGCACCGAGUCGGUGC | | ACUUGGCACCGAGUCGG*mU*mG*mC |
| G015680 | 50 | ACACAAAUACCAGUCCAGCGGUUUUAGAGCUAG | 150 | mA*mC*mA*CAAAUACCAGUCCAGCGGUUUUAGAGCU |
| | | AAAUAGCAAGUUAAAAUAAGGCUGUCCGUUAU | | AGAAAUAGCAAGUUAAAAUAAGGCUGUCCGUUAUCA |
| | | CAACUUGGCACCGAGUCGGUGC | | ACUUGGCACCGAGUCGG*mU*mG*mC |
| G015681 | 51 | ACACAAAUACCAGUCCAGCGGUUUUAGAGCUAG | 151 | mA*mC*mA*CAAAUACCAGUCCAGCGGUUUUAGAGCU |
| | | AAAUAGCAAGUUAAAAUAAGGCGUCCGUUAUC | | AGAAAUAGCAAGUUAAAAUAAGGCGUCCGUUAUCAA |
| | | AACUUGGCACCGAGUCGGUGC | | CUUGGCACCGAGUCGG*mU*mG*mC |
| G015682 | 52 | ACACAAAUACCAGUCCAGCGGUUUUAGAGCUAG | 152 | mA*mC*mA*CAAAUACCAGUCCAGCGGUUUUAGAGCU |
| | | AAAUAGCAAGUUAAAAUAAGGUAUCCGUUAUC | | AGAAAUAGCAAGUUAAAAUAAGGUAUCCGUUAUCAA |
| | | AACUUGGCACCGAGUCGGUGC | | CUUGGCACCGAGUCGG*mU*mG*mC |
| G015683 | 53 | ACACAAAUACCAGUCCAGCGGUUUUAGAGCUAG | 153 | mA*mC*mA*CAAAUACCAGUCCAGCGGUUUUAGAGCU |
| | | AAAUAGCAAGUUAAAAUAAGGUUCCGUUAUCA | | AGAAAUAGCAAGUUAAAAUAAGGUUCCGUUAUCAAC |
| | | ACUUGGCACCGAGUCGGUGC | | UUGGCACCGAGUCGG*mU*mG*mC |
| G015684 | 54 | ACACAAAUACCAGUCCAGCGGUUUUAGAGCUAG | 154 | mA*mC*mA*CAAAUACCAGUCCAGCGGUUUUAGAGCU |
| | | AAAUAGCAAGUUAAAAUAAGGAUCCGUUAUCA | | AGAAAUAGCAAGUUAAAAUAAGGAUCCGUUAUCAAC |
| | | ACUUGGCACCGAGUCGGUGC | | UUGGCACCGAGUCGG*mU*mG*mC |
| G015685 | 55 | ACACAAAUACCAGUCCAGCGGUUUUCGAGCUAG | 155 | mA*mC*mA*CAAAUACCAGUCCAGCGGUUUUCGAGCU |
| | | AAAUAGCAAGUGAAAAUAAGGCUAGUCCGUUA | | AGAAAUAGCAAGUGAAAAUAAGGCUAGUCCGUUAUC |
| | | UCAACUUGGCACCGAGUCGGUGC | | AACUUGGCACCGAGUCGG*mU*mG*mC |
| G015686 | 56 | ACACAAAUACCAGUCCAGCGGUUUUUGAGCUAG | 156 | mA*mC*mA*CAAAUACCAGUCCAGCGGUUUUUGAGCU |
| | | AAAUAGCAAGUAAAAAUAAGGCUAGUCCGUUA | | AGAAAUAGCAAGUAAAAAUAAGGCUAGUCCGUUAUC |
| | | UCAACUUGGCACCGAGUCGGUGC | | AACUUGGCACCGAGUCGG*mU*mG*mC |
| G015687 | 57 | ACACAAAUACCAGUCCAGCGGUUUUAGAGCGAG | 157 | mA*mC*mA*CAAAUACCAGUCCAGCGGUUUUAGAGCG |
| | | AAAUCGCAAGUUAAAAUAAGGCUAGUCCGUUA | | AGAAAUCGCAAGUUAAAAUAAGGCUAGUCCGUUAUC |
| | | UCAACUUGGCACCGAGUCGGUGC | | AACUUGGCACCGAGUCGG*mU*mG*mC |
| G015688 | 58 | ACACAAAUACCAGUCCAGCGGUUUUAGAGCUAG | 158 | mA*mC*mA*CAAAUACCAGUCCAGCGGUUUUAGAGCU |
| | | AAAUAGCGAGUUAAAAUAAGGCUAGUCCGUUA | | AGAAAUAGCGAGUUAAAAUAAGGCUAGUCCGUUAUC |
| | | UCAACUUGGCACCGAGUCGGUGC | | AACUUGGCACCGAGUCGG*mU*mG*mC |
| G015689 | 59 | ACACAAAUACCAGUCCAGCGGUUUUAGAGCUAG | 159 | mA*mC*mA*CAAAUACCAGUCCAGCGGUUUUAGAGCU |
| | | AAAUAGCCGGUUAAAAUAAGGCUAGUCCGUUA | | AGAAAUAGCCGGUUAAAAUAAGGCUAGUCCGUUAUC |
| | | UCAACUUGGCACCGAGUCGGUGC | | AACUUGGCACCGAGUCGG*mU*mG*mC |
| G015690 | 60 | ACACAAAUACCAGUCCAGCGGUUUUAGAGCUAG | 160 | mA*mC*mA*CAAAUACCAGUCCAGCGGUUUUAGAGCU |
| | | AAAUAGCAAGUUAAAAUGAGGCUAGUCCGUUA | | AGAAAUAGCAAGUUAAAAUGAGGCUAGUCCGUUAUC |
| | | UCAACUUGGCACCGAGUCGGUGC | | AACUUGGCACCGAGUCGG*mU*mG*mC |
| G015691 | 61 | ACACAAAUACCAGUCCAGCGGUUUUAGAGCUAG | 161 | mA*mC*mA*CAAAUACCAGUCCAGCGGUUUUAGAGCU |
| | | AAAUAGCAAGUUAAAAUAAGGCUGGUCCGUUA | | AGAAAUAGCAAGUUAAAAUAAGGCUGGUCCGUUAUC |
| | | UCAACUUGGCACCGAGUCGGUGC | | AACUUGGCACCGAGUCGG*mU*mG*mC |
| G015692 | 62 | ACACAAAUACCAGUCCAGCGGUUUUAGAGCUAG | 162 | mA*mC*mA*CAAAUACCAGUCCAGCGGUUUUAGAGCU |
| | | AAAUAGCAAGUUAAAAUAAGGCUCGUCCGUUA | | AGAAAUAGCAAGUUAAAAUAAGGCUCGUCCGUUAUC |
| | | UCAACUUGGCACCGAGUCGGUGC | | AACUUGGCACCGAGUCGG*mU*mG*mC |
| G015693 | 63 | ACACAAAUACCAGUCCAGCGGUUUUAGAGCUAG | 163 | mA*mC*mA*CAAAUACCAGUCCAGCGGUUUUAGAGCU |
| | | AAAUAGCAAGUUAAAAUAAGGCUUGUCCGUUA | | AGAAAUAGCAAGUUAAAAUAAGGCUUGUCCGUUAUC |
| | | UCAACUUGGCACCGAGUCGGUGC | | AACUUGGCACCGAGUCGG*mU*mG*mC |
| G015694 | 64 | ACACAAAUACCAGUCCAGCGGUUUUAGAGCUAG | 164 | mA*mC*mA*CAAAUACCAGUCCAGCGGUUUUAGAGCU |
| | | AAAUAGCAAGUUAAAAUAAGGCUAGUCCGUUG | | AGAAAUAGCAAGUUAAAAUAAGGCUAGUCCGUUGUC |
| | | UCAACUUGGCACCGAGUCGGUGC | | AACUUGGCACCGAGUCGG*mU*mG*mC |
| G015695 | 65 | ACACAAAUACCAGUCCAGCGGUUUUAGAGCUAG | 165 | mA*mC*mA*CAAAUACCAGUCCAGCGGUUUUAGAGCU |
| | | AAAUAGCAAGUUAAAAUAAGGCUAGUCCGUUC | | AGAAAUAGCAAGUUAAAAUAAGGCUAGUCCGUUCUC |
| | | UCAACUUGGCACCGAGUCGGUGC | | AACUUGGCACCGAGUCGG*mU*mG*mC |
| G015696 | 66 | ACACAAAUACCAGUCCAGCGGUUUUAGAGCUAG | 166 | mA*mC*mA*CAAAUACCAGUCCAGCGGUUUUAGAGCU |
| | | AAAUAGCAAGUUAAAAUAAGGCUAGUCCGUUU | | AGAAAUAGCAAGUUAAAAUAAGGCUAGUCCGUUUUC |
| | | UCAACUUGGCACCGAGUCGGUGC | | AACUUGGCACCGAGUCGG*mU*mG*mC |
| G015697 | 67 | ACACAAAUACCAGUCCAGCGGUUUUAGAGCUAG | 167 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUG |
| | | UGAACUUGGCACCGAGUCGGUGC | | AACUUGGCACCGAGUCGG*mU*mG*mC |
| G015698 | 68 | ACACAAAUACCAGUCCAGCGGUUUUAGAGCUAG | 168 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGGACCGAGUCGGUCC | | AACUUGGGACCGAGUCGG*mU*mC*mC |
| G015699 | 69 | ACACAAAUACCAGUCCAGCGGUUUUAGAGCUAG | 169 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGAACCGAGUCGGUUC | | AACUUGGAACCGAGUCGG*mU*mU*mC |
| G015700 | 70 | ACACAAAUACCAGUCCAGCGGUUUUCGAGCGAG | 170 | mA*mC*mA*CAAAUACCAGUCCAGCGGUUUUCGAGCG |
| | | AAAUCGCGAGUGAAAAUGAGGCUGGUCCGUUG | | AGAAAUCGCGAGUGAAAAUGAGGCUGGUCCGUUGUG |
| | | UGAACUUGGAACCGAGUCGGUUC | | AACUUGGAACCGAGUCGG*mU*mU*mC |
| G015701 | 71 | ACACAAAUACCAGUCCAGCGGUUUUUGAGCGAG | 171 | mA*mC*mA*CAAAUACCAGUCCAGCGGUUUUUGAGCG |
| | | AAAUCGCAAGUAAAAAUAAGGCUCGUCCGUUCU | | AGAAAUCGCAAGUAAAAAUAAGGCUCGUCCGUUCUG |
| | | GAACUUGGAACCGAGUCGGUUC | | AACUUGGAACCGAGUCGG*mU*mU*mC |
| G015702 | 72 | ACACAAAUACCAGUCCAGCGGUUUCGGAGCCGG | 172 | mA*mC*mA*CAAAUACCAGUCCAGCGGUUUCGGAGCC |
| | | AAACGGCGAGUCGAAAUGAGGCUGGUCCGUUG | | GGAAACGGCGAGUCGAAAUGAGGCUGGUCCGUUGUC |
| | | UCGGCUCGGAACCGAGUCGGUUC | | GGCUCGGAACCGAGUCGG*mU*mU*mC |
| G015703 | 73 | CUCACUGAAAAGUGAGUCUGGAGAGCUGCAGU | 173 | mC*mU*mC*ACUGAAAAGUGAGUCUGGAGAGCUGCAG |
| | | UUUAGAGCUAGAAAUAGCAAGUUAAAAUAAGG | | UUUUAGAGCUAGAAAUAGCAAGUUAAAAUAAGGCUA |
| | | CUAGUCCGUUAUCAACUUGGCACCGAGUCGGUG | | GUCCGUUAUCAACUUGGCACCGAGUCGG*mU*mG*mC |
| | | C | | |
| G015704 | 74 | CACUGAAAAGUGAGUCUGGAGAGCUGCAGUUU | 174 | mC*mA*mC*UGAAAAGUGAGUCUGGAGAGCUGCAGU |
| | | UAGAGCUAGAAAUAGCAAGUUAAAAUAAGGCU | | UUUAGAGCUAGAAAUAGCAAGUUAAAAUAAGGCUAG |
| | | AGUCCGUUAUCAACUUGGCACCGAGUCGGUGC | | UCCGUUAUCAACUUGGCACCGAGUCGG*mU*mG*mC |
| G015705 | 75 | CUGAAAAGUGAGUCUGGAGAGCUGCAGUUUUA | 175 | mC*mU*mG*AAAAGUGAGUCUGGAGAGCUGCAGUUU |
| | | GAGCUAGAAAUAGCAAGUUAAAAUAAGGCUAG | | UAGAGCUAGAAAUAGCAAGUUAAAAUAAGGCUAGUC |
| | | UCCGUUAUCAACUUGGCACCGAGUCGGUGC | | CGUUAUCAACUUGGCACCGAGUCGG*mU*mG*mC |
| G015706 | 76 | UCUGGAGAGCUGCAGUUUUAGAGCUAGAAAUA | 176 | mU*mC*mU*GGAGAGCUGCAGUUUUAGAGCUAGAAA |
| | | GCAAGUUAAAAUAAGGCUAGUCCGUUAUCAAC | | UAGCAAGUUAAAAUAAGGCUAGUCCGUUAUCAACUU |
| | | UUGGCACCGAGUCGGUGC | | GGCACCGAGUCGG*mU*mG*mC |
| G015707 | 77 | AGUCUGGAGAGCUGCAGUUUUAGAGCUAGAAA | 177 | mA*mG*mU*CUGGAGAGCUGCAGUUUUAGAGCUAGA |
| | | UAGCAAGUUAAAAUAAGGCUAGUCCGUUAUCA | | AAUAGCAAGUUAAAAUAAGGCUAGUCCGUUAUCAAC |
| | | ACUUGGCACCGAGUCGGUGC | | UUGGCACCGAGUCGG*mU*mG*mC |
| G015708 | 78 | UGAGUCUGGAGAGCUGCAGUUUUAGAGCUAGA | 178 | mU*mG*mA*GUCUGGAGAGCUGCAGUUUUAGAGCUA |
| | | AAUAGCAAGUUAAAAUAAGGCUAGUCCGUUAU | | GAAAUAGCAAGUUAAAAUAAGGCUAGUCCGUUAUCA |
| | | CAACUUGGCACCGAGUCGGUGC | | ACUUGGCACCGAGUCGG*mU*mG*mC |
| G015709 | 79 | GAAAAGUGAGUCUGGAGAGCUGCAGUUUUAGA | 179 | mG*mA*mA*AAGUGAGUCUGGAGAGCUGCAGUUUUA |
| | | GCUAGAAAUAGCAAGUUAAAAUAAGGCUAGUC | | GAGCUAGAAAUAGCAAGUUAAAAUAAGGCUAGUCCG |
| | | CGUUAUCAACUUGGCACCGAGUCGGUGC | | UUAUCAACUUGGCACCGAGUCGG*mU*mG*mC |
| G017275 | 80 | ACACAAAUACCAGUCCAGCGGUUUUAGAGCUAG | 180 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGGCACCGAGUCGGUGC | | AACUUGGCACCGAGUCGG*mU*mG*mC |
| G017276 | 81 | ACACAAAUACCAGUCCAGCGGUUUUAGAGCUAG | 181 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G017277 | 82 | ACACAAAUACCAGUCCAGCGGUUUUAGAGCUAG | 182 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAAUGGCACCGAGUCGGUGC | | GCUAGUCCGUUAUCAAAAAUGGCACCGAGUCGG*mU* |
| | | | | mG*mC |
| G017278 | 83 | ACACAAAUACCAGUCCAGCGGUUUUAGAGCUAG | 183 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACAAGGGCACCGAGUCGGUGC | | GCUAGUCCGUUAUCACAAGGGCACCGAGUCGG*mU*m |
| | | | | G*mC |
| G017279 | 84 | ACACAAAUACCAGUCCAGCGGUUUUAGAGCUAG | 184 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAUGGCACCGAGUCGGUGC | | GCUAGUCCGUUAUCAAAAUGGCACCGAGUCGG*mU* |
| | | | | mG*mC |
| G017280 | 85 | ACACAAAUACCAGUCCAGCGGUUUUAGAGCGCG | 185 | mA*mC*mA*CAAAUACCAGUCCAGCGGUUUUAGAGCG |
| | | AAGCGCAAGUUAAAAUAAGGCUAGUCCGUUAU | | CGAAGCGCAAGUUAAAAUAAGGCUAGUCCGUUAUCA |
| | | CAAAAUGGCACCGAGUCGGUGC | | AAAUGGCACCGAGUCGG*mU*mG*mC |
| G017281 | 86 | ACACAAAUACCAGUCCAGCGGUUUUAGAGCUGA | 186 | mA*mC*mA*CAAAUACCAGUCCAGCGGUUUUAGAGCU |
| | | AAAGCAAGUUAAAAUAAGGCUAGUCCGUUAUC | | GAAAAGCAAGUUAAAAUAAGGCUAGUCCGUUAUCAA |
| | | AAAAUGGCACCGAGUCGGUGC | | AAUGGCACCGAGUCGG*mU*mG*mC |
| G017282 | 87 | ACACAAAUACCAGUCCAGCGGUUUUAGAGCGAA | 187 | mA*mC*mA*CAAAUACCAGUCCAGCGGUUUUAGAGCG |
| | | AGCAAGUUAAAAUAAGGCUAGUCCGUUAUCAA | | AAAGCAAGUUAAAAUAAGGCUAGUCCGUUAUCAAAA |
| | | AAUGGCACCGAGUCGGUGC | | UGGCACCGAGUCGG*mU*mG*mC |
| G017283 | 88 | ACACAAAUACCAGUCCAGCGGUUUUAGAGCAAA | 188 | mA*mC*mA*CAAAUACCAGUCCAGCGGUUUUAGAGCA |
| | | GCAAGUUAAAAUAAGGCUAGUCCGUUAUCAAA | | AAGCAAGUUAAAAUAAGGCUAGUCCGUUAUCAAAAU |
| | | AUGGCACCGAGUCGGUGC | | GGCACCGAGUCGG*mU*mG*mC |
| G013773 | 89 | ACACAAAUACCAGUCCAGCGGUUUUAGAGCGAA | 189 | mA*mC*mA*CAAAUACCAGUCCAGCGGUUUUAGAmG |
| | | AGCAAGUUAAAAUAAGGCUAGUCCGUUAUCAA | | mCmGmAmAmAmGmCAAGUUAAAAUAAGGCUAGUCC |
| | | CUUGGCACCGAGUCGGUGC | | GUUAUCAACUUGGCACCGAGUCGG*mU*mG*mC |
| G013776 | 90 | ACACAAAUACCAGUCCAGCGGUUUUAGAGCGAA | 190 | mA*mC*mA*CAAAUACCAGUCCAGCGGUUUUAGAmG |
| | | AGCAAGUUAAAAUAAGGCUAGUCCGUUAUCAA | | mCmGmAmAmAmGmCAAGUUAAAAUAAGGCUAGUCC |
| | | GAAAUGGCACCGAGUCGGUGC | | GUUAUCAAGAAAUGGCACCGAGUCGG*mU*mG*mC |
| G000502 | 91 | ACACAAAUACCAGUCCAGCGGUUUUAGAGCUAG | 191 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGAAAAAGUGGCACCGAGUCGGUGCUU | | GCUAGUCCGUUAUCAmAmCmUmUmGmAmAmAmAmA |
| | | UU | | mGmUmGmGmCmAmCmCmGmAmGmUmCmGmGmUmG |
| | | | | mCmU*mU*mU*mU |
| G009978 | 92 | ACACAAAUACCAGUCCAGCGGUUUUAGAGCUAG | 192 | mA*mC*mA*CAAAUACCAGUCCAGCGGUUUUAGAGCU |
| | | AAAUAGCAAGUUAAAAUAAGGCUAGUCCGUUA | | AGAAAUAGCAAGUUAAAAUAAGGCUAGUCCGUUAUC |
| | | UCAACUUGAAAAAGUGGCACCGAGUCGGUGCUU | | AACUUGAAAAAGUGGCACCGAGUCGGUGCU*mU*mU* |
| | | UU | | mU |
| G010039 | 93 | ACACAAAUACCAGUCCAGCGGUUUUAGAGCUAG | 193 | mA*mC*mA*mCAA*A*fU*fA*fC*fCAfGfUCC*fA |
| | | AAAUAGCAAGUUAAAAUAAGGCUAGUCCGUUA | | fGCGmGUUUfUAGmAmGmCmUmAmGmAmAmAmUmAmGm |
| | | UCAACUUGAAAAAGUGGCACCGAGUCGGUGCUU | | CmAmAGUfUmAfAmAfAmUAmAmGmGmCmUmAGUmCmC |
| | | UU | | GUfUAmUmCAmAmCmUmUmGmAmAmAmAmAmGmUmG |
| | | | | mGmCmAmCmCmGmAmGmUmCmGmGmUmGmCmU*mU |
| | | | | *mU*mU |
| G012401 | 94 | ACACAAAUACCAGUCCAGCGGUUUUAGAGCUAG | 194 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGGCACCGAGUCGGUGC | | GCUAGUCCGUUAUCAACUUGGCACCGAGUCGG*mU*m |
| | | | | G*mC |
| G012402 | 95 | ACACAAAUACCAGUCCAGCGGUUUUAGAGCUAG | 195 | mA*mC*mA*CAA*A*fU*fA*fC*fCAfGfUCCfAfG |
| | | AAAUAGCAAGUUAAAAUAAGGCUAGUCCGUUA | | CGGUUUUAGAmGmCmUmAmGmAmAmAmUmAmGmCAAG |
| | | UCAACUUGGCACCGAGUCGGUGC | | UUAAAAUAAGGCUAGUCCGUUAUCAACUUGGCACCGA |
| | | | | GUCGG*mU*mG*mC |
| G013772 | 96 | ACACAAAUACCAGUCCAGCGGUUUUAGAGCUAG | 196 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAGCACCGAGUCGGUGC | | GCUAGUCCGUUAUCAAGCACCGAGUCGG*mU*mG*mC |
| G013774 | 97 | ACACAAAUACCAGUCCAGCGGUUUUAGAGCGAA | 197 | mA*mC*mA*CAAAUACCAGUCCAGCGGUUUUAGAmG |
| | | AGCAAGUUAAAAUAAGGCUAGUCCGUUAUCAA | | mCmGmAmAmAmGmCAAGUUAAAAUAAGGCUAGUCC |
| | | GCACCGAGUCGGUGC | | GUUAUCAAGCACCGAGUCGG*mU*mG*mC |
| G013775 | 98 | ACACAAAUACCAGUCCAGCGGUUUUCGAGCGAG | 198 | mA*mC*mA*CAAAUACCAGUCCAGCGGUUUUCGAmGm |
| | | AAAUCGCGAGUGAAAAUGAGGCUGGUCCGUGA | | CmGmAmGmAmAmAmUmCmGmCGAGUGAAAAUGAGG |
| | | UGAACUUGAAAAAGUGGGACCGAGUCGGUCCU | | CUGGUCCGUGAUGAmAmCmUmUmGmAmAmAmAmAm |
| | | UUU | | GmUmGmGmGmAmCmCmGmAmGmUmCmGmGmUmCm |
| | | | | CmU*mU*mU*mU |
| | 99- | Not Used | 199- | Not Used |
| | 100 | | 200 | |
| C- | 201 | GUUUUAGAGCUAGAAAUAGCAAGUUAAAAUAA | 301 | GUUUUAGAGCUAGAAAUAGCAAGUUAAAAUAAGGCU |
| G015631 | | GGCUAGUCCGUUAUCAACUAAGCACCGAGUCGG | | AGUCCGUUAUCAACUAAGCACCGAGUCGG*mU*mG*m |
| | | UGC | | C |
| C- | 202 | GUUUUAGAGCUAGAAAUAGCAAGUUAAAAUAA | 302 | GUUUUAGAGCUAGAAAUAGCAAGUUAAAAUAAGGCU |
| G015632 | | GGCUAGUCCGUUAUCAACUCAGCACCGAGUCGG | | AGUCCGUUAUCAACUCAGCACCGAGUCGG*mU*mG*m |
| | | UGC | | C |
| C- | 203 | GUUUUAGAGCUAGAAAUAGCAAGUUAAAAUAA | 303 | GUUUUAGAGCUAGAAAUAGCAAGUUAAAAUAAGGCU |
| G015633 | | GGCUAGUCCGUUAUCCACUUGGCACCGAGUCGG | | AGUCCGUUAUCCACUUGGCACCGAGUCGG*mU*mG*m |
| | | UGC | | C |
| C- | 204 | GUUUUAGAGCUAGAAAUAGCAAGUUAAAAUAC | 304 | GUUUUAGAGCUAGAAAUAGCAAGUUAAAAUACGGCU |
| G015634 | | GGCUAGUCCGUUAUCAACUUGGCACCGAGUCGG | | AGUCCGUUAUCAACUUGGCACCGAGUCGG*mU*mG*m |
| | | UGC | | C |
| C- | 205 | GUUUUAGAGCUAGAAAUAGCAAGUUAAAAUAA | 305 | GUUUUAGAGCUAGAAAUAGCAAGUUAAAAUAAGGCU |
| G015635 | | GGCUAGUCCGUUAUCAAGAGCUGGCACCGAGUC | | AGUCCGUUAUCAAGAGCUGGCACCGAGUCGG*mU*m |
| | | GGUGC | | G*mC |
| C- | 206 | GUUUUAGAGCUAGAAAUAGCAAGUUAAAAUAA | 306 | GUUUUAGAGCUAGAAAUAGCAAGUUAAAAUAAGGCU |
| G015636 | | GGCUAGUCCGUUAUCAAGAAAUGGCACCGAGUC | | AGUCCGUUAUCAAGAAAUGGCACCGAGUCGG*mU*m |
| | | GGUGC | | G*mC |
| C- | 207 | GUUUUAGAGCUAGAAAUAGCAAGUUAAAAUAA | 307 | GUUUUAGAGCUAGAAAUAGCAAGUUAAAAUAAGGCU |
| G015637 | | GGCUAGUCCGUUAUCACGAAAGGGCACCGAGUC | | AGUCCGUUAUCACGAAAGGGCACCGAGUCGG*mU*m |
| | | GGUGC | | G*mC |
| C- | 208 | GUUUUAGAGCUAGAAAUAGCAAGUUAAAAUAA | 308 | GUUUUAGAGCUAGAAAUAGCAAGUUAAAAUAAGGCU |
| G015638 | | GGCUAGUCCGUUAUCAAAAAUGGCACCGAGUCG | | AGUCCGUUAUCAAAAAUGGCACCGAGUCGG*mU*mG* |
| | | GUGC | | mC |
| C- | 209 | GUUUUAGAGCUAGAAAUAGCAAGUUAAAAUAA | 309 | GUUUUAGAGCUAGAAAUAGCAAGUUAAAAUAAGGCU |
| G015639 | | GGCUAGUCCGUUAUCAAAAGUGGCACCGAGUCG | | AGUCCGUUAUCAAAAGUGGCACCGAGUCGG*mU*mG* |
| | | GUGC | | mC |
| C- | 210 | GUUUUAGAGCUAGAAAUAGCAAGUUAAAAUAA | 310 | GUUUUAGAGCUAGAAAUAGCAAGUUAAAAUAAGGCU |
| G015640 | | GGCUAGUCCGUUAUCAACAGUGGCACCGAGUCG | | AGUCCGUUAUCAACAGUGGCACCGAGUCGG*mU*mG* |
| | | GUGC | | mC |
| C- | 211 | GUUUUAGAGCUAGAAAUAGCAAGUUAAAAUAA | 311 | GUUUUAGAGCUAGAAAUAGCAAGUUAAAAUAAGGCU |
| G015641 | | GGCUAGUCCGUUAUCACAAGGGCACCGAGUCGG | | AGUCCGUUAUCACAAGGGCACCGAGUCGG*mU*mG*m |
| | | UGC | | C |
| C- | 212 | GUUUUAGAGCUAGAAAUAGCAAGUUAAAAUAA | 312 | GUUUUAGAGCUAGAAAUAGCAAGUUAAAAUAAGGCU |
| G015642 | | GGCUAGUCCGUUAUCAAAAUGGCACCGAGUCGG | | AGUCCGUUAUCAAAAUGGCACCGAGUCGG*mU*mG*m |
| | | UGC | | C |
| C- | 213 | GUUUUAGAGCUAGAAAUAGCAAGUUAAAAUAA | 313 | GUUUUAGAGCUAGAAAUAGCAAGUUAAAAUAAGGCU |
| G015643 | | GGCUAGUCCGUUAUCAAAGGCACCGAGUCGGUG | | AGUCCGUUAUCAAAGGCACCGAGUCGG*mU*mG*mC |
| | | C | | |
| C- | 214 | GUUUUAGAGCUAGAAAUAGCAAGUUAAAAUAA | 314 | GUUUUAGAGCUAGAAAUAGCAAGUUAAAAUAAGGCU |
| G015644 | | GGCUAGUCCGUUAUCAAGGGCACCGAGUCGGUG | | AGUCCGUUAUCAAGGGCACCGAGUCGG*mU*mG*mC |
| | | C | | |
| C- | 215 | GUUUUAGAGCUAGAAAUAGCAAGUUAAAAUAA | 315 | GUUUUAGAGCUAGAAAUAGCAAGUUAAAAUAAGGCU |
| G015645 | | GGCUAGUCCGUUAUCAGGCACCGAGUCGGUGC | | AGUCCGUUAUCAGGCACCGAGUCGG*mU*mG*mC |
| C- | 216 | GUUUUAGAGCUAGAAUAGCAAGUUAAAAUAAG | 316 | GUUUUAGAGCUAGAAUAGCAAGUUAAAAUAAGGCUA |
| G015646 | | GCUAGUCCGUUAUCAACUUGGCACCGAGUCGGU | | GUCCGUUAUCAACUUGGCACCGAGUCGG*mU*mG*mC |
| | | GC | | |
| C- | 217 | GUUUUAGAGCGCAAAGCGCAAGUUAAAAUAAG | 317 | GUUUUAGAGCGCAAAGCGCAAGUUAAAAUAAGGCUA |
| G015647 | | GCUAGUCCGUUAUCAACUUGGCACCGAGUCGGU | | GUCCGUUAUCAACUUGGCACCGAGUCGG*mU*mG*mC |
| | | GC | | |
| C- | 218 | GUUUUAGAGCGCGAAGCGCAAGUUAAAAUAAG | 318 | GUUUUAGAGCGCGAAGCGCAAGUUAAAAUAAGGCUA |
| G015648 | | GCUAGUCCGUUAUCAACUUGGCACCGAGUCGGU | | GUCCGUUAUCAACUUGGCACCGAGUCGG*mU*mG*mC |
| | | GC | | |
| C- | 219 | GUUUUAGAGCGGAAACGCAAGUUAAAAUAAGG | 319 | GUUUUAGAGCGGAAACGCAAGUUAAAAUAAGGCUAG |
| G015649 | | CUAGUCCGUUAUCAACUUGGCACCGAGUCGGUG | | UCCGUUAUCAACUUGGCACCGAGUCGGU*mG*mC*mU |
| | | CU | | |
| C- | 220 | GUUUUAGAGCGGAAACGCAAGUUAAAAUAAGG | 320 | GUUUUAGAGCGGAAACGCAAGUUAAAAUAAGGCUAG |
| G015650 | | CUAGUCCGUUAUCAACUUGGCACCGAGUCGGUG | | UCCGUUAUCAACUUGGCACCGAGUCGG*mU*mG*mC |
| | | C | | |
| C- | 221 | GUUUUAGAGCCGAAAGGCAAGUUAAAAUAAGG | 321 | GUUUUAGAGCCGAAAGGCAAGUUAAAAUAAGGCUAG |
| G015651 | | CUAGUCCGUUAUCAACUUGGCACCGAGUCGGUG | | UCCGUUAUCAACUUGGCACCGAGUCGG*mU*mG*mC |
| | | C | | |
| C- | 222 | GUUUUAGAGCUGAAAAGCAAGUUAAAAUAAGG | 322 | GUUUUAGAGCUGAAAAGCAAGUUAAAAUAAGGCUAG |
| G015652 | | CUAGUCCGUUAUCAACUUGGCACCGAGUCGGUG | | UCCGUUAUCAACUUGGCACCGAGUCGG*mU*mG*mC |
| | | C | | |
| C- | 223 | GUUUUAGAGCGAAAGCAAGUUAAAAUAAGGCU | 323 | GUUUUAGAGCGAAAGCAAGUUAAAAUAAGGCUAGUC |
| G015653 | | AGUCCGUUAUCAACUUGGCACCGAGUCGGUGC | | CGUUAUCAACUUGGCACCGAGUCGG*mU*mG*mC |
| C- | 224 | GUUUUAGAGCGAAGCAAGUUAAAAUAAGGCUA | 324 | GUUUUAGAGCGAAGCAAGUUAAAAUAAGGCUAGUCC |
| G015654 | | GUCCGUUAUCAACUUGGCACCGAGUCGGUGC | | GUUAUCAACUUGGCACCGAGUCGG*mU*mG*mC |
| C- | 225 | GUUUUAGAGCAAAGCAAGUUAAAAUAAGGCUA | 325 | GUUUUAGAGCAAAGCAAGUUAAAAUAAGGCUAGUCC |
| G015655 | | GUCCGUUAUCAACUUGGCACCGAGUCGGUGC | | GUUAUCAACUUGGCACCGAGUCGG*mU*mG*mC |
| C- | 226 | GUUUUAGAGGAAACAAGUUAAAAUAAGGCUAG | 326 | GUUUUAGAGGAAACAAGUUAAAAUAAGGCUAGUCCG |
| G015656 | | UCCGUUAUCAACUUGGCACCGAGUCGGUGC | | UUAUCAACUUGGCACCGAGUCGG*mU*mG*mC |
| C- | 227 | GUUUUAGAGCAAGCAAGUUAAAAUAAGGCUAG | 327 | GUUUUAGAGCAAGCAAGUUAAAAUAAGGCUAGUCCG |
| G015657 | | UCCGUUAUCAACUUGGCACCGAGUCGGUGC | | UUAUCAACUUGGCACCGAGUCGG*mU*mG*mC |
| C- | 228 | GUUUUAGAGCGAGCAAGUUAAAAUAAGGCUAG | 328 | GUUUUAGAGCGAGCAAGUUAAAAUAAGGCUAGUCCG |
| G015658 | | UCCGUUAUCAACUUGGCACCGAGUCGGUGC | | UUAUCAACUUGGCACCGAGUCGG*mU*mG*mC |
| C- | 229 | GUUUUAGAGCGCAAGUUAAAAUAAGGCUAGUC | 329 | GUUUUAGAGCGCAAGUUAAAAUAAGGCUAGUCCGUU |
| G015659 | | CGUUAUCAACUUGGCACCGAGUCGGUGC | | AUCAACUUGGCACCGAGUCGG*mU*mG*mC |
| C- | 230 | GUUUUAGAGAACAAGUUAAAAUAAGGCUAGUC | 330 | GUUUUAGAGAACAAGUUAAAAUAAGGCUAGUCCGUU |
| G015660 | | CGUUAUCAACUUGGCACCGAGUCGGUGC | | AUCAACUUGGCACCGAGUCGG*mU*mG*mC |
| C- | 231 | GUUUUAGAGACAAGUUAAAAUAAGGCUAGUCC | 331 | GUUUUAGAGACAAGUUAAAAUAAGGCUAGUCCGUUA |
| G015661 | | GUUAUCAACUUGGCACCGAGUCGGUGC | | UCAACUUGGCACCGAGUCGG*mU*mG*mC |
| C- | 232 | GUUUUAGAGCAAGUUAAAAUAAGGCUAGUCCG | 332 | GUUUUAGAGCAAGUUAAAAUAAGGCUAGUCCGUUAU |
| G015662 | | UUAUCAACUUGGCACCGAGUCGGUGC | | CAACUUGGCACCGAGUCGG*mU*mG*mC |
| C- | 233 | GUUUUAGAAAAAGUUAAAAUAAGGCUAGUCCG | 333 | GUUUUAGAAAAAGUUAAAAUAAGGCUAGUCCGUUAU |
| G015663 | | UUAUCAACUUGGCACCGAGUCGGUGC | | CAACUUGGCACCGAGUCGG*mU*mG*mC |
| C- | 234 | GUUUUAGAAAAGUUAAAAUAAGGCUAGUCCGU | 334 | GUUUUAGAAAAGUUAAAAUAAGGCUAGUCCGUUAUC |
| G015664 | | UAUCAACUUGGCACCGAGUCGGUGC | | AACUUGGCACCGAGUCGG*mU*mG*mC |
| C- | 235 | GUUUUAGAGCUAGAAAUAGCAAGUUAAAAUAA | 335 | GUUUUAGAGCUAGAAAUAGCAAGUUAAAAUAAGGCU |
| G015665 | | GGCUAGUCCGUUAUCAACUUGGCACCGAGUCGG | | AGUCCGUUAUCAACUUGGCACCGAGUCG*mG*mU*mG |
| | | UG | | |
| C- | 236 | GUUUUAGAGCUAGAAAUAGCAAGUUAAAAUAA | 336 | GUUUUAGAGCUAGAAAUAGCAAGUUAAAAUAAGGCU |
| G015666 | | GGCUAGUCCGUUAUCAACUUGGCACCGAGUCGG | | AGUCCGUUAUCAACUUGGCACCGAGUC*mG*mG*mU |
| | | U | | |
| C- | 237 | GUUUUAGAGCUAGAAAUAGCAAGUUAAAAUAA | 337 | GUUUUAGAGCUAGAAAUAGCAAGUUAAAAUAAGGCU |
| G015667 | | GGCUAGUCCGUUAUCAACUUGGCACCGAGUCGG | | AGUCCGUUAUCAACUUGGCACCGAGU*mC*mG*mG |
| C- | 238 | GUUUUAGAGCUAGAAAUAGCAAGUUAAAAUAA | 338 | GUUUUAGAGCUAGAAAUAGCAAGUUAAAAUAAGGCU |
| G015668 | | GGCUAGUCCGUUAUCAACUUGGCACCGAGUCG | | AGUCCGUUAUCAACUUGGCACCGAG*mU*mC*mG |
| C- | 239 | GUUUUAGAGCUAGAAAUAGCAAGUUAAAAUAA | 339 | GUUUUAGAGCUAGAAAUAGCAAGUUAAAAUAAGGCU |
| G015669 | | GGCUAGUCCGUUAUCAACUUGGCACCGAGUC | | AGUCCGUUAUCAACUUGGCACCGA*mG*mU*mC |
| C- | 240 | GUUUUAGAGCUAGAAAUAGCAAGUUAAAAUAA | 340 | GUUUUAGAGCUAGAAAUAGCAAGUUAAAAUAAGGCU |
| G015670 | | GGCUAGUCCGUUAUCAACUUGGCACCGAGU | | AGUCCGUUAUCAACUUGGCACCG*mA*mG*mU |
| C- | 241 | GUUUUAGAGCGAAAGCAAGUUAAAAUAAGGCU | 341 | GUUUUAGAGCGAAAGCAAGUUAAAAUAAGGCUAGUC |
| G015671 | | AGUCCGUUAUCACUGGCACCGAGUCGGUGC | | CGUUAUCACUGGCACCGAGUCGG*mU*mG*mC |
| C- | 242 | GUUUUAGAGCGAAAGCAAGUUAAAAUAAGGCU | 342 | GUUUUAGAGCGAAAGCAAGUUAAAAUAAGGCUAGUC |
| G015672 | | AGUCCGUUAUCAGGCACCGAGUCGGUGC | | CGUUAUCAGGCACCGAGUCGG*mU*mG*mC |
| C- | 243 | GUUUUAGAGAAAAAGUUAAAAUAAGGCUAGUC | 343 | GUUUUAGAGAAAAAGUUAAAAUAAGGCUAGUCCGUU |
| G015673 | | CGUUAUCAACUUGGCACCGAGUCGGUGC | | AUCAACUUGGCACCGAGUCGG*mU*mG*mC |
| C- | 244 | GUUUUAGAGGAAACAAGUUAAAAUAAGGCUAG | 344 | GUUUUAGAGGAAACAAGUUAAAAUAAGGCUAGUCCG |
| G015674 | | UCCGUUAUCAAUGGCACCGAGUCGGUGC | | UUAUCAAUGGCACCGAGUCGG*mU*mG*mC |
| C- | 245 | GUUUUAGAGAAAAAGUUAAAAUAAGGCUAGUC | 345 | GUUUUAGAGAAAAAGUUAAAAUAAGGCUAGUCCGUU |
| G015675 | | CGUUAUCAAUGGCACCGAGUCGGUGC | | AUCAAUGGCACCGAGUCGG*mU*mG*mC |
| C- | 246 | GUUUUAGAGGAAACAAGUUAAAAUAAGGCUAG | 346 | GUUUUAGAGGAAACAAGUUAAAAUAAGGCUAGUCCG |
| G015676 | | UCCGUUAUCACUGGCACCGAGUCGGUGC | | UUAUCACUGGCACCGAGUCGG*mU*mG*mC |
| C- | 247 | GUUUUAGAGAAAAAGUUAAAAUAAGGCUAGUC | 347 | GUUUUAGAGAAAAAGUUAAAAUAAGGCUAGUCCGUU |
| G015677 | | CGUUAUCAGGCACCGAGUCGGUGC | | AUCAGGCACCGAGUCGG*mU*mG*mC |
| C- | 248 | GUUUUAGAGCGAAAGCAAGUUAAAAUAAGGCU | 348 | GUUUUAGAGCGAAAGCAAGUUAAAAUAAGGCUAGUC |
| G015678 | | AGUCCGUUAUCAAGCUAUGGCACCGAGUCGGUG | | CGUUAUCAAGCUAUGGCACCGAGUCGG*mU*mG*mC |
| | | C | | |
| C- | 249 | GUUUUAGAGCUAGAAAUAGCAAGUUAAAAUAA | 349 | GUUUUAGAGCUAGAAAUAGCAAGUUAAAAUAAGGCA |
| G015679 | | GGCAGUCCGUUAUCAACUUGGCACCGAGUCGGU | | GUCCGUUAUCAACUUGGCACCGAGUCGG*mU*mG*mC |
| | | GC | | |
| C- | 250 | GUUUUAGAGCUAGAAAUAGCAAGUUAAAAUAA | 350 | GUUUUAGAGCUAGAAAUAGCAAGUUAAAAUAAGGCU |
| G015680 | | GGCUGUCCGUUAUCAACUUGGCACCGAGUCGGU | | GUCCGUUAUCAACUUGGCACCGAGUCGG*mU*mG*mC |
| | | GC | | |
| C- | 251 | GUUUUAGAGCUAGAAAUAGCAAGUUAAAAUAA | 351 | GUUUUAGAGCUAGAAAUAGCAAGUUAAAAUAAGGCG |
| G015681 | | GGCGUCCGUUAUCAACUUGGCACCGAGUCGGUG | | UCCGUUAUCAACUUGGCACCGAGUCGG*mU*mG*mC |
| | | C | | |
| C- | 252 | GUUUUAGAGCUAGAAAUAGCAAGUUAAAAUAA | 352 | GUUUUAGAGCUAGAAAUAGCAAGUUAAAAUAAGGUA |
| G015682 | | GGUAUCCGUUAUCAACUUGGCACCGAGUCGGUG | | UCCGUUAUCAACUUGGCACCGAGUCGG*mU*mG*mC |
| | | C | | |
| C- | 253 | GUUUUAGAGCUAGAAAUAGCAAGUUAAAAUAA | 353 | GUUUUAGAGCUAGAAAUAGCAAGUUAAAAUAAGGUU |
| G015683 | | GGUUCCGUUAUCAACUUGGCACCGAGUCGGUGC | | CCGUUAUCAACUUGGCACCGAGUCGG*mU*mG*mC |
| C- | 254 | GUUUUAGAGCUAGAAAUAGCAAGUUAAAAUAA | 354 | GUUUUAGAGCUAGAAAUAGCAAGUUAAAAUAAGGAU |
| G015684 | | GGAUCCGUUAUCAACUUGGCACCGAGUCGGUGC | | CCGUUAUCAACUUGGCACCGAGUCGG*mU*mG*mC |
| C- | 255 | GUUUUCGAGCUAGAAAUAGCAAGUGAAAAUAA | 355 | GUUUUCGAGCUAGAAAUAGCAAGUGAAAAUAAGGCU |
| G015685 | | GGCUAGUCCGUUAUCAACUUGGCACCGAGUCGG | | AGUCCGUUAUCAACUUGGCACCGAGUCGG*mU*mG*m |
| | | UGC | | C |
| C- | 256 | GUUUUUGAGCUAGAAAUAGCAAGUAAAAAUAA | 356 | GUUUUUGAGCUAGAAAUAGCAAGUAAAAAUAAGGCU |
| G015686 | | GGCUAGUCCGUUAUCAACUUGGCACCGAGUCGG | | AGUCCGUUAUCAACUUGGCACCGAGUCGG*mU*mG*m |
| | | UGC | | C |
| C- | 257 | GUUUUAGAGCGAGAAAUCGCAAGUUAAAAUAA | 357 | GUUUUAGAGCGAGAAAUCGCAAGUUAAAAUAAGGCU |
| G015687 | | GGCUAGUCCGUUAUCAACUUGGCACCGAGUCGG | | AGUCCGUUAUCAACUUGGCACCGAGUCGG*mU*mG*m |
| | | UGC | | C |
| C- | 258 | GUUUUAGAGCUAGAAAUAGCGAGUUAAAAUAA | 358 | GUUUUAGAGCUAGAAAUAGCGAGUUAAAAUAAGGCU |
| G015688 | | GGCUAGUCCGUUAUCAACUUGGCACCGAGUCGG | | AGUCCGUUAUCAACUUGGCACCGAGUCGG*mU*mG*m |
| | | UGC | | C |
| C- | 259 | GUUUUAGAGCUAGAAAUAGCCGGUUAAAAUAA | 359 | GUUUUAGAGCUAGAAAUAGCCGGUUAAAAUAAGGCU |
| G015689 | | GGCUAGUCCGUUAUCAACUUGGCACCGAGUCGG | | AGUCCGUUAUCAACUUGGCACCGAGUCGG*mU*mG*m |
| | | UGC | | C |
| C- | 260 | GUUUUAGAGCUAGAAAUAGCAAGUUAAAAUGA | 360 | GUUUUAGAGCUAGAAAUAGCAAGUUAAAAUGAGGCU |
| G015690 | | GGCUAGUCCGUUAUCAACUUGGCACCGAGUCGG | | AGUCCGUUAUCAACUUGGCACCGAGUCGG*mU*mG*m |
| | | UGC | | C |
| C- | 261 | GUUUUAGAGCUAGAAAUAGCAAGUUAAAAUAA | 361 | GUUUUAGAGCUAGAAAUAGCAAGUUAAAAUAAGGCU |
| G015691 | | GGCUGGUCCGUUAUCAACUUGGCACCGAGUCGG | | GGUCCGUUAUCAACUUGGCACCGAGUCGG*mU*mG*m |
| | | UGC | | C |
| C- | 262 | GUUUUAGAGCUAGAAAUAGCAAGUUAAAAUAA | 362 | GUUUUAGAGCUAGAAAUAGCAAGUUAAAAUAAGGCU |
| G015692 | | GGCUCGUCCGUUAUCAACUUGGCACCGAGUCGG | | CGUCCGUUAUCAACUUGGCACCGAGUCGG*mU*mG*m |
| | | UGC | | C |
| C- | 263 | GUUUUAGAGCUAGAAAUAGCAAGUUAAAAUAA | 363 | GUUUUAGAGCUAGAAAUAGCAAGUUAAAAUAAGGCU |
| G015693 | | GGCUUGUCCGUUAUCAACUUGGCACCGAGUCGG | | UGUCCGUUAUCAACUUGGCACCGAGUCGG*mU*mG*m |
| | | UGC | | c |
| C- | 264 | GUUUUAGAGCUAGAAAUAGCAAGUUAAAAUAA | 364 | GUUUUAGAGCUAGAAAUAGCAAGUUAAAAUAAGGCU |
| G015694 | | GGCUAGUCCGUUGUCAACUUGGCACCGAGUCGG | | AGUCCGUUGUCAACUUGGCACCGAGUCGG*mU*mG*m |
| | | UGC | | c |
| C- | 265 | GUUUUAGAGCUAGAAAUAGCAAGUUAAAAUAA | 365 | GUUUUAGAGCUAGAAAUAGCAAGUUAAAAUAAGGCU |
| G015695 | | GGCUAGUCCGUUCUCAACUUGGCACCGAGUCGG | | AGUCCGUUCUCAACUUGGCACCGAGUCGG*mU*mG*m |
| | | UGC | | c |
| C- | 266 | GUUUUAGAGCUAGAAAUAGCAAGUUAAAAUAA | 366 | GUUUUAGAGCUAGAAAUAGCAAGUUAAAAUAAGGCU |
| G015696 | | GGCUAGUCCGUUUUCAACUUGGCACCGAGUCGG | | AGUCCGUUUUCAACUUGGCACCGAGUCGG*mU*mG*m |
| | | UGC | | C |
| C- | 267 | GUUUUAGAGCUAGAAAUAGCAAGUUAAAAUAA | 367 | GUUUUAGAGCUAGAAAUAGCAAGUUAAAAUAAGGCU |
| G015697 | | GGCUAGUCCGUUAUGAACUUGGCACCGAGUCGG | | AGUCCGUUAUGAACUUGGCACCGAGUCGG*mU*mG*m |
| | | UGC | | C |
| C- | 268 | GUUUUAGAGCUAGAAAUAGCAAGUUAAAAUAA | 368 | GUUUUAGAGCUAGAAAUAGCAAGUUAAAAUAAGGCU |
| G015698 | | GGCUAGUCCGUUAUCAACUUGGGACCGAGUCGG | | AGUCCGUUAUCAACUUGGGACCGAGUCGG*mU*mC*m |
| | | UCC | | C |
| C- | 269 | GUUUUAGAGCUAGAAAUAGCAAGUUAAAAUAA | 369 | GUUUUAGAGCUAGAAAUAGCAAGUUAAAAUAAGGCU |
| G015699 | | GGCUAGUCCGUUAUCAACUUGGAACCGAGUCGG | | AGUCCGUUAUCAACUUGGAACCGAGUCGG*mU*mU*m |
| | | UUC | | C |
| C- | 270 | GUUUUCGAGCGAGAAAUCGCGAGUGAAAAUGA | 370 | GUUUUCGAGCGAGAAAUCGCGAGUGAAAAUGAGGCU |
| G015700 | | GGCUGGUCCGUUGUGAACUUGGAACCGAGUCGG | | GGUCCGUUGUGAACUUGGAACCGAGUCGG*mU*mU*m |
| | | UUC | | C |
| C- | 271 | GUUUUUGAGCGAGAAAUCGCAAGUAAAAAUAA | 371 | GUUUUUGAGCGAGAAAUCGCAAGUAAAAAUAAGGCU |
| G015701 | | GGCUCGUCCGUUCUGAACUUGGAACCGAGUCGG | | CGUCCGUUCUGAACUUGGAACCGAGUCGG*mU*mU*m |
| | | UUC | | C |
| C- | 272 | GUUUCGGAGCCGGAAACGGCGAGUCGAAAUGA | 372 | GUUUCGGAGCCGGAAACGGCGAGUCGAAAUGAGGCU |
| G015702 | | GGCUGGUCCGUUGUCGGCUCGGAACCGAGUCGG | | GGUCCGUUGUCGGCUCGGAACCGAGUCGG*mU*mU*m |
| | | UUC | | C |
| C- | 273 | GUUUUAGAGCUAGAAAUAGCAAGUUAAAAUAA | 373 | GUUUUAGAGCUAGAAAUAGCAAGUUAAAAUAAGGCU |
| G015703 | | GGCUAGUCCGUUAUCAACUUGGCACCGAGUCGG | | AGUCCGUUAUCAACUUGGCACCGAGUCGG*mU*mG*m |
| | | UGC | | C |
| C- | 274 | GUUUUAGAGCUAGAAAUAGCAAGUUAAAAUAA | 374 | GUUUUAGAGCUAGAAAUAGCAAGUUAAAAUAAGGCU |
| G015704 | | GGCUAGUCCGUUAUCAACUUGGCACCGAGUCGG | | AGUCCGUUAUCAACUUGGCACCGAGUCGG*mU*mG*m |
| | | UGC | | C |
| C- | 275 | GUUUUAGAGCUAGAAAUAGCAAGUUAAAAUAA | 375 | GUUUUAGAGCUAGAAAUAGCAAGUUAAAAUAAGGCU |
| G015705 | | GGCUAGUCCGUUAUCAACUUGGCACCGAGUCGG | | AGUCCGUUAUCAACUUGGCACCGAGUCGG*mU*mG*m |
| | | UGC | | C |
| C- | 276 | GUUUUAGAGCUAGAAAUAGCAAGUUAAAAUAA | 376 | GUUUUAGAGCUAGAAAUAGCAAGUUAAAAUAAGGCU |
| G015706 | | GGCUAGUCCGUUAUCAACUUGGCACCGAGUCGG | | AGUCCGUUAUCAACUUGGCACCGAGUCGG*mU*mG*m |
| | | UGC | | C |
| C- | 277 | GUUUUAGAGCUAGAAAUAGCAAGUUAAAAUAA | 377 | GUUUUAGAGCUAGAAAUAGCAAGUUAAAAUAAGGCU |
| G015707 | | GGCUAGUCCGUUAUCAACUUGGCACCGAGUCGG | | AGUCCGUUAUCAACUUGGCACCGAGUCGG*mU*mG*m |
| | | UGC | | C |
| C- | 278 | GUUUUAGAGCUAGAAAUAGCAAGUUAAAAUAA | 378 | GUUUUAGAGCUAGAAAUAGCAAGUUAAAAUAAGGCU |
| G015708 | | GGCUAGUCCGUUAUCAACUUGGCACCGAGUCGG | | AGUCCGUUAUCAACUUGGCACCGAGUCGG*mU*mG*m |
| | | UGC | | C |
| C- | 279 | GUUUUAGAGCUAGAAAUAGCAAGUUAAAAUAA | 379 | GUUUUAGAGCUAGAAAUAGCAAGUUAAAAUAAGGCU |
| G015709 | | GGCUAGUCCGUUAUCAACUUGGCACCGAGUCGG | | AGUCCGUUAUCAACUUGGCACCGAGUCGG*mU*mG*m |
| | | UGC | | C |
| C- | 280 | GUUUUAGAGCUAGAAAUAGCAAGUUAAAAUAA | 380 | GUUUUAGAGCUAGAAAUAGCAAGUUAAAAUAAGGCU |
| G017275 | | GGCUAGUCCGUUAUCAACUUGGCACCGAGUCGG | | AGUCCGUUAUCAACUUGGCACCGAGUCGG*mU*mG*m |
| | | UGC | | C |
| C- | 281 | GUUUUAGAGCUAGAAAUAGCAAGUUAAAAUAA | 381 | GUUUUAGAmGmCmUmAmGmAmAmAmUmAmGmCAAG |
| G017276 | | GGCUAGUCCGUUAUCACGAAAGGGCACCGAGUC | | UUAAAAUAAGGCUAGUCCGUUAUCACGAAAGGGCAC |
| | | GGUGC | | CGAGUCGG*mU*mG*mC |
| C- | 282 | GUUUUAGAGCUAGAAAUAGCAAGUUAAAAUAA | 382 | GUUUUAGAmGmCmUmAmGmAmAmAmUmAmGmCAAG |
| G017277 | | GGCUAGUCCGUUAUCAAAAAUGGCACCGAGUCG | | UUAAAAUAAGGCUAGUCCGUUAUCAAAAAUGGCACC |
| | | GUGC | | GAGUCGG*mU*mG*mC |
| C- | 283 | GUUUUAGAGCUAGAAAUAGCAAGUUAAAAUAA | 383 | GUUUUAGAmGmCmUmAmGmAmAmAmUmAmGmCAAG |
| G017278 | | GGCUAGUCCGUUAUCACAAGGGCACCGAGUCGG | | UUAAAAUAAGGCUAGUCCGUUAUCACAAGGGCACCG |
| | | UGC | | AGUCGG*mU*mG*mC |
| C- | 284 | GUUUUAGAGCUAGAAAUAGCAAGUUAAAAUAA | 384 | GUUUUAGAmGmCmUmAmGmAmAmAmUmAmGmCAAG |
| G017279 | | GGCUAGUCCGUUAUCAAAAUGGCACCGAGUCGG | | UUAAAAUAAGGCUAGUCCGUUAUCAAAAUGGCACCG |
| | | UGC | | AGUCGG*mU*mG*mC |
| C- | 285 | GUUUUAGAGCGCGAAGCGCAAGUUAAAAUAAG | 385 | GUUUUAGAGCGCGAAGCGCAAGUUAAAAUAAGGCUA |
| G017280 | | GCUAGUCCGUUAUCAAAAUGGCACCGAGUCGGU | | GUCCGUUAUCAAAAUGGCACCGAGUCGG*mU*mG*mC |
| | | GC | | |
| C- | 286 | GUUUUAGAGCUGAAAAGCAAGUUAAAAUAAGG | 386 | GUUUUAGAGCUGAAAAGCAAGUUAAAAUAAGGCUAG |
| G017281 | | CUAGUCCGUUAUCAAAAUGGCACCGAGUCGGUG | | UCCGUUAUCAAAAUGGCACCGAGUCGG*mU*mG*mC |
| | | C | | |
| C- | 287 | GUUUUAGAGCGAAAGCAAGUUAAAAUAAGGCU | 387 | GUUUUAGAGCGAAAGCAAGUUAAAAUAAGGCUAGUC |
| G017282 | | AGUCCGUUAUCAAAAUGGCACCGAGUCGGUGC | | CGUUAUCAAAAUGGCACCGAGUCGG*mU*mG*mC |
| C- | 288 | GUUUUAGAGCAAAGCAAGUUAAAAUAAGGCUA | 388 | GUUUUAGAGCAAAGCAAGUUAAAAUAAGGCUAGUCC |
| G017283 | | GUCCGUUAUCAAAAUGGCACCGAGUCGGUGC | | GUUAUCAAAAUGGCACCGAGUCGG*mU*mG*mC |
| C- | 289 | GUUUUAGAGCGAAAGCAAGUUAAAAUAAGGCU | 389 | GUUUUAGAmGmCmGmAmAmAmGmCAAGUUAAAAUA |
| G013773 | | AGUCCGUUAUCAACUUGGCACCGAGUCGGUGC | | AGGCUAGUCCGUUAUCAACUUGGCACCGAGUCGG*m |
| | | | | U*mG*mC |
| C- | 290 | GUUUUAGAGCGAAAGCAAGUUAAAAUAAGGCU | 390 | GUUUUAGAmGmCmGmAmAmAmGmCAAGUUAAAAUA |
| G013776 | | AGUCCGUUAUCAAGAAAUGGCACCGAGUCGGUG | | AGGCUAGUCCGUUAUCAAGAAAUGGCACCGAGUCGG |
| | | C | | *mU*mG*mC |
| C-SM07-6 | 291 | GUUUUAGAGCUAGAAAUAGCAAGUUAAAAUAA | 391 | GUUUUAGAmGmCmUmAmGmAmAmAmUmAmGmCAAG |
| | | GGCUAGUCCGUUAUCACGAAAGGGCACCGAGUC | | UUAAAAUAAGGCUAGUCCGUUAUCAmCmGmAmAmA |
| | | GGUGC | | mGmGmGmCmAmCmCmGmAmGmUmCmGmG*mU*mG* |
| | | | | mC |
| C-SM12-6 | 292 | GUUUUAGAGCUAGAAAUAGCAAGUUAAAAUAA | 392 | GUUUUAGAmGmCmUmAmGmAmAmAmUmAmGmCAAG |
| | | GGCUAGUCCGUUAUCAAAAUGGCACCGAGUCGG | | UUAAAAUAAGGCUAGUCCGUUAUCAmAmAmAmUmG |
| | | UGC | | mGmCmAmCmCmGmAmGmUmCmGmG*mU*mG*mC |
| C-SM07- | 293 | GUUUUAGAGCUAGAAAUAGCAAGUUAAAAUAA | 393 | GUUUUAGAmGmCmUmAmGmAmAmAmUmAmGmCAAG |
| 50 | | GGCUAGUCCGUUAUCACGAAAGGGCACCGAGUC | | UUAAAAUAAGGCUAGUCCGUUAUCACmGmAmAmAm |
| | | GGUGC | | GmGmGmCmAmCmCmGmAmGmUmCmGmG*mU*mG*m |
| | | | | C |
| C-SM12- | 294 | GUUUUAGAGCUAGAAAUAGCAAGUUAAAAUAA | 394 | GUUUUAGAmGmCmUmAmGmAmAmAmUmAmGmCAAG |
| 50 | | GGCUAGUCCGUUAUCAAAAUGGCACCGAGUCGG | | UUAAAAUAAGGCUAGUCCGUUAUCAAmAmAmUmGm |
| | | UGC | | GmCmAmCmCmGmAmGmUmCmGmG*mU*mG*mC |
| | 295- | Not Used | 395- | Not Used |
| | 300 | | 399 | |
| | 400 | See Table 2 | | |
| Nx- | 401 | N₃NₓGUUUUAGAGCUAGAAAUAGCAAGUUAAAA | 501 | (mN*)₃NₓGUUUUAGAGCUAGAAAUAGCAAGUUAAAAU |
| G015631 | | UAAGGCUAGUCCGUUAUCAACUAAGCACCGAGU | | AAGGCUAGUCCGUUAUCAACUAAGCACCGAGUCGG* |
| | | CGGUGC | | mU*mG*mC |
| Nx- | 402 | N₃NₓGUUUUAGAGCUAGAAAUAGCAAGUUAAAA | 502 | (mN*)₃NₓGUUUUAGAGCUAGAAAUAGCAAGUUAAAAU |
| G015632 | | UAAGGCUAGUCCGUUAUCAACUCAGCACCGAGU | | AAGGCUAGUCCGUUAUCAACUCAGCACCGAGUCGG* |
| | | CGGUGC | | mU*mG*mC |
| Nx- | 403 | N₃NₓGUUUUAGAGCUAGAAAUAGCAAGUUAAAA | 503 | (mN*)₃NₓGUUUUAGAGCUAGAAAUAGCAAGUUAAAAU |
| G015633 | | UAAGGCUAGUCCGUUAUCCACUUGGCACCGAGU | | AAGGCUAGUCCGUUAUCCACUUGGCACCGAGUCGG* |
| | | CGGUGC | | mU*mG*mC |
| Nx- | 404 | N₃NₓGUUUUAGAGCUAGAAAUAGCAAGUUAAAA | 504 | (mN*)₃NₓGUUUUAGAGCUAGAAAUAGCAAGUUAAAAU |
| G015634 | | UACGGCUAGUCCGUUAUCAACUUGGCACCGAGU | | ACGGCUAGUCCGUUAUCAACUUGGCACCGAGUCGG* |
| | | CGGUGC | | mU*mG*mC |
| Nx- | 405 | N₃NₓGUUUUAGAGCUAGAAAUAGCAAGUUAAAA | 505 | (mN*)₃NₓGUUUUAGAGCUAGAAAUAGCAAGUUAAAAU |
| G015635 | | UAAGGCUAGUCCGUUAUCAAGAGCUGGCACCGA | | AAGGCUAGUCCGUUAUCAAGAGCUGGCACCGAGUCG |
| | | GUCGGUGC | | G*mU*mG*mC |
| Nx- | 406 | N₃NₓGUUUUAGAGCUAGAAAUAGCAAGUUAAAA | 506 | (mN*)₃NₓGUUUUAGAGCUAGAAAUAGCAAGUUAAAAU |
| G015636 | | UAAGGCUAGUCCGUUAUCAAGAAAUGGCACCGA | | AAGGCUAGUCCGUUAUCAAGAAAUGGCACCGAGUCG |
| | | GUCGGUGC | | G*mU*mG*mC |
| Nx- | 407 | N₃NₓGUUUUAGAGCUAGAAAUAGCAAGUUAAAA | 507 | (mN*)₃NₓGUUUUAGAGCUAGAAAUAGCAAGUUAAAAU |
| G015637 | | UAAGGCUAGUCCGUUAUCACGAAAGGGCACCGA | | AAGGCUAGUCCGUUAUCACGAAAGGGCACCGAGUCG |
| | | GUCGGUGC | | G*mU*mG*mC |
| Nx- | 408 | N₃NₓGUUUUAGAGCUAGAAAUAGCAAGUUAAAA | 508 | (mN*)₃NₓGUUUUAGAGCUAGAAAUAGCAAGUUAAAAU |
| G015638 | | UAAGGCUAGUCCGUUAUCAAAAAUGGCACCGAG | | AAGGCUAGUCCGUUAUCAAAAAUGGCACCGAGUCGG |
| | | UCGGUGC | | *mU*mG*mC |
| Nx- | 409 | N₃NₓGUUUUAGAGCUAGAAAUAGCAAGUUAAAA | 509 | (mN*)₃NₓGUUUUAGAGCUAGAAAUAGCAAGUUAAAAU |
| G015639 | | UAAGGCUAGUCCGUUAUCAAAAGUGGCACCGAG | | AAGGCUAGUCCGUUAUCAAAAGUGGCACCGAGUCGG |
| | | UCGGUGC | | *mU*mG*mC |
| Nx- | 410 | N₃NₓGUUUUAGAGCUAGAAAUAGCAAGUUAAAA | 510 | (mN*)₃NₓGUUUUAGAGCUAGAAAUAGCAAGUUAAAAU |
| G015640 | | UAAGGCUAGUCCGUUAUCAACAGUGGCACCGAG | | AAGGCUAGUCCGUUAUCAACAGUGGCACCGAGUCGG |
| | | UCGGUGC | | *mU*mG*mC |
| Nx- | 411 | N₃NₓGUUUUAGAGCUAGAAAUAGCAAGUUAAAA | 511 | (mN*)₃NₓGUUUUAGAGCUAGAAAUAGCAAGUUAAAAU |
| G015641 | | UAAGGCUAGUCCGUUAUCACAAGGGCACCGAGU | | AAGGCUAGUCCGUUAUCACAAGGGCACCGAGUCGG* |
| | | CGGUGC | | mU*mG*mC |
| Nx- | 412 | N₃NₓGUUUUAGAGCUAGAAAUAGCAAGUUAAAA | 512 | (mN*)₃NₓGUUUUAGAGCUAGAAAUAGCAAGUUAAAAU |
| G015642 | | UAAGGCUAGUCCGUUAUCAAAAUGGCACCGAGU | | AAGGCUAGUCCGUUAUCAAAAUGGCACCGAGUCGG* |
| | | CGGUGC | | mU*mG*mC |
| Nx- | 413 | N₃NₓGUUUUAGAGCUAGAAAUAGCAAGUUAAAA | 513 | (mN*)₃NₓGUUUUAGAGCUAGAAAUAGCAAGUUAAAAU |
| G015643 | | UAAGGCUAGUCCGUUAUCAAAGGCACCGAGUCG | | AAGGCUAGUCCGUUAUCAAAGGCACCGAGUCGG*mU* |
| | | GUGC | | mG*mC |
| Nx- | 414 | N₃NₓGUUUUAGAGCUAGAAAUAGCAAGUUAAAA | 514 | (mN*)₃NₓGUUUUAGAGCUAGAAAUAGCAAGUUAAAAU |
| G015644 | | UAAGGCUAGUCCGUUAUCAAGGGCACCGAGUCG | | AAGGCUAGUCCGUUAUCAAGGGCACCGAGUCGG*mU* |
| | | GUGC | | mG*mC |
| Nx- | 415 | N₃NₓGUUUUAGAGCUAGAAAUAGCAAGUUAAAA | 515 | (mN*)₃NₓGUUUUAGAGCUAGAAAUAGCAAGUUAAAAU |
| G015645 | | UAAGGCUAGUCCGUUAUCAGGCACCGAGUCGGU | | AAGGCUAGUCCGUUAUCAGGCACCGAGUCGG*mU*m |
| | | GC | | G*mC |
| Nx- | 416 | N₃NₓGUUUUAGAGCUAGAAUAGCAAGUUAAAAU | 516 | (mN*)₃NₓGUUUUAGAGCUAGAAUAGCAAGUUAAAAUA |
| G015646 | | AAGGCUAGUCCGUUAUCAACUUGGCACCGAGUC | | AGGCUAGUCCGUUAUCAACUUGGCACCGAGUCGG*m |
| | | GGUGC | | U*mG*mC |
| Nx- | 417 | N₃NₓGUUUUAGAGCGCAAAGCGCAAGUUAAAAU | 517 | (mN*)₃NₓGUUUUAGAGCGCAAAGCGCAAGUUAAAAUA |
| G015647 | | AAGGCUAGUCCGUUAUCAACUUGGCACCGAGUC | | AGGCUAGUCCGUUAUCAACUUGGCACCGAGUCGG*m |
| | | GGUGC | | U*mG*mC |
| Nx- | 418 | N₃NₓGUUUUAGAGCGCGAAGCGCAAGUUAAAAU | 518 | (mN*)₃NₓGUUUUAGAGCGCGAAGCGCAAGUUAAAAUA |
| G015648 | | AAGGCUAGUCCGUUAUCAACUUGGCACCGAGUC | | AGGCUAGUCCGUUAUCAACUUGGCACCGAGUCGG*m |
| | | GGUGC | | U*mG*mC |
| Nx- | 419 | N₃NₓGUUUUAGAGCGGAAACGCAAGUUAAAAUA | 519 | (mN*)₃NₓGUUUUAGAGCGGAAACGCAAGUUAAAAUAA |
| G015649 | | AGGCUAGUCCGUUAUCAACUUGGCACCGAGUCG | | GGCUAGUCCGUUAUCAACUUGGCACCGAGUCGGU*m |
| | | GUGCU | | G*mC*mU |
| Nx- | 420 | N₃NₓGUUUUAGAGCGGAAACGCAAGUUAAAAUA | 520 | (mN*)₃NₓGUUUUAGAGCGGAAACGCAAGUUAAAAUAA |
| G015650 | | AGGCUAGUCCGUUAUCAACUUGGCACCGAGUCG | | GGCUAGUCCGUUAUCAACUUGGCACCGAGUCGG*mU* |
| | | GUGC | | mG*mC |
| Nx- | 421 | N₃NₓGUUUUAGAGCCGAAAGGCAAGUUAAAAUA | 521 | (mN*)₃NₓGUUUUAGAGCCGAAAGGCAAGUUAAAAUAA |
| G015651 | | AGGCUAGUCCGUUAUCAACUUGGCACCGAGUCG | | GGCUAGUCCGUUAUCAACUUGGCACCGAGUCGG*mU* |
| | | GUGC | | mG*mC |
| Nx- | 422 | N₃NₓGUUUUAGAGCUGAAAAGCAAGUUAAAAUA | 522 | (mN*)₃NₓGUUUUAGAGCUGAAAAGCAAGUUAAAAUAA |
| G015652 | | AGGCUAGUCCGUUAUCAACUUGGCACCGAGUCG | | GGCUAGUCCGUUAUCAACUUGGCACCGAGUCGG*mU* |
| | | GUGC | | mG*mC |
| Nx- | 423 | N₃NₓGUUUUAGAGCGAAAGCAAGUUAAAAUAAG | 523 | (mN*)₃NₓGUUUUAGAGCGAAAGCAAGUUAAAAUAAGG |
| G015653 | | GCUAGUCCGUUAUCAACUUGGCACCGAGUCGGU | | CUAGUCCGUUAUCAACUUGGCACCGAGUCGG*mU*mG |
| | | GC | | *mC |
| Nx- | 424 | N₃NₓGUUUUAGAGCGAAGCAAGUUAAAAUAAGG | 524 | (mN*)₃NₓGUUUUAGAGCGAAGCAAGUUAAAAUAAGGC |
| G015654 | | CUAGUCCGUUAUCAACUUGGCACCGAGUCGGUG | | UAGUCCGUUAUCAACUUGGCACCGAGUCGG*mU*mG* |
| | | C | | mC |
| Nx- | 425 | N₃NₓGUUUUAGAGCAAAGCAAGUUAAAAUAAGG | 525 | (mN*)₃NₓGUUUUAGAGCAAAGCAAGUUAAAAUAAGGC |
| G015655 | | CUAGUCCGUUAUCAACUUGGCACCGAGUCGGUG | | UAGUCCGUUAUCAACUUGGCACCGAGUCGG*mU*mG* |
| | | C | | mC |
| Nx- | 426 | N₃NₓGUUUUAGAGGAAACAAGUUAAAAUAAGGC | 526 | (mN*)₃NₓGUUUUAGAGGAAACAAGUUAAAAUAAGGCU |
| G015656 | | UAGUCCGUUAUCAACUUGGCACCGAGUCGGUGC | | AGUCCGUUAUCAACUUGGCACCGAGUCGG*mU*mG*m |
| | | | | C |
| Nx- | 427 | N₃NₓGUUUUAGAGCAAGCAAGUUAAAAUAAGGC | 527 | (mN*)₃NₓGUUUUAGAGCAAGCAAGUUAAAAUAAGGCU |
| G015657 | | UAGUCCGUUAUCAACUUGGCACCGAGUCGGUGC | | AGUCCGUUAUCAACUUGGCACCGAGUCGG*mU*mG*m |
| | | | | C |
| Nx- | 428 | N₃NₓGUUUUAGAGCGAGCAAGUUAAAAUAAGGC | 528 | (mN*)₃NₓGUUUUAGAGCGAGCAAGUUAAAAUAAGGCU |
| G015658 | | UAGUCCGUUAUCAACUUGGCACCGAGUCGGUGC | | AGUCCGUUAUCAACUUGGCACCGAGUCGG*mU*mG*m |
| | | | | C |
| Nx- | 429 | N₃NₓGUUUUAGAGCGCAAGUUAAAAUAAGGCUA | 529 | (mN*)₃NₓGUUUUAGAGCGCAAGUUAAAAUAAGGCUAG |
| G015659 | | GUCCGUUAUCAACUUGGCACCGAGUCGGUGC | | UCCGUUAUCAACUUGGCACCGAGUCGG*mU*mG*mC |
| Nx- | 430 | N₃NₓGUUUUAGAGAACAAGUUAAAAUAAGGCUA | 530 | (mN*)₃NₓGUUUUAGAGAACAAGUUAAAAUAAGGCUAG |
| G015660 | | GUCCGUUAUCAACUUGGCACCGAGUCGGUGC | | UCCGUUAUCAACUUGGCACCGAGUCGG*mU*mG*mC |
| Nx- | 431 | N₃NₓGUUUUAGAGACAAGUUAAAAUAAGGCUAG | 531 | (mN*)₃NₓGUUUUAGAGACAAGUUAAAAUAAGGCUAGU |
| G015661 | | UCCGUUAUCAACUUGGCACCGAGUCGGUGC | | CCGUUAUCAACUUGGCACCGAGUCGG*mU*mG*mC |
| Nx- | 432 | N₃NₓGUUUUAGAGCAAGUUAAAAUAAGGCUAGU | 532 | (mN*)₃NₓGUUUUAGAGCAAGUUAAAAUAAGGCUAGUC |
| G015662 | | CCGUUAUCAACUUGGCACCGAGUCGGUGC | | CGUUAUCAACUUGGCACCGAGUCGG*mU*mG*mC |
| Nx- | 433 | N₃NₓGUUUUAGAAAAAGUUAAAAUAAGGCUAGU | 533 | (mN*)₃NₓGUUUUAGAAAAAGUUAAAAUAAGGCUAGUC |
| G015663 | | CCGUUAUCAACUUGGCACCGAGUCGGUGC | | CGUUAUCAACUUGGCACCGAGUCGG*mU*mG*mC |
| Nx- | 434 | N₃NₓGUUUUAGAAAAGUUAAAAUAAGGCUAGUC | 534 | (mN*)₃NₓGUUUUAGAAAAGUUAAAAUAAGGCUAGUCC |
| G015664 | | CGUUAUCAACUUGGCACCGAGUCGGUGC | | GUUAUCAACUUGGCACCGAGUCGG*mU*mG*mC |
| Nx- | 435 | N₃NₓGUUUUAGAGCUAGAAAUAGCAAGUUAAAA | 535 | (mN*)₃NₓGUUUUAGAGCUAGAAAUAGCAAGUUAAAAU |
| G015665 | | UAAGGCUAGUCCGUUAUCAACUUGGCACCGAGU | | AAGGCUAGUCCGUUAUCAACUUGGCACCGAGUCG*m |
| | | CGGUG | | G*mU*mG |
| Nx- | 436 | N₃NₓGUUUUAGAGCUAGAAAUAGCAAGUUAAAA | 536 | (mN*)₃NₓGUUUUAGAGCUAGAAAUAGCAAGUUAAAAU |
| G015666 | | UAAGGCUAGUCCGUUAUCAACUUGGCACCGAGU | | AAGGCUAGUCCGUUAUCAACUUGGCACCGAGUC*mG* |
| | | CGGU | | mG*mU |
| Nx- | 437 | N₃NₓGUUUUAGAGCUAGAAAUAGCAAGUUAAAA | 537 | (mN*)₃NₓGUUUUAGAGCUAGAAAUAGCAAGUUAAAAU |
| G015667 | | UAAGGCUAGUCCGUUAUCAACUUGGCACCGAGU | | AAGGCUAGUCCGUUAUCAACUUGGCACCGAGU*mC*m |
| | | CGG | | G*mG |
| Nx- | 438 | N₃NₓGUUUUAGAGCUAGAAAUAGCAAGUUAAAA | 538 | (mN*)₃NₓGUUUUAGAGCUAGAAAUAGCAAGUUAAAAU |
| G015668 | | UAAGGCUAGUCCGUUAUCAACUUGGCACCGAGU | | AAGGCUAGUCCGUUAUCAACUUGGCACCGAG*mU*mC |
| | | CG | | *mG |
| Nx- | 439 | N₃NₓGUUUUAGAGCUAGAAAUAGCAAGUUAAAA | 539 | (mN*)₃NₓGUUUUAGAGCUAGAAAUAGCAAGUUAAAAU |
| G015669 | | UAAGGCUAGUCCGUUAUCAACUUGGCACCGAGU | | AAGGCUAGUCCGUUAUCAACUUGGCACCGA*mG*mU* |
| | | C | | mC |
| Nx- | 440 | N₃NₓGUUUUAGAGCUAGAAAUAGCAAGUUAAAA | 540 | (mN*)₃NₓGUUUUAGAGCUAGAAAUAGCAAGUUAAAAU |
| G015670 | | UAAGGCUAGUCCGUUAUCAACUUGGCACCGAGU | | AAGGCUAGUCCGUUAUCAACUUGGCACCG*mA*mG*m |
| | | | | U |
| Nx- | 441 | N₃NₓGUUUUAGAGCGAAAGCAAGUUAAAAUAAG | 541 | (mN*)₃NₓGUUUUAGAGCGAAAGCAAGUUAAAAUAAGG |
| G015671 | | GCUAGUCCGUUAUCACUGGCACCGAGUCGGUGC | | CUAGUCCGUUAUCACUGGCACCGAGUCGG*mU*mG*m |
| | | | | C |
| Nx- | 442 | N₃NₓGUUUUAGAGCGAAAGCAAGUUAAAAUAAG | 542 | (mN*)₃NₓGUUUUAGAGCGAAAGCAAGUUAAAAUAAGG |
| G015672 | | GCUAGUCCGUUAUCAGGCACCGAGUCGGUGC | | CUAGUCCGUUAUCAGGCACCGAGUCGG*mU*mG*mC |
| Nx- | 443 | N₃NₓGUUUUAGAGAAAAAGUUAAAAUAAGGCUA | 543 | (mN*)₃NₓGUUUUAGAGAAAAAGUUAAAAUAAGGCUAG |
| G015673 | | GUCCGUUAUCAACUUGGCACCGAGUCGGUGC | | UCCGUUAUCAACUUGGCACCGAGUCGG*mU*mG*mC |
| Nx- | 444 | N₃NₓGUUUUAGAGGAAACAAGUUAAAAUAAGGC | 544 | (mN*)₃NₓGUUUUAGAGGAAACAAGUUAAAAUAAGGCU |
| G015674 | | UAGUCCGUUAUCAAUGGCACCGAGUCGGUGC | | AGUCCGUUAUCAAUGGCACCGAGUCGG*mU*mG*mC |
| Nx- | 445 | N₃NₓGUUUUAGAGAAAAAGUUAAAAUAAGGCUA | 545 | (mN*)₃NₓGUUUUAGAGAAAAAGUUAAAAUAAGGCUAG |
| G015675 | | GUCCGUUAUCAAUGGCACCGAGUCGGUGC | | UCCGUUAUCAAUGGCACCGAGUCGG*mU*mG*mC |
| Nx- | 446 | N₃NₓGUUUUAGAGGAAACAAGUUAAAAUAAGGC | 546 | (mN*)₃NₓGUUUUAGAGGAAACAAGUUAAAAUAAGGCU |
| G015676 | | UAGUCCGUUAUCACUGGCACCGAGUCGGUGC | | AGUCCGUUAUCACUGGCACCGAGUCGG*mU*mG*mC |
| Nx- | 447 | N₃NₓGUUUUAGAGAAAAAGUUAAAAUAAGGCUA | 547 | (mN*)₃NₓGUUUUAGAGAAAAAGUUAAAAUAAGGCUAG |
| G015677 | | GUCCGUUAUCAGGCACCGAGUCGGUGC | | UCCGUUAUCAGGCACCGAGUCGG*mU*mG*mC |
| Nx- | 448 | N₃NₓGUUUUAGAGCGAAAGCAAGUUAAAAUAAG | 548 | (mN*)₃NₓGUUUUAGAGCGAAAGCAAGUUAAAAUAAGG |
| G015678 | | GCUAGUCCGUUAUCAAGCUAUGGCACCGAGUCG | | CUAGUCCGUUAUCAAGCUAUGGCACCGAGUCGG*mU* |
| | | GUGC | | mG*mC |
| Nx- | 449 | N₃NₓGUUUUAGAGCUAGAAAUAGCAAGUUAAAA | 549 | (mN*)₃NₓGUUUUAGAGCUAGAAAUAGCAAGUUAAAAU |
| G015679 | | UAAGGCAGUCCGUUAUCAACUUGGCACCGAGUC | | AAGGCAGUCCGUUAUCAACUUGGCACCGAGUCGG*m |
| | | GGUGC | | U*mG*mC |
| Nx- | 450 | N₃NₓGUUUUAGAGCUAGAAAUAGCAAGUUAAAA | 550 | (mN*)₃NₓGUUUUAGAGCUAGAAAUAGCAAGUUAAAAU |
| G015680 | | UAAGGCUGUCCGUUAUCAACUUGGCACCGAGUC | | AAGGCUGUCCGUUAUCAACUUGGCACCGAGUCGG*m |
| | | GGUGC | | U*mG*mC |
| Nx- | 451 | N₃NₓGUUUUAGAGCUAGAAAUAGCAAGUUAAAA | 551 | (mN*)₃NₓGUUUUAGAGCUAGAAAUAGCAAGUUAAAAU |
| G015681 | | UAAGGCGUCCGUUAUCAACUUGGCACCGAGUCG | | AAGGCGUCCGUUAUCAACUUGGCACCGAGUCGG*mU* |
| | | GUGC | | mG*mC |
| Nx- | 452 | N₃NₓGUUUUAGAGCUAGAAAUAGCAAGUUAAAA | 552 | (mN*)₃NₓGUUUUAGAGCUAGAAAUAGCAAGUUAAAAU |
| G015682 | | UAAGGUAUCCGUUAUCAACUUGGCACCGAGUCG | | AAGGUAUCCGUUAUCAACUUGGCACCGAGUCGG*mU* |
| | | GUGC | | mG*mC |
| Nx- | 453 | N₃NₓGUUUUAGAGCUAGAAAUAGCAAGUUAAAA | 553 | (mN*)₃NₓGUUUUAGAGCUAGAAAUAGCAAGUUAAAAU |
| G015683 | | UAAGGUUCCGUUAUCAACUUGGCACCGAGUCGG | | AAGGUUCCGUUAUCAACUUGGCACCGAGUCGG*mU* |
| | | UGC | | mG*mC |
| Nx- | 454 | N₃NₓGUUUUAGAGCUAGAAAUAGCAAGUUAAAA | 554 | (mN*)₃NₓGUUUUAGAGCUAGAAAUAGCAAGUUAAAAU |
| G015684 | | UAAGGAUCCGUUAUCAACUUGGCACCGAGUCGG | | AAGGAUCCGUUAUCAACUUGGCACCGAGUCGG*mU* |
| | | UGC | | mG*mC |
| Nx- | 455 | N₃NₓGUUUUCGAGCUAGAAAUAGCAAGUGAAAA | 555 | (mN*)₃NₓGUUUUCGAGCUAGAAAUAGCAAGUGAAAAU |
| G015685 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 456 | N₃NₓGUUUUUGAGCUAGAAAUAGCAAGUAAAAA | 556 | (mN*)₃NₓGUUUUUGAGCUAGAAAUAGCAAGUAAAAAU |
| G015686 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 457 | N₃NₓGUUUUAGAGCGAGAAAUCGCAAGUUAAAA | 557 | (mN*)₃NₓGUUUUAGAGCGAGAAAUCGCAAGUUAAAAU |
| G015687 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 458 | N₃NₓGUUUUAGAGCUAGAAAUAGCGAGUUAAAA | 558 | (mN*)₃NₓGUUUUAGAGCUAGAAAUAGCGAGUUAAAAU |
| G015688 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 459 | N₃NₓGUUUUAGAGCUAGAAAUAGCCGGUUAAAA | 559 | (mN*)₃NₓGUUUUAGAGCUAGAAAUAGCCGGUUAAAAU |
| G015689 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 460 | N₃NₓGUUUUAGAGCUAGAAAUAGCAAGUUAAAA | 560 | (mN*)₃NₓGUUUUAGAGCUAGAAAUAGCAAGUUAAAAU |
| G015690 | | UGAGGCUAGUCCGUUAUCAACUUGGCACCGAGU | | GAGGCUAGUCCGUUAUCAACUUGGCACCGAGUCGG* |
| | | CGGUGC | | mU*mG*mC |
| Nx- | 461 | N₃NₓGUUUUAGAGCUAGAAAUAGCAAGUUAAAA | 561 | (mN*)₃NₓGUUUUAGAGCUAGAAAUAGCAAGUUAAAAU |
| G015691 | | UAAGGCUGGUCCGUUAUCAACUUGGCACCGAGU | | AAGGCUGGUCCGUUAUCAACUUGGCACCGAGUCGG* |
| | | CGGUGC | | mU*mG*mC |
| Nx- | 462 | N₃NₓGUUUUAGAGCUAGAAAUAGCAAGUUAAAA | 562 | (mN*)₃NₓGUUUUAGAGCUAGAAAUAGCAAGUUAAAAU |
| G015692 | | UAAGGCUCGUCCGUUAUCAACUUGGCACCGAGU | | AAGGCUCGUCCGUUAUCAACUUGGCACCGAGUCGG* |
| | | CGGUGC | | mU*mG*mC |
| Nx- | 463 | N₃NₓGUUUUAGAGCUAGAAAUAGCAAGUUAAAA | 563 | (mN*)₃NₓGUUUUAGAGCUAGAAAUAGCAAGUUAAAAU |
| G015693 | | UAAGGCUUGUCCGUUAUCAACUUGGCACCGAGU | | AAGGCUUGUCCGUUAUCAACUUGGCACCGAGUCGG* |
| | | CGGUGC | | mU*mG*mC |
| Nx- | 464 | N₃NₓGUUUUAGAGCUAGAAAUAGCAAGUUAAAA | 564 | (mN*)₃NₓGUUUUAGAGCUAGAAAUAGCAAGUUAAAAU |
| G015694 | | UAAGGCUAGUCCGUUGUCAACUUGGCACCGAGU | | AAGGCUAGUCCGUUGUCAACUUGGCACCGAGUCGG* |
| | | CGGUGC | | mU*mG*mC |
| Nx- | 465 | N₃NₓGUUUUAGAGCUAGAAAUAGCAAGUUAAAA | 565 | (mN*)₃NₓGUUUUAGAGCUAGAAAUAGCAAGUUAAAAU |
| G015695 | | UAAGGCUAGUCCGUUCUCAACUUGGCACCGAGU | | AAGGCUAGUCCGUUCUCAACUUGGCACCGAGUCGG* |
| | | CGGUGC | | mU*mG*mC |
| Nx- | 466 | N₃NₓGUUUUAGAGCUAGAAAUAGCAAGUUAAAA | 566 | (mN*)₃NₓGUUUUAGAGCUAGAAAUAGCAAGUUAAAAU |
| G015696 | | UAAGGCUAGUCCGUUUUCAACUUGGCACCGAGU | | AAGGCUAGUCCGUUUUCAACUUGGCACCGAGUCGG* |
| | | CGGUGC | | mU*mG*mC |
| Nx- | 467 | N₃NₓGUUUUAGAGCUAGAAAUAGCAAGUUAAAA | 567 | (mN*)₃NₓGUUUUAGAGCUAGAAAUAGCAAGUUAAAAU |
| G015697 | | UAAGGCUAGUCCGUUAUGAACUUGGCACCGAGU | | AAGGCUAGUCCGUUAUGAACUUGGCACCGAGUCGG* |
| | | CGGUGC | | mU*mG*mC |
| Nx- | 468 | N₃NₓGUUUUAGAGCUAGAAAUAGCAAGUUAAAA | 568 | (mN*)₃NₓGUUUUAGAGCUAGAAAUAGCAAGUUAAAAU |
| G015698 | | UAAGGCUAGUCCGUUAUCAACUUGGGACCGAGU | | AAGGCUAGUCCGUUAUCAACUUGGGACCGAGUCGG* |
| | | CGGUCC | | mU*mC*mC |
| Nx- | 469 | N₃NₓGUUUUAGAGCUAGAAAUAGCAAGUUAAAA | 569 | (mN*)₃NₓGUUUUAGAGCUAGAAAUAGCAAGUUAAAAU |
| G015699 | | UAAGGCUAGUCCGUUAUCAACUUGGAACCGAGU | | AAGGCUAGUCCGUUAUCAACUUGGAACCGAGUCGG* |
| | | CGGUUC | | mU*mU*mC |
| Nx- | 470 | N₃NₓGUUUUCGAGCGAGAAAUCGCGAGUGAAAA | 570 | (mN*)₃NₓGUUUUCGAGCGAGAAAUCGCGAGUGAAAAU |
| G015700 | | UGAGGCUGGUCCGUUGUGAACUUGGAACCGAG | | GAGGCUGGUCCGUUGUGAACUUGGAACCGAGUCGG* |
| | | UCGGUUC | | mU*mU*mC |
| Nx- | 471 | N₃NₓGUUUUUGAGCGAGAAAUCGCAAGUAAAAA | 571 | (mN*)₃NₓGUUUUUGAGCGAGAAAUCGCAAGUAAAAAU |
| G015701 | | UAAGGCUCGUCCGUUCUGAACUUGGAACCGAGU | | AAGGCUCGUCCGUUCUGAACUUGGAACCGAGUCGG* |
| | | CGGUUC | | mU*mU*mC |
| Nx- | 472 | N₃NₓGUUUCGGAGCCGGAAACGGCGAGUCGAAAU | 572 | (mN*)₃NₓGUUUCGGAGCCGGAAACGGCGAGUCGAAAU |
| G015702 | | GAGGCUGGUCCGUUGUCGGCUCGGAACCGAGUC | | GAGGCUGGUCCGUUGUCGGCUCGGAACCGAGUCGG* |
| | | GGUUC | | mU*mU*mC |
| Nx- | 473 | N₃NₓGUUUUAGAGCUAGAAAUAGCAAGUUAAAA | 573 | (mN*)₃NₓGUUUUAGAGCUAGAAAUAGCAAGUUAAAAU |
| G015703 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 474 | N₃NₓGUUUUAGAGCUAGAAAUAGCAAGUUAAAA | 574 | (mN*)₃NₓGUUUUAGAGCUAGAAAUAGCAAGUUAAAAU |
| G015704 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 475 | N₃NₓGUUUUAGAGCUAGAAAUAGCAAGUUAAAA | 575 | (mN*)₃NₓGUUUUAGAGCUAGAAAUAGCAAGUUAAAAU |
| G015705 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 476 | N₃NₓGUUUUAGAGCUAGAAAUAGCAAGUUAAAA | 576 | (mN*)₃NₓGUUUUAGAGCUAGAAAUAGCAAGUUAAAAU |
| G015706 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 477 | N₃NₓGUUUUAGAGCUAGAAAUAGCAAGUUAAAA | 577 | (mN*)₃NₓGUUUUAGAGCUAGAAAUAGCAAGUUAAAAU |
| G015707 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 478 | N₃NₓGUUUUAGAGCUAGAAAUAGCAAGUUAAAA | 578 | (mN*)₃NₓGUUUUAGAGCUAGAAAUAGCAAGUUAAAAU |
| G015708 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 479 | N₃NₓGUUUUAGAGCUAGAAAUAGCAAGUUAAAA | 579 | (mN*)₃NₓGUUUUAGAGCUAGAAAUAGCAAGUUAAAAU |
| G015709 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 480 | N₃NₓGUUUUAGAGCUAGAAAUAGCAAGUUAAAA | 580 | (mN*)₃NₓGUUUUAGAGCUAGAAAUAGCAAGUUAAAAU |
| G017275 | | UAAGGCUAGUCCGUUAUCAACUUGGCACCGAGU | | AAGGCUAGUCCGUUAUCAACUUGGCACCGAGUCGG* |
| | | CGGUGC | | mU*mG*mC |
| Nx- | 481 | N₃NₓGUUUUAGAGCUAGAAAUAGCAAGUUAAAA | 581 | (mN*)₃NₓGUUUUAGAmGmCmUmAmGmAmAmAmUmAm |
| G017276 | | UAAGGCUAGUCCGUUAUCACGAAAGGGCACCGA | | GmCAAGUUAAAAUAAGGCUAGUCCGUUAUCACGAAA |
| | | GUCGGUGC | | GGGCACCGAGUCGG*mU*mG*mC |
| Nx- | 482 | N₃NₓGUUUUAGAGCUAGAAAUAGCAAGUUAAAA | 582 | (mN*)NₓGUUUUAGAmGmCmUmAmGmAmAmAmUmAm |
| G017277 | | UAAGGCUAGUCCGUUAUCAAAAAUGGCACCGAG | | GmCAAGUUAAAAUAAGGCUAGUCCGUUAUCAAAAAU |
| | | UCGGUGC | | GGCACCGAGUCGG*mU*mG*mC |
| Nx- | 483 | N₃NₓGUUUUAGAGCUAGAAAUAGCAAGUUAAAA | 583 | (mN*)NₓGUUUUAGAmGmCmUmAmGmAmAmAmUmAm |
| G017278 | | UAAGGCUAGUCCGUUAUCACAAGGGCACCGAGU | | GmCAAGUUAAAAUAAGGCUAGUCCGUUAUCACAAGG |
| | | CGGUGC | | GCACCGAGUCGG*mU*mG*mC |
| Nx- | 484 | N₃NₓGUUUUAGAGCUAGAAAUAGCAAGUUAAAA | 584 | (mN*)₃NₓGUUUUAGAmGmCmUmAmGmAmAmAmUmAm |
| G017279 | | UAAGGCUAGUCCGUUAUCAAAAUGGCACCGAGU | | GmCAAGUUAAAAUAAGGCUAGUCCGUUAUCAAAAUG |
| | | CGGUGC | | GCACCGAGUCGG*mU*mG*mC |
| Nx- | 485 | N₃NₓGUUUUAGAGCGCGAAGCGCAAGUUAAAAU | 585 | (mN*)₃NₓGUUUUAGAGCGCGAAGCGCAAGUUAAAAUA |
| G017280 | | AAGGCUAGUCCGUUAUCAAAAUGGCACCGAGUC | | AGGCUAGUCCGUUAUCAAAAUGGCACCGAGUCGG*m |
| | | GGUGC | | U*mG*mC |
| Nx- | 486 | N₃NₓGUUUUAGAGCUGAAAAGCAAGUUAAAAUA | 586 | (mN*)₃NₓGUUUUAGAGCUGAAAAGCAAGUUAAAAUAA |
| G017281 | | AGGCUAGUCCGUUAUCAAAAUGGCACCGAGUCG | | GGCUAGUCCGUUAUCAAAAUGGCACCGAGUCGG*mU* |
| | | GUGC | | mG*mC |
| Nx- | 487 | N₃NₓGUUUUAGAGCGAAAGCAAGUUAAAAUAAG | 587 | (mN*)₃NₓGUUUUAGAGCGAAAGCAAGUUAAAAUAAGG |
| G017282 | | GCUAGUCCGUUAUCAAAAUGGCACCGAGUCGGU | | CUAGUCCGUUAUCAAAAUGGCACCGAGUCGG*mU*m |
| | | GC | | G*mC |
| Nx- | 488 | N₃NₓGUUUUAGAGCAAAGCAAGUUAAAAUAAGG | 588 | (mN*)₃NₓGUUUUAGAGCAAAGCAAGUUAAAAUAAGGC |
| G017283 | | CUAGUCCGUUAUCAAAAUGGCACCGAGUCGGUG | | UAGUCCGUUAUCAAAAUGGCACCGAGUCGG*mU*mG* |
| | | C | | mC |
| Nx- | 489 | N₃NₓGUUUUAGAGCGAAAGCAAGUUAAAAUAAG | 589 | (mN*)₃NₓGUUUUAGAmGmCmGmAmAmAmGmCAAGUU |
| G013773 | | GCUAGUCCGUUAUCAACUUGGCACCGAGUCGGU | | AAAAUAAGGCUAGUCCGUUAUCAACUUGGCACCGAG |
| | | GC | | UCGG*mU*mG*mC |
| Nx- | 490 | N₃NₓGUUUUAGAGCGAAAGCAAGUUAAAAUAAG | 590 | (mN*)₃NₓGUUUUAGAmGmCmGmAmAmAmGmCAAGUU |
| G013776 | | GCUAGUCCGUUAUCAAGAAAUGGCACCGAGUCG | | AAAAUAAGGCUAGUCCGUUAUCAAGAAAUGGCACCG |
| | | GUGC | | AGUCGG*mU*mG*mC |
| Nx-SM07- | 491 | N₃NₓGUUUUAGAGCUAGAAAUAGCAAGUUAAAA | 591 | (mN*)₃NₓGUUUUAGAmGmCmUmAmGmAmAmAmUmAm |
| 6 | | UAAGGCUAGUCCGUUAUCACGAAAGGGCACCGA | | GmCAAGUUAAAAUAAGGCUAGUCCGUUAUCAmCmGm |
| | | GUCGGUGC | | AmAmAmGmGmGmCmAmCmCmGmAmGmUmCmGmG*m |
| | | | | U*mG*mC |
| Nx-SM12- | 492 | N₃NₓGUUUUAGAGCUAGAAAUAGCAAGUUAAAA | 592 | (mN*)₃NₓGUUUUAGAmGmCmUmAmGmAmAmAmUmAm |
| 6 | | UAAGGCUAGUCCGUUAUCAAAAUGGCACCGAGU | | GmCAAGUUAAAAUAAGGCUAGUCCGUUAUCAmAmA |
| | | CGGUGC | | mAmUmGmGmCmAmCmCmGmAmGmUmCmGmG*mU*m |
| | | | | G*mC |
| Nx-SM07- | 493 | N₃NₓGUUUUAGAGCUAGAAAUAGCAAGUUAAAA | 593 | (mN*)NₓGUUUUAGAmGmCmUmAmGmAmAmAmUmAm |
| 50 | | UAAGGCUAGUCCGUUAUCACGAAAGGGCACCGA | | GmCAAGUUAAAAUAAGGCUAGUCCGUUAUCACmGmA |
| | | GUCGGUGC | | mAmAmGmGmGmCmAmCmCmGmAmGmUmCmGmG*mU |
| | | | | *mG*mC |
| Nx-SM12- | 494 | N₃NₓGUUUUAGAGCUAGAAAUAGCAAGUUAAAA | 594 | (mN*)₃NₓGUUUUAGAmGmCmUmAmGmAmAmAmUmAm |
| 50 | | UAAGGCUAGUCCGUUAUCAAAAUGGCACCGAGU | | GmCAAGUUAAAAUAAGGCUAGUCCGUUAUCAAmAmA |
| | | CGGUGC | | mUmGmGmCmAmCmCmGmAmGmUmCmGmG*mU*mG* |
| | | | | mC |
| | 495- | Not Used | 595- | Not Used |
| | 500 | | 600 | |
| N20- | 601 | N₂₀GUUUUAGAGCUAGAAAUAGCAAGUUAAAAU | 701 | (mN*)₃N₁₇GUUUUAGAGCUAGAAAUAGCAAGUUAAAA |
| G015631 | | AAGGCUAGUCCGUUAUCAACUAAGCACCGAGUC | | UAAGGCUAGUCCGUUAUCAACUAAGCACCGAGUCGG |
| | | GGUGC | | *mU*mG*mC |
| N20- | 602 | N₂₀GUUUUAGAGCUAGAAAUAGCAAGUUAAAAU | 702 | (mN*)₃N₁₇GUUUUAGAGCUAGAAAUAGCAAGUUAAAA |
| G015632 | | AAGGCUAGUCCGUUAUCAACUCAGCACCGAGUC | | UAAGGCUAGUCCGUUAUCAACUCAGCACCGAGUCGG |
| | | GGUGC | | *mU*mG*mC |
| N20- | 603 | N₂₀GUUUUAGAGCUAGAAAUAGCAAGUUAAAAU | 703 | (mN*)₃N₁₇GUUUUAGAGCUAGAAAUAGCAAGUUAAAA |
| G015633 | | AAGGCUAGUCCGUUAUCCACUUGGCACCGAGUC | | UAAGGCUAGUCCGUUAUCCACUUGGCACCGAGUCGG |
| | | GGUGC | | *mU*mG*mC |
| N20- | 604 | N₂₀GUUUUAGAGCUAGAAAUAGCAAGUUAAAAU | 704 | (mN*)₃N₁₇GUUUUAGAGCUAGAAAUAGCAAGUUAAAA |
| G015634 | | ACGGCUAGUCCGUUAUCAACUUGGCACCGAGUC | | UACGGCUAGUCCGUUAUCAACUUGGCACCGAGUCGG |
| | | GGUGC | | *mU*mG*mC |
| N20- | 605 | N₂₀GUUUUAGAGCUAGAAAUAGCAAGUUAAAAU | 705 | (mN*)₃N₁₇GUUUUAGAGCUAGAAAUAGCAAGUUAAAA |
| G015635 | | AAGGCUAGUCCGUUAUCAAGAGCUGGCACCGAG | | UAAGGCUAGUCCGUUAUCAAGAGCUGGCACCGAGUC |
| | | UCGGUGC | | GG*mU*mG*mC |
| N20- | 606 | N₂₀GUUUUAGAGCUAGAAAUAGCAAGUUAAAAU | 706 | (mN*)₃N₁₇GUUUUAGAGCUAGAAAUAGCAAGUUAAAA |
| G015636 | | AAGGCUAGUCCGUUAUCAAGAAAUGGCACCGAG | | UAAGGCUAGUCCGUUAUCAAGAAAUGGCACCGAGUC |
| | | UCGGUGC | | GG*mU*mG*mC |
| N20- | 607 | N₂₀GUUUUAGAGCUAGAAAUAGCAAGUUAAAAU | 707 | (mN*)₃N₁₇GUUUUAGAGCUAGAAAUAGCAAGUUAAAA |
| G015637 | | AAGGCUAGUCCGUUAUCACGAAAGGGCACCGAG | | UAAGGCUAGUCCGUUAUCACGAAAGGGCACCGAGUC |
| | | UCGGUGC | | GG*mU*mG*mC |
| N20- | 608 | N₂₀GUUUUAGAGCUAGAAAUAGCAAGUUAAAAU | 708 | (mN*)₃N₁₇GUUUUAGAGCUAGAAAUAGCAAGUUAAAA |
| G015638 | | AAGGCUAGUCCGUUAUCAAAAAUGGCACCGAGU | | UAAGGCUAGUCCGUUAUCAAAAAUGGCACCGAGUCG |
| | | CGGUGC | | G*mU*mG*mC |
| N20- | 609 | N₂₀GUUUUAGAGCUAGAAAUAGCAAGUUAAAAU | 709 | (mN*)₃N₁₇GUUUUAGAGCUAGAAAUAGCAAGUUAAAA |
| G015639 | | AAGGCUAGUCCGUUAUCAAAAGUGGCACCGAGU | | UAAGGCUAGUCCGUUAUCAAAAGUGGCACCGAGUCG |
| | | CGGUGC | | G*mU*mG*mC |
| N20- | 610 | N₂₀GUUUUAGAGCUAGAAAUAGCAAGUUAAAAU | 710 | (mN*)₃N₁₇GUUUUAGAGCUAGAAAUAGCAAGUUAAAA |
| G015640 | | AAGGCUAGUCCGUUAUCAACAGUGGCACCGAGU | | UAAGGCUAGUCCGUUAUCAACAGUGGCACCGAGUCG |
| | | CGGUGC | | G*mU*mG*mC |
| N20- | 611 | N₂₀GUUUUAGAGCUAGAAAUAGCAAGUUAAAAU | 711 | (mN*)₃N₁₇GUUUUAGAGCUAGAAAUAGCAAGUUAAAA |
| G015641 | | AAGGCUAGUCCGUUAUCACAAGGGCACCGAGUC | | UAAGGCUAGUCCGUUAUCACAAGGGCACCGAGUCGG |
| | | GGUGC | | *mU*mG*mC |
| N20- | 612 | N₂₀GUUUUAGAGCUAGAAAUAGCAAGUUAAAAU | 712 | (mN*)₃N₁₇GUUUUAGAGCUAGAAAUAGCAAGUUAAAA |
| G015642 | | AAGGCUAGUCCGUUAUCAAAAUGGCACCGAGUC | | UAAGGCUAGUCCGUUAUCAAAAUGGCACCGAGUCGG |
| | | GGUGC | | *mU*mG*mC |
| N20- | 613 | N₂₀GUUUUAGAGCUAGAAAUAGCAAGUUAAAAU | 713 | (mN*)₃N₁₇GUUUUAGAGCUAGAAAUAGCAAGUUAAAA |
| G015643 | | AAGGCUAGUCCGUUAUCAAAGGCACCGAGUCGG | | UAAGGCUAGUCCGUUAUCAAAGGCACCGAGUCGG*m |
| | | UGC | | U*mG*mC |
| N20- | 614 | N₂₀GUUUUAGAGCUAGAAAUAGCAAGUUAAAAU | 714 | (mN*)₃N₁₇GUUUUAGAGCUAGAAAUAGCAAGUUAAAA |
| G015644 | | AAGGCUAGUCCGUUAUCAAGGGCACCGAGUCGG | | UAAGGCUAGUCCGUUAUCAAGGGCACCGAGUCGG*m |
| | | UGC | | U*mG*mC |
| N20- | 615 | N₂₀GUUUUAGAGCUAGAAAUAGCAAGUUAAAAU | 715 | (mN*)₃N₁₇GUUUUAGAGCUAGAAAUAGCAAGUUAAAA |
| G015645 | | AAGGCUAGUCCGUUAUCAGGCACCGAGUCGGUG | | UAAGGCUAGUCCGUUAUCAGGCACCGAGUCGG*mU* |
| | | C | | mG*mC |
| N20- | 616 | N₂₀GUUUUAGAGCUAGAAUAGCAAGUUAAAAUA | 716 | (mN*)₃N₁₇GUUUUAGAGCUAGAAUAGCAAGUUAAAAU |
| G015646 | | AGGCUAGUCCGUUAUCAACUUGGCACCGAGUCG | | AAGGCUAGUCCGUUAUCAACUUGGCACCGAGUCGG* |
| | | GUGC | | mU*mG*mC |
| N20- | 617 | N₂₀GUUUUAGAGCGCAAAGCGCAAGUUAAAAUA | 717 | (mN*)₃N₁₇GUUUUAGAGCGCAAAGCGCAAGUUAAAAUA |
| G015647 | | AGGCUAGUCCGUUAUCAACUUGGCACCGAGUCG | | AGGCUAGUCCGUUAUCAACUUGGCACCGAGUCGG*m |
| | | GUGC | | U*mG*mC |
| N20- | 618 | N₂₀GUUUUAGAGCGCGAAGCGCAAGUUAAAAUA | 718 | (mN*)₃N₁₇GUUUUAGAGCGCGAAGCGCAAGUUAAAAUA |
| G015648 | | AGGCUAGUCCGUUAUCAACUUGGCACCGAGUCG | | AGGCUAGUCCGUUAUCAACUUGGCACCGAGUCGG*m |
| | | GUGC | | U*mG*mC |
| N20- | 619 | N₂₀GUUUUAGAGCGGAAACGCAAGUUAAAAUAA | 719 | (mN*)₃N₁₇GUUUUAGAGCGGAAACGCAAGUUAAAAUAA |
| G015649 | | GGCUAGUCCGUUAUCAACUUGGCACCGAGUCGG | | GGCUAGUCCGUUAUCAACUUGGCACCGAGUCGGU*m |
| | | UGCU | | G*mC*mU |
| N20- | 620 | N₂₀GUUUUAGAGCGGAAACGCAAGUUAAAAUAA | 720 | (mN*)₃N₁₇GUUUUAGAGCGGAAACGCAAGUUAAAAUAA |
| G015650 | | GGCUAGUCCGUUAUCAACUUGGCACCGAGUCGG | | GGCUAGUCCGUUAUCAACUUGGCACCGAGUCGG*mU* |
| | | UGC | | mG*mC |
| N20- | 621 | N₂₀GUUUUAGAGCCGAAAGGCAAGUUAAAAUAA | 721 | (mN*)₃N₁₇GUUUUAGAGCCGAAAGGCAAGUUAAAAUAA |
| G015651 | | GGCUAGUCCGUUAUCAACUUGGCACCGAGUCGG | | GGCUAGUCCGUUAUCAACUUGGCACCGAGUCGG*mU* |
| | | UGC | | mG*mC |
| N20- | 622 | N₂₀GUUUUAGAGCUGAAAAGCAAGUUAAAAUAA | 722 | (mN*)₃N₁₇GUUUUAGAGCUGAAAAGCAAGUUAAAAUA |
| G015652 | | GGCUAGUCCGUUAUCAACUUGGCACCGAGUCGG | | AGGCUAGUCCGUUAUCAACUUGGCACCGAGUCGG*m |
| | | UGC | | U*mG*mC |
| N20- | 623 | N₂₀GUUUUAGAGCGAAAGCAAGUUAAAAUAAGG | 723 | (mN*)₃N₁₇GUUUUAGAGCGAAAGCAAGUUAAAAUAAG |
| G015653 | | CUAGUCCGUUAUCAACUUGGCACCGAGUCGGUG | | GCUAGUCCGUUAUCAACUUGGCACCGAGUCGG*mU*m |
| | | C | | G*mC |
| N20- | 624 | N₂₀GUUUUAGAGCGAAGCAAGUUAAAAUAAGGC | 724 | (mN*)₃N₁₇GUUUUAGAGCGAAGCAAGUUAAAAUAAGGC |
| G015654 | | UAGUCCGUUAUCAACUUGGCACCGAGUCGGUGC | | UAGUCCGUUAUCAACUUGGCACCGAGUCGG*mU*mG* |
| | | | | mC |
| N20- | 625 | N₂₀GUUUUAGAGCAAAGCAAGUUAAAAUAAGGC | 725 | (mN*)₃N₁₇GUUUUAGAGCAAAGCAAGUUAAAAUAAGGC |
| G015655 | | UAGUCCGUUAUCAACUUGGCACCGAGUCGGUGC | | UAGUCCGUUAUCAACUUGGCACCGAGUCGG*mU*mG* |
| | | | | mC |
| N20- | 626 | N₂₀GUUUUAGAGGAAACAAGUUAAAAUAAGGCU | 726 | (mN*)₃N₁₇GUUUUAGAGGAAACAAGUUAAAAUAAGGC |
| G015656 | | AGUCCGUUAUCAACUUGGCACCGAGUCGGUGC | | UAGUCCGUUAUCAACUUGGCACCGAGUCGG*mU*mG* |
| | | | | mC |
| N20- | 627 | N₂₀GUUUUAGAGCAAGCAAGUUAAAAUAAGGCU | 727 | (mN*)₃N₁₇GUUUUAGAGCAAGCAAGUUAAAAUAAGGCU |
| G015657 | | AGUCCGUUAUCAACUUGGCACCGAGUCGGUGC | | AGUCCGUUAUCAACUUGGCACCGAGUCGG*mU*mG*m |
| | | | | C |
| N20- | 628 | N₂₀GUUUUAGAGCGAGCAAGUUAAAAUAAGGCU | 728 | (mN*)₃N₁₇GUUUUAGAGCGAGCAAGUUAAAAUAAGGCU |
| G015658 | | AGUCCGUUAUCAACUUGGCACCGAGUCGGUGC | | AGUCCGUUAUCAACUUGGCACCGAGUCGG*mU*mG*m |
| | | | | C |
| N20- | 629 | N₂₀GUUUUAGAGCGCAAGUUAAAAUAAGGCUAG | 729 | (mN*)₃N₁₇GUUUUAGAGCGCAAGUUAAAAUAAGGCUAG |
| G015659 | | UCCGUUAUCAACUUGGCACCGAGUCGGUGC | | UCCGUUAUCAACUUGGCACCGAGUCGG*mU*mG*mC |
| N20- | 630 | N₂₀GUUUUAGAGAACAAGUUAAAAUAAGGCUAG | 730 | (mN*)₃N₁₇GUUUUAGAGAACAAGUUAAAAUAAGGCUA |
| G015660 | | UCCGUUAUCAACUUGGCACCGAGUCGGUGC | | GUCCGUUAUCAACUUGGCACCGAGUCGG*mU*mG*mC |
| N20- | 631 | N₂₀GUUUUAGAGACAAGUUAAAAUAAGGCUAGU | 731 | (mN*)₃N₁₇GUUUUAGAGACAAGUUAAAAUAAGGCUAG |
| G015661 | | CCGUUAUCAACUUGGCACCGAGUCGGUGC | | UCCGUUAUCAACUUGGCACCGAGUCGG*mU*mG*mC |
| N20- | 632 | N₂₀GUUUUAGAGCAAGUUAAAAUAAGGCUAGUC | 732 | (mN*)₃N₁₇GUUUUAGAGCAAGUUAAAAUAAGGCUAGUC |
| G015662 | | CGUUAUCAACUUGGCACCGAGUCGGUGC | | CGUUAUCAACUUGGCACCGAGUCGG*mU*mG*mC |
| N20- | 633 | N₂₀GUUUUAGAAAAAGUUAAAAUAAGGCUAGUC | 733 | (mN*)₃N₁₇GUUUUAGAAAAAGUUAAAAUAAGGCUAGU |
| G015663 | | CGUUAUCAACUUGGCACCGAGUCGGUGC | | CCGUUAUCAACUUGGCACCGAGUCGG*mU*mG*mC |
| N20- | 634 | N₂₀GUUUUAGAAAAGUUAAAAUAAGGCUAGUCC | 734 | (mN*)₃N₁₇GUUUUAGAAAAGUUAAAAUAAGGCUAGUCC |
| G015664 | | GUUAUCAACUUGGCACCGAGUCGGUGC | | GUUAUCAACUUGGCACCGAGUCGG*mU*mG*mC |
| N20- | 635 | N₂₀GUUUUAGAGCUAGAAAUAGCAAGUUAAAAU | 735 | (mN*)₃N₁₇GUUUUAGAGCUAGAAAUAGCAAGUUAAAA |
| G015665 | | AAGGCUAGUCCGUUAUCAACUUGGCACCGAGUC | | UAAGGCUAGUCCGUUAUCAACUUGGCACCGAGUCG* |
| | | GGUG | | mG*mU*mG |
| N20- | 636 | N₂₀GUUUUAGAGCUAGAAAUAGCAAGUUAAAAU | 736 | (mN*)₃N₁₇GUUUUAGAGCUAGAAAUAGCAAGUUAAAA |
| G015666 | | AAGGCUAGUCCGUUAUCAACUUGGCACCGAGUC | | UAAGGCUAGUCCGUUAUCAACUUGGCACCGAGUC*m |
| | | GGU | | G*mG*mU |
| N20- | 637 | N₂₀GUUUUAGAGCUAGAAAUAGCAAGUUAAAAU | 737 | (mN*)₃N₁₇GUUUUAGAGCUAGAAAUAGCAAGUUAAAA |
| G015667 | | AAGGCUAGUCCGUUAUCAACUUGGCACCGAGUC | | UAAGGCUAGUCCGUUAUCAACUUGGCACCGAGU*mC* |
| | | GG | | mG*mG |
| N20- | 638 | N₂₀GUUUUAGAGCUAGAAAUAGCAAGUUAAAAU | 738 | (mN*)₃N₁₇GUUUUAGAGCUAGAAAUAGCAAGUUAAAA |
| G015668 | | AAGGCUAGUCCGUUAUCAACUUGGCACCGAGUC | | UAAGGCUAGUCCGUUAUCAACUUGGCACCGAG*mU* |
| | | G | | mC*mG |
| N20- | 639 | N₂₀GUUUUAGAGCUAGAAAUAGCAAGUUAAAAU | 739 | (mN*)₃N₁₇GUUUUAGAGCUAGAAAUAGCAAGUUAAAA |
| G015669 | | AAGGCUAGUCCGUUAUCAACUUGGCACCGAGUC | | UAAGGCUAGUCCGUUAUCAACUUGGCACCGA*mG*m |
| | | | | U*mC |
| N20- | 640 | N₂₀GUUUUAGAGCUAGAAAUAGCAAGUUAAAAU | 740 | (mN*)₃N₁₇GUUUUAGAGCUAGAAAUAGCAAGUUAAAA |
| G015670 | | AAGGCUAGUCCGUUAUCAACUUGGCACCGAGU | | UAAGGCUAGUCCGUUAUCAACUUGGCACCG*mA*mG* |
| | | | | mU |
| N20- | 641 | N₂₀GUUUUAGAGCGAAAGCAAGUUAAAAUAAGG | 741 | (mN*)₃N₁₇GUUUUAGAGCGAAAGCAAGUUAAAAUAAG |
| G015671 | | CUAGUCCGUUAUCACUGGCACCGAGUCGGUGC | | GCUAGUCCGUUAUCACUGGCACCGAGUCGG*mU*mG* |
| | | | | mC |
| N20- | 642 | N₂₀GUUUUAGAGCGAAAGCAAGUUAAAAUAAGG | 742 | (mN*)₃N₁₇GUUUUAGAGCGAAAGCAAGUUAAAAUAAG |
| G015672 | | CUAGUCCGUUAUCAGGCACCGAGUCGGUGC | | GCUAGUCCGUUAUCAGGCACCGAGUCGG*mU*mG*mC |
| N20- | 643 | N₂₀GUUUUAGAGAAAAAGUUAAAAUAAGGCUAG | 743 | (mN*)₃N₁₇GUUUUAGAGAAAAAGUUAAAAUAAGGCUA |
| G015673 | | UCCGUUAUCAACUUGGCACCGAGUCGGUGC | | GUCCGUUAUCAACUUGGCACCGAGUCGG*mU*mG*mC |
| N20- | 644 | N₂₀GUUUUAGAGGAAACAAGUUAAAAUAAGGCU | 744 | (mN*)₃N₁₇GUUUUAGAGGAAACAAGUUAAAAUAAGGC |
| G015674 | | AGUCCGUUAUCAAUGGCACCGAGUCGGUGC | | UAGUCCGUUAUCAAUGGCACCGAGUCGG*mU*mG*mC |
| N20- | 645 | N₂₀GUUUUAGAGAAAAAGUUAAAAUAAGGCUAG | 745 | (mN*)₃N₁₇GUUUUAGAGAAAAAGUUAAAAUAAGGCUA |
| G015675 | | UCCGUUAUCAAUGGCACCGAGUCGGUGC | | GUCCGUUAUCAAUGGCACCGAGUCGG*mU*mG*mC |
| N20- | 646 | N₂₀GUUUUAGAGGAAACAAGUUAAAAUAAGGCU | 746 | (mN*)₃N₁₇GUUUUAGAGGAAACAAGUUAAAAUAAGGC |
| G015676 | | AGUCCGUUAUCACUGGCACCGAGUCGGUGC | | UAGUCCGUUAUCACUGGCACCGAGUCGG*mU*mG*mC |
| N20- | 647 | N₂₀GUUUUAGAGAAAAAGUUAAAAUAAGGCUAG | 747 | (mN*)₃N₁₇GUUUUAGAGAAAAAGUUAAAAUAAGGCUA |
| G015677 | | UCCGUUAUCAGGCACCGAGUCGGUGC | | GUCCGUUAUCAGGCACCGAGUCGG*mU*mG*mC |
| N20- | 648 | N₂₀GUUUUAGAGCGAAAGCAAGUUAAAAUAAGG | 748 | (mN*)₃N₁₇GUUUUAGAGCGAAAGCAAGUUAAAAUAAG |
| G015678 | | CUAGUCCGUUAUCAAGCUAUGGCACCGAGUCGG | | GCUAGUCCGUUAUCAAGCUAUGGCACCGAGUCGG*m |
| | | UGC | | U*mG*mC |
| N20- | 649 | N₂₀GUUUUAGAGCUAGAAAUAGCAAGUUAAAAU | 749 | (mN*)₃N₁₇GUUUUAGAGCUAGAAAUAGCAAGUUAAAA |
| G015679 | | AAGGCAGUCCGUUAUCAACUUGGCACCGAGUCG | | UAAGGCAGUCCGUUAUCAACUUGGCACCGAGUCGG* |
| | | GUGC | | mU*mG*mC |
| N20- | 650 | N₂₀GUUUUAGAGCUAGAAAUAGCAAGUUAAAAU | 750 | (mN*)₃N₁₇GUUUUAGAGCUAGAAAUAGCAAGUUAAAA |
| G015680 | | AAGGCUGUCCGUUAUCAACUUGGCACCGAGUCG | | UAAGGCUGUCCGUUAUCAACUUGGCACCGAGUCGG* |
| | | GUGC | | mU*mG*mC |
| N20- | 651 | N₂₀GUUUUAGAGCUAGAAAUAGCAAGUUAAAAU | 751 | (mN*)₃N₁₇GUUUUAGAGCUAGAAAUAGCAAGUUAAAA |
| G015681 | | AAGGCGUCCGUUAUCAACUUGGCACCGAGUCGG | | UAAGGCGUCCGUUAUCAACUUGGCACCGAGUCGG*m |
| | | UGC | | U*mG*mC |
| N20- | 652 | N₂₀GUUUUAGAGCUAGAAAUAGCAAGUUAAAAU | 752 | (mN*)₃N₁₇GUUUUAGAGCUAGAAAUAGCAAGUUAAAA |
| G015682 | | AAGGUAUCCGUUAUCAACUUGGCACCGAGUCGG | | UAAGGUAUCCGUUAUCAACUUGGCACCGAGUCGG*m |
| | | UGC | | U*mG*mC |
| N20- | 653 | N₂₀GUUUUAGAGCUAGAAAUAGCAAGUUAAAAU | 753 | (mN*)₃N₁₇GUUUUAGAGCUAGAAAUAGCAAGUUAAAA |
| G015683 | | AAGGUUCCGUUAUCAACUUGGCACCGAGUCGGU | | UAAGGUUCCGUUAUCAACUUGGCACCGAGUCGG*mU* |
| | | GC | | mG*mC |
| N20- | 654 | N₂₀GUUUUAGAGCUAGAAAUAGCAAGUUAAAAU | 754 | (mN*)₃N₁₇GUUUUAGAGCUAGAAAUAGCAAGUUAAAA |
| G015684 | | AAGGAUCCGUUAUCAACUUGGCACCGAGUCGGU | | UAAGGAUCCGUUAUCAACUUGGCACCGAGUCGG*mU* |
| | | GC | | mG*mC |
| N20- | 655 | N₂₀GUUUUCGAGCUAGAAAUAGCAAGUGAAAAU | 755 | (mN*)₃N₁₇GUUUUCGAGCUAGAAAUAGCAAGUGAAAAU |
| G015685 | | AAGGCUAGUCCGUUAUCAACUUGGCACCGAGUC | | AAGGCUAGUCCGUUAUCAACUUGGCACCGAGUCGG* |
| | | GGUGC | | mU*mG*mC |
| N20- | 656 | N₂₀GUUUUUGAGCUAGAAAUAGCAAGUAAAAAU | 756 | (mN*)₃N₁₇GUUUUUGAGCUAGAAAUAGCAAGUAAAAA |
| G015686 | | AAGGCUAGUCCGUUAUCAACUUGGCACCGAGUC | | UAAGGCUAGUCCGUUAUCAACUUGGCACCGAGUCGG |
| | | GGUGC | | *mU*mG*mC |
| N20- | 657 | N₂₀GUUUUAGAGCGAGAAAUCGCAAGUUAAAAU | 757 | (mN*)₃N₁₇GUUUUAGAGCGAGAAAUCGCAAGUUAAAAU |
| G015687 | | AAGGCUAGUCCGUUAUCAACUUGGCACCGAGUC | | AAGGCUAGUCCGUUAUCAACUUGGCACCGAGUCGG* |
| | | GGUGC | | mU*mG*mC |
| N20- | 658 | N₂₀GUUUUAGAGCUAGAAAUAGCGAGUUAAAAU | 758 | (mN*)₃N₁₇GUUUUAGAGCUAGAAAUAGCGAGUUAAAA |
| G015688 | | AAGGCUAGUCCGUUAUCAACUUGGCACCGAGUC | | UAAGGCUAGUCCGUUAUCAACUUGGCACCGAGUCGG |
| | | GGUGC | | *mU*mG*mC |
| N20- | 659 | N₂₀GUUUUAGAGCUAGAAAUAGCCGGUUAAAAU | 759 | (mN*)₃N₁₇GUUUUAGAGCUAGAAAUAGCCGGUUAAAAU |
| G015689 | | AAGGCUAGUCCGUUAUCAACUUGGCACCGAGUC | | AAGGCUAGUCCGUUAUCAACUUGGCACCGAGUCGG* |
| | | GGUGC | | mU*mG*mC |
| N20- | 660 | N₂₀GUUUUAGAGCUAGAAAUAGCAAGUUAAAAU | 760 | (mN*)₃N₁₇GUUUUAGAGCUAGAAAUAGCAAGUUAAAA |
| G015690 | | GAGGCUAGUCCGUUAUCAACUUGGCACCGAGUC | | UGAGGCUAGUCCGUUAUCAACUUGGCACCGAGUCGG |
| | | GGUGC | | *mU*mG*mC |
| N20- | 661 | N₂₀GUUUUAGAGCUAGAAAUAGCAAGUUAAAAU | 761 | (mN*)₃N₁₇GUUUUAGAGCUAGAAAUAGCAAGUUAAAA |
| G015691 | | AAGGCUGGUCCGUUAUCAACUUGGCACCGAGUC | | UAAGGCUGGUCCGUUAUCAACUUGGCACCGAGUCGG |
| | | GGUGC | | *mU*mG*mC |
| N20- | 662 | N₂₀GUUUUAGAGCUAGAAAUAGCAAGUUAAAAU | 762 | (mN*)₃N₁₇GUUUUAGAGCUAGAAAUAGCAAGUUAAAA |
| G015692 | | AAGGCUCGUCCGUUAUCAACUUGGCACCGAGUC | | UAAGGCUCGUCCGUUAUCAACUUGGCACCGAGUCGG |
| | | GGUGC | | *mU*mG*mC |
| N20- | 663 | N₂₀GUUUUAGAGCUAGAAAUAGCAAGUUAAAAU | 763 | (mN*)₃N₁₇GUUUUAGAGCUAGAAAUAGCAAGUUAAAA |
| G015693 | | AAGGCUUGUCCGUUAUCAACUUGGCACCGAGUC | | UAAGGCUUGUCCGUUAUCAACUUGGCACCGAGUCGG |
| | | GGUGC | | *mU*mG*mC |
| N20- | 664 | N₂₀GUUUUAGAGCUAGAAAUAGCAAGUUAAAAU | 764 | (mN*)₃N₁₇GUUUUAGAGCUAGAAAUAGCAAGUUAAAA |
| G015694 | | AAGGCUAGUCCGUUGUCAACUUGGCACCGAGUC | | UAAGGCUAGUCCGUUGUCAACUUGGCACCGAGUCGG |
| | | GGUGC | | *mU*mG*mC |
| N20- | 665 | N₂₀GUUUUAGAGCUAGAAAUAGCAAGUUAAAAU | 765 | (mN*)₃N₁₇GUUUUAGAGCUAGAAAUAGCAAGUUAAAA |
| G015695 | | AAGGCUAGUCCGUUCUCAACUUGGCACCGAGUC | | UAAGGCUAGUCCGUUCUCAACUUGGCACCGAGUCGG |
| | | GGUGC | | *mU*mG*mC |
| N20- | 666 | N₂₀GUUUUAGAGCUAGAAAUAGCAAGUUAAAAU | 766 | (mN*)₃N₁₇GUUUUAGAGCUAGAAAUAGCAAGUUAAAA |
| G015696 | | AAGGCUAGUCCGUUUUCAACUUGGCACCGAGUC | | UAAGGCUAGUCCGUUUUCAACUUGGCACCGAGUCGG |
| | | GGUGC | | *mU*mG*mC |
| N20- | 667 | N₂₀GUUUUAGAGCUAGAAAUAGCAAGUUAAAAU | 767 | (mN*)₃N₁₇GUUUUAGAGCUAGAAAUAGCAAGUUAAAA |
| G015697 | | AAGGCUAGUCCGUUAUGAACUUGGCACCGAGUC | | UAAGGCUAGUCCGUUAUGAACUUGGCACCGAGUCGG |
| | | GGUGC | | *mU*mG*mC |
| N20- | 668 | N₂₀GUUUUAGAGCUAGAAAUAGCAAGUUAAAAU | 768 | (mN*)₃N₁₇GUUUUAGAGCUAGAAAUAGCAAGUUAAAA |
| G015698 | | AAGGCUAGUCCGUUAUCAACUUGGGACCGAGUC | | UAAGGCUAGUCCGUUAUCAACUUGGGACCGAGUCGG |
| | | GGUCC | | *mU*mC*mC |
| N20- | 669 | N₂₀GUUUUAGAGCUAGAAAUAGCAAGUUAAAAU | 769 | (mN*)₃N₁₇GUUUUAGAGCUAGAAAUAGCAAGUUAAAA |
| G015699 | | AAGGCUAGUCCGUUAUCAACUUGGAACCGAGUC | | UAAGGCUAGUCCGUUAUCAACUUGGAACCGAGUCGG |
| | | GGUUC | | *mU*mU*mC |
| N20- | 670 | N₂₀GUUUUCGAGCGAGAAAUCGCGAGUGAAAAU | 770 | (mN*)₃N₁₇GUUUUCGAGCGAGAAAUCGCGAGUGAAAAU |
| G015700 | | GAGGCUGGUCCGUUGUGAACUUGGAACCGAGUC | | GAGGCUGGUCCGUUGUGAACUUGGAACCGAGUCGG* |
| | | GGUUC | | mU*mU*mC |
| N20- | 671 | N₂₀GUUUUUGAGCGAGAAAUCGCAAGUAAAAAU | 771 | (mN*)₃N₁₇GUUUUUGAGCGAGAAAUCGCAAGUAAAAAU |
| G015701 | | AAGGCUCGUCCGUUCUGAACUUGGAACCGAGUC | | AAGGCUCGUCCGUUCUGAACUUGGAACCGAGUCGG* |
| | | GGUUC | | mU*mU*mC |
| N20- | 672 | N₂₀GUUUCGGAGCCGGAAACGGCGAGUCGAAAUG | 772 | (mN*)₃N₁₇GUUUCGGAGCCGGAAACGGCGAGUCGAAAU |
| G015702 | | AGGCUGGUCCGUUGUCGGCUCGGAACCGAGUCG | | GAGGCUGGUCCGUUGUCGGCUCGGAACCGAGUCGG* |
| | | GUUC | | mU*mU*mC |
| N20- | 673 | N₂₀GUUUUAGAGCUAGAAAUAGCAAGUUAAAAU | 773 | mC*mU*mC*ACUGAAAAGUGAGUCUGGAGAGCUGCAG |
| G015703 | | AAGGCUAGUCCGUUAUCAACUUGGCACCGAGUC | | UUUUAGAGCUAGAAAUAGCAAGUUAAAAUAAGGCUA |
| | | GGUGC | | GUCCGUUAUCAACUUGGCACCGAGUCGG*mU*mG*mC |
| N20- | 674 | N₂₀GUUUUAGAGCUAGAAAUAGCAAGUUAAAAU | 774 | mC*mA*mC*UGAAAAGUGAGUCUGGAGAGCUGCAGU |
| G015704 | | AAGGCUAGUCCGUUAUCAACUUGGCACCGAGUC | | UUUAGAGCUAGAAAUAGCAAGUUAAAAUAAGGCUAG |
| | | GGUGC | | UCCGUUAUCAACUUGGCACCGAGUCGG*mU*mG*mC |
| N20- | 675 | N₂₀GUUUUAGAGCUAGAAAUAGCAAGUUAAAAU | 775 | mC*mU*mG*AAAAGUGAGUCUGGAGAGCUGCAGUUU |
| G015705 | | AAGGCUAGUCCGUUAUCAACUUGGCACCGAGUC | | UAGAGCUAGAAAUAGCAAGUUAAAAUAAGGCUAGUC |
| | | GGUGC | | CGUUAUCAACUUGGCACCGAGUCGG*mU*mG*mC |
| N20- | 676 | N₂₀GUUUUAGAGCUAGAAAUAGCAAGUUAAAAU | 776 | mU*mC*mU*GGAGAGCUGCAGUUUUAGAGCUAGAAA |
| G015706 | | AAGGCUAGUCCGUUAUCAACUUGGCACCGAGUC | | UAGCAAGUUAAAAUAAGGCUAGUCCGUUAUCAACUU |
| | | GGUGC | | GGCACCGAGUCGG*mU*mG*mC |
| N20- | 677 | N₂₀GUUUUAGAGCUAGAAAUAGCAAGUUAAAAU | 777 | mA*mG*mU*CUGGAGAGCUGCAGUUUUAGAGCUAGA |
| G015707 | | AAGGCUAGUCCGUUAUCAACUUGGCACCGAGUC | | AAUAGCAAGUUAAAAUAAGGCUAGUCCGUUAUCAAC |
| | | GGUGC | | UUGGCACCGAGUCGG*mU*mG*mC |
| N20- | 678 | N₂₀GUUUUAGAGCUAGAAAUAGCAAGUUAAAAU | 778 | mU*mG*mA*GUCUGGAGAGCUGCAGUUUUAGAGCUA |
| G015708 | | AAGGCUAGUCCGUUAUCAACUUGGCACCGAGUC | | GAAAUAGCAAGUUAAAAUAAGGCUAGUCCGUUAUCA |
| | | GGUGC | | ACUUGGCACCGAGUCGG*mU*mG*mC |
| N20- | 679 | N₂₀GUUUUAGAGCUAGAAAUAGCAAGUUAAAAU | 779 | mG*mA*mA*AAGUGAGUCUGGAGAGCUGCAGUUUUA |
| G015709 | | AAGGCUAGUCCGUUAUCAACUUGGCACCGAGUC | | GAGCUAGAAAUAGCAAGUUAAAAUAAGGCUAGUCCG |
| | | GGUGC | | UUAUCAACUUGGCACCGAGUCGG*mU*mG*mC |
| N20- | 680 | N₂₀GUUUUAGAGCUAGAAAUAGCAAGUUAAAAU | 780 | (mN*)₃N₁₇GUUUUAGAGCUAGAAAUAGCAAGUUAAAA |
| G017275 | | AAGGCUAGUCCGUUAUCAACUUGGCACCGAGUC | | UAAGGCUAGUCCGUUAUCAACUUGGCACCGAGUCGG |
| | | GGUGC | | *mU*mG*mC |
| N20- | 681 | N₂₀GUUUUAGAGCUAGAAAUAGCAAGUUAAAAU | 781 | (mN*)N₁₇GUUUUAGAmGmCmUmAmGmAmAmAmUmAm |
| G017276 | | AAGGCUAGUCCGUUAUCACGAAAGGGCACCGAG | | GmCAAGUUAAAAUAAGGCUAGUCCGUUAUCACGAAA |
| | | UCGGUGC | | GGGCACCGAGUCGG*mU*mG*mC |
| N20- | 682 | N₂₀GUUUUAGAGCUAGAAAUAGCAAGUUAAAAU | 782 | (mN*)N₁₇GUUUUAGAmGmCmUmAmGmAmAmAmUmAm |
| G017277 | | AAGGCUAGUCCGUUAUCAAAAAUGGCACCGAGU | | GmCAAGUUAAAAUAAGGCUAGUCCGUUAUCAAAAAU |
| | | CGGUGC | | GGCACCGAGUCGG*mU*mG*mC |
| N20- | 683 | N₂₀GUUUUAGAGCUAGAAAUAGCAAGUUAAAAU | 783 | (mN*)N₁₇GUUUUAGAmGmCmUmAmGmAmAmAmUmAm |
| G017278 | | AAGGCUAGUCCGUUAUCACAAGGGCACCGAGUC | | GmCAAGUUAAAAUAAGGCUAGUCCGUUAUCACAAGG |
| | | GGUGC | | GCACCGAGUCGG*mU*mG*mC |
| N20- | 684 | N₂₀GUUUUAGAGCUAGAAAUAGCAAGUUAAAAU | 784 | (mN*)N₁₇GUUUUAGAmGmCmUmAmGmAmAmAmUmAm |
| G017279 | | AAGGCUAGUCCGUUAUCAAAAUGGCACCGAGUC | | GmCAAGUUAAAAUAAGGCUAGUCCGUUAUCAAAAUG |
| | | GGUGC | | GCACCGAGUCGG*mU*mG*mC |
| N20- | 685 | N₂₀GUUUUAGAGCGCGAAGCGCAAGUUAAAAUA | 785 | (mN*)₃N₁₇GUUUUAGAGCGCGAAGCGCAAGUUAAAAUA |
| G017280 | | AGGCUAGUCCGUUAUCAAAAUGGCACCGAGUCG | | AGGCUAGUCCGUUAUCAAAAUGGCACCGAGUCGG*m |
| | | GUGC | | U*mG*mC |
| N20- | 686 | N₂₀GUUUUAGAGCUGAAAAGCAAGUUAAAAUAA | 786 | (mN*)₃N₁₇GUUUUAGAGCUGAAAAGCAAGUUAAAAUA |
| G017281 | | GGCUAGUCCGUUAUCAAAAUGGCACCGAGUCGG | | AGGCUAGUCCGUUAUCAAAAUGGCACCGAGUCGG*m |
| | | UGC | | U*mG*mC |
| N20- | 687 | N₂₀GUUUUAGAGCGAAAGCAAGUUAAAAUAAGG | 787 | (mN*)₃N₁₇GUUUUAGAGCGAAAGCAAGUUAAAAUAAG |
| G017282 | | CUAGUCCGUUAUCAAAAUGGCACCGAGUCGGUG | | GCUAGUCCGUUAUCAAAAUGGCACCGAGUCGG*mU* |
| | | C | | mG*mC |
| N20- | 688 | N₂₀GUUUUAGAGCAAAGCAAGUUAAAAUAAGGC | 788 | (mN*)₃N₁₇GUUUUAGAGCAAAGCAAGUUAAAAUAAGGC |
| G017283 | | UAGUCCGUUAUCAAAAUGGCACCGAGUCGGUGC | | UAGUCCGUUAUCAAAAUGGCACCGAGUCGG*mU*mG* |
| | | | | mC |
| N20- | 689 | N₂₀GUUUUAGAGCGAAAGCAAGUUAAAAUAAGG | 789 | (mN*)₃N₁₇GUUUUAGAmGmCmGmAmAmAmGmCAAGUU |
| G013773 | | CUAGUCCGUUAUCAACUUGGCACCGAGUCGGUG | | AAAAUAAGGCUAGUCCGUUAUCAACUUGGCACCGAG |
| | | C | | UCGG*mU*mG*mC |
| N20- | 690 | N₂₀GUUUUAGAGCGAAAGCAAGUUAAAAUAAGG | 790 | (mN*)₃N₁₇GUUUUAGAmGmCmGmAmAmAmGmCAAGUU |
| G013776 | | CUAGUCCGUUAUCAAGAAAUGGCACCGAGUCGG | | AAAAUAAGGCUAGUCCGUUAUCAAGAAAUGGCACCG |
| | | UGC | | AGUCGG*mU*mG*mC |
| N20- | 691 | N₃NₓGUUUUAGAGCUAGAAAUAGCAAGUUAAAA | 791 | (mN*)N₁₇GUUUUAGAmGmCmUmAmGmAmAmAmUmAm |
| SMO7-6 | | UAAGGCUAGUCCGUUAUCACGAAAGGGCACCGA | | GmCAAGUUAAAAUAAGGCUAGUCCGUUAUCAmCmGm |
| | | GUCGGUGC | | AmAmAmGmGmGmCmAmCmCmGmAmGmUmCmGmG*m |
| | | | | U*mG*mC |
| N20- | 692 | N₃NₓGUUUUAGAGCUAGAAAUAGCAAGUUAAAA | 792 | (mN*)N₁₇GUUUUAGAmGmCmUmAmGmAmAmAmUmAm |
| SM12-6 | | UAAGGCUAGUCCGUUAUCAAAAUGGCACCGAGU | | GmCAAGUUAAAAUAAGGCUAGUCCGUUAUCAmAmA |
| | | CGGUGC | | mAmUmGmGmCmAmCmCmGmAmGmUmCmGmG*mU*m |
| | | | | G*mC |
| N20- | 693 | N₃NₓGUUUUAGAGCUAGAAAUAGCAAGUUAAAA | 793 | (mN*)₃N₁₇GUUUUAGAmGmCmUmAmGmAmAmAmUmAm |
| SM07-50 | | UAAGGCUAGUCCGUUAUCACGAAAGGGCACCGA | | GmCAAGUUAAAAUAAGGCUAGUCCGUUAUCACmGmA |
| | | GUCGGUGC | | mAmAmGmGmGmCmAmCmCmGmAmGmUmCmGmG*mU |
| | | | | *mG*mC |
| N20- | 694 | N₃NₓGUUUUAGAGCUAGAAAUAGCAAGUUAAAA | 794 | (mN*)₃N₁₇GUUUUAGAmGmCmUmAmGmAmAmAmUmAm |
| SM12-50 | | UAAGGCUAGUCCGUUAUCAAAAUGGCACCGAGU | | GmCAAGUUAAAAUAAGGCUAGUCCGUUAUCAAmAmA |
| | | CGGUGC | | mUmGmGmCmAmCmCmGmAmGmUmCmGmG*mU*mG* |
| | | | | mC |
| SM07-6 | 695 | ACACAAAUACCAGUCCAGCGGUUUUAGAGCUAG | 795 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCAmCmGmAmAmAmGmGmGmCmA |
| | | | | mCmCmGmAmGmUmCmGmG*mU*mG*mC |
| SM12-6 | 696 | ACACAAAUACCAGUCCAGCGGUUUUAGAGCUAG | 796 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAUGGCACCGAGUCGGUGC | | GCUAGUCCGUUAUCAmAmAmAmUmGmGmCmAmCmC |
| | | | | mGmAmGmUmCmGmG*mU*mG*mC |
| SM07-50 | 697 | ACACAAAUACCAGUCCAGCGGUUUUAGAGCUAG | 797 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACmGmAmAmAmGmGmGmCmAm |
| | | | | CmCmGmAmGmUmCmGmG*mU*mG*mC |
| SM12-50 | 698 | ACACAAAUACCAGUCCAGCGGUUUUAGAGCUAG | 798 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAUGGCACCGAGUCGGUGC | | GCUAGUCCGUUAUCAAmAmAmUmGmGmCmAmCmCm |
| | | | | GmAmGmUmCmGmG*mU*mG*mC |
| | 699- | Not Used | 799- | Not Used |
| | 700 | | 800 | |
| G000390 | 801 | GCCGAGUCUGGAGAGCUGCAGUUUUAGAGCUA | 901 | mG*mC*mC*GAGUCUGGAGAGCUGCAGUUUUAGAmG |
| | | GAAAUAGCAAGUUAAAAUAAGGCUAGUCCGUU | | mCmUmAmGmAmAmAmUmAmGmCAAGUUAAAAUAAG |
| | | AUCAACUUGAAAAAGUGGCACCGAGUCGGUGCU | | GCUAGUCCGUUAUCAmAmCmUmUmGmAmAmAmAmA |
| | | UUU | | mGmUmGmGmCmAmCmCmGmAmGmUmCmGmGmUmG |
| | | | | mCmU*mU*mU*mU |
| G000531 | 802 | UCACAGGACCACUCACCCCAGUUUUAGAGCUAG | 902 | mU*mC*mA*CAGGACCACUCACCCCAGUUUUAGAmGm |
| | | AAAUAGCAAGUUAAAAUAAGGCUAGUCCGUUA | | CmUmAmGmAmAmAmUmAmGmCAAGUUAAAAUAAGG |
| | | UCAACUUGAAAAAGUGGCACCGAGUCGGUGCUU | | CUAGUCCGUUAUCAmAmCmUmUmGmAmAmAmAmAm |
| | | UU | | GmUmGmGmCmAmCmCmGmAmGmUmCmGmGmUmGm |
| | | | | CmU*mU*mU*mU |
| G000532 | 803 | UGCUCUGUAAGCUUACCCAGGUUUUAGAGCUAG | 903 | mU*mG*mC*UCUGUAAGCUUACCCAGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGAAAAAGUGGCACCGAGUCGGUGCUU | | GCUAGUCCGUUAUCAmAmCmUmUmGmAmAmAmAmA |
| | | UU | | mGmUmGmGmCmAmCmCmGmAmGmUmCmGmGmUmG |
| | | | | mCmU*mU*mU*mU |
| G000533 | 804 | GGACACCAAAUCGUACUGGAGUUUUAGAGCUA | 904 | mG*mG*mA*CACCAAAUCGUACUGGAGUUUUAGAmG |
| | | GAAAUAGCAAGUUAAAAUAAGGCUAGUCCGUU | | mCmUmAmGmAmAmAmUmAmGmCAAGUUAAAAUAAG |
| | | AUCAACUUGAAAAAGUGGCACCGAGUCGGUGCU | | GCUAGUCCGUUAUCAmAmCmUmUmGmAmAmAmAmA |
| | | UUU | | mGmUmGmGmCmAmCmCmGmAmGmUmCmGmGmUmG |
| | | | | mCmU*mU*mU*mU |
| G000534 | 805 | ACGCAAAUAUCAGUCCAGCGGUUUUAGAGCUAG | 905 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGAAAAAGUGGCACCGAGUCGGUGCUU | | GCUAGUCCGUUAUCAmAmCmUmUmGmAmAmAmAmA |
| | | UU | | mGmUmGmGmCmAmCmCmGmAmGmUmCmGmGmUmG |
| | | | | mCmU*mU*mU*mU |
| G000535 | 806 | AAAGUCCUGGAUGCUGUCCGGUUUUAGAGCUA | 906 | mA*mA*mA*GUCCUGGAUGCUGUCCGGUUUUAGAmG |
| | | GAAAUAGCAAGUUAAAAUAAGGCUAGUCCGUU | | mCmUmAmGmAmAmAmUmAmGmCAAGUUAAAAUAAG |
| | | AUCAACUUGAAAAAGUGGCACCGAGUCGGUGCU | | GCUAGUCCGUUAUCAmAmCmUmUmGmAmAmAmAmA |
| | | UUU | | mGmUmGmGmCmAmCmCmGmAmGmUmCmGmGmUmG |
| | | | | mCmU*mU*mU*mU |
| G000536 | 807 | AACUGGACACCAAAUCGUACGUUUUAGAGCUAG | 907 | mA*mA*mC*UGGACACCAAAUCGUACGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGAAAAAGUGGCACCGAGUCGGUGCUU | | GCUAGUCCGUUAUCAmAmCmUmUmGmAmAmAmAmA |
| | | UU | | mGmUmGmGmCmAmCmCmGmAmGmUmCmGmGmUmG |
| | | | | mCmU*mU*mU*mU |
| G000694 | 808 | ACGCAAAUAUCAGUCCAGCGGUUUUAGAGCUAG | 908 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGGCACCGAGUCGGUGC | | GCUAGUCCGUUAUCAACUUGGCACCGAGUCGG*mU*m |
| | | | | G*mC |
| G018631 | 809 | ACGCAAAUAUCAGUCCAGCGGUUUUAGAGCUAG | 909 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G018632 | 810 | ACGCAAAUAUCAGUCCAGCGGUUUUAGAGCUAG | 910 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAUGGCACCGAGUCGGUGC | | GCUAGUCCGUUAUCAAAAUGGCACCGAGUCGG*mU* |
| | | | | mG*mC |
| G018633 | 811 | ACGCAAAUAUCAGUCCAGCGGUUUUAGAGCUAG | 911 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCAmCmGmAmAmAmGmGmGmCmA |
| | | | | mCmCmGmAmGmUmCmGmG*mU*mG*mC |
| G018634 | 812 | ACGCAAAUAUCAGUCCAGCGGUUUUAGAGCUAG | 912 | mA*mC*mG*CAAAUAUCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAAAAUGGCACCGAGUCGGUGC | | GCUAGUCCGUUAUCAmAmAmAmUmGmGmCmAmCmC |
| | | | | mGmAmGmUmCmGmG*mU*mG*mC |
| G018635 | 813 | GCCGAGUCUGGAGAGCUGCAGUUUUAGAGCUA | 913 | mG*mC*mC*GAGUCUGGAGAGCUGCAGUUUUAGAmG |
| | | GAAAUAGCAAGUUAAAAUAAGGCUAGUCCGUU | | mCmUmAmGmAmAmAmUmAmGmCAAGUUAAAAUAAG |
| | | AUCACGAAAGGGCACCGAGUCGGUGC | | GCUAGUCCGUUAUCACGAAAGGGCACCGAGUCGG*m |
| | | | | U*mG*mC |
| G018639 | 814 | UGCUCUGUAAGCUUACCCAGGUUUUAGAGCUAG | 914 | mU*mG*mC*UCUGUAAGCUUACCCAGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G018643 | 815 | GCCGAGUCUGGAGAGCUGCAGUUUUAGAGCUA | 915 | mG*mC*mC*GAGUCUGGAGAGCUGCAGUUUUAGAmG |
| | | GAAAUAGCAAGUUAAAAUAAGGCUAGUCCGUU | | mCmUmAmGmAmAmAmUmAmGmCAAGUUAAAAUAAG |
| | | AUCAACUUGGCACCGAGUCGGUGC | | GCUAGUCCGUUAUCAACUUGGCACCGAGUCGG*mU*m |
| | | | | G*mC |
| G018644 | 816 | UGCUCUGUAAGCUUACCCAGGUUUUAGAGCUAG | 916 | mU*mG*mC*UCUGUAAGCUUACCCAGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCAACUUGGCACCGAGUCGGUGC | | GCUAGUCCGUUAUCAACUUGGCACCGAGUCGG*mU*m |
| | | | | G*mC |
| G018804 | 817 | ACACAAAUACCAGUCCAGCGGUUUUAGAGCUAG | 917 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCAmCmGmAmAmAmGmGmGmCmA |
| | | | | mCmCmGmAmGmUmCmGmG*mU*mG*mC |
| G018805 | 818 | ACACAAAUACCAGUCCAGCGGUUUUAGAGCUAG | 918 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACmGmAmAmAmGmGmGmCmAm |
| | | | | CmCmGmAmGmUmCmGmG*mU*mG*mC |
| G019874 | 819 | ACACAAAUACCAGUCCAGCGGUUUUAGAGCUAG | 919 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCAACUUGAAAAAGUGGCACCGAGUCGGUGCUU | | AU*AAG*G*C*UmAGUCmCGUUA*UCA*A*mCmUmUm |
| | | UU | | GmAmAmAmAmAmGmUmGmGmCmAmCmCmGmAmGm |
| | | | | UmCmGmGmUmGmCmU*mU*mU*mU |
| G019875 | 820 | ACACAAAUACCAGUCCAGCGGUUUUAGAGCUAG | 920 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCAACUUGGCACCGAGUCGGUGC | | AU*AAG*G*C*UmAGUCmCGUUA*UCA*A*mCmUmUm |
| | | | | GmGmCmAmCmCmGmAmGmUmCmGmG*mU*mG*mC |
| G019876 | 821 | ACACAAAUACCAGUCCAGCGGUUUUAGAGCUAG | 921 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACmGmAmAmAmGmGGCACCGAG |
| | | | | UCGG*mU*mG*mC |
| G019877 | 822 | ACACAAAUACCAGUCCAGCGGUUUUAGAGCUAG | 922 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGmGmCmAmCmCmGm |
| | | | | AmGmUmCmGmG*mU*mG*mC |
| G019878 | 823 | ACACAAAUACCAGUCCAGCGGUUUUAGAGCUAG | 923 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCACGAAAGGGCACCGAGUCGGUGC | | AU*AAG*G*C*UmAGUCmCGUUA*UCA*C*GAAAGGGC |
| | | | | ACCGAGUCGG*mU*mG*mC |
| G019879 | 824 | ACACAAAUACCAGUCCAGCGGUUUUAGAGCUAG | 924 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCACGAAAGGGCACCGAGUCGGUGC | | AU*AAG*G*C*UmAGUCmCGUUA*UCA*C*mGmAmAm |
| | | | | AmGmGmGmCmAmCmCmGmAmGmUmCmGmG*mU*mG |
| | | | | *mC |
| G019880 | 825 | ACACAAAUACCAGUCCAGCGGUUUUAGAGCUAG | 925 | mA*mC*ACAA*AUACC*AGUCCAGCGGUUUUAGAmGm |
| | | AAAUAGCAAGUUAAAAUAAGGCUAGUCCGUUA | | CmUmAmGmAmAmAmUmAmGmCAAGUUAAAAUAAG* |
| | | UCACGAAAGGGCACCGAGUCGGUGC | | G*C*UmAGUCmCGUUA*UCA*C*mGmAmAmAmGmGmG |
| | | | | mCmAmCmCmGmAmGmUmCmGmG*mU*mG*mC |
| G019881 | 826 | ACACAAAUACCAGUCCAGCGGUUUUAGAGCUAG | 926 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCACGAAAGGGCACCGAGUCGGUGC | | AU*AAGGCUAGUCCGUUAUCA*C*mGmAmAmAmGmG |
| | | | | mGmCmAmCmCmGmAmGmUmCmGmG*mU*mG*mC |
| G019882 | 827 | ACACAAAUACCAGUCCAGCGGUUUUAGAGCUAG | 927 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCACGAAAGGGCACCGAGUCGGUGC | | AU*AAG*G*C*UmAGUCmCGUUA*UCACmGmAmAmAm |
| | | | | GmGmGmCmAmCmCmGmAmGmUmCmGmG*mU*mG*m |
| | | | | C |
| G019883 | 828 | ACACAAAUACCAGUCCAGCGGUUUUAGAGCUAG | 928 | mA*mC*ACAAAUACCAGUCCAGCGGUUUUAGAmGmC |
| | | AAAUAGCAAGUUAAAAUAAGGCUAGUCCGUUA | | mUmAmGmAmAmAmUmAmGmCAAGUUAAAAUAAGGC |
| | | UCACGAAAGGGCACCGAGUCGGUGC | | UAGUCCGUUAUCACGAAAGGGCACCGAGUCGG*mU* |
| | | | | mG*mC |
| G019884 | 829 | ACACAAAUACCAGUCCAGCGGUUUUAGAGCUAG | 929 | mA*mC*mA*CAA*AUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G019885 | 830 | ACACAAAUACCAGUCCAGCGGUUUUAGAGCUAG | 930 | mA*mC*mA*CAAAUACC*AGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G019886 | 831 | ACACAAAUACCAGUCCAGCGGUUUUAGAGCUAG | 931 | mA*mC*mA*CAAAUACCAGUCCAGCGmGUUUUAGAm |
| | | AAAUAGCAAGUUAAAAUAAGGCUAGUCCGUUA | | GmCmUmAmGmAmAmAmUmAmGmCAAGUUAAAAUAA |
| | | UCACGAAAGGGCACCGAGUCGGUGC | | GGCUAGUCCGUUAUCACGAAAGGGCACCGAGUCGG* |
| | | | | mU*mG*mC |
| G019887 | 832 | ACACAAAUACCAGUCCAGCGGUUUUAGAGCUAG | 932 | mA*mC*mA*CAAAUACCAGUCCAGCGGU*UUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G019888 | 833 | ACACAAAUACCAGUCCAGCGGUUUUAGAGCUAG | 933 | mA*mC*mA*CAAAUACCAGUCCAGCGGUUUUAG*AmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G019889 | 834 | ACACAAAUACCAGUCCAGCGGUUUUAGAGCUAG | 934 | mA*mC*mA*CAAAUACCAGUCCAGCGGUUUUAGA*mG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGGCACCGAGUCGG*m |
| | | | | U*mG*mC |
| G019890 | 835 | ACACAAAUACCAGUCCAGCGGUUUUAGAGCUAG | 935 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCmAAGUUAAAAUAA |
| | | UCACGAAAGGGCACCGAGUCGGUGC | | GGCUAGUCCGUUAUCACGAAAGGGCACCGAGUCGG* |
| | | | | mU*mG*mC |
| G019891 | 836 | ACACAAAUACCAGUCCAGCGGUUUUAGAGCUAG | 936 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAA*GUUAAAAUAA |
| | | UCACGAAAGGGCACCGAGUCGGUGC | | GGCUAGUCCGUUAUCACGAAAGGGCACCGAGUCGG* |
| | | | | mU*mG*mC |
| G019892 | 837 | ACACAAAUACCAGUCCAGCGGUUUUAGAGCUAG | 937 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAG*UUAAAAUAA |
| | | UCACGAAAGGGCACCGAGUCGGUGC | | GGCUAGUCCGUUAUCACGAAAGGGCACCGAGUCGG* |
| | | | | mU*mG*mC |
| G019893 | 838 | ACACAAAUACCAGUCCAGCGGUUUUAGAGCUAG | 938 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAU*AA |
| | | UCACGAAAGGGCACCGAGUCGGUGC | | GGCUAGUCCGUUAUCACGAAAGGGCACCGAGUCGG* |
| | | | | mU*mG*mC |
| G019894 | 839 | ACACAAAUACCAGUCCAGCGGUUUUAGAGCUAG | 939 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | *GCUAGUCCGUUAUCACGAAAGGGCACCGAGUCGG*m |
| | | | | U*mG*mC |
| G019895 | 840 | ACACAAAUACCAGUCCAGCGGUUUUAGAGCUAG | 940 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | G*CUAGUCCGUUAUCACGAAAGGGCACCGAGUCGG*m |
| | | | | U*mG*mC |
| G019896 | 841 | ACACAAAUACCAGUCCAGCGGUUUUAGAGCUAG | 941 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GC*UAGUCCGUUAUCACGAAAGGGCACCGAGUCGG*m |
| | | | | U*mG*mC |
| G019897 | 842 | ACACAAAUACCAGUCCAGCGGUUUUAGAGCUAG | 942 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUmAGUCCGUUAUCACGAAAGGGCACCGAGUCGG* |
| | | | | mU*mG*mC |
| G019898 | 843 | ACACAAAUACCAGUCCAGCGGUUUUAGAGCUAG | 943 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCmCGUUAUCACGAAAGGGCACCGAGUCGG* |
| | | | | mU*mG*mC |
| G019899 | 844 | ACACAAAUACCAGUCCAGCGGUUUUAGAGCUAG | 944 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUA*UCACGAAAGGGCACCGAGUCGG*m |
| | | | | U*mG*mC |
| G019900 | 845 | ACACAAAUACCAGUCCAGCGGUUUUAGAGCUAG | 945 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCA*CGAAAGGGCACCGAGUCGG*m |
| | | | | U*mG*mC |
| G019901 | 846 | ACACAAAUACCAGUCCAGCGGUUUUAGAGCUAG | 946 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCAC*GAAAGGGCACCGAGUCGG*m |
| | | | | U*mG*mC |
| G019902 | 847 | ACACAAAUACCAGUCCAGCGGUUUUAGAGCUAG | 947 | mA*mC*ACAA*AUACC*AGUCCAGCGGUUUUAGAmGm |
| | | AAAUAGCAAGUUAAAAUAAGGCUAGUCCGUUA | | CmUmAmGmAmAmAmUmAmGmCAAGUUAAAAUAAGG |
| | | UCACGAAAGGGCACCGAGUCGGUGC | | CUAGUCCGUUAUCACGAAAGGGCACCGAGUCGG*mU* |
| | | | | mG*mC |
| G019903 | 848 | ACACAAAUACCAGUCCAGCGGUUUUAGAGCUAG | 948 | mA*mC*mA*CAAAUACCAGUCCAGCGmGU*UUUAG*A |
| | | AAAUAGCAAGUUAAAAUAAGGCUAGUCCGUUA | | *mGmCmUmAmGmAmAmAmUmAmGmCmAA*G*UUAA |
| | | UCACGAAAGGGCACCGAGUCGGUGC | | AAU*AAGGCUAGUCCGUUAUCACGAAAGGGCACCGA |
| | | | | GUCGG*mU*mG*mC |
| G019904 | 849 | ACACAAAUACCAGUCCAGCGGUUUUAGAGCUAG | 949 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | *G*C*UmAGUCmCGUUA*UCACGAAAGGGCACCGAGU |
| | | | | CGG*mU*mG*mC |
| G019905 | 850 | ACACAAAUACCAGUCCAGCGGUUUUAGAGCUAG | 950 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCA*C*GAAAGGGCACCGAGUCGG* |
| | | | | mU*mG*mC |
| G019906 | 851 | ACACAAAUACCAGUCCAGCGGUUUUAGAGCUAG | 951 | mA*mC*mA*CAAAUACCAGUCCAGCGGUUUUCGAmGm |
| | | AAAUAGCAAGUUAAAAUAAGGCUAGUCCGUUA | | CmUmAmGmAmAmAmUmAmGmCAAGUGAAAAUAAGG |
| | | UCACGAAAGGGCACCGAGUCGGUGC | | CUAGUCCGUUAUCACGAAAGGGCACCGAGUCGG*mU* |
| | | | | mG*mC |
| G019907 | 852 | ACACAAAUACCAGUCCAGCGGUUUUCGAGCUAG | 952 | mA*mC*mA*CAAAUACCAGUCCAGCGGUUUUAGAmG |
| | | AAAUAGCAAGUGAAAAUAAGGCUAGUCCGUUA | | mCmUmAmGmAmAmAmUmAmGmCAAGUUAAAAUGAG |
| | | UCACGAAAGGGCACCGAGUCGGUGC | | GCUAGUCCGUUAUCACGAAAGGGCACCGAGUCGG*m |
| | | | | U*mG*mC |
| G019908 | 853 | ACACAAAUACCAGUCCAGCGGUUUUAGAGCUAG | 953 | mA*mC*ACAA*AUACC*AGUCCAGCGmGU*UUUAG*A* |
| | | AAAUAGCAAGUUAAAAUGAGGCUAGUCCGUUA | | mGmCmUmAmGmAmAmAmUmAmGmCmAA*G*UUAAA |
| | | UCACGAAAGGGCACCGAGUCGGUGC | | AU*GAG*G*C*UmAGUCmCGUUA*UCA*C*mGmAmAm |
| | | | | AmGmGmGmCmAmCmCmGmAmGmUmCmGmG*mU*mG |
| | | | | *mC |
| G019909 | 854 | ACACAAAUACCAGUCCAGCGGUUUUAGAGCUAG | 954 | mA*mC*mA*CAAAUACCAGUCCAGCGGUUUUAGAmG |
| | | AAAUAGCAAGUUAAAAUGAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUGAUCACGAAAGGGCACCGAGUCGG*m |
| | | | | U*mG*mC |
| G019910 | 855 | ACACAAAUACCAGUCCAGCGGUUUUAGAGCUAG | 955 | mA*mC*ACAA*AUACC*AGUCCAGCGmGU*UUUAG*A* |
| | | AAAUAGCAAGUUAAAAUAAGGCUAGUCCGUGA | | mGmCmUmAmGmAmAmAmUmAmGmCmAA*G*UUAAA |
| | | UCACGAAAGGGCACCGAGUCGGUGC | | AU*AAG*G*C*UmAGUCmCGUGA*UCA*C*mGmAmAm |
| | | | | AmGmGmGmCmAmCmCmGmAmGmUmCmGmG*mU*mG |
| | | | | *mC |
| G019911 | 856 | ACACAAAUACCAGUCCAGCGGUUUUAGAGCUAG | 956 | mA*mC*mA*CAAAUACCAGUCCAGCGGUUUUAGAmG |
| | | AAAUAGCAAGUUAAAAUAAGGCUAGUCCGUGA | | mCmUmAmGmAmAmAmUmAmGmCAAGUUAAAAUAAG |
| | | UCACGAAAGGGCACCGAGUCGGUGC | | GCUAGUCCGUUAUCCCGAAAGGGCACCGAGUCGG*m |
| | | | | U*mG*mC |
| G019912 | 857 | ACACAAAUACCAGUCCAGCGGUUUUAGAGCUAG | 957 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCCCGAAAGGGCACCGAGUCGGUGC | | AU*AAG*G*C*UmAGUCmCGUUA*UCC*C*mGmAmAmA |
| | | | | mGmGmGmCmAmCmCmGmAmGmUmCmGmG*mU*mG* |
| | | | | mC |
| G019913 | 858 | ACACAAAUACCAGUCCAGCGGUUUUAGAGCUAG | 958 | mA*mC*mA*CAAAUACCAGUCCAGCGGUUUUCGAmGm |
| | | AAAUAGCAAGUUAAAAUAAGGCUAGUCCGUUA | | CmUmAmGmAmAmAmUmAmGmCAAGUGAAAAUGAGG |
| | | UCCCGAAAGGGCACCGAGUCGGUGC | | CUAGUCCGUGAUCCCGAAAGGGCACCGAGUCGG*mU* |
| | | | | mG*mC |
| G019914 | 859 | ACACAAAUACCAGUCCAGCGGUUUUCGAGCUAG | 959 | mA*mC*ACAA*AUACC*AGUCCAGCGmGU*UUUCG*A* |
| | | AAAUAGCAAGUGAAAAUGAGGCUAGUCCGUGA | | mGmCmUmAmGmAmAmAmUmAmGmCmAA*G*UGAAA |
| | | UCCCGAAAGGGCACCGAGUCGGUGC | | AU*GAG*G*C*UmAGUCmCGUGA*UCC*C*mGmAmAmA |
| | | | | mGmGmGmCmAmCmCmGmAmGmUmCmGmG*mU*mG* |
| | | | | mC |
| G019915 | 860 | ACACAAAUACCAGUCCAGCGGUUUUCGAGCUAG | 960 | mA*mC*ACAA*AUACC*AGUCCAGCGmGU*UUU*AG*A |
| | | AAAUAGCAAGUGAAAAUGAGGCUAGUCCGUGA | | *mGmCmUmAmGmAmAmAmUmAmGmCmAA*G*UU*AA |
| | | UCCCGAAAGGGCACCGAGUCGGUGC | | AAU*AAG*G*C*UmAGUCmCGUU*A*UC*A*C*mGmAm |
| | | | | AmAmGmGmGmCmAmCmCmGmAmGmUmCmGmG*mU* |
| | | | | mG*mC |
| G019916 | 861 | ACACAAAUACCAGUCCAGCGGUUUUAGAGCUAG | 961 | mA*mC*ACAA*AUACC*AGUCCAGCGmGU*UUU*AG*A |
| | | AAAUAGCAAGUUAAAAUAAGGCUAGUCCGUUA | | *mGmCmUmAmGmAmAmAmUmAmGmCmAA*G*UU*AA |
| | | UCACGAAAGGGCACCGAGUCGGUGC | | AAU*AAG*G*C*UmAGUCmCGUU*A*UC*A*C*mGmAm |
| | | | | AmAmGmGmGmCmAmCmCmGmAmGmUmCmGmG*mU* |
| | | | | mG*mC |
| G020022 | 862 | ACACAAAUACCAGUCCAGCGGUUUUAGAGCUAG | 962 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACmGmAmAmAmGGGCACCGAGU |
| | | | | CGG*mU*mG*mC |
| G020023 | 863 | ACACAAAUACCAGUCCAGCGGUUUUAGAGCUAG | 963 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGmGmCmAmCmCmGfAf |
| | | | | GfUfCmGmG*mU*mG*mC |
| G020024 | 864 | ACACAAAUACCAGUCCAGCGGUUUUAGAGCUAG | 964 | mA*mC*mA*CAAAUACCAGUCCAGCGGUUUUAGAmG |
| | | AAAUAGCAAGUUAAAAUAAGGCUAGUCCGUUA | | mCmUmAmGmAmAmAmUmAmGmCAAGUUAAAAUAAG |
| | | UCACGAAAGGGCACCGAGUCGGUGC | | GCUAGUCCGUUAUCACGAAAGGmGmCmAmCmCmGAG |
| | | | | UCmGmG*mU*mG*mC |
| G020025 | 865 | ACACAAAUACCAGUCCAGCGGUUUUAGAGCUAG | 965 | mA*mC*mACAAAUACCAGUCCAGCGmGUUUUAGAmG |
| | | AAAUAGCAAGUUAAAAUAAGGCUAGUCCGUUA | | mCmUmAmGmAmAmAmUmAmGmCmAAGUUAAAAUAA |
| | | UCACGAAAGGGCACCGAGUCGGUGC | | GGCUmAGUCmCGUUAUCACmGmAmAmAmGG*mGmC |
| | | | | mAmCmCmGAGUCmGmG*mU*mG*mC |
| G020026 | 866 | ACACAAAUACCAGUCCAGCGGUUUUAGAGCUAG | 966 | mA*mC*fAfCAfA*AfUfAfCfC*AfGUCCfAfGCfGm |
| | | AAAUAGCAAGUUAAAAUAAGGCUAGUCCGUUA | | GU*UUUAG*fA*mGmCmUmAmGmAmAmAmUmAmGmCmA |
| | | UCACGAAAGGGCACCGAGUCGGUGC | | fA*G*UUfAAfAAfU*fAfAfG*fG*fC*fUmAGUCmC |
| | | | | fGUUA*fUfCA*C*fG*mAmAmAmGfG*mGmCmAmCmC |
| | | | | mGfA*fG*fUfCmGmGmU*mG*mC |
| G020027 | 867 | ACACAAAUACCAGUCCAGCGGUUUUAGAGCUAG | 967 | mA*mC*ACAA*AUACC*AGUCCAGCGmGU*UUUAG*A* |
| | | AAAUAGCAAGUUAAAAUAAGGCUAGUCCGUUA | | mGmCmUmAmGmAmAmAmUmAmGmCmAA*G*UUAAA |
| | | UCACGAAAGGGCACCGAGUCGGUGC | | AU*AAG*G*C*UmAGUCmCGUUA*UCA*C*G*mAmAmA |
| | | | | mGG*mGmCmAmCmCmGA*G*UCmGmGmU*mG*mC |
| G020028 | 868 | ACACAAAUACCAGUCCAGCGGUUUUAGAGCUAG | 968 | mA*mC*fAfCAfAAfUfAfCfCAfGUCCfAfGCfGmG |
| | | AAAUAGCAAGUUAAAAUAAGGCUAGUCCGUUA | | U*UUUAG*fAmGmCmUmAmGmAmAmAmUmAmGmCmAfA |
| | | UCACGAAAGGGCACCGAGUCGGUGC | | G*UUfAAfAAfUfAfAfGfGfCfUmAGUCmCfGUUA* |
| | | | | fUfCA*C*fGmAmAmAmGfGmGmCmAmCmCmGfAfGfU |
| | | | | fCmGmGmU*mG*mC |
| G020029 | 869 | ACACAAAUACCAGUCCAGCGGUUUUAGAGCUAG | 969 | mA*mC*ACAAAUACCAGUCCAGCGmGU*UUUAG*fA*m |
| | | AAAUAGCAAGUUAAAAUAAGGCUAGUCCGUGA | | GmCmUmAmGmAmAmAmUmAmGmCmAfA*G*UUfAAfA |
| | | UCACGAAAGGGCACCGAGUCGGUGC | | AfU*fAfAfG*fG*fC*fUmAGUCmCfGUGA*fUfCA* |
| | | | | C*fG*mAmAmAmGfG*mGmCmAmCmCmGfA*fG*fUfC |
| | | | | mGmGmU*mG*mC |
| G020030 | 870 | ACACAAAUACCAGUCCAGCGGUUUUAGAGCUAG | 970 | mA*mC*fAfCAfA*AfUfAfCfC*AfGUCCfAfGCfGm |
| | | AAAUAGCAAGUUAAAAUAAGGCUAGUCCGUUA | | GU*UUUfAfG*fA*mGmCmUmAmGmAmAmAmUmAmGmC |
| | | UCACGAAAGGGCACCGAGUCGGUGC | | mAfA*fG*fUfUfAAfAfAfU*fAfAfG*fG*fC*fUm |
| | | | | AGfUCmCfGfUUfA*fUfCfA*fC*fG*mAmAmAmGf |
| | | | | G*mGmCmAmCmCmGfA*fG*fUfCmGmGmU*mG*mC |
| G020349 | 871 | ACACAAAUACCAGUCCAGCGGUUUUAGAGCUAG | 971 | mA*mC*mA*mCAA*A*fU*fA*fC*fCAfGfUCC*fAf |
| | | AAAUAGCAAGUUAAAAUAAGGCUAGUCCGUUA | | GCGmGUUUfUAGmAmGmCmUmAmGmAmAmAmUmAmGmC |
| | | UCACGAAAGGGCACCGAGUCGGUGC | | mAmAGUfUmAfAmAfAmUAmAmGmGmCmUmAGUmCmCG |
| | | | | UfUAmUmCAmCmGmAmAmAmGmGmGmCmAmCmCmG |
| | | | | mAmGmUmCmGmG*mU*mG*mC |
| G020350 | 872 | ACACAAAUACCAGUCCAGCGGUUUUAGAGCUAG | 972 | mA*mC*mA*mCAA*A*fU*fA*fC*fCAfGfUCC*fA |
| | | AAAUAGCAAGUUAAAAUAAGGCUAGUCCGUUA | | fGCGGUUUUAGAmGmCmUmAmGmAmAmAmUmAmGmCAA |
| | | UCACGAAAGGGCACCGAGUCGGUGC | | GUUAAAAUAAGGCUAGUCCGUUAUCACGAAAGGGCACC |
| | | | | GAGUCGG*mU*mG*mC |
| G020351 | 873 | ACACAAAUACCAGUCCAGCGGUUUUAGAGCUAG | 973 | mA*mC*mA*mCAAAfU*ACfCAfGUCC*AGCGmGUUUf |
| | | AAAUAGCAAGUUAAAAUAAGGCUAGUCCGUUA | | UAGmAmGmCmUmAmGmAmAmAmUmAmGmCmAmAGUf |
| | | UCACGAAAGGGCACCGAGUCGGUGC | | UAAAAmUAAGGCmUAGUCCGUfUAUmCACGAAAGGm |
| | | | | GmCmAmCmCmGmAmGmUmCmGmG*mU*mG*mC |
| G020352 | 874 | ACACAAAUACCAGUCCAGCGGUUUUAGAGCUAG | 974 | mA*mC*mA*mCAAAfU*ACfCAfGUCC*AGCGmGUUUU |
| | | AAAUAGCAAGUUAAAAUAAGGCUAGUCCGUUA | | AmGmAmGCmUAmGmAmAmAmUAmGCmAmAGUfUAAA |
| | | UCACGAAAGGGCACCGAGUCGGUGC | | AmUAAGGCmUAGUCCGUfUA*mUmCA*CmGmAmAmA |
| | | | | mGmGGmCAmCCGfAfGfUCmGG*mU*mG*mC |
| G020353 | 875 | ACACAAAUACCAGUCCAGCGGUUUUAGAGCUAG | 975 | mA*mC*mA*CAAAUACCAGUCCAGCGGUUUUAGAmGC |
| | | AAAUAGCAAGUUAAAAUAAGGCUAGUCCGUUA | | mUAmGmAmAmAmUAmGCAAGUUAAAAUAAGGCUAG |
| | | UCACGAAAGGGCACCGAGUCGGUGC | | UCCGUUAUCACGAAAGGGmCAmCCGfAfGfUCmGG*mU |
| | | | | *mG*mC |
| | 876- | Not Used | 976- | Not Used |
| | 900 | | 1000 | |

In Table 1A, (N)ₓ represents x contiguous nucleotides, where x is 5, 6, 7, 8, 9, 10, 11, 12, 13, 14, 15, 16, 17, 18, 19, 20, 21, 22, 23, 24, or 25. (mN*)₃ represents three consecutive nucleotides each having any base, a 2’-OMe, and a 3’ PS linkage to the next nucleotide. N₁₇ and N₂₀ represent 17 and 20 consecutive N (any nucleotide), respectively. “C-” appended to a Guide ID indicates the conserved portion of an sgRNA.
Nucleotide modifications are indicated in Table 1A as follows: m: 2’-OMe; *: PS linkage; f: 2’-fluoro; (invd): inverted abasic; moe: 2’-moe; e: ENA; d: deoxyribonucleotide (also note that T is always a deoxyribonucleotide); x: UNA. Thus, for example, mA represents 2’-O-methyl adenosine; xA represents a UNA nucleotide with an adenine nucleobase; eA represents an ENA nucleotide with an adenine nucleobase; and dA represents an adenosine deoxyribonucleotide.

TABLE 1B
(Table of RNA-Guided DNA Binding Agent Sequences):

| SEQ ID NO | Name | Sequence |
|---|---|---|
| 1099 | Cas9 mRNA | GGGUCCCGCAGUCGGCGUCCAGCGGCUCUGCUUGUUCGUGUGUGUGUCGUUGCAGGCCUUAUUCGGAUC |
| | sequence | CGCCACCAUGGACAAGAAGUACAGCAUCGGACUGGACAUCGGAACAAACAGCGUCGGAUGGGCAGUCAU |
| | | CACAGACGAAUACAAGGUCCCGAGCAAGAAGUUCAAGGUCCUGGGAAACACAGACAGACACAGCAUCAA |
| | | GAAGAACCUGAUCGGAGCACUGCUGUUCGACAGCGGAGAAACAGCAGAAGCAACAAGACUGAAGAGAAC |
| | | AGCAAGAAGAAGAUACACAAGAAGAAAGAACAGAAUCUGCUACCUGCAGGAAAUCUUCAGCAACGAAAU |
| | | GGCAAAGGUCGACGACAGCUUCUUCCACAGACUGGAAGAAAGCUUCCUGGUCGAAGAAGACAAGAAGCA |
| | | CGAAAGACACCCGAUCUUCGGAAACAUCGUCGACGAAGUCGCAUACCACGAAAAGUACCCGACAAUCUAC |
| | | CACCUGAGAAAGAAGCUGGUCGACAGCACAGACAAGGCAGACCUGAGACUGAUCUACCUGGCACUGGCA |
| | | CACAUGAUCAAGUUCAGAGGACACUUCCUGAUCGAAGGAGACCUGAACCCGGACAACAGCGACGUCGAC |
| | | AAGCUGUUCAUCCAGCUGGUCCAGACAUACAACCAGCUGUUCGAAGAAAACCCGAUCAACGCAAGCGGA |
| | | GUCGACGCAAAGGCAAUCCUGAGCGCAAGACUGAGCAAGAGCAGAAGACUGGAAAACCUGAUCGCACAG |
| | | CUGCCGGGAGAAAAGAAGAACGGACUGUUCGGAAACCUGAUCGCACUGAGCCUGGGACUGACACCGAAC |
| | | UUCAAGAGCAACUUCGACCUGGCAGAAGACGCAAAGCUGCAGCUGAGCAAGGACACAUACGACGACGAC |
| | | CUGGACAACCUGCUGGCACAGAUCGGAGACCAGUACGCAGACCUGUUCCUGGCAGCAAAGAACCUGAGC |
| | | GACGCAAUCCUGCUGAGCGACAUCCUGAGAGUCAACACAGAAAUCACAAAGGCACCGCUGAGCGCAAGC |
| | | AUGAUCAAGAGAUACGACGAACACCACCAGGACCUGACACUGCUGAAGGCACUGGUCAGACAGCAGCUG |
| | | CCGGAAAAGUACAAGGAAAUCUUCUUCGACCAGAGCAAGAACGGAUACGCAGGAUACAUCGACGGAGGA |
| | | GCAAGCCAGGAAGAAUUCUACAAGUUCAUCAAGCCGAUCCUGGAAAAGAUGGACGGAACAGAAGAACUG |
| | | CUGGUCAAGCUGAACAGAGAAGACCUGCUGAGAAAGCAGAGAACAUUCGACAACGGAAGCAUCCCGCAC |
| | | CAGAUCCACCUGGGAGAACUGCACGCAAUCCUGAGAAGACAGGAAGACUUCUACCCGUUCCUGAAGGAC |
| | | AACAGAGAAAAGAUCGAAAAGAUCCUGACAUUCAGAAUCCCGUACUACGUCGGACCGCUGGCAAGAGGA |
| | | AACAGCAGAUUCGCAUGGAUGACAAGAAAGAGCGAAGAAACAAUCACACCGUGGAACUUCGAAGAAGUC |
| | | GUCGACAAGGGAGCAAGCGCACAGAGCUUCAUCGAAAGAAUGACAAACUUCGACAAGAACCUGCCGAAC |
| | | GAAAAGGUCCUGCCGAAGCACAGCCUGCUGUACGAAUACUUCACAGUCUACAACGAACUGACAAAGGUC |
| | | AAGUACGUCACAGAAGGAAUGAGAAAGCCGGCAUUCCUGAGCGGAGAACAGAAGAAGGCAAUCGUCGAC |
| | | CUGCUGUUCAAGACAAACAGAAAGGUCACAGUCAAGCAGCUGAAGGAAGACUACUUCAAGAAGAUCGAA |
| | | UGCUUCGACAGCGUCGAAAUCAGCGGAGUCGAAGACAGAUUCAACGCAAGCCUGGGAACAUACCACGAC |
| | | CUGCUGAAGAUCAUCAAGGACAAGGACUUCCUGGACAACGAAGAAAACGAAGACAUCCUGGAAGACAUC |
| | | GUCCUGACACUGACACUGUUCGAAGACAGAGAAAUGAUCGAAGAAAGACUGAAGACAUACGCACACCUG |
| | | UUCGACGACAAGGUCAUGAAGCAGCUGAAGAGAAGAAGAUACACAGGAUGGGGAAGACUGAGCAGAAAG |
| | | CUGAUCAACGGAAUCAGAGACAAGCAGAGCGGAAAGACAAUCCUGGACUUCCUGAAGAGCGACGGAUUC |
| | | GCAAACAGAAACUUCAUGCAGCUGAUCCACGACGACAGCCUGACAUUCAAGGAAGACAUCCAGAAGGCA |
| | | CAGGUCAGCGGACAGGGAGACAGCCUGCACGAACACAUCGCAAACCUGGCAGGAAGCCCGGCAAUCAAG |
| | | AAGGGAAUCCUGCAGACAGUCAAGGUCGUCGACGAACUGGUCAAGGUCAUGGGAAGACACAAGCCGGAA |
| | | AACAUCGUCAUCGAAAUGGCAAGAGAAAACCAGACAACACAGAAGGGACAGAAGAACAGCAGAGAAAGA |
| | | AUGAAGAGAAUCGAAGAAGGAAUCAAGGAACUGGGAAGCCAGAUCCUGAAGGAACACCCGGUCGAAAAC |
| | | ACACAGCUGCAGAACGAAAAGCUGUACCUGUACUACCUGCAGAACGGAAGAGACAUGUACGUCGACCAG |
| | | GAACUGGACAUCAACAGACUGAGCGACUACGACGUCGACCACAUCGUCCCGCAGAGCUUCCUGAAGGACG |
| | | ACAGCAUCGACAACAAGGUCCUGACAAGAAGCGACAAGAACAGAGGAAAGAGCGACAACGUCCCGAGCG |
| | | AAGAAGUCGUCAAGAAGAUGAAGAACUACUGGAGACAGCUGCUGAACGCAAAGCUGAUCACACAGAGAA |
| | | AGUUCGACAACCUGACAAAGGCAGAGAGAGGAGGACUGAGCGAACUGGACAAGGCAGGAUUCAUCAAGA |
| | | GACAGCUGGUCGAAACAAGACAGAUCACAAAGCACGUCGCACAGAUCCUGGACAGCAGAAUGAACACAA |
| | | AGUACGACGAAAACGACAAGCUGAUCAGAGAAGUCAAGGUCAUCACACUGAAGAGCAAGCUGGUCAGCG |
| | | ACUUCAGAAAGGACUUCCAGUUCUACAAGGUCAGAGAAAUCAACAACUACCACCACGCACACGACGCAU |
| | | ACCUGAACGCAGUCGUCGGAACAGCACUGAUCAAGAAGUACCCGAAGCUGGAAAGCGAAUUCGUCUACG |
| | | GAGACUACAAGGUCUACGACGUCAGAAAGAUGAUCGCAAAGAGCGAACAGGAAAUCGGAAAGGCAACAG |
| | | CAAAGUACUUCUUCUACAGCAACAUCAUGAACUUCUUCAAGACAGAAAUCACACUGGCAAACGGAGAAA |
| | | UCAGAAAGAGACCGCUGAUCGAAACAAACGGAGAAACAGGAGAAAUCGUCUGGGACAAGGGAAGAGACU |
| | | UCGCAACAGUCAGAAAGGUCCUGAGCAUGCCGCAGGUCAACAUCGUCAAGAAGACAGAAGUCCAGACAG |
| | | GAGGAUUCAGCAAGGAAAGCAUCCUGCCGAAGAGAAACAGCGACAAGCUGAUCGCAAGAAAGAAGGACU |
| | | GGGACCCGAAGAAGUACGGAGGAUUCGACAGCCCGACAGUCGCAUACAGCGUCCUGGUCGUCGCAAAGG |
| | | UCGAAAAGGGAAAGAGCAAGAAGCUGAAGAGCGUCAAGGAACUGCUGGGAAUCACAAUCAUGGAAAGAA |
| | | GCAGCUUCGAAAAGAACCCGAUCGACUUCCUGGAAGCAAAGGGAUACAAGGAAGUCAAGAAGGACCUGA |
| | | UCAUCAAGCUGCCGAAGUACAGCCUGUUCGAACUGGAAAACGGAAGAAAGAGAAUGCUGGCAAGCGCAG |
| | | GAGAACUGCAGAAGGGAAACGAACUGGCACUGCCGAGCAAGUACGUCAACUUCCUGUACCUGGCAAGCC |
| | | ACUACGAAAAGCUGAAGGGAAGCCCGGAAGACAACGAACAGAAGCAGCUGUUCGUCGAACAGCACAAGC |
| | | ACUACCUGGACGAAAUCAUCGAACAGAUCAGCGAAUUCAGCAAGAGAGUCAUCCUGGCAGACGCAAACC |
| | | UGGACAAGGUCCUGAGCGCAUACAACAAGCACAGAGACAAGCCGAUCAGAGAACAGGCAGAAAACAUCA |
| | | UCCACCUGUUCACACUGACAAACCUGGGAGCACCGGCAGCAUUCAAGUACUUCGACACAACAAUCGACAG |
| | | AAAGAGAUACACAAGCACAAAGGAAGUCCUGGACGCAACACUGAUCCACCAGAGCAUCACAGGACUGUA |
| | | CGAAACAAGAAUCGACCUGAGCCAGCUGGGAGGAGACGGAGGAGGAAGCCCGAAGAAGAAGAGAAAGGU |
| | | CUAGCUAGCCAUCACAUUUAAAAGCAUCUCAGCCUACCAUGAGAAUAAGAGAAAGAAAAUGAAGAUCAA |
| | | UAGCUUAUUCAUCUCUUUUUCUUUUUCGUUGGUGUAAAGCCAACACCCUGUCUAAAAAACAUAAAUUUC |
| | | UUUAAUCAUUUUGCCUCUUUUCUCUGUGCUUCAAUUAAUAAAAAAUGGAAAGAACCUCGAGAAAAAAAA |
| | | AAAAAAAAAAAAAAAAAAAAAAAAAAAAAAAAAAAAAAAAAAAAAAAAAAAAAAAAAAAAAAAAAAAA |
| | | AAAAAAAAAAAAAA |
| 1100 | Cas9 mRNA sequence | GGGUCCCGCAGUCGGCGUCCAGCGGCUCUGCUUGUUCGUGUGUGUGUCGUUGCAGGCCUUAUUCGGAUC |
| | | CAUGGAUAAGAAGUACUCAAUCGGGCUGGAUAUCGGAACUAAUUCCGUGGGUUGGGCAGUGAUCACGGA |
| | | UGAAUACAAAGUGCCGUCCAAGAAGUUCAAGGUCCUGGGGAACACCGAUAGACACAGCAUCAAGAAAAA |
| | | UCUCAUCGGAGCCCUGCUGUUUGACUCCGGCGAAACCGCAGAAGCGACCCGGCUCAAACGUACCGCGAGG |
| | | CGACGCUACACCCGGCGGAAGAAUCGCAUCUGCUAUCUGCAAGAGAUCUUUUCGAACGAAAUGGCAAAG |
| | | GUCGACGACAGCUUCUUCCACCGCCUGGAAGAAUCUUUCCUGGUGGAGGAGGACAAGAAGCAUGAACGG |
| | | CAUCCUAUCUUUGGAAACAUCGUCGACGAAGUGGCGUACCACGAAAAGUACCCGACCAUCUACCAUCUGC |
| | | GGAAGAAGUUGGUUGACUCAACUGACAAGGCCGACCUCAGAUUGAUCUACUUGGCCCUCGCCCAUAUGA |
| | | UCAAAUUCCGCGGACACUUCCUGAUCGAAGGCGAUCUGAACCCUGAUAACUCCGACGUGGAUAAGCUUU |
| | | UCAUUCAACUGGUGCAGACCUACAACCAACUGUUCGAAGAAAACCCAAUCAAUGCUAGCGGCGUCGAUG |
| | | CCAAGGCCAUCCUGUCCGCCCGGCUGUCGAAGUCGCGGCGCCUCGAAAACCUGAUCGCACAGCUGCCGGG |
| | | AGAGAAAAAGAACGGACUUUUCGGCAACUUGAUCGCUCUCUCACUGGGACUCACUCCCAAUUUCAAGUC |
| | | CAAUUUUGACCUGGCCGAGGACGCGAAGCUGCAACUCUCAAAGGACACCUACGACGACGACUUGGACAA |
| | | UUUGCUGGCACAAAUUGGCGAUCAGUACGCGGAUCUGUUCCUUGCCGCUAAGAACCUUUCGGACGCAAU |
| | | CUUGCUGUCCGAUAUCCUGCGCGUGAACACCGAAAUAACCAAAGCGCCGCUUAGCGCCUCGAUGAUUAA |
| | | GCGGUACGACGAGCAUCACCAGGAUCUCACGCUGCUCAAAGCGCUCGUGAGACAGCAACUGCCUGAAAA |
| | | GUACAAGGAGAUCUUCUUCGACCAGUCCAAGAAUGGGUACGCAGGGUACAUCGAUGGAGGCGCUAGCCA |
| | | GGAAGAGUUCUAUAAGUUCAUCAAGCCAAUCCUGGAAAAGAUGGACGGAACCGAAGAACUGCUGGUCAA |
| | | GCUGAACAGGGAGGAUCUGCUCCGGAAACAGAGAACCUUUGACAACGGAUCCAUUCCCCACCAGAUCCA |
| | | UCUGGGUGAGCUGCACGCCAUCUUGCGGCGCCAGGAGGACUUUUACCCAUUCCUCAAGGACAACCGGGA |
| | | AAAGAUCGAGAAAAUUCUGACGUUCCGCAUCCCGUAUUACGUGGGCCCACUGGCGCGCGGCAAUUCGCG |
| | | CUUCGCGUGGAUGACUAGAAAAUCAGAGGAAACCAUCACUCCUUGGAAUUUCGAGGAAGUUGUGGAUAA |
| | | GGGAGCUUCGGCACAAAGCUUCAUCGAACGAAUGACCAACUUCGACAAGAAUCUCCCAAACGAGAAGGU |
| | | GCUUCCUAAGCACAGCCUCCUUUACGAAUACUUCACUGUCUACAACGAACUGACUAAAGUGAAAUACGU |
| | | UACUGAAGGAAUGAGGAAGCCGGCCUUUCUGUCCGGAGAACAGAAGAAAGCAAUUGUCGAUCUGCUGUU |
| | | CAAGACCAACCGCAAGGUGACCGUCAAGCAGCUUAAAGAGGACUACUUCAAGAAGAUCGAGUGUUUCGA |
| | | CUCAGUGGAAAUCAGCGGGGUGGAGGACAGAUUCAACGCUUCGCUGGGAACCUAUCAUGAUCUCCUGAA |
| | | GAUCAUCAAGGACAAGGACUUCCUUGACAACGAGGAGAACGAGGACAUCCUGGAAGAUAUCGUCCUGAC |
| | | CUUGACCCUUUUCGAGGAUCGCGAGAUGAUCGAGGAGAGGCUUAAGACCUACGCUCAUCUCUUCGACGA |
| | | UAAGGUCAUGAAACAACUCAAGCGCCGCCGGUACACUGGUUGGGGCCGCCUCUCCCGCAAGCUGAUCAAC |
| | | GGUAUUCGCGAUAAACAGAGCGGUAAAACUAUCCUGGAUUUCCUCAAAUCGGAUGGCUUCGCUAAUCGU |
| | | AACUUCAUGCAAUUGAUCCACGACGACAGCCUGACCUUUAAGGAGGACAUCCAAAAAGCACAAGUGUCC |
| | | GGACAGGGAGACUCACUCCAUGAACACAUCGCGAAUCUGGCCGGUUCGCCGGCGAUUAAGAAGGGAAUU |
| | | CUGCAAACUGUGAAGGUGGUCGACGAGCUGGUGAAGGUCAUGGGACGGCACAAACCGGAGAAUAUCGUG |
| | | AUUGAAAUGGCCCGAGAAAACCAGACUACCCAGAAGGGCCAGAAAAACUCCCGCGAAAGGAUGAAGCGG |
| | | AUCGAAGAAGGAAUCAAGGAGCUGGGCAGCCAGAUCCUGAAAGAGCACCCGGUGGAAAACACGCAGCUG |
| | | CAGAACGAGAAGCUCUACCUGUACUAUUUGCAAAAUGGACGGGACAUGUACGUGGACCAAGAGCUGGAC |
| | | AUCAAUCGGUUGUCUGAUUACGACGUGGACCACAUCGUUCCACAGUCCUUUCUGAAGGAUGACUCGAUC |
| | | GAUAACAAGGUGUUGACUCGCAGCGACAAGAACAGAGGGAAGUCAGAUAAUGUGCCAUCGGAGGAGGUC |
| | | GUGAAGAAGAUGAAGAAUUACUGGCGGCAGCUCCUGAAUGCGAAGCUGAUUACCCAGAGAAAGUUUGAC |
| | | AAUCUCACUAAAGCCGAGCGCGGCGGACUCUCAGAGCUGGAUAAGGCUGGAUUCAUCAAACGGCAGCUG |
| | | GUCGAGACUCGGCAGAUUACCAAGCACGUGGCGCAGAUCUUGGACUCCCGCAUGAACACUAAAUACGAC |
| | | GAGAACGAUAAGCUCAUCCGGGAAGUGAAGGUGAUUACCCUGAAAAGCAAACUUGUGUCGGACUUUCGG |
| | | AAGGACUUUCAGUUUUACAAAGUGAGAGAAAUCAACAACUACCAUCACGCGCAUGACGCAUACCUCAAC |
| | | GCUGUGGUCGGUACCGCCCUGAUCAAAAAGUACCCUAAACUUGAAUCGGAGUUUGUGUACGGAGACUAC |
| | | AAGGUCUACGACGUGAGGAAGAUGAUAGCCAAGUCCGAACAGGAAAUCGGGAAAGCAACUGCGAAAUAC |
| | | UUCUUUUACUCAAACAUCAUGAACUUUUUCAAGACUGAAAUUACGCUGGCCAAUGGAGAAAUCAGGAAG |
| | | AGGCCACUGAUCGAAACUAACGGAGAAACGGGCGAAAUCGUGUGGGACAAGGGCAGGGACUUCGCAACU |
| | | GUUCGCAAAGUGCUCUCUAUGCCGCAAGUCAAUAUUGUGAAGAAAACCGAAGUGCAAACCGGCGGAUUU |
| | | UCAAAGGAAUCGAUCCUCCCAAAGAGAAAUAGCGACAAGCUCAUUGCACGCAAGAAAGACUGGGACCCG |
| | | AAGAAGUACGGAGGAUUCGAUUCGCCGACUGUCGCAUACUCCGUCCUCGUGGUGGCCAAGGUGGAGAAG |
| | | GGAAAGAGCAAAAAGCUCAAAUCCGUCAAAGAGCUGCUGGGGAUUACCAUCAUGGAACGAUCCUCGUUC |
| | | GAGAAGAACCCGAUUGAUUUCCUCGAGGCGAAGGGUUACAAGGAGGUGAAGAAGGAUCUGAUCAUCAAA |
| | | CUCCCCAAGUACUCACUGUUCGAACUGGAAAAUGGUCGGAAGCGCAUGCUGGCUUCGGCCGGAGAACUC |
| | | CAAAAAGGAAAUGAGCUGGCCUUGCCUAGCAAGUACGUCAACUUCCUCUAUCUUGCUUCGCACUACGAA |
| | | AAACUCAAAGGGUCACCGGAAGAUAACGAACAGAAGCAGCUUUUCGUGGAGCAGCACAAGCAUUAUCUG |
| | | GAUGAAAUCAUCGAACAAAUCUCCGAGUUUUCAAAGCGCGUGAUCCUCGCCGACGCCAACCUCGACAAA |
| | | GUCCUGUCGGCCUACAAUAAGCAUAGAGAUAAGCCGAUCAGAGAACAGGCCGAGAACAUUAUCCACUUG |
| | | UUCACCCUGACUAACCUGGGAGCCCCAGCCGCCUUCAAGUACUUCGAUACUACUAUCGAUCGCAAAAGAU |
| | | ACACGUCCACCAAGGAAGUUCUGGACGCGACCCUGAUCCACCAAAGCAUCACUGGACUCUACGAAACUAG |
| | | GAUCGAUCUGUCGCAGCUGGGUGGCGAUGGCGGUGGAUCUCCGAAAAAGAAGAGAAAGGUGUAAUGAGC |
| | | UAGCCAUCACAUUUAAAAGCAUCUCAGCCUACCAUGAGAAUAAGAGAAAGAAAAUGAAGAUCAAUAGCU |
| | | UAUUCAUCUCUUUUUCUUUUUCGUUGGUGUAAAGCCAACACCCUGUCUAAAAAACAUAAAUUUCUUUAA |
| | | UCAUUUUGCCUCUUUUCUCUGUGCUUCAAUUAAUAAAAAAUGGAAAGAACCUCGAGAAAAAAAAAAAAA |
| | | AAAAAAAAAAAAAAAAAAAAAAAAAAAAAAAAAAAAAAAAAAAAAAAAAAAAAAAAAAAAAAAAAAAA |
| | | AAAAAAAAA |
| 1101 | DNA coding | GGGTCCCGCAGTCGGCGTCCAGCGGCTCTGCTTGTTCGTGTGTGTGTCGTTGCAGGCCTTATTCGGATCCGCC |
| | sequence for | ACCATGGACAAGAAGTACAGCATCGGACTGGACATCGGAACAAACAGCGTCGGATGGGCAGTCATCACAGA |
| | Cas9 transcript | CGAATACAAGGTCCCGAGCAAGAAGTTCAAGGTCCTGGGAAACACAGACAGACACAGCATCAAGAAGAAC |
| | | CTGATCGGAGCACTGCTGTTCGACAGCGGAGAAACAGCAGAAGCAACAAGACTGAAGAGAACAGCAAGAA |
| | | GAAGATACACAAGAAGAAAGAACAGAATCTGCTACCTGCAGGAAATCTTCAGCAACGAAATGGCAAAGGTC |
| | | GACGACAGCTTCTTCCACAGACTGGAAGAAAGCTTCCTGGTCGAAGAAGACAAGAAGCACGAAAGACACCC |
| | | GATCTTCGGAAACATCGTCGACGAAGTCGCATACCACGAAAAGTACCCGACAATCTACCACCTGAGAAAGA |
| | | AGCTGGTCGACAGCACAGACAAGGCAGACCTGAGACTGATCTACCTGGCACTGGCACACATGATCAAGTTC |
| | | AGAGGACACTTCCTGATCGAAGGAGACCTGAACCCGGACAACAGCGACGTCGACAAGCTGTTCATCCAGCT |
| | | GGTCCAGACATACAACCAGCTGTTCGAAGAAAACCCGATCAACGCAAGCGGAGTCGACGCAAAGGCAATCC |
| | | TGAGCGCAAGACTGAGCAAGAGCAGAAGACTGGAAAACCTGATCGCACAGCTGCCGGGAGAAAAGAAGAA |
| | | CGGACTGTTCGGAAACCTGATCGCACTGAGCCTGGGACTGACACCGAACTTCAAGAGCAACTTCGACCTGGC |
| | | AGAAGACGCAAAGCTGCAGCTGAGCAAGGACACATACGACGACGACCTGGACAACCTGCTGGCACAGATC |
| | | GGAGACCAGTACGCAGACCTGTTCCTGGCAGCAAAGAACCTGAGCGACGCAATCCTGCTGAGCGACATCCT |
| | | GAGAGTCAACACAGAAATCACAAAGGCACCGCTGAGCGCAAGCATGATCAAGAGATACGACGAACACCAC |
| | | CAGGACCTGACACTGCTGAAGGCACTGGTCAGACAGCAGCTGCCGGAAAAGTACAAGGAAATCTTCTTCGA |
| | | CCAGAGCAAGAACGGATACGCAGGATACATCGACGGAGGAGCAAGCCAGGAAGAATTCTACAAGTTCATCA |
| | | AGCCGATCCTGGAAAAGATGGACGGAACAGAAGAACTGCTGGTCAAGCTGAACAGAGAAGACCTGCTGAG |
| | | AAAGCAGAGAACATTCGACAACGGAAGCATCCCGCACCAGATCCACCTGGGAGAACTGCACGCAATCCTGA |
| | | GAAGACAGGAAGACTTCTACCCGTTCCTGAAGGACAACAGAGAAAAGATCGAAAAGATCCTGACATTCAGA |
| | | ATCCCGTACTACGTCGGACCGCTGGCAAGAGGAAACAGCAGATTCGCATGGATGACAAGAAAGAGCGAAGA |
| | | AACAATCACACCGTGGAACTTCGAAGAAGTCGTCGACAAGGGAGCAAGCGCACAGAGCTTCATCGAAAGAA |
| | | TGACAAACTTCGACAAGAACCTGCCGAACGAAAAGGTCCTGCCGAAGCACAGCCTGCTGTACGAATACTTC |
| | | ACAGTCTACAACGAACTGACAAAGGTCAAGTACGTCACAGAAGGAATGAGAAAGCCGGCATTCCTGAGCGG |
| | | AGAACAGAAGAAGGCAATCGTCGACCTGCTGTTCAAGACAAACAGAAAGGTCACAGTCAAGCAGCTGAAG |
| | | GAAGACTACTTCAAGAAGATCGAATGCTTCGACAGCGTCGAAATCAGCGGAGTCGAAGACAGATTCAACGC |
| | | AAGCCTGGGAACATACCACGACCTGCTGAAGATCATCAAGGACAAGGACTTCCTGGACAACGAAGAAAACG |
| | | AAGACATCCTGGAAGACATCGTCCTGACACTGACACTGTTCGAAGACAGAGAAATGATCGAAGAAAGACTG |
| | | AAGACATACGCACACCTGTTCGACGACAAGGTCATGAAGCAGCTGAAGAGAAGAAGATACACAGGATGGG |
| | | GAAGACTGAGCAGAAAGCTGATCAACGGAATCAGAGACAAGCAGAGCGGAAAGACAATCCTGGACTTCCT |
| | | GAAGAGCGACGGATTCGCAAACAGAAACTTCATGCAGCTGATCCACGACGACAGCCTGACATTCAAGGAAG |
| | | ACATCCAGAAGGCACAGGTCAGCGGACAGGGAGACAGCCTGCACGAACACATCGCAAACCTGGCAGGAAG |
| | | CCCGGCAATCAAGAAGGGAATCCTGCAGACAGTCAAGGTCGTCGACGAACTGGTCAAGGTCATGGGAAGAC |
| | | ACAAGCCGGAAAACATCGTCATCGAAATGGCAAGAGAAAACCAGACAACACAGAAGGGACAGAAGAACAG |
| | | CAGAGAAAGAATGAAGAGAATCGAAGAAGGAATCAAGGAACTGGGAAGCCAGATCCTGAAGGAACACCCG |
| | | GTCGAAAACACACAGCTGCAGAACGAAAAGCTGTACCTGTACTACCTGCAGAACGGAAGAGACATGTACGT |
| | | CGACCAGGAACTGGACATCAACAGACTGAGCGACTACGACGTCGACCACATCGTCCCGCAGAGCTTCCTGA |
| | | AGGACGACAGCATCGACAACAAGGTCCTGACAAGAAGCGACAAGAACAGAGGAAAGAGCGACAACGTCCC |
| | | GAGCGAAGAAGTCGTCAAGAAGATGAAGAACTACTGGAGACAGCTGCTGAACGCAAAGCTGATCACACAG |
| | | AGAAAGTTCGACAACCTGACAAAGGCAGAGAGAGGAGGACTGAGCGAACTGGACAAGGCAGGATTCATCA |
| | | AGAGACAGCTGGTCGAAACAAGACAGATCACAAAGCACGTCGCACAGATCCTGGACAGCAGAATGAACAC |
| | | AAAGTACGACGAAAACGACAAGCTGATCAGAGAAGTCAAGGTCATCACACTGAAGAGCAAGCTGGTCAGC |
| | | GACTTCAGAAAGGACTTCCAGTTCTACAAGGTCAGAGAAATCAACAACTACCACCACGCACACGACGCATA |
| | | CCTGAACGCAGTCGTCGGAACAGCACTGATCAAGAAGTACCCGAAGCTGGAAAGCGAATTCGTCTACGGAG |
| | | ACTACAAGGTCTACGACGTCAGAAAGATGATCGCAAAGAGCGAACAGGAAATCGGAAAGGCAACAGCAAA |
| | | GTACTTCTTCTACAGCAACATCATGAACTTCTTCAAGACAGAAATCACACTGGCAAACGGAGAAATCAGAAA |
| | | GAGACCGCTGATCGAAACAAACGGAGAAACAGGAGAAATCGTCTGGGACAAGGGAAGAGACTTCGCAACA |
| | | GTCAGAAAGGTCCTGAGCATGCCGCAGGTCAACATCGTCAAGAAGACAGAAGTCCAGACAGGAGGATTCAG |
| | | CAAGGAAAGCATCCTGCCGAAGAGAAACAGCGACAAGCTGATCGCAAGAAAGAAGGACTGGGACCCGAAG |
| | | AAGTACGGAGGATTCGACAGCCCGACAGTCGCATACAGCGTCCTGGTCGTCGCAAAGGTCGAAAAGGGAAA |
| | | GAGCAAGAAGCTGAAGAGCGTCAAGGAACTGCTGGGAATCACAATCATGGAAAGAAGCAGCTTCGAAAAG |
| | | AACCCGATCGACTTCCTGGAAGCAAAGGGATACAAGGAAGTCAAGAAGGACCTGATCATCAAGCTGCCGAA |
| | | GTACAGCCTGTTCGAACTGGAAAACGGAAGAAAGAGAATGCTGGCAAGCGCAGGAGAACTGCAGAAGGGA |
| | | AACGAACTGGCACTGCCGAGCAAGTACGTCAACTTCCTGTACCTGGCAAGCCACTACGAAAAGCTGAAGGG |
| | | AAGCCCGGAAGACAACGAACAGAAGCAGCTGTTCGTCGAACAGCACAAGCACTACCTGGACGAAATCATCG |
| | | AACAGATCAGCGAATTCAGCAAGAGAGTCATCCTGGCAGACGCAAACCTGGACAAGGTCCTGAGCGCATAC |
| | | AACAAGCACAGAGACAAGCCGATCAGAGAACAGGCAGAAAACATCATCCACCTGTTCACACTGACAAACCT |
| | | GGGAGCACCGGCAGCATTCAAGTACTTCGACACAACAATCGACAGAAAGAGATACACAAGCACAAAGGAA |
| | | GTCCTGGACGCAACACTGATCCACCAGAGCATCACAGGACTGTACGAAACAAGAATCGACCTGAGCCAGCT |
| | | GGGAGGAGACGGAGGAGGAAGCCCGAAGAAGAAGAGAAAGGTCTAGCTAGCCATCACATTTAAAAGCATC |
| | | TCAGCCTACCATGAGAATAAGAGAAAGAAAATGAAGATCAATAGCTTATTCATCTCTTTTTCTTTTTCGTTGG |
| | | TGTAAAGCCAACACCCTGTCTAAAAAACATAAATTTCTTTAATCATTTTGCCTCTTTTCTCTGTGCTTCAATT |
| | | AATAAAAAATGGAAAGAACCTCGAG |
| 1102 | Cas9 DNA | ATGGACAAGAAGTACAGCATCGGACTGGACATCGGAACAAACAGCGTCGGATGGGCAGTCATCACAGACGA |
| | coding sequence | ATACAAGGTCCCGAGCAAGAAGTTCAAGGTCCTGGGAAACACAGACAGACACAGCATCAAGAAGAACCTG |
| | | ATCGGAGCACTGCTGTTCGACAGCGGAGAAACAGCAGAAGCAACAAGACTGAAGAGAACAGCAAGAAGAA |
| | | GATACACAAGAAGAAAGAACAGAATCTGCTACCTGCAGGAAATCTTCAGCAACGAAATGGCAAAGGTCGAC |
| | | GACAGCTTCTTCCACAGACTGGAAGAAAGCTTCCTGGTCGAAGAAGACAAGAAGCACGAAAGACACCCGAT |
| | | CTTCGGAAACATCGTCGACGAAGTCGCATACCACGAAAAGTACCCGACAATCTACCACCTGAGAAAGAAGC |
| | | TGGTCGACAGCACAGACAAGGCAGACCTGAGACTGATCTACCTGGCACTGGCACACATGATCAAGTTCAGA |
| | | GGACACTTCCTGATCGAAGGAGACCTGAACCCGGACAACAGCGACGTCGACAAGCTGTTCATCCAGCTGGT |
| | | CCAGACATACAACCAGCTGTTCGAAGAAAACCCGATCAACGCAAGCGGAGTCGACGCAAAGGCAATCCTGA |
| | | GCGCAAGACTGAGCAAGAGCAGAAGACTGGAAAACCTGATCGCACAGCTGCCGGGAGAAAAGAAGAACGG |
| | | ACTGTTCGGAAACCTGATCGCACTGAGCCTGGGACTGACACCGAACTTCAAGAGCAACTTCGACCTGGCAG |
| | | AAGACGCAAAGCTGCAGCTGAGCAAGGACACATACGACGACGACCTGGACAACCTGCTGGCACAGATCGG |
| | | AGACCAGTACGCAGACCTGTTCCTGGCAGCAAAGAACCTGAGCGACGCAATCCTGCTGAGCGACATCCTGA |
| | | GAGTCAACACAGAAATCACAAAGGCACCGCTGAGCGCAAGCATGATCAAGAGATACGACGAACACCACCA |
| | | GGACCTGACACTGCTGAAGGCACTGGTCAGACAGCAGCTGCCGGAAAAGTACAAGGAAATCTTCTTCGACC |
| | | AGAGCAAGAACGGATACGCAGGATACATCGACGGAGGAGCAAGCCAGGAAGAATTCTACAAGTTCATCAA |
| | | GCCGATCCTGGAAAAGATGGACGGAACAGAAGAACTGCTGGTCAAGCTGAACAGAGAAGACCTGCTGAGA |
| | | AAGCAGAGAACATTCGACAACGGAAGCATCCCGCACCAGATCCACCTGGGAGAACTGCACGCAATCCTGAG |
| | | AAGACAGGAAGACTTCTACCCGTTCCTGAAGGACAACAGAGAAAAGATCGAAAAGATCCTGACATTCAGAA |
| | | TCCCGTACTACGTCGGACCGCTGGCAAGAGGAAACAGCAGATTCGCATGGATGACAAGAAAGAGCGAAGAA |
| | | ACAATCACACCGTGGAACTTCGAAGAAGTCGTCGACAAGGGAGCAAGCGCACAGAGCTTCATCGAAAGAAT |
| | | GACAAACTTCGACAAGAACCTGCCGAACGAAAAGGTCCTGCCGAAGCACAGCCTGCTGTACGAATACTTCA |
| | | CAGTCTACAACGAACTGACAAAGGTCAAGTACGTCACAGAAGGAATGAGAAAGCCGGCATTCCTGAGCGGA |
| | | GAACAGAAGAAGGCAATCGTCGACCTGCTGTTCAAGACAAACAGAAAGGTCACAGTCAAGCAGCTGAAGG |
| | | AAGACTACTTCAAGAAGATCGAATGCTTCGACAGCGTCGAAATCAGCGGAGTCGAAGACAGATTCAACGCA |
| | | AGCCTGGGAACATACCACGACCTGCTGAAGATCATCAAGGACAAGGACTTCCTGGACAACGAAGAAAACGA |
| | | AGACATCCTGGAAGACATCGTCCTGACACTGACACTGTTCGAAGACAGAGAAATGATCGAAGAAAGACTGA |
| | | AGACATACGCACACCTGTTCGACGACAAGGTCATGAAGCAGCTGAAGAGAAGAAGATACACAGGATGGGG |
| | | AAGACTGAGCAGAAAGCTGATCAACGGAATCAGAGACAAGCAGAGCGGAAAGACAATCCTGGACTTCCTG |
| | | AAGAGCGACGGATTCGCAAACAGAAACTTCATGCAGCTGATCCACGACGACAGCCTGACATTCAAGGAAGA |
| | | CATCCAGAAGGCACAGGTCAGCGGACAGGGAGACAGCCTGCACGAACACATCGCAAACCTGGCAGGAAGC |
| | | CCGGCAATCAAGAAGGGAATCCTGCAGACAGTCAAGGTCGTCGACGAACTGGTCAAGGTCATGGGAAGACA |
| | | CAAGCCGGAAAACATCGTCATCGAAATGGCAAGAGAAAACCAGACAACACAGAAGGGACAGAAGAACAGC |
| | | AGAGAAAGAATGAAGAGAATCGAAGAAGGAATCAAGGAACTGGGAAGCCAGATCCTGAAGGAACACCCGG |
| | | TCGAAAACACACAGCTGCAGAACGAAAAGCTGTACCTGTACTACCTGCAGAACGGAAGAGACATGTACGTC |
| | | GACCAGGAACTGGACATCAACAGACTGAGCGACTACGACGTCGACCACATCGTCCCGCAGAGCTTCCTGAA |
| | | GGACGACAGCATCGACAACAAGGTCCTGACAAGAAGCGACAAGAACAGAGGAAAGAGCGACAACGTCCCG |
| | | AGCGAAGAAGTCGTCAAGAAGATGAAGAACTACTGGAGACAGCTGCTGAACGCAAAGCTGATCACACAGA |
| | | GAAAGTTCGACAACCTGACAAAGGCAGAGAGAGGAGGACTGAGCGAACTGGACAAGGCAGGATTCATCAA |
| | | GAGACAGCTGGTCGAAACAAGACAGATCACAAAGCACGTCGCACAGATCCTGGACAGCAGAATGAACACA |
| | | AAGTACGACGAAAACGACAAGCTGATCAGAGAAGTCAAGGTCATCACACTGAAGAGCAAGCTGGTCAGCG |
| | | ACTTCAGAAAGGACTTCCAGTTCTACAAGGTCAGAGAAATCAACAACTACCACCACGCACACGACGCATAC |
| | | CTGAACGCAGTCGTCGGAACAGCACTGATCAAGAAGTACCCGAAGCTGGAAAGCGAATTCGTCTACGGAGA |
| | | CTACAAGGTCTACGACGTCAGAAAGATGATCGCAAAGAGCGAACAGGAAATCGGAAAGGCAACAGCAAAG |
| | | TACTTCTTCTACAGCAACATCATGAACTTCTTCAAGACAGAAATCACACTGGCAAACGGAGAAATCAGAAAG |
| | | AGACCGCTGATCGAAACAAACGGAGAAACAGGAGAAATCGTCTGGGACAAGGGAAGAGACTTCGCAACAG |
| | | TCAGAAAGGTCCTGAGCATGCCGCAGGTCAACATCGTCAAGAAGACAGAAGTCCAGACAGGAGGATTCAGC |
| | | AAGGAAAGCATCCTGCCGAAGAGAAACAGCGACAAGCTGATCGCAAGAAAGAAGGACTGGGACCCGAAGA |
| | | AGTACGGAGGATTCGACAGCCCGACAGTCGCATACAGCGTCCTGGTCGTCGCAAAGGTCGAAAAGGGAAAG |
| | | AGCAAGAAGCTGAAGAGCGTCAAGGAACTGCTGGGAATCACAATCATGGAAAGAAGCAGCTTCGAAAAGA |
| | | ACCCGATCGACTTCCTGGAAGCAAAGGGATACAAGGAAGTCAAGAAGGACCTGATCATCAAGCTGCCGAAG |
| | | TACAGCCTGTTCGAACTGGAAAACGGAAGAAAGAGAATGCTGGCAAGCGCAGGAGAACTGCAGAAGGGAA |
| | | ACGAACTGGCACTGCCGAGCAAGTACGTCAACTTCCTGTACCTGGCAAGCCACTACGAAAAGCTGAAGGGA |
| | | AGCCCGGAAGACAACGAACAGAAGCAGCTGTTCGTCGAACAGCACAAGCACTACCTGGACGAAATCATCGA |
| | | ACAGATCAGCGAATTCAGCAAGAGAGTCATCCTGGCAGACGCAAACCTGGACAAGGTCCTGAGCGCATACA |
| | | ACAAGCACAGAGACAAGCCGATCAGAGAACAGGCAGAAAACATCATCCACCTGTTCACACTGACAAACCTG |
| | | GGAGCACCGGCAGCATTCAAGTACTTCGACACAACAATCGACAGAAAGAGATACACAAGCACAAAGGAAG |
| | | TCCTGGACGCAACACTGATCCACCAGAGCATCACAGGACTGTACGAAACAAGAATCGACCTGAGCCAGCTG |
| | | GGAGGAGACGGAGGAGGAAGCCCGAAGAAGAAGAGAAAGGTCTAG |
| 1103 | Cas9 DNA coding | ATGGATAAGAAGTACTCAATCGGGCTGGATATCGGAACTAATTCCGTGGGTTGGGCAGTGATCACGGATGA |
| | sequence 1 | ATACAAAGTGCCGTCCAAGAAGTTCAAGGTCCTGGGGAACACCGATAGACACAGCATCAAGAAAAATCTCA |
| | | TCGGAGCCCTGCTGTTTGACTCCGGCGAAACCGCAGAAGCGACCCGGCTCAAACGTACCGCGAGGCGACGC |
| | | TACACCCGGCGGAAGAATCGCATCTGCTATCTGCAAGAGATCTTTTCGAACGAAATGGCAAAGGTCGACGA |
| | | CAGCTTCTTCCACCGCCTGGAAGAATCTTTCCTGGTGGAGGAGGACAAGAAGCATGAACGGCATCCTATCTT |
| | | TGGAAACATCGTCGACGAAGTGGCGTACCACGAAAAGTACCCGACCATCTACCATCTGCGGAAGAAGTTGG |
| | | TTGACTCAACTGACAAGGCCGACCTCAGATTGATCTACTTGGCCCTCGCCCATATGATCAAATTCCGCGGAC |
| | | ACTTCCTGATCGAAGGCGATCTGAACCCTGATAACTCCGACGTGGATAAGCTTTTCATTCAACTGGTGCAGA |
| | | CCTACAACCAACTGTTCGAAGAAAACCCAATCAATGCTAGCGGCGTCGATGCCAAGGCCATCCTGTCCGCCC |
| | | GGCTGTCGAAGTCGCGGCGCCTCGAAAACCTGATCGCACAGCTGCCGGGAGAGAAAAAGAACGGACTTTTC |
| | | GGCAACTTGATCGCTCTCTCACTGGGACTCACTCCCAATTTCAAGTCCAATTTTGACCTGGCCGAGGACGCG |
| | | AAGCTGCAACTCTCAAAGGACACCTACGACGACGACTTGGACAATTTGCTGGCACAAATTGGCGATCAGTAC |
| | | GCGGATCTGTTCCTTGCCGCTAAGAACCTTTCGGACGCAATCTTGCTGTCCGATATCCTGCGCGTGAACACCG |
| | | AAATAACCAAAGCGCCGCTTAGCGCCTCGATGATTAAGCGGTACGACGAGCATCACCAGGATCTCACGCTG |
| | | CTCAAAGCGCTCGTGAGACAGCAACTGCCTGAAAAGTACAAGGAGATCTTCTTCGACCAGTCCAAGAATGG |
| | | GTACGCAGGGTACATCGATGGAGGCGCTAGCCAGGAAGAGTTCTATAAGTTCATCAAGCCAATCCTGGAAA |
| | | AGATGGACGGAACCGAAGAACTGCTGGTCAAGCTGAACAGGGAGGATCTGCTCCGGAAACAGAGAACCTTT |
| | | GACAACGGATCCATTCCCCACCAGATCCATCTGGGTGAGCTGCACGCCATCTTGCGGCGCCAGGAGGACTTT |
| | | TACCCATTCCTCAAGGACAACCGGGAAAAGATCGAGAAAATTCTGACGTTCCGCATCCCGTATTACGTGGGC |
| | | CCACTGGCGCGCGGCAATTCGCGCTTCGCGTGGATGACTAGAAAATCAGAGGAAACCATCACTCCTTGGAAT |
| | | TTCGAGGAAGTTGTGGATAAGGGAGCTTCGGCACAAAGCTTCATCGAACGAATGACCAACTTCGACAAGAA |
| | | TCTCCCAAACGAGAAGGTGCTTCCTAAGCACAGCCTCCTTTACGAATACTTCACTGTCTACAACGAACTGAC |
| | | TAAAGTGAAATACGTTACTGAAGGAATGAGGAAGCCGGCCTTTCTGTCCGGAGAACAGAAGAAAGCAATTG |
| | | TCGATCTGCTGTTCAAGACCAACCGCAAGGTGACCGTCAAGCAGCTTAAAGAGGACTACTTCAAGAAGATC |
| | | GAGTGTTTCGACTCAGTGGAAATCAGCGGGGTGGAGGACAGATTCAACGCTTCGCTGGGAACCTATCATGAT |
| | | CTCCTGAAGATCATCAAGGACAAGGACTTCCTTGACAACGAGGAGAACGAGGACATCCTGGAAGATATCGT |
| | | CCTGACCTTGACCCTTTTCGAGGATCGCGAGATGATCGAGGAGAGGCTTAAGACCTACGCTCATCTCTTCGA |
| | | CGATAAGGTCATGAAACAACTCAAGCGCCGCCGGTACACTGGTTGGGGCCGCCTCTCCCGCAAGCTGATCA |
| | | ACGGTATTCGCGATAAACAGAGCGGTAAAACTATCCTGGATTTCCTCAAATCGGATGGCTTCGCTAATCGTA |
| | | ACTTCATGCAATTGATCCACGACGACAGCCTGACCTTTAAGGAGGACATCCAAAAAGCACAAGTGTCCGGA |
| | | CAGGGAGACTCACTCCATGAACACATCGCGAATCTGGCCGGTTCGCCGGCGATTAAGAAGGGAATTCTGCA |
| | | AACTGTGAAGGTGGTCGACGAGCTGGTGAAGGTCATGGGACGGCACAAACCGGAGAATATCGTGATTGAAA |
| | | TGGCCCGAGAAAACCAGACTACCCAGAAGGGCCAGAAAAACTCCCGCGAAAGGATGAAGCGGATCGAAGA |
| | | AGGAATCAAGGAGCTGGGCAGCCAGATCCTGAAAGAGCACCCGGTGGAAAACACGCAGCTGCAGAACGAG |
| | | AAGCTCTACCTGTACTATTTGCAAAATGGACGGGACATGTACGTGGACCAAGAGCTGGACATCAATCGGTTG |
| | | TCTGATTACGACGTGGACCACATCGTTCCACAGTCCTTTCTGAAGGATGACTCGATCGATAACAAGGTGTTG |
| | | ACTCGCAGCGACAAGAACAGAGGGAAGTCAGATAATGTGCCATCGGAGGAGGTCGTGAAGAAGATGAAGA |
| | | ATTACTGGCGGCAGCTCCTGAATGCGAAGCTGATTACCCAGAGAAAGTTTGACAATCTCACTAAAGCCGAGC |
| | | GCGGCGGACTCTCAGAGCTGGATAAGGCTGGATTCATCAAACGGCAGCTGGTCGAGACTCGGCAGATTACC |
| | | AAGCACGTGGCGCAGATCTTGGACTCCCGCATGAACACTAAATACGACGAGAACGATAAGCTCATCCGGGA |
| | | AGTGAAGGTGATTACCCTGAAAAGCAAACTTGTGTCGGACTTTCGGAAGGACTTTCAGTTTTACAAAGTGAG |
| | | AGAAATCAACAACTACCATCACGCGCATGACGCATACCTCAACGCTGTGGTCGGTACCGCCCTGATCAAAA |
| | | AGTACCCTAAACTTGAATCGGAGTTTGTGTACGGAGACTACAAGGTCTACGACGTGAGGAAGATGATAGCC |
| | | AAGTCCGAACAGGAAATCGGGAAAGCAACTGCGAAATACTTCTTTTACTCAAACATCATGAACTTTTTCAAG |
| | | ACTGAAATTACGCTGGCCAATGGAGAAATCAGGAAGAGGCCACTGATCGAAACTAACGGAGAAACGGGCG |
| | | AAATCGTGTGGGACAAGGGCAGGGACTTCGCAACTGTTCGCAAAGTGCTCTCTATGCCGCAAGTCAATATTG |
| | | TGAAGAAAACCGAAGTGCAAACCGGCGGATTTTCAAAGGAATCGATCCTCCCAAAGAGAAATAGCGACAAG |
| | | CTCATTGCACGCAAGAAAGACTGGGACCCGAAGAAGTACGGAGGATTCGATTCGCCGACTGTCGCATACTC |
| | | CGTCCTCGTGGTGGCCAAGGTGGAGAAGGGAAAGAGCAAAAAGCTCAAATCCGTCAAAGAGCTGCTGGGGA |
| | | TTACCATCATGGAACGATCCTCGTTCGAGAAGAACCCGATTGATTTCCTCGAGGCGAAGGGTTACAAGGAGG |
| | | TGAAGAAGGATCTGATCATCAAACTCCCCAAGTACTCACTGTTCGAACTGGAAAATGGTCGGAAGCGCATGC |
| | | TGGCTTCGGCCGGAGAACTCCAAAAAGGAAATGAGCTGGCCTTGCCTAGCAAGTACGTCAACTTCCTCTATC |
| | | TTGCTTCGCACTACGAAAAACTCAAAGGGTCACCGGAAGATAACGAACAGAAGCAGCTTTTCGTGGAGCAG |
| | | CACAAGCATTATCTGGATGAAATCATCGAACAAATCTCCGAGTTTTCAAAGCGCGTGATCCTCGCCGACGCC |
| | | AACCTCGACAAAGTCCTGTCGGCCTACAATAAGCATAGAGATAAGCCGATCAGAGAACAGGCCGAGAACAT |
| | | TATCCACTTGTTCACCCTGACTAACCTGGGAGCCCCAGCCGCCTTCAAGTACTTCGATACTACTATCGATCGC |
| | | AAAAGATACACGTCCACCAAGGAAGTTCTGGACGCGACCCTGATCCACCAAAGCATCACTGGACTCTACGA |
| | | AACTAGGATCGATCTGTCGCAGCTGGGTGGCGATGGCGGTGGATCTCCGAAAAAGAAGAGAAAGGTGTAAT |
| | | GA |
| 1104 | Cas9 mRNA open | AUGGACAAGAAGUACAGCAUCGGACUGGACAUCGGAACAAACAGCGUCGGAUGGGCAGUCAUCACAGAC |
| | reading | GAAUACAAGGUCCCGAGCAAGAAGUUCAAGGUCCUGGGAAACACAGACAGACACAGCAUCAAGAAGAAC |
| | frame (ORF) 2 | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAGGAGGAAGCCCGAAGAAGAAGAGAAAGGUCUAG |
| 1105 | Cas9 mRNA | AUGGAUAAGAAGUACUCAAUCGGGCUGGAUAUCGGAACUAAUUCCGUGGGUUGGGCAGUGAUCACGGAU |
| | ORF 1 | GAAUACAAAGUGCCGUCCAAGAAGUUCAAGGUCCUGGGGAACACCGAUAGACACAGCAUCAAGAAAAAU |
| | | CUCAUCGGAGCCCUGCUGUUUGACUCCGGCGAAACCGCAGAAGCGACCCGGCUCAAACGUACCGCGAGGC |
| | | GACGCUACACCCGGCGGAAGAAUCGCAUCUGCUAUCUGCAAGAGAUCUUUUCGAACGAAAUGGCAAAGG |
| | | UCGACGACAGCUUCUUCCACCGCCUGGAAGAAUCUUUCCUGGUGGAGGAGGACAAGAAGCAUGAACGGC |
| | | AUCCUAUCUUUGGAAACAUCGUCGACGAAGUGGCGUACCACGAAAAGUACCCGACCAUCUACCAUCUGC |
| | | GGAAGAAGUUGGUUGACUCAACUGACAAGGCCGACCUCAGAUUGAUCUACUUGGCCCUCGCCCAUAUGA |
| | | UCAAAUUCCGCGGACACUUCCUGAUCGAAGGCGAUCUGAACCCUGAUAACUCCGACGUGGAUAAGCUUU |
| | | UCAUUCAACUGGUGCAGACCUACAACCAACUGUUCGAAGAAAACCCAAUCAAUGCUAGCGGCGUCGAUG |
| | | CCAAGGCCAUCCUGUCCGCCCGGCUGUCGAAGUCGCGGCGCCUCGAAAACCUGAUCGCACAGCUGCCGGG |
| | | AGAGAAAAAGAACGGACUUUUCGGCAACUUGAUCGCUCUCUCACUGGGACUCACUCCCAAUUUCAAGUC |
| | | CAAUUUUGACCUGGCCGAGGACGCGAAGCUGCAACUCUCAAAGGACACCUACGACGACGACUUGGACAA |
| | | UUUGCUGGCACAAAUUGGCGAUCAGUACGCGGAUCUGUUCCUUGCCGCUAAGAACCUUUCGGACGCAAU |
| | | CUUGCUGUCCGAUAUCCUGCGCGUGAACACCGAAAUAACCAAAGCGCCGCUUAGCGCCUCGAUGAUUAA |
| | | GCGGUACGACGAGCAUCACCAGGAUCUCACGCUGCUCAAAGCGCUCGUGAGACAGCAACUGCCUGAAAA |
| | | GUACAAGGAGAUCUUCUUCGACCAGUCCAAGAAUGGGUACGCAGGGUACAUCGAUGGAGGCGCUAGCCA |
| | | GGAAGAGUUCUAUAAGUUCAUCAAGCCAAUCCUGGAAAAGAUGGACGGAACCGAAGAACUGCUGGUCAA |
| | | GCUGAACAGGGAGGAUCUGCUCCGGAAACAGAGAACCUUUGACAACGGAUCCAUUCCCCACCAGAUCCA |
| | | UCUGGGUGAGCUGCACGCCAUCUUGCGGCGCCAGGAGGACUUUUACCCAUUCCUCAAGGACAACCGGGA |
| | | AAAGAUCGAGAAAAUUCUGACGUUCCGCAUCCCGUAUUACGUGGGCCCACUGGCGCGCGGCAAUUCGCG |
| | | CUUCGCGUGGAUGACUAGAAAAUCAGAGGAAACCAUCACUCCUUGGAAUUUCGAGGAAGUUGUGGAUAA |
| | | GGGAGCUUCGGCACAAAGCUUCAUCGAACGAAUGACCAACUUCGACAAGAAUCUCCCAAACGAGAAGGU |
| | | GCUUCCUAAGCACAGCCUCCUUUACGAAUACUUCACUGUCUACAACGAACUGACUAAAGUGAAAUACGU |
| | | UACUGAAGGAAUGAGGAAGCCGGCCUUUCUGUCCGGAGAACAGAAGAAAGCAAUUGUCGAUCUGCUGUU |
| | | CAAGACCAACCGCAAGGUGACCGUCAAGCAGCUUAAAGAGGACUACUUCAAGAAGAUCGAGUGUUUCGA |
| | | CUCAGUGGAAAUCAGCGGGGUGGAGGACAGAUUCAACGCUUCGCUGGGAACCUAUCAUGAUCUCCUGAA |
| | | GAUCAUCAAGGACAAGGACUUCCUUGACAACGAGGAGAACGAGGACAUCCUGGAAGAUAUCGUCCUGAC |
| | | CUUGACCCUUUUCGAGGAUCGCGAGAUGAUCGAGGAGAGGCUUAAGACCUACGCUCAUCUCUUCGACGA |
| | | UAAGGUCAUGAAACAACUCAAGCGCCGCCGGUACACUGGUUGGGGCCGCCUCUCCCGCAAGCUGAUCAAC |
| | | GGUAUUCGCGAUAAACAGAGCGGUAAAACUAUCCUGGAUUUCCUCAAAUCGGAUGGCUUCGCUAAUCGU |
| | | AACUUCAUGCAAUUGAUCCACGACGACAGCCUGACCUUUAAGGAGGACAUCCAAAAAGCACAAGUGUCC |
| | | GGACAGGGAGACUCACUCCAUGAACACAUCGCGAAUCUGGCCGGUUCGCCGGCGAUUAAGAAGGGAAUU |
| | | CUGCAAACUGUGAAGGUGGUCGACGAGCUGGUGAAGGUCAUGGGACGGCACAAACCGGAGAAUAUCGUG |
| | | AUUGAAAUGGCCCGAGAAAACCAGACUACCCAGAAGGGCCAGAAAAACUCCCGCGAAAGGAUGAAGCGG |
| | | AUCGAAGAAGGAAUCAAGGAGCUGGGCAGCCAGAUCCUGAAAGAGCACCCGGUGGAAAACACGCAGCUG |
| | | CAGAACGAGAAGCUCUACCUGUACUAUUUGCAAAAUGGACGGGACAUGUACGUGGACCAAGAGCUGGAC |
| | | AUCAAUCGGUUGUCUGAUUACGACGUGGACCACAUCGUUCCACAGUCCUUUCUGAAGGAUGACUCGAUC |
| | | GAUAACAAGGUGUUGACUCGCAGCGACAAGAACAGAGGGAAGUCAGAUAAUGUGCCAUCGGAGGAGGUC |
| | | GUGAAGAAGAUGAAGAAUUACUGGCGGCAGCUCCUGAAUGCGAAGCUGAUUACCCAGAGAAAGUUUGAC |
| | | AAUCUCACUAAAGCCGAGCGCGGCGGACUCUCAGAGCUGGAUAAGGCUGGAUUCAUCAAACGGCAGCUG |
| | | GUCGAGACUCGGCAGAUUACCAAGCACGUGGCGCAGAUCUUGGACUCCCGCAUGAACACUAAAUACGAC |
| | | GAGAACGAUAAGCUCAUCCGGGAAGUGAAGGUGAUUACCCUGAAAAGCAAACUUGUGUCGGACUUUCGG |
| | | AAGGACUUUCAGUUUUACAAAGUGAGAGAAAUCAACAACUACCAUCACGCGCAUGACGCAUACCUCAAC |
| | | GCUGUGGUCGGUACCGCCCUGAUCAAAAAGUACCCUAAACUUGAAUCGGAGUUUGUGUACGGAGACUAC |
| | | AAGGUCUACGACGUGAGGAAGAUGAUAGCCAAGUCCGAACAGGAAAUCGGGAAAGCAACUGCGAAAUAC |
| | | UUCUUUUACUCAAACAUCAUGAACUUUUUCAAGACUGAAAUUACGCUGGCCAAUGGAGAAAUCAGGAAG |
| | | AGGCCACUGAUCGAAACUAACGGAGAAACGGGCGAAAUCGUGUGGGACAAGGGCAGGGACUUCGCAACU |
| | | GUUCGCAAAGUGCUCUCUAUGCCGCAAGUCAAUAUUGUGAAGAAAACCGAAGUGCAAACCGGCGGAUUU |
| | | UCAAAGGAAUCGAUCCUCCCAAAGAGAAAUAGCGACAAGCUCAUUGCACGCAAGAAAGACUGGGACCCG |
| | | AAGAAGUACGGAGGAUUCGAUUCGCCGACUGUCGCAUACUCCGUCCUCGUGGUGGCCAAGGUGGAGAAG |
| | | GGAAAGAGCAAAAAGCUCAAAUCCGUCAAAGAGCUGCUGGGGAUUACCAUCAUGGAACGAUCCUCGUUC |
| | | GAGAAGAACCCGAUUGAUUUCCUCGAGGCGAAGGGUUACAAGGAGGUGAAGAAGGAUCUGAUCAUCAAA |
| | | CUCCCCAAGUACUCACUGUUCGAACUGGAAAAUGGUCGGAAGCGCAUGCUGGCUUCGGCCGGAGAACUC |
| | | CAAAAAGGAAAUGAGCUGGCCUUGCCUAGCAAGUACGUCAACUUCCUCUAUCUUGCUUCGCACUACGAA |
| | | AAACUCAAAGGGUCACCGGAAGAUAACGAACAGAAGCAGCUUUUCGUGGAGCAGCACAAGCAUUAUCUG |
| | | GAUGAAAUCAUCGAACAAAUCUCCGAGUUUUCAAAGCGCGUGAUCCUCGCCGACGCCAACCUCGACAAA |
| | | GUCCUGUCGGCCUACAAUAAGCAUAGAGAUAAGCCGAUCAGAGAACAGGCCGAGAACAUUAUCCACUUG |
| | | UUCACCCUGACUAACCUGGGAGCCCCAGCCGCCUUCAAGUACUUCGAUACUACUAUCGAUCGCAAAAGAU |
| | | ACACGUCCACCAAGGAAGUUCUGGACGCGACCCUGAUCCACCAAAGCAUCACUGGACUCUACGAAACUAG |
| | | GAUCGAUCUGUCGCAGCUGGGUGGCGAUGGCGGUGGAUCUCCGAAAAAGAAGAGAAAGGUGUAAUGA |
| 1106 | Cas9 nickase | AUGGACAAGAAGUACAGCAUCGGACUGGCAAUCGGAACAAACAGCGUCGGAUGGGCAGUCAUCACAGAC |
| | (D10A) mRNA | GAAUACAAGGUCCCGAGCAAGAAGUUCAAGGUCCUGGGAAACACAGACAGACACAGCAUCAAGAAGAAC |
| | ORF | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAGGAGGAAGCCCGAAGAAGAAGAGAAAGGUCUAG |
| 1107 | dCas9 (D10A | AUGGACAAGAAGUACAGCAUCGGACUGGCAAUCGGAACAAACAGCGUCGGAUGGGCAGUCAUCACAGAC |
| | H840A) mRNA | GAAUACAAGGUCCCGAGCAAGAAGUUCAAGGUCCUGGGAAACACAGACAGACACAGCAUCAAGAAGAAC |
| | ORF | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACGCAAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAGGAGGAAGCCCGAAGAAGAAGAGAAAGGUCUAG |
| 1108 | Cas9 coding | GACAAGAAGUACAGCAUCGGACUGGACAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without | AUCGGAGCACUGCUGUUCGACAGCGGAGAAACAGCAGAAGCAACAAGACUGAAGAGAACAGCAAGAAGA |
| | start or stop | AGAUACACAAGAAGAAAGAACAGAAUCUGCUACCUGCAGGAAAUCUUCAGCAACGAAAUGGCAAAGGUC |
| | codons | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGACGGAGGAGGAAGCCCGAAGAAGAAGAGAAAGGUC |
| 1109 | Cas9 nickase | GACAAGAAGUACAGCAUCGGACUGGCAAUCGGAACAAACAGCGUCGGAUGGGCAGUCAUCACAGACGAA |
| | coding sequence, | UACAAGGUCCCGAGCAAGAAGUUCAAGGUCCUGGGAAACACAGACAGACACAGCAUCAAGAAGAACCUG |
| | without stop or | AUCGGAGCACUGCUGUUCGACAGCGGAGAAACAGCAGAAGCAACAAGACUGAAGAGAACAGCAAGAAGA |
| | start codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGACGGAGGAGGAAGCCCGAAGAAGAAGAGAAAGGUC |
| 1110 | dCas9 coding | GACAAGAAGUACAGCAUCGGACUGGCAAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without start | AUCGGAGCACUGCUGUUCGACAGCGGAGAAACAGCAGAAGCAACAAGACUGAAGAGAACAGCAAGAAGA |
| | or stop codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACGCAAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGACGGAGGAGGAAGCCCGAAGAAGAAGAGAAAGGUC |
| 1111 | Cas9 mRNA ORF | AUGGACAAGAAGUACAGCAUCGGACUGGACAUCGGAACAAACAGCGUCGGAUGGGCAGUCAUCACAGAC |
| | | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACUAG |
| 1112 | Cas9 coding | GACAAGAAGUACAGCAUCGGACUGGACAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without | AUCGGAGCACUGCUGUUCGACAGCGGAGAAACAGCAGAAGCAACAAGACUGAAGAGAACAGCAAGAAGA |
| | start or stop | AGAUACACAAGAAGAAAGAACAGAAUCUGCUACCUGCAGGAAAUCUUCAGCAACGAAAUGGCAAAGGUC |
| | codons | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGAC |
| 1113 | Cas9 nickase | AUGGACAAGAAGUACAGCAUCGGACUGGCAAUCGGAACAAACAGCGUCGGAUGGGCAGUCAUCACAGAC |
| | mRNA ORF | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACUAG |
| 1114 | Cas9 nickase | GACAAGAAGUACAGCAUCGGACUGGCAAUCGGAACAAACAGCGUCGGAUGGGCAGUCAUCACAGACGAA |
| | coding sequence, | UACAAGGUCCCGAGCAAGAAGUUCAAGGUCCUGGGAAACACAGACAGACACAGCAUCAAGAAGAACCUG |
| | without start | AUCGGAGCACUGCUGUUCGACAGCGGAGAAACAGCAGAAGCAACAAGACUGAAGAGAACAGCAAGAAGA |
| | or stop codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGAC |
| 1115 | dCas9 mRNA ORF | AUGGACAAGAAGUACAGCAUCGGACUGGCAAUCGGAACAAACAGCGUCGGAUGGGCAGUCAUCACAGAC |
| | | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACGCAAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACUAG |
| 1116 | dCas9 coding | GACAAGAAGUACAGCAUCGGACUGGCAAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without start | AUCGGAGCACUGCUGUUCGACAGCGGAGAAACAGCAGAAGCAACAAGACUGAAGAGAACAGCAAGAAGA |
| | or stop codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACGCAAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGACGGAGGAGGAAGC |
| 1117 | Cas9 mRNA ORF | AUGGACAAGAAGUACAGCAUCGGACUGGACAUCGGAACAAACAGCGUCGGAUGGGCAGUCAUCACAGAC |
| | | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAAGCGGAAGCCCGAAGAAGAAGAGAAAGGUCGACGGAAG |
| | | CCCGAAGAAGAAGAGAAAGGUCGACAGCGGAUAG |
| 1118 | Cas9 coding | GACAAGAAGUACAGCAUCGGACUGGACAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without | AUCGGAGCACUGCUGUUCGACAGCGGAGAAACAGCAGAAGCAACAAGACUGAAGAGAACAGCAAGAAGA |
| | start or stop | AGAUACACAAGAAGAAAGAACAGAAUCUGCUACCUGCAGGAAAUCUUCAGCAACGAAAUGGCAAAGGUC |
| | codons | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGACGGAAGCGGAAGCCCGAAGAAGAAGAGAAAGGUCGACGGAAGC |
| | | CCGAAGAAGAAGAGAAAGGUCGACAGCGGA |
| 1119 | Cas9 nickase | AUGGACAAGAAGUACAGCAUCGGACUGGCAAUCGGAACAAACAGCGUCGGAUGGGCAGUCAUCACAGAC |
| | mRNA ORF | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACCACAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGACGGAAGCGGAAGCCCGAAGAAGAAGAGAAAGGUCGACGGAAG |
| | | CCCGAAGAAGAAGAGAAAGGUCGACAGCGGAUAG |
| 1120 | Cas9 nickase | GACAAGAAGUACAGCAUCGGACUGGCAAUCGGAACAAACAGCGUCGGAUGGGCAGUCAUCACAGACGAA |
| | coding sequence, | UACAAGGUCCCGAGCAAGAAGUUCAAGGUCCUGGGAAACACAGACAGACACAGCAUCAAGAAGAACCUG |
| | without start | AUCGGAGCACUGCUGUUCGACAGCGGAGAAACAGCAGAAGCAACAAGACUGAAGAGAACAGCAAGAAGA |
| | or stop codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACCACAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGAC |
| | | GGAAGCGGAAGCCCGAAGAAGAAGAGAAAGGUCGACGGAAGCCCGAAGAAGAAGAGAAAGGUCGACAGC |
| | | GGA |
| 1121 | dCas9 mRNA ORF | AUGGACAAGAAGUACAGCAUCGGACUGGCAAUCGGAACAAACAGCGUCGGAUGGGCAGUCAUCACAGAC |
| | | GAAUACAAGGUCCCGAGCAAGAAGUUCAAGGUCCUGGGAAACACAGACAGACACAGCAUCAAGAAGAAC |
| | | CUGAUCGGAGCACUGCUGUUCGACAGCGGAGAAACAGCAGAAGCAACAAGACUGAAGAGAACAGCAAGA |
| | | AGAAGAUACACAAGAAGAAAGAACAGAAUCUGCUACCUGCAGGAAAUCUUCAGCAACGAAAUGGCAAAG |
| | | GUCGACGACAGCUUCUUCCACAGACUGGAAGAAAGCUUCCUGGUCGAAGAAGACAAGAAGCACGAAAGA |
| | | CACCCGAUCUUCGGAAACAUCGUCGACGAAGUCGCAUACCACGAAAAGUACCCGACAAUCUACCACCUGA |
| | | GAAAGAAGCUGGUCGACAGCACAGACAAGGCAGACCUGAGACUGAUCUACCUGGCACUGGCACACAUGA |
| | | UCAAGUUCAGAGGACACUUCCUGAUCGAAGGAGACCUGAACCCGGACAACAGCGACGUCGACAAGCUGU |
| | | UCAUCCAGCUGGUCCAGACAUACAACCAGCUGUUCGAAGAAAACCCGAUCAACGCAAGCGGAGUCGACGC |
| | | AAAGGCAAUCCUGAGCGCAAGACUGAGCAAGAGCAGAAGACUGGAAAACCUGAUCGCACAGCUGCCGGG |
| | | AGAAAAGAAGAACGGACUGUUCGGAAACCUGAUCGCACUGAGCCUGGGACUGACACCGAACUUCAAGAG |
| | | CAACUUCGACCUGGCAGAAGACGCAAAGCUGCAGCUGAGCAAGGACACAUACGACGACGACCUGGACAA |
| | | CCUGCUGGCACAGAUCGGAGACCAGUACGCAGACCUGUUCCUGGCAGCAAAGAACCUGAGCGACGCAAUC |
| | | CUGCUGAGCGACAUCCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGCAGCUGCCGGAAAAG |
| | | UACAAGGAAAUCUUCUUCGACCAGAGCAAGAACGGAUACGCAGGAUACAUCGACGGAGGAGCAAGCCAG |
| | | GAAGAAUUCUACAAGUUCAUCAAGCCGAUCCUGGAAAAGAUGGACGGAACAGAAGAACUGCUGGUCAAG |
| | | CUGAACAGAGAAGACCUGCUGAGAAAGCAGAGAACAUUCGACAACGGAAGCAUCCCGCACCAGAUCCAC |
| | | CUGGGAGAACUGCACGCAAUCCUGAGAAGACAGGAAGACUUCUACCCGUUCCUGAAGGACAACAGAGAA |
| | | AAGAUCGAAAAGAUCCUGACAUUCAGAAUCCCGUACUACGUCGGACCGCUGGCAAGAGGAAACAGCAGA |
| | | UUCGCAUGGAUGACAAGAAAGAGCGAAGAAACAAUCACACCGUGGAACUUCGAAGAAGUCGUCGACAAG |
| | | GGAGCAAGCGCACAGAGCUUCAUCGAAAGAAUGACAAACUUCGACAAGAACCUGCCGAACGAAAAGGUC |
| | | CUGCCGAAGCACAGCCUGCUGUACGAAUACUUCACAGUCUACAACGAACUGACAAAGGUCAAGUACGUC |
| | | ACAGAAGGAAUGAGAAAGCCGGCAUUCCUGAGCGGAGAACAGAAGAAGGCAAUCGUCGACCUGCUGUUC |
| | | AAGACAAACAGAAAGGUCACAGUCAAGCAGCUGAAGGAAGACUACUUCAAGAAGAUCGAAUGCUUCGAC |
| | | AGCGUCGAAAUCAGCGGAGUCGAAGACAGAUUCAACGCAAGCCUGGGAACAUACCACGACCUGCUGAAG |
| | | AUCAUCAAGGACAAGGACUUCCUGGACAACGAAGAAAACGAAGACAUCCUGGAAGACAUCGUCCUGACA |
| | | CUGACACUGUUCGAAGACAGAGAAAUGAUCGAAGAAAGACUGAAGACAUACGCACACCUGUUCGACGAC |
| | | AAGGUCAUGAAGCAGCUGAAGAGAAGAAGAUACACAGGAUGGGGAAGACUGAGCAGAAAGCUGAUCAAC |
| | | GGAAUCAGAGACAAGCAGAGCGGAAAGACAAUCCUGGACUUCCUGAAGAGCGACGGAUUCGCAAACAGA |
| | | AACUUCAUGCAGCUGAUCCACGACGACAGCCUGACAUUCAAGGAAGACAUCCAGAAGGCACAGGUCAGC |
| | | GGACAGGGAGACAGCCUGCACGAACACAUCGCAAACCUGGCAGGAAGCCCGGCAAUCAAGAAGGGAAUC |
| | | CUGCAGACAGUCAAGGUCGUCGACGAACUGGUCAAGGUCAUGGGAAGACACAAGCCGGAAAACAUCGUC |
| | | AUCGAAAUGGCAAGAGAAAACCAGACAACACAGAAGGGACAGAAGAACAGCAGAGAAAGAAUGAAGAGA |
| | | AUCGAAGAAGGAAUCAAGGAACUGGGAAGCCAGAUCCUGAAGGAACACCCGGUCGAAAACACACAGCUG |
| | | CAGAACGAAAAGCUGUACCUGUACUACCUGCAGAACGGAAGAGACAUGUACGUCGACCAGGAACUGGAC |
| | | AUCAACAGACUGAGCGACUACGACGUCGACGCAAUCGUCCCGCAGAGCUUCCUGAAGGACGACAGCAUCG |
| | | ACAACAAGGUCCUGACAAGAAGCGACAAGAACAGAGGAAAGAGCGACAACGUCCCGAGCGAAGAAGUCG |
| | | UCAAGAAGAUGAAGAACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAUCAAGAGACAGCUGG |
| | | UCGAAACAAGACAGAUCACAAAGCACGUCGCACAGAUCCUGGACAGCAGAAUGAACACAAAGUACGACG |
| | | AAAACGACAAGCUGAUCAGAGAAGUCAAGGUCAUCACACUGAAGAGCAAGCUGGUCAGCGACUUCAGAA |
| | | AGGACUUCCAGUUCUACAAGGUCAGAGAAAUCAACAACUACCACCACGCACACGACGCAUACCUGAACGC |
| | | AGUCGUCGGAACAGCACUGAUCAAGAAGUACCCGAAGCUGGAAAGCGAAUUCGUCUACGGAGACUACAA |
| | | GGUCUACGACGUCAGAAAGAUGAUCGCAAAGAGCGAACAGGAAAUCGGAAAGGCAACAGCAAAGUACUU |
| | | CUUCUACAGCAACAUCAUGAACUUCUUCAAGACAGAAAUCACACUGGCAAACGGAGAAAUCAGAAAGAG |
| | | ACCGCUGAUCGAAACAAACGGAGAAACAGGAGAAAUCGUCUGGGACAAGGGAAGAGACUUCGCAACAGU |
| | | CAGAAAGGUCCUGAGCAUGCCGCAGGUCAACAUCGUCAAGAAGACAGAAGUCCAGACAGGAGGAUUCAG |
| | | CAAGGAAAGCAUCCUGCCGAAGAGAAACAGCGACAAGCUGAUCGCAAGAAAGAAGGACUGGGACCCGAA |
| | | GAAGUACGGAGGAUUCGACAGCCCGACAGUCGCAUACAGCGUCCUGGUCGUCGCAAAGGUCGAAAAGGG |
| | | AAAGAGCAAGAAGCUGAAGAGCGUCAAGGAACUGCUGGGAAUCACAAUCAUGGAAAGAAGCAGCUUCGA |
| | | AAAGAACCCGAUCGACUUCCUGGAAGCAAAGGGAUACAAGGAAGUCAAGAAGGACCUGAUCAUCAAGCU |
| | | GCCGAAGUACAGCCUGUUCGAACUGGAAAACGGAAGAAAGAGAAUGCUGGCAAGCGCAGGAGAACUGCA |
| | | GAAGGGAAACGAACUGGCACUGCCGAGCAAGUACGUCAACUUCCUGUACCUGGCAAGCCACUACGAAAA |
| | | GCUGAAGGGAAGCCCGGAAGACAACGAACAGAAGCAGCUGUUCGUCGAACAGCACAAGCACUACCUGGA |
| | | CGAAAUCAUCGAACAGAUCAGCGAAUUCAGCAAGAGAGUCAUCCUGGCAGACGCAAACCUGGACAAGGU |
| | | CCUGAGCGCAUACAACAAGCACAGAGACAAGCCGAUCAGAGAACAGGCAGAAAACAUCAUCCACCUGUU |
| | | CACACUGACAAACCUGGGAGCACCGGCAGCAUUCAAGUACUUCGACACAACAAUCGACAGAAAGAGAUA |
| | | CACAAGCACAAAGGAAGUCCUGGACGCAACACUGAUCCACCAGAGCAUCACAGGACUGUACGAAACAAG |
| | | AAUCGACCUGAGCCAGCUGGGAGGAGAC |
| | | GGAAGCGGAAGCCCGAAGAAGAAGAGAAAGGUCGACGGAAGCCCGAAGAAGAAGAGAAAGGUCGACAGC |
| | | GGAUAG |
| 1122 | dCas9 coding | GACAAGAAGUACAGCAUCGGACUGGCAAUCGGAACAAACAGCGUCGGAUGGGCAGUCAUCACAGACGAA |
| | sequence, | UACAAGGUCCCGAGCAAGAAGUUCAAGGUCCUGGGAAACACAGACAGACACAGCAUCAAGAAGAACCUG |
| | without start | AUCGGAGCACUGCUGUUCGACAGCGGAGAAACAGCAGAAGCAACAAGACUGAAGAGAACAGCAAGAAGA |
| | or stop codons | AGAUACACAAGAAGAAAGAACAGAAUCUGCUACCUGCAGGAAAUCUUCAGCAACGAAAUGGCAAAGGUC |
| | | GACGACAGCUUCUUCCACAGACUGGAAGAAAGCUUCCUGGUCGAAGAAGACAAGAAGCACGAAAGACAC |
| | | CCGAUCUUCGGAAACAUCGUCGACGAAGUCGCAUACCACGAAAAGUACCCGACAAUCUACCACCUGAGAA |
| | | AGAAGCUGGUCGACAGCACAGACAAGGCAGACCUGAGACUGAUCUACCUGGCACUGGCACACAUGAUCA |
| | | AGUUCAGAGGACACUUCCUGAUCGAAGGAGACCUGAACCCGGACAACAGCGACGUCGACAAGCUGUUCA |
| | | UCCAGCUGGUCCAGACAUACAACCAGCUGUUCGAAGAAAACCCGAUCAACGCAAGCGGAGUCGACGCAA |
| | | AGGCAAUCCUGAGCGCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACCGAACUUCAAGAGCA |
| | | ACUUCGACCUGGCAGAAGACGCAAAGCUGCAGCUGAGCAAGGACACAUACGACGACGACCUGGACAACC |
| | | UGCUGGCACAGAUCGGAGACCAGUACGCAGACCUGUUCCUGGCAGCAAAGAACCUGAGCGACGCAAUCC |
| | | UGCUGAGCGACAUCCUGAGAGUCAACACAGAAAUCACAAAGGCACCGCUGAGCGCAAGCAUGAUCAAGA |
| | | GAUACGACGAACACCACCAGGACCUGACACUGCUGAAGGCACUGGUCAGACAGCAGCUGCCGGAAAAGU |
| | | ACAAGGAAAUCUUCUUCGACCAGAGCAAGAACGGAUACGCAGGAUACAUCGACGGAGGAGCAAGCCAGG |
| | | AAGAAUUCUACAAGUUCAUCAAGCCGAUCCUGGAAAAGAUGGACGGAACAGAAGAACUGCUGGUCAAGC |
| | | UGAACAGAGAAGACCUGCUGAGAAAGCAGAGAACAUUCGACAACGGAAGCAUCCCGCACCAGAUCCACC |
| | | UGGGAGAACUGCACGCAAUCCUGAGAAGACAGGAAGACUUCUACCCGUUCCUGAAGGACAACAGAGAAA |
| | | AGAUCGAAAAGAUCCUGACAUUCAGAAUCCCGUACUACGUCGGACCGCUGGCAAGAGGAAACAGCAGAU |
| | | UCGCAUGGAUGACAAGAAAGAGCGAAGAAACAAUCACACCGUGGAACUUCGAAGAAGUCGUCGACAAGG |
| | | GAGCAAGCGCACAGAGCUUCAUCGAAAGAAUGACAAACUUCGACAAGAACCUGCCGAACGAAAAGGUCC |
| | | UGCCGAAGCACAGCCUGCUGUACGAAUACUUCACAGUCUACAACGAACUGACAAAGGUCAAGUACGUCA |
| | | CAGAAGGAAUGAGAAAGCCGGCAUUCCUGAGCGGAGAACAGAAGAAGGCAAUCGUCGACCUGCUGUUCA |
| | | AGACAAACAGAAAGGUCACAGUCAAGCAGCUGAAGGAAGACUACUUCAAGAAGAUCGAAUGCUUCGACA |
| | | GCGUCGAAAUCAGCGGAGUCGAAGACAGAUUCAACGCAAGCCUGGGAACAUACCACGACCUGCUGAAGA |
| | | UCAUCAAGGACAAGGACUUCCUGGACAACGAAGAAAACGAAGACAUCCUGGAAGACAUCGUCCUGACAC |
| | | UGACACUGUUCGAAGACAGAGAAAUGAUCGAAGAAAGACUGAAGACAUACGCACACCUGUUCGACGACA |
| | | AGGUCAUGAAGCAGCUGAAGAGAAGAAGAUACACAGGAUGGGGAAGACUGAGCAGAAAGCUGAUCAACG |
| | | GAAUCAGAGACAAGCAGAGCGGAAAGACAAUCCUGGACUUCCUGAAGAGCGACGGAUUCGCAAACAGAA |
| | | ACUUCAUGCAGCUGAUCCACGACGACAGCCUGACAUUCAAGGAAGACAUCCAGAAGGCACAGGUCAGCG |
| | | GACAGGGAGACAGCCUGCACGAACACAUCGCAAACCUGGCAGGAAGCCCGGCAAUCAAGAAGGGAAUCC |
| | | UGCAGACAGUCAAGGUCGUCGACGAACUGGUCAAGGUCAUGGGAAGACACAAGCCGGAAAACAUCGUCA |
| | | UCGAAAUGGCAAGAGAAAACCAGACAACACAGAAGGGACAGAAGAACAGCAGAGAAAGAAUGAAGAGAA |
| | | UCGAAGAAGGAAUCAAGGAACUGGGAAGCCAGAUCCUGAAGGAACACCCGGUCGAAAACACACAGCUGC |
| | | AGAACGAAAAGCUGUACCUGUACUACCUGCAGAACGGAAGAGACAUGUACGUCGACCAGGAACUGGACA |
| | | UCAACAGACUGAGCGACUACGACGUCGACGCAAUCGUCCCGCAGAGCUUCCUGAAGGACGACAGCAUCGA |
| | | CAACAAGGUCCUGACAAGAAGCGACAAGAACAGAGGAAAGAGCGACAACGUCCCGAGCGAAGAAGUCGU |
| | | CAAGAAGAUGAAGAACUACUGGAGACAGCUGCUGAACGCAAAGCUGAUCACACAGAGAAAGUUCGACAA |
| | | CCUGACAAAGGCAGAGAGAGGAGGACUGAGCGAACUGGACAAGGCAGGAUUCAUCAAGAGACAGCUGGU |
| | | CGAAACAAGACAGAUCACAAAGCACGUCGCACAGAUCCUGGACAGCAGAAUGAACACAAAGUACGACGA |
| | | AAACGACAAGCUGAUCAGAGAAGUCAAGGUCAUCACACUGAAGAGCAAGCUGGUCAGCGACUUCAGAAA |
| | | GGACUUCCAGUUCUACAAGGUCAGAGAAAUCAACAACUACCACCACGCACACGACGCAUACCUGAACGCA |
| | | GUCGUCGGAACAGCACUGAUCAAGAAGUACCCGAAGCUGGAAAGCGAAUUCGUCUACGGAGACUACAAG |
| | | GUCUACGACGUCAGAAAGAUGAUCGCAAAGAGCGAACAGGAAAUCGGAAAGGCAACAGCAAAGUACUUC |
| | | UUCUACAGCAACAUCAUGAACUUCUUCAAGACAGAAAUCACACUGGCAAACGGAGAAAUCAGAAAGAGA |
| | | CCGCUGAUCGAAACAAACGGAGAAACAGGAGAAAUCGUCUGGGACAAGGGAAGAGACUUCGCAACAGUC |
| | | AGAAAGGUCCUGAGCAUGCCGCAGGUCAACAUCGUCAAGAAGACAGAAGUCCAGACAGGAGGAUUCAGC |
| | | AAGGAAAGCAUCCUGCCGAAGAGAAACAGCGACAAGCUGAUCGCAAGAAAGAAGGACUGGGACCCGAAG |
| | | AAGUACGGAGGAUUCGACAGCCCGACAGUCGCAUACAGCGUCCUGGUCGUCGCAAAGGUCGAAAAGGGA |
| | | AAGAGCAAGAAGCUGAAGAGCGUCAAGGAACUGCUGGGAAUCACAAUCAUGGAAAGAAGCAGCUUCGAA |
| | | AAGAACCCGAUCGACUUCCUGGAAGCAAAGGGAUACAAGGAAGUCAAGAAGGACCUGAUCAUCAAGCUG |
| | | CCGAAGUACAGCCUGUUCGAACUGGAAAACGGAAGAAAGAGAAUGCUGGCAAGCGCAGGAGAACUGCAG |
| | | AAGGGAAACGAACUGGCACUGCCGAGCAAGUACGUCAACUUCCUGUACCUGGCAAGCCACUACGAAAAG |
| | | CUGAAGGGAAGCCCGGAAGACAACGAACAGAAGCAGCUGUUCGUCGAACAGCACAAGCACUACCUGGAC |
| | | GAAAUCAUCGAACAGAUCAGCGAAUUCAGCAAGAGAGUCAUCCUGGCAGACGCAAACCUGGACAAGGUC |
| | | CUGAGCGCAUACAACAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCGACAGAAAGAGAUAC |
| | | ACAAGCACAAAGGAAGUCCUGGACGCAACACUGAUCCACCAGAGCAUCACAGGACUGUACGAAACAAGA |
| | | AUCGACCUGAGCCAGCUGGGAGGAGAC |
| | | GGAAGCGGAAGCCCGAAGAAGAAGAGAAAGGUCGACGGAAGCCCGAAGAAGAAGAGAAAGGUCGACAGC |
| | | GGA |
| 1123 | DNA coding | GGGTCCCGCAGTCGGCGTCCAGCGGCTCTGCTTGTTCGTGTGTGTGTCGTTGCAGGCCTTATTCGGATCCGCC |
| | sequence for | ACCATGGACAAGAAGTACAGCATCGGACTGGACATCGGAACAAACAGCGTCGGATGGGCAGTCATCACAGA |
| | Cas9 transcript | CGAATACAAGGTCCCGAGCAAGAAGTTCAAGGTCCTGGGAAACACAGACAGACACAGCATCAAGAAGAAC |
| | | CTGATCGGAGCACTGCTGTTCGACAGCGGAGAAACAGCAGAAGCAACAAGACTGAAGAGAACAGCAAGAA |
| | | GAAGATACACAAGAAGAAAGAACAGAATCTGCTACCTGCAGGAAATCTTCAGCAACGAAATGGCAAAGGTC |
| | | GACGACAGCTTCTTCCACAGACTGGAAGAAAGCTTCCTGGTCGAAGAAGACAAGAAGCACGAAAGACACCC |
| | | GATCTTCGGAAACATCGTCGACGAAGTCGCATACCACGAAAAGTACCCGACAATCTACCACCTGAGAAAGA |
| | | AGCTGGTCGACAGCACAGACAAGGCAGACCTGAGACTGATCTACCTGGCACTGGCACACATGATCAAGTTC |
| | | AGAGGACACTTCCTGATCGAAGGAGACCTGAACCCGGACAACAGCGACGTCGACAAGCTGTTCATCCAGCT |
| | | GGTCCAGACATACAACCAGCTGTTCGAAGAAAACCCGATCAACGCAAGCGGAGTCGACGCAAAGGCAATCC |
| | | TGAGCGCAAGACTGAGCAAGAGCAGAAGACTGGAAAACCTGATCGCACAGCTGCCGGGAGAAAAGAAGAA |
| | | CGGACTGTTCGGAAACCTGATCGCACTGAGCCTGGGACTGACACCGAACTTCAAGAGCAACTTCGACCTGGC |
| | | AGAAGACGCAAAGCTGCAGCTGAGCAAGGACACATACGACGACGACCTGGACAACCTGCTGGCACAGATC |
| | | GGAGACCAGTACGCAGACCTGTTCCTGGCAGCAAAGAACCTGAGCGACGCAATCCTGCTGAGCGACATCCT |
| | | GAGAGTCAACACAGAAATCACAAAGGCACCGCTGAGCGCAAGCATGATCAAGAGATACGACGAACACCAC |
| | | CAGGACCTGACACTGCTGAAGGCACTGGTCAGACAGCAGCTGCCGGAAAAGTACAAGGAAATCTTCTTCGA |
| | | CCAGAGCAAGAACGGATACGCAGGATACATCGACGGAGGAGCAAGCCAGGAAGAATTCTACAAGTTCATCA |
| | | AGCCGATCCTGGAAAAGATGGACGGAACAGAAGAACTGCTGGTCAAGCTGAACAGAGAAGACCTGCTGAG |
| | | AAAGCAGAGAACATTCGACAACGGAAGCATCCCGCACCAGATCCACCTGGGAGAACTGCACGCAATCCTGA |
| | | GAAGACAGGAAGACTTCTACCCGTTCCTGAAGGACAACAGAGAAAAGATCGAAAAGATCCTGACATTCAGA |
| | | ATCCCGTACTACGTCGGACCGCTGGCAAGAGGAAACAGCAGATTCGCATGGATGACAAGAAAGAGCGAAGA |
| | | AACAATCACACCGTGGAACTTCGAAGAAGTCGTCGACAAGGGAGCAAGCGCACAGAGCTTCATCGAAAGAA |
| | | TGACAAACTTCGACAAGAACCTGCCGAACGAAAAGGTCCTGCCGAAGCACAGCCTGCTGTACGAATACTTC |
| | | ACAGTCTACAACGAACTGACAAAGGTCAAGTACGTCACAGAAGGAATGAGAAAGCCGGCATTCCTGAGCGG |
| | | AGAACAGAAGAAGGCAATCGTCGACCTGCTGTTCAAGACAAACAGAAAGGTCACAGTCAAGCAGCTGAAG |
| | | GAAGACTACTTCAAGAAGATCGAATGCTTCGACAGCGTCGAAATCAGCGGAGTCGAAGACAGATTCAACGC |
| | | AAGCCTGGGAACATACCACGACCTGCTGAAGATCATCAAGGACAAGGACTTCCTGGACAACGAAGAAAACG |
| | | AAGACATCCTGGAAGACATCGTCCTGACACTGACACTGTTCGAAGACAGAGAAATGATCGAAGAAAGACTG |
| | | AAGACATACGCACACCTGTTCGACGACAAGGTCATGAAGCAGCTGAAGAGAAGAAGATACACAGGATGGG |
| | | GAAGACTGAGCAGAAAGCTGATCAACGGAATCAGAGACAAGCAGAGCGGAAAGACAATCCTGGACTTCCT |
| | | GAAGAGCGACGGATTCGCAAACAGAAACTTCATGCAGCTGATCCACGACGACAGCCTGACATTCAAGGAAG |
| | | ACATCCAGAAGGCACAGGTCAGCGGACAGGGAGACAGCCTGCACGAACACATCGCAAACCTGGCAGGAAG |
| | | CCCGGCAATCAAGAAGGGAATCCTGCAGACAGTCAAGGTCGTCGACGAACTGGTCAAGGTCATGGGAAGAC |
| | | ACAAGCCGGAAAACATCGTCATCGAAATGGCAAGAGAAAACCAGACAACACAGAAGGGACAGAAGAACAG |
| | | CAGAGAAAGAATGAAGAGAATCGAAGAAGGAATCAAGGAACTGGGAAGCCAGATCCTGAAGGAACACCCG |
| | | GTCGAAAACACACAGCTGCAGAACGAAAAGCTGTACCTGTACTACCTGCAGAACGGAAGAGACATGTACGT |
| | | CGACCAGGAACTGGACATCAACAGACTGAGCGACTACGACGTCGACCACATCGTCCCGCAGAGCTTCCTGA |
| | | AGGACGACAGCATCGACAACAAGGTCCTGACAAGAAGCGACAAGAACAGAGGAAAGAGCGACAACGTCCC |
| | | GAGCGAAGAAGTCGTCAAGAAGATGAAGAACTACTGGAGACAGCTGCTGAACGCAAAGCTGATCACACAG |
| | | AGAAAGTTCGACAACCTGACAAAGGCAGAGAGAGGAGGACTGAGCGAACTGGACAAGGCAGGATTCATCA |
| | | AGAGACAGCTGGTCGAAACAAGACAGATCACAAAGCACGTCGCACAGATCCTGGACAGCAGAATGAACAC |
| | | AAAGTACGACGAAAACGACAAGCTGATCAGAGAAGTCAAGGTCATCACACTGAAGAGCAAGCTGGTCAGC |
| | | GACTTCAGAAAGGACTTCCAGTTCTACAAGGTCAGAGAAATCAACAACTACCACCACGCACACGACGCATA |
| | | CCTGAACGCAGTCGTCGGAACAGCACTGATCAAGAAGTACCCGAAGCTGGAAAGCGAATTCGTCTACGGAG |
| | | ACTACAAGGTCTACGACGTCAGAAAGATGATCGCAAAGAGCGAACAGGAAATCGGAAAGGCAACAGCAAA |
| | | GTACTTCTTCTACAGCAACATCATGAACTTCTTCAAGACAGAAATCACACTGGCAAACGGAGAAATCAGAAA |
| | | GAGACCGCTGATCGAAACAAACGGAGAAACAGGAGAAATCGTCTGGGACAAGGGAAGAGACTTCGCAACA |
| | | GTCAGAAAGGTCCTGAGCATGCCGCAGGTCAACATCGTCAAGAAGACAGAAGTCCAGACAGGAGGATTCAG |
| | | CAAGGAAAGCATCCTGCCGAAGAGAAACAGCGACAAGCTGATCGCAAGAAAGAAGGACTGGGACCCGAAG |
| | | AAGTACGGAGGATTCGACAGCCCGACAGTCGCATACAGCGTCCTGGTCGTCGCAAAGGTCGAAAAGGGAAA |
| | | GAGCAAGAAGCTGAAGAGCGTCAAGGAACTGCTGGGAATCACAATCATGGAAAGAAGCAGCTTCGAAAAG |
| | | AACCCGATCGACTTCCTGGAAGCAAAGGGATACAAGGAAGTCAAGAAGGACCTGATCATCAAGCTGCCGAA |
| | | GTACAGCCTGTTCGAACTGGAAAACGGAAGAAAGAGAATGCTGGCAAGCGCAGGAGAACTGCAGAAGGGA |
| | | AACGAACTGGCACTGCCGAGCAAGTACGTCAACTTCCTGTACCTGGCAAGCCACTACGAAAAGCTGAAGGG |
| | | AAGCCCGGAAGACAACGAACAGAAGCAGCTGTTCGTCGAACAGCACAAGCACTACCTGGACGAAATCATCG |
| | | AACAGATCAGCGAATTCAGCAAGAGAGTCATCCTGGCAGACGCAAACCTGGACAAGGTCCTGAGCGCATAC |
| | | AACAAGCACAGAGACAAGCCGATCAGAGAACAGGCAGAAAACATCATCCACCTGTTCACACTGACAAACCT |
| | | GGGAGCACCGGCAGCATTCAAGTACTTCGACACAACAATCGACAGAAAGAGATACACAAGCACAAAGGAA |
| | | GTCCTGGACGCAACACTGATCCACCAGAGCATCACAGGACTGTACGAAACAAGAATCGACCTGAGCCAGCT |
| | | GGGAGGAGACGGAGGAGGAAGCCCGAAGAAGAAGAGAAAGGTCTAGCTAGCCATCACATTTAAAAGCATC |
| | | TCAGCCTACCATGAGAATAAGAGAAAGAAAATGAAGATCAATAGCTTATTCATCTCTTTTTCTTTTTCGTTGG |
| | | TGTAAAGCCAACACCCTGTCTAAAAAACATAAATTTCTTTAATCATTTTGCCTCTTTTCTCTGTGCTTCAATTA |
| | | ATAAAAAATGGAAAGAACCTCGAG |
| 1124 | DNA coding | GGGTCCCGCAGTCGGCGTCCAGCGGCTCTGCTTGTTCGTGTGTGTGTCGTTGCAGGCCTTATTCGGATCCATG |
| | sequence for | GACAAGAAGTACAGCATCGGACTGGACATCGGAACAAACAGCGTCGGATGGGCAGTCATCACAGACGAATA |
| | Cas9 transcript | CAAGGTCCCGAGCAAGAAGTTCAAGGTCCTGGGAAACACAGACAGACACAGCATCAAGAAGAACCTGATC |
| | | GGAGCACTGCTGTTCGACAGCGGAGAAACAGCAGAAGCAACAAGACTGAAGAGAACAGCAAGAAGAAGAT |
| | | ACACAAGAAGAAAGAACAGAATCTGCTACCTGCAGGAAATCTTCAGCAACGAAATGGCAAAGGTCGACGAC |
| | | AGCTTCTTCCACAGACTGGAAGAAAGCTTCCTGGTCGAAGAAGACAAGAAGCACGAAAGACACCCGATCTT |
| | | CGGAAACATCGTCGACGAAGTCGCATACCACGAAAAGTACCCGACAATCTACCACCTGAGAAAGAAGCTGG |
| | | TCGACAGCACAGACAAGGCAGACCTGAGACTGATCTACCTGGCACTGGCACACATGATCAAGTTCAGAGGA |
| | | CACTTCCTGATCGAAGGAGACCTGAACCCGGACAACAGCGACGTCGACAAGCTGTTCATCCAGCTGGTCCA |
| | | GACATACAACCAGCTGTTCGAAGAAAACCCGATCAACGCAAGCGGAGTCGACGCAAAGGCAATCCTGAGCG |
| | | CAAGACTGAGCAAGAGCAGAAGACTGGAAAACCTGATCGCACAGCTGCCGGGAGAAAAGAAGAACGGACT |
| | | GTTCGGAAACCTGATCGCACTGAGCCTGGGACTGACACCGAACTTCAAGAGCAACTTCGACCTGGCAGAAG |
| | | ACGCAAAGCTGCAGCTGAGCAAGGACACATACGACGACGACCTGGACAACCTGCTGGCACAGATCGGAGAC |
| | | CAGTACGCAGACCTGTTCCTGGCAGCAAAGAACCTGAGCGACGCAATCCTGCTGAGCGACATCCTGAGAGT |
| | | CAACACAGAAATCACAAAGGCACCGCTGAGCGCAAGCATGATCAAGAGATACGACGAACACCACCAGGAC |
| | | CTGACACTGCTGAAGGCACTGGTCAGACAGCAGCTGCCGGAAAAGTACAAGGAAATCTTCTTCGACCAGAG |
| | | CAAGAACGGATACGCAGGATACATCGACGGAGGAGCAAGCCAGGAAGAATTCTACAAGTTCATCAAGCCGA |
| | | TCCTGGAAAAGATGGACGGAACAGAAGAACTGCTGGTCAAGCTGAACAGAGAAGACCTGCTGAGAAAGCA |
| | | GAGAACATTCGACAACGGAAGCATCCCGCACCAGATCCACCTGGGAGAACTGCACGCAATCCTGAGAAGAC |
| | | AGGAAGACTTCTACCCGTTCCTGAAGGACAACAGAGAAAAGATCGAAAAGATCCTGACATTCAGAATCCCG |
| | | TACTACGTCGGACCGCTGGCAAGAGGAAACAGCAGATTCGCATGGATGACAAGAAAGAGCGAAGAAACAA |
| | | TCACACCGTGGAACTTCGAAGAAGTCGTCGACAAGGGAGCAAGCGCACAGAGCTTCATCGAAAGAATGACA |
| | | AACTTCGACAAGAACCTGCCGAACGAAAAGGTCCTGCCGAAGCACAGCCTGCTGTACGAATACTTCACAGT |
| | | CTACAACGAACTGACAAAGGTCAAGTACGTCACAGAAGGAATGAGAAAGCCGGCATTCCTGAGCGGAGAAC |
| | | AGAAGAAGGCAATCGTCGACCTGCTGTTCAAGACAAACAGAAAGGTCACAGTCAAGCAGCTGAAGGAAGA |
| | | CTACTTCAAGAAGATCGAATGCTTCGACAGCGTCGAAATCAGCGGAGTCGAAGACAGATTCAACGCAAGCC |
| | | TGGGAACATACCACGACCTGCTGAAGATCATCAAGGACAAGGACTTCCTGGACAACGAAGAAAACGAAGAC |
| | | ATCCTGGAAGACATCGTCCTGACACTGACACTGTTCGAAGACAGAGAAATGATCGAAGAAAGACTGAAGAC |
| | | ATACGCACACCTGTTCGACGACAAGGTCATGAAGCAGCTGAAGAGAAGAAGATACACAGGATGGGGAAGA |
| | | CTGAGCAGAAAGCTGATCAACGGAATCAGAGACAAGCAGAGCGGAAAGACAATCCTGGACTTCCTGAAGA |
| | | GCGACGGATTCGCAAACAGAAACTTCATGCAGCTGATCCACGACGACAGCCTGACATTCAAGGAAGACATC |
| | | CAGAAGGCACAGGTCAGCGGACAGGGAGACAGCCTGCACGAACACATCGCAAACCTGGCAGGAAGCCCGG |
| | | CAATCAAGAAGGGAATCCTGCAGACAGTCAAGGTCGTCGACGAACTGGTCAAGGTCATGGGAAGACACAAG |
| | | CCGGAAAACATCGTCATCGAAATGGCAAGAGAAAACCAGACAACACAGAAGGGACAGAAGAACAGCAGAG |
| | | AAAGAATGAAGAGAATCGAAGAAGGAATCAAGGAACTGGGAAGCCAGATCCTGAAGGAACACCCGGTCGA |
| | | AAACACACAGCTGCAGAACGAAAAGCTGTACCTGTACTACCTGCAGAACGGAAGAGACATGTACGTCGACC |
| | | AGGAACTGGACATCAACAGACTGAGCGACTACGACGTCGACCACATCGTCCCGCAGAGCTTCCTGAAGGAC |
| | | GACAGCATCGACAACAAGGTCCTGACAAGAAGCGACAAGAACAGAGGAAAGAGCGACAACGTCCCGAGCG |
| | | AAGAAGTCGTCAAGAAGATGAAGAACTACTGGAGACAGCTGCTGAACGCAAAGCTGATCACACAGAGAAA |
| | | GTTCGACAACCTGACAAAGGCAGAGAGAGGAGGACTGAGCGAACTGGACAAGGCAGGATTCATCAAGAGA |
| | | CAGCTGGTCGAAACAAGACAGATCACAAAGCACGTCGCACAGATCCTGGACAGCAGAATGAACACAAAGT |
| | | ACGACGAAAACGACAAGCTGATCAGAGAAGTCAAGGTCATCACACTGAAGAGCAAGCTGGTCAGCGACTTC |
| | | AGAAAGGACTTCCAGTTCTACAAGGTCAGAGAAATCAACAACTACCACCACGCACACGACGCATACCTGAA |
| | | CGCAGTCGTCGGAACAGCACTGATCAAGAAGTACCCGAAGCTGGAAAGCGAATTCGTCTACGGAGACTACA |
| | | AGGTCTACGACGTCAGAAAGATGATCGCAAAGAGCGAACAGGAAATCGGAAAGGCAACAGCAAAGTACTT |
| | | CTTCTACAGCAACATCATGAACTTCTTCAAGACAGAAATCACACTGGCAAACGGAGAAATCAGAAAGAGAC |
| | | CGCTGATCGAAACAAACGGAGAAACAGGAGAAATCGTCTGGGACAAGGGAAGAGACTTCGCAACAGTCAG |
| | | AAAGGTCCTGAGCATGCCGCAGGTCAACATCGTCAAGAAGACAGAAGTCCAGACAGGAGGATTCAGCAAGG |
| | | AAAGCATCCTGCCGAAGAGAAACAGCGACAAGCTGATCGCAAGAAAGAAGGACTGGGACCCGAAGAAGTA |
| | | CGGAGGATTCGACAGCCCGACAGTCGCATACAGCGTCCTGGTCGTCGCAAAGGTCGAAAAGGGAAAGAGCA |
| | | AGAAGCTGAAGAGCGTCAAGGAACTGCTGGGAATCACAATCATGGAAAGAAGCAGCTTCGAAAAGAACCC |
| | | GATCGACTTCCTGGAAGCAAAGGGATACAAGGAAGTCAAGAAGGACCTGATCATCAAGCTGCCGAAGTACA |
| | | GCCTGTTCGAACTGGAAAACGGAAGAAAGAGAATGCTGGCAAGCGCAGGAGAACTGCAGAAGGGAAACGA |
| | | ACTGGCACTGCCGAGCAAGTACGTCAACTTCCTGTACCTGGCAAGCCACTACGAAAAGCTGAAGGGAAGCC |
| | | CGGAAGACAACGAACAGAAGCAGCTGTTCGTCGAACAGCACAAGCACTACCTGGACGAAATCATCGAACAG |
| | | ATCAGCGAATTCAGCAAGAGAGTCATCCTGGCAGACGCAAACCTGGACAAGGTCCTGAGCGCATACAACAA |
| | | GCACAGAGACAAGCCGATCAGAGAACAGGCAGAAAACATCATCCACCTGTTCACACTGACAAACCTGGGAG |
| | | CACCGGCAGCATTCAAGTACTTCGACACAACAATCGACAGAAAGAGATACACAAGCACAAAGGAAGTCCTG |
| | | GACGCAACACTGATCCACCAGAGCATCACAGGACTGTACGAAACAAGAATCGACCTGAGCCAGCTGGGAGG |
| | | AGACGGAGGAGGAAGCCCGAAGAAGAAGAGAAAGGTCTAGCTAGCCATCACATTTAAAAGCATCTCAGCCT |
| | | ACCATGAGAATAAGAGAAAGAAAATGAAGATCAATAGCTTATTCATCTCTTTTTCTTTTTCGTTGGTGTAAA |
| | | GCCAACACCCTGTCTAAAAAACATAAATTTCTTTAATCATTTTGCCTCTTTTCTCTGTGCTTCAATTAATAAA |
| | | AAATGGAAAGAACCTCGAG |
| 1125 | Cas9 ORF | ATGGACAAGAAGTACAGCATCGGACTGGACATCGGAACAAACAGCGTCGGATGGGCAGTCATCACAGACGA |
| | | ATACAAGGTCCCGAGCAAGAAGTTCAAGGTCCTGGGAAACACAGACAGACACAGCATCAAGAAGAACCTG |
| | | ATCGGAGCACTGCTGTTCGACAGCGGAGAAACAGCAGAAGCAACAAGACTGAAGAGAACAGCAAGAAGAA |
| | | GATACACAAGAAGAAAGAACAGAATCTGCTACCTGCAGGAAATCTTCAGCAACGAAATGGCAAAGGTCGAC |
| | | GACAGCTTCTTCCACcggCTGGAAGAAAGCTTCCTGGTCGAAGAAGACAAGAAGCACGAAAGACACCCGATC |
| | | TTCGGAAACATCGTCGACGAAGTCGCATACCACGAAAAGTACCCGACAATCTACCACCTGAGAAAGAAGCT |
| | | GGTCGACAGCACAGACAAGGCAGACCTGAGACTGATCTACCTGGCACTGGCACACATGATCAAGTTCAGAG |
| | | GACACTTCCTGATCGAAGGAGACCTGAACCCGGACAACAGCGACGTCGACAAGCTGTTCATCCAGCTGGTC |
| | | CAGACATACAACCAGCTGTTCGAAGAAAACCCGATCAACGCAAGCGGAGTCGACGCAAAGGCAATCCTGAG |
| | | CGCAAGACTGAGCAAGAGCAGAAGACTGGAAAACCTGATCGCACAGCTGCCGGGAGAAAAGAAGAACGGA |
| | | CTGTTCGGAAACCTGATCGCACTGAGCCTGGGACTGACACCGAACTTCAAGAGCAACTTCGACCTGGCAGA |
| | | AGACGCAAAGCTGCAGCTGAGCAAGGACACATACGACGACGACCTGGACAACCTGCTGGCACAGATCGGA |
| | | GACCAGTACGCAGACCTGTTCCTGGCAGCAAAGAACCTGAGCGACGCAATCCTGCTGAGCGACATCCTGAG |
| | | AGTCAACACAGAAATCACAAAGGCACCGCTGAGCGCAAGCATGATCAAGAGATACGACGAACACCACCAG |
| | | GACCTGACACTGCTGAAGGCACTGGTCAGACAGCAGCTGCCGGAAAAGTACAAGGAAATCTTCTTCGACCA |
| | | GAGCAAGAACGGATACGCAGGATACATCGACGGAGGAGCAAGCCAGGAAGAATTCTACAAGTTCATCAAG |
| | | CCGATCCTGGAAAAGATGGACGGAACAGAAGAACTGCTGGTCAAGCTGAACAGAGAAGACCTGCTGAGAA |
| | | AGCAGAGAACATTCGACAACGGAAGCATCCCGCACCAGATCCACCTGGGAGAACTGCACGCAATCCTGAGA |
| | | AGACAGGAAGACTTCTACCCGTTCCTGAAGGACAACAGAGAAAAGATCGAAAAGATCCTGACATTCAGAAT |
| | | CCCGTACTACGTCGGACCGCTGGCAAGAGGAAACAGCAGATTCGCATGGATGACAAGAAAGAGCGAAGAA |
| | | ACAATCACACCGTGGAACTTCGAAGAAGTCGTCGACAAGGGAGCAAGCGCACAGAGCTTCATCGAAAGAAT |
| | | GACAAACTTCGACAAGAACCTGCCGAACGAAAAGGTCCTGCCGAAGCACAGCCTGCTGTACGAATACTTCA |
| | | CAGTCTACAACGAACTGACAAAGGTCAAGTACGTCACAGAAGGAATGAGAAAGCCGGCATTCCTGAGCGGA |
| | | GAACAGAAGAAGGCAATCGTCGACCTGCTGTTCAAGACAAACAGAAAGGTCACAGTCAAGCAGCTGAAGG |
| | | AAGACTACTTCAAGAAGATCGAATGCTTCGACAGCGTCGAAATCAGCGGAGTCGAAGACAGATTCAACGCA |
| | | AGCCTGGGAACATACCACGACCTGCTGAAGATCATCAAGGACAAGGACTTCCTGGACAACGAAGAAAACGA |
| | | AGACATCCTGGAAGACATCGTCCTGACACTGACACTGTTCGAAGACAGAGAAATGATCGAAGAAAGACTGA |
| | | AGACATACGCACACCTGTTCGACGACAAGGTCATGAAGCAGCTGAAGAGAAGAAGATACACAGGATGGGG |
| | | AAGACTGAGCAGAAAGCTGATCAACGGAATCAGAGACAAGCAGAGCGGAAAGACAATCCTGGACTTCCTG |
| | | AAGAGCGACGGATTCGCAAACAGAAACTTCATGCAGCTGATCCACGACGACAGCCTGACATTCAAGGAAGA |
| | | CATCCAGAAGGCACAGGTCAGCGGACAGGGAGACAGCCTGCACGAACACATCGCAAACCTGGCAGGAAGC |
| | | CCGGCAATCAAGAAGGGAATCCTGCAGACAGTCAAGGTCGTCGACGAACTGGTCAAGGTCATGGGAAGACA |
| | | CAAGCCGGAAAACATCGTCATCGAAATGGCAAGAGAAAACCAGACAACACAGAAGGGACAGAAGAACAGC |
| | | AGAGAAAGAATGAAGAGAATCGAAGAAGGAATCAAGGAACTGGGAAGCCAGATCCTGAAGGAACACCCGG |
| | | TCGAAAACACACAGCTGCAGAACGAAAAGCTGTACCTGTACTACCTGCAaAACGGAAGAGACATGTACGTC |
| | | GACCAGGAACTGGACATCAACAGACTGAGCGACTACGACGTCGACCACATCGTCCCGCAGAGCTTCCTGAA |
| | | GGACGACAGCATCGACAACAAGGTCCTGACAAGAAGCGACAAGAACAGAGGAAAGAGCGACAACGTCCCG |
| | | AGCGAAGAAGTCGTCAAGAAGATGAAGAACTACTGGAGACAGCTGCTGAACGCAAAGCTGATCACACAGA |
| | | GAAAGTTCGACAACCTGACAAAGGCAGAGAGAGGAGGACTGAGCGAACTGGACAAGGCAGGATTCATCAA |
| | | GAGACAGCTGGTCGAAACAAGACAGATCACAAAGCACGTCGCACAGATCCTGGACAGCAGAATGAACACA |
| | | AAGTACGACGAAAACGACAAGCTGATCAGAGAAGTCAAGGTCATCACACTGAAGAGCAAGCTGGTCAGCG |
| | | ACTTCAGAAAGGACTTCCAGTTCTACAAGGTCAGAGAAATCAACAACTACCACCACGCACACGACGCATAC |
| | | CTGAACGCAGTCGTCGGAACAGCACTGATCAAGAAGTACCCGAAGCTGGAAAGCGAATTCGTCTACGGAGA |
| | | CTACAAGGTCTACGACGTCAGAAAGATGATCGCAAAGAGCGAACAGGAAATCGGAAAGGCAACAGCAAAG |
| | | TACTTCTTCTACAGCAACATCATGAACTTCTTCAAGACAGAAATCACACTGGCAAACGGAGAAATCAGAAAG |
| | | AGACCGCTGATCGAAACAAACGGAGAAACAGGAGAAATCGTCTGGGACAAGGGAAGAGACTTCGCAACAG |
| | | TCAGAAAGGTCCTGAGCATGCCGCAGGTCAACATCGTCAAGAAGACAGAAGTCCAGACAGGAGGATTCAGC |
| | | AAGGAAAGCATCCTGCCGAAGAGAAACAGCGACAAGCTGATCGCAAGAAAGAAGGACTGGGACCCGAAGA |
| | | AGTACGGAGGATTCGACAGCCCGACAGTCGCATACAGCGTCCTGGTCGTCGCAAAGGTCGAAAAGGGAAAG |
| | | AGCAAGAAGCTGAAGAGCGTCAAGGAACTGCTGGGAATCACAATCATGGAAAGAAGCAGCTTCGAAAAGA |
| | | ACCCGATCGACTTCCTGGAAGCAAAGGGATACAAGGAAGTCAAGAAGGACCTGATCATCAAGCTGCCGAAG |
| | | TACAGCCTGTTCGAACTGGAAAACGGAAGAAAGAGAATGCTGGCAAGCGCAGGAGAACTGCAGAAGGGAA |
| | | ACGAACTGGCACTGCCGAGCAAGTACGTCAACTTCCTGTACCTGGCAAGCCACTACGAAAAGCTGAAGGGA |
| | | AGCCCGGAAGACAACGAACAGAAGCAGCTGTTCGTCGAACAGCACAAGCACTACCTGGACGAAATCATCGA |
| | | ACAGATCAGCGAATTCAGCAAGAGAGTCATCCTGGCAGACGCAAACCTGGACAAGGTCCTGAGCGCATACA |
| | | ACAAGCACAGAGACAAGCCGATCAGAGAACAGGCAGAAAACATCATCCACCTGTTCACACTGACAAACCTG |
| | | GGAGCACCGGCAGCATTCAAGTACTTCGACACAACAATCGACAGAAAGAGATACACAAGCACAAAGGAAG |
| | | TCCTGGACGCAACACTGATCCACCAGAGCATCACAGGACTGTACGAAACAAGAATCGACCTGAGCCAGCTG |
| | | GGAGGAGACGGAGGAGGAAGCCCGAAGAAGAAGAGAAAGGTCTAG |
| 1126 | Cas9 ORF | ATGGACAAGAAGTACAGCATCGGCCTGGACATCGGCACCAACAGCGTGGGCTGGGCCGTGATCACCGACGA |
| | | GTACAAGGTGCCCAGCAAGAAGTTCAAGGTGCTGGGCAACACCGACAGACACAGCATCAAGAAGAACCTG |
| | | ATCGGCGCCCTGCTGTTCGACAGCGGCGAGACCGCCGAGGCCACCAGACTGAAGAGAACCGCCAGAAGAAG |
| | | ATACACCAGAAGAAAGAACAGAATCTGCTACCTGCAGGAGATCTTCAGCAACGAGATGGCCAAGGTGGACG |
| | | ACAGCTTCTTCCACAGACTGGAGGAGAGCTTCCTGGTGGAGGAGGACAAGAAGCACGAGAGACACCCCATC |
| | | TTCGGCAACATCGTGGACGAGGTGGCCTACCACGAGAAGTACCCCACCATCTACCACCTGAGAAAGAAGCT |
| | | GGTGGACAGCACCGACAAGGCCGACCTGAGACTGATCTACCTGGCCCTGGCCCACATGATCAAGTTCAGAG |
| | | GCCACTTCCTGATCGAGGGCGACCTGAACCCCGACAACAGCGACGTGGACAAGCTGTTCATCCAGCTGGTGC |
| | | AGACCTACAACCAGCTGTTCGAGGAGAACCCCATCAACGCCAGCGGCGTGGACGCCAAGGCCATCCTGAGC |
| | | GCCAGACTGAGCAAGAGCAGAAGACTGGAGAACCTGATCGCCCAGCTGCCCGGCGAGAAGAAGAACGGCC |
| | | TGTTCGGCAACCTGATCGCCCTGAGCCTGGGCCTGACCCCCAACTTCAAGAGCAACTTCGACCTGGCCGAGG |
| | | ACGCCAAGCTGCAGCTGAGCAAGGACACCTACGACGACGACCTGGACAACCTGCTGGCCCAGATCGGCGAC |
| | | CAGTACGCCGACCTGTTCCTGGCCGCCAAGAACCTGAGCGACGCCATCCTGCTGAGCGACATCCTGAGAGTG |
| | | AACACCGAGATCACCAAGGCCCCCCTGAGCGCCAGCATGATCAAGAGATACGACGAGCACCACCAGGACCT |
| | | GACCCTGCTGAAGGCCCTGGTGAGACAGCAGCTGCCCGAGAAGTACAAGGAGATCTTCTTCGACCAGAGCA |
| | | AGAACGGCTACGCCGGCTACATCGACGGCGGCGCCAGCCAGGAGGAGTTCTACAAGTTCATCAAGCCCATC |
| | | CTGGAGAAGATGGACGGCACCGAGGAGCTGCTGGTGAAGCTGAACAGAGAGGACCTGCTGAGAAAGCAGA |
| | | GAACCTTCGACAACGGCAGCATCCCCCACCAGATCCACCTGGGCGAGCTGCACGCCATCCTGAGAAGACAG |
| | | GAGGACTTCTACCCCTTCCTGAAGGACAACAGAGAGAAGATCGAGAAGATCCTGACCTTCAGAATCCCCTA |
| | | CTACGTGGGCCCCCTGGCCAGAGGCAACAGCAGATTCGCCTGGATGACCAGAAAGAGCGAGGAGACCATCA |
| | | CCCCCTGGAACTTCGAGGAGGTGGTGGACAAGGGCGCCAGCGCCCAGAGCTTCATCGAGAGAATGACCAAC |
| | | TTCGACAAGAACCTGCCCAACGAGAAGGTGCTGCCCAAGCACAGCCTGCTGTACGAGTACTTCACCGTGTAC |
| | | AACGAGCTGACCAAGGTGAAGTACGTGACCGAGGGCATGAGAAAGCCCGCCTTCCTGAGCGGCGAGCAGA |
| | | AGAAGGCCATCGTGGACCTGCTGTTCAAGACCAACAGAAAGGTGACCGTGAAGCAGCTGAAGGAGGACTAC |
| | | TTCAAGAAGATCGAGTGCTTCGACAGCGTGGAGATCAGCGGCGTGGAGGACAGATTCAACGCCAGCCTGGG |
| | | CACCTACCACGACCTGCTGAAGATCATCAAGGACAAGGACTTCCTGGACAACGAGGAGAACGAGGACATCC |
| | | TGGAGGACATCGTGCTGACCCTGACCCTGTTCGAGGACAGAGAGATGATCGAGGAGAGACTGAAGACCTAC |
| | | GCCCACCTGTTCGACGACAAGGTGATGAAGCAGCTGAAGAGAAGAAGATACACCGGCTGGGGCAGACTGAG |
| | | CAGAAAGCTGATCAACGGCATCAGAGACAAGCAGAGCGGCAAGACCATCCTGGACTTCCTGAAGAGCGACG |
| | | GCTTCGCCAACAGAAACTTCATGCAGCTGATCCACGACGACAGCCTGACCTTCAAGGAGGACATCCAGAAG |
| | | GCCCAGGTGAGCGGCCAGGGCGACAGCCTGCACGAGCACATCGCCAACCTGGCCGGCAGCCCCGCCATCAA |
| | | GAAGGGCATCCTGCAGACCGTGAAGGTGGTGGACGAGCTGGTGAAGGTGATGGGCAGACACAAGCCCGAG |
| | | AACATCGTGATCGAGATGGCCAGAGAGAACCAGACCACCCAGAAGGGCCAGAAGAACAGCAGAGAGAGAA |
| | | TGAAGAGAATCGAGGAGGGCATCAAGGAGCTGGGCAGCCAGATCCTGAAGGAGCACCCCGTGGAGAACAC |
| | | CCAGCTGCAGAACGAGAAGCTGTACCTGTACTACCTGCAGAACGGCAGAGACATGTACGTGGACCAGGAGC |
| | | TGGACATCAACAGACTGAGCGACTACGACGTGGACCACATCGTGCCCCAGAGCTTCCTGAAGGACGACAGC |
| | | ATCGACAACAAGGTGCTGACCAGAAGCGACAAGAACAGAGGCAAGAGCGACAACGTGCCCAGCGAGGAGG |
| | | TGGTGAAGAAGATGAAGAACTACTGGAGACAGCTGCTGAACGCCAAGCTGATCACCCAGAGAAAGTTCGAC |
| | | AACCTGACCAAGGCCGAGAGAGGCGGCCTGAGCGAGCTGGACAAGGCCGGCTTCATCAAGAGACAGCTGGT |
| | | GGAGACCAGACAGATCACCAAGCACGTGGCCCAGATCCTGGACAGCAGAATGAACACCAAGTACGACGAG |
| | | AACGACAAGCTGATCAGAGAGGTGAAGGTGATCACCCTGAAGAGCAAGCTGGTGAGCGACTTCAGAAAGG |
| | | ACTTCCAGTTCTACAAGGTGAGAGAGATCAACAACTACCACCACGCCCACGACGCCTACCTGAACGCCGTG |
| | | GTGGGCACCGCCCTGATCAAGAAGTACCCCAAGCTGGAGAGCGAGTTCGTGTACGGCGACTACAAGGTGTA |
| | | CGACGTGAGAAAGATGATCGCCAAGAGCGAGCAGGAGATCGGCAAGGCCACCGCCAAGTACTTCTTCTACA |
| | | GCAACATCATGAACTTCTTCAAGACCGAGATCACCCTGGCCAACGGCGAGATCAGAAAGAGACCCCTGATC |
| | | GAGACCAACGGCGAGACCGGCGAGATCGTGTGGGACAAGGGCAGAGACTTCGCCACCGTGAGAAAGGTGC |
| | | TGAGCATGCCCCAGGTGAACATCGTGAAGAAGACCGAGGTGCAGACCGGCGGCTTCAGCAAGGAGAGCATC |
| | | CTGCCCAAGAGAAACAGCGACAAGCTGATCGCCAGAAAGAAGGACTGGGACCCCAAGAAGTACGGCGGCT |
| | | TCGACAGCCCCACCGTGGCCTACAGCGTGCTGGTGGTGGCCAAGGTGGAGAAGGGCAAGAGCAAGAAGCTG |
| | | AAGAGCGTGAAGGAGCTGCTGGGCATCACCATCATGGAGAGAAGCAGCTTCGAGAAGAACCCCATCGACTT |
| | | CCTGGAGGCCAAGGGCTACAAGGAGGTGAAGAAGGACCTGATCATCAAGCTGCCCAAGTACAGCCTGTTCG |
| | | AGCTGGAGAACGGCAGAAAGAGAATGCTGGCCAGCGCCGGCGAGCTGCAGAAGGGCAACGAGCTGGCCCT |
| | | GCCCAGCAAGTACGTGAACTTCCTGTACCTGGCCAGCCACTACGAGAAGCTGAAGGGCAGCCCCGAGGACA |
| | | ACGAGCAGAAGCAGCTGTTCGTGGAGCAGCACAAGCACTACCTGGACGAGATCATCGAGCAGATCAGCGAG |
| | | TTCAGCAAGAGAGTGATCCTGGCCGACGCCAACCTGGACAAGGTGCTGAGCGCCTACAACAAGCACAGAGA |
| | | CAAGCCCATCAGAGAGCAGGCCGAGAACATCATCCACCTGTTCACCCTGACCAACCTGGGCGCCCCCGCCG |
| | | CCTTCAAGTACTTCGACACCACCATCGACAGAAAGAGATACACCAGCACCAAGGAGGTGCTGGACGCCACC |
| | | CTGATCCACCAGAGCATCACCGGCCTGTACGAGACCAGAATCGACCTGAGCCAGCTGGGCGGCGACGGCGG |
| | | CGGCAGCCCCAAGAAGAAGAGAAAGGTGTGA |
| 1127 | DNA coding | GGGTCCCGCAGTCGGCGTCCAGCGGCTCTGCTTGTTCGTGTGTGTGTCGTTGCAGGCCTTATTCGGATCCGCC |
| | sequence for | ACCATGGACAAGAAGTACAGCATCGGCCTGGACATCGGCACCAACAGCGTGGGCTGGGCCGTGATCACCGA |
| | Cas9 transcript | CGAGTACAAGGTGCCCAGCAAGAAGTTCAAGGTGCTGGGCAACACCGACAGACACAGCATCAAGAAGAAC |
| | | CTGATCGGCGCCCTGCTGTTCGACAGCGGCGAGACCGCCGAGGCCACCAGACTGAAGAGAACCGCCAGAAG |
| | | AAGATACACCAGAAGAAAGAACAGAATCTGCTACCTGCAGGAGATCTTCAGCAACGAGATGGCCAAGGTGG |
| | | ACGACAGCTTCTTCCACAGACTGGAGGAGAGCTTCCTGGTGGAGGAGGACAAGAAGCACGAGAGACACCCC |
| | | ATCTTCGGCAACATCGTGGACGAGGTGGCCTACCACGAGAAGTACCCCACCATCTACCACCTGAGAAAGAA |
| | | GCTGGTGGACAGCACCGACAAGGCCGACCTGAGACTGATCTACCTGGCCCTGGCCCACATGATCAAGTTCA |
| | | GAGGCCACTTCCTGATCGAGGGCGACCTGAACCCCGACAACAGCGACGTGGACAAGCTGTTCATCCAGCTG |
| | | GTGCAGACCTACAACCAGCTGTTCGAGGAGAACCCCATCAACGCCAGCGGCGTGGACGCCAAGGCCATCCT |
| | | GAGCGCCAGACTGAGCAAGAGCAGAAGACTGGAGAACCTGATCGCCCAGCTGCCCGGCGAGAAGAAGAAC |
| | | GGCCTGTTCGGCAACCTGATCGCCCTGAGCCTGGGCCTGACCCCCAACTTCAAGAGCAACTTCGACCTGGCC |
| | | GAGGACGCCAAGCTGCAGCTGAGCAAGGACACCTACGACGACGACCTGGACAACCTGCTGGCCCAGATCGG |
| | | CGACCAGTACGCCGACCTGTTCCTGGCCGCCAAGAACCTGAGCGACGCCATCCTGCTGAGCGACATCCTGAG |
| | | AGTGAACACCGAGATCACCAAGGCCCCCCTGAGCGCCAGCATGATCAAGAGATACGACGAGCACCACCAGG |
| | | ACCTGACCCTGCTGAAGGCCCTGGTGAGACAGCAGCTGCCCGAGAAGTACAAGGAGATCTTCTTCGACCAG |
| | | AGCAAGAACGGCTACGCCGGCTACATCGACGGCGGCGCCAGCCAGGAGGAGTTCTACAAGTTCATCAAGCC |
| | | CATCCTGGAGAAGATGGACGGCACCGAGGAGCTGCTGGTGAAGCTGAACAGAGAGGACCTGCTGAGAAAG |
| | | CAGAGAACCTTCGACAACGGCAGCATCCCCCACCAGATCCACCTGGGCGAGCTGCACGCCATCCTGAGAAG |
| | | ACAGGAGGACTTCTACCCCTTCCTGAAGGACAACAGAGAGAAGATCGAGAAGATCCTGACCTTCAGAATCC |
| | | CCTACTACGTGGGCCCCCTGGCCAGAGGCAACAGCAGATTCGCCTGGATGACCAGAAAGAGCGAGGAGACC |
| | | ATCACCCCCTGGAACTTCGAGGAGGTGGTGGACAAGGGCGCCAGCGCCCAGAGCTTCATCGAGAGAATGAC |
| | | CAACTTCGACAAGAACCTGCCCAACGAGAAGGTGCTGCCCAAGCACAGCCTGCTGTACGAGTACTTCACCGT |
| | | GTACAACGAGCTGACCAAGGTGAAGTACGTGACCGAGGGCATGAGAAAGCCCGCCTTCCTGAGCGGCGAGC |
| | | AGAAGAAGGCCATCGTGGACCTGCTGTTCAAGACCAACAGAAAGGTGACCGTGAAGCAGCTGAAGGAGGA |
| | | CTACTTCAAGAAGATCGAGTGCTTCGACAGCGTGGAGATCAGCGGCGTGGAGGACAGATTCAACGCCAGCC |
| | | TGGGCACCTACCACGACCTGCTGAAGATCATCAAGGACAAGGACTTCCTGGACAACGAGGAGAACGAGGAC |
| | | ATCCTGGAGGACATCGTGCTGACCCTGACCCTGTTCGAGGACAGAGAGATGATCGAGGAGAGACTGAAGAC |
| | | CTACGCCCACCTGTTCGACGACAAGGTGATGAAGCAGCTGAAGAGAAGAAGATACACCGGCTGGGGCAGAC |
| | | TGAGCAGAAAGCTGATCAACGGCATCAGAGACAAGCAGAGCGGCAAGACCATCCTGGACTTCCTGAAGAGC |
| | | GACGGCTTCGCCAACAGAAACTTCATGCAGCTGATCCACGACGACAGCCTGACCTTCAAGGAGGACATCCA |
| | | GAAGGCCCAGGTGAGCGGCCAGGGCGACAGCCTGCACGAGCACATCGCCAACCTGGCCGGCAGCCCCGCCA |
| | | TCAAGAAGGGCATCCTGCAGACCGTGAAGGTGGTGGACGAGCTGGTGAAGGTGATGGGCAGACACAAGCCC |
| | | GAGAACATCGTGATCGAGATGGCCAGAGAGAACCAGACCACCCAGAAGGGCCAGAAGAACAGCAGAGAGA |
| | | GAATGAAGAGAATCGAGGAGGGCATCAAGGAGCTGGGCAGCCAGATCCTGAAGGAGCACCCCGTGGAGAA |
| | | CACCCAGCTGCAGAACGAGAAGCTGTACCTGTACTACCTGCAGAACGGCAGAGACATGTACGTGGACCAGG |
| | | AGCTGGACATCAACAGACTGAGCGACTACGACGTGGACCACATCGTGCCCCAGAGCTTCCTGAAGGACGAC |
| | | AGCATCGACAACAAGGTGCTGACCAGAAGCGACAAGAACAGAGGCAAGAGCGACAACGTGCCCAGCGAGG |
| | | AGGTGGTGAAGAAGATGAAGAACTACTGGAGACAGCTGCTGAACGCCAAGCTGATCACCCAGAGAAAGTTC |
| | | GACAACCTGACCAAGGCCGAGAGAGGCGGCCTGAGCGAGCTGGACAAGGCCGGCTTCATCAAGAGACAGC |
| | | TGGTGGAGACCAGACAGATCACCAAGCACGTGGCCCAGATCCTGGACAGCAGAATGAACACCAAGTACGAC |
| | | GAGAACGACAAGCTGATCAGAGAGGTGAAGGTGATCACCCTGAAGAGCAAGCTGGTGAGCGACTTCAGAA |
| | | AGGACTTCCAGTTCTACAAGGTGAGAGAGATCAACAACTACCACCACGCCCACGACGCCTACCTGAACGCC |
| | | GTGGTGGGCACCGCCCTGATCAAGAAGTACCCCAAGCTGGAGAGCGAGTTCGTGTACGGCGACTACAAGGT |
| | | GTACGACGTGAGAAAGATGATCGCCAAGAGCGAGCAGGAGATCGGCAAGGCCACCGCCAAGTACTTCTTCT |
| | | ACAGCAACATCATGAACTTCTTCAAGACCGAGATCACCCTGGCCAACGGCGAGATCAGAAAGAGACCCCTG |
| | | ATCGAGACCAACGGCGAGACCGGCGAGATCGTGTGGGACAAGGGCAGAGACTTCGCCACCGTGAGAAAGG |
| | | TGCTGAGCATGCCCCAGGTGAACATCGTGAAGAAGACCGAGGTGCAGACCGGCGGCTTCAGCAAGGAGAGC |
| | | ATCCTGCCCAAGAGAAACAGCGACAAGCTGATCGCCAGAAAGAAGGACTGGGACCCCAAGAAGTACGGCG |
| | | GCTTCGACAGCCCCACCGTGGCCTACAGCGTGCTGGTGGTGGCCAAGGTGGAGAAGGGCAAGAGCAAGAAG |
| | | CTGAAGAGCGTGAAGGAGCTGCTGGGCATCACCATCATGGAGAGAAGCAGCTTCGAGAAGAACCCCATCGA |
| | | CTTCCTGGAGGCCAAGGGCTACAAGGAGGTGAAGAAGGACCTGATCATCAAGCTGCCCAAGTACAGCCTGT |
| | | TCGAGCTGGAGAACGGCAGAAAGAGAATGCTGGCCAGCGCCGGCGAGCTGCAGAAGGGCAACGAGCTGGC |
| | | CCTGCCCAGCAAGTACGTGAACTTCCTGTACCTGGCCAGCCACTACGAGAAGCTGAAGGGCAGCCCCGAGG |
| | | ACAACGAGCAGAAGCAGCTGTTCGTGGAGCAGCACAAGCACTACCTGGACGAGATCATCGAGCAGATCAGC |
| | | GAGTTCAGCAAGAGAGTGATCCTGGCCGACGCCAACCTGGACAAGGTGCTGAGCGCCTACAACAAGCACAG |
| | | AGACAAGCCCATCAGAGAGCAGGCCGAGAACATCATCCACCTGTTCACCCTGACCAACCTGGGCGCCCCCG |
| | | CCGCCTTCAAGTACTTCGACACCACCATCGACAGAAAGAGATACACCAGCACCAAGGAGGTGCTGGACGCC |
| | | ACCCTGATCCACCAGAGCATCACCGGCCTGTACGAGACCAGAATCGACCTGAGCCAGCTGGGCGGCGACGG |
| | | CGGCGGCAGCCCCAAGAAGAAGAGAAAGGTGTGACTAGCCATCACATTTAAAAGCATCTCAGCCTACCATG |
| | | AGAATAAGAGAAAGAAAATGAAGATCAATAGCTTATTCATCTCTTTTTCTTTTTCGTTGGTGTAAAGCCAAC |
| | | ACCCTGTCTAAAAAACATAAATTTCTTTAATCATTTTGCCTCTTTTCTCTGTGCTTCAATTAATAAAAAATGG |
| | | AAAGAACCTCGAG |
| 1128 | | Not Used |
| 1129 | Cas9 ORF | ATGGACAAGAAGTACTCTATCGGTTTGGACATCGGTACCAACTCTGTCGGTTGGGCCGTCATCACCGACGAA |
| | | TACAAGGTCCCATCTAAGAAGTTCAAGGTCTTGGGTAACACCGACAGACACTCTATCAAGAAGAACTTGATC |
| | | GGTGCCTTGTTGTTCGACTCTGGTGAAACCGCCGAAGCCACCAGATTGAAGAGAACCGCCAGAAGAAGATA |
| | | CACCAGAAGAAAGAACAGAATCTGCTACTTGCAAGAAATCTTCTCTAACGAAATGGCCAAGGTCGACGACT |
| | | CTTTCTTCCACAGATTGGAAGAATCTTTCTTGGTCGAAGAAGACAAGAAGCACGAAAGACACCCAATCTTCG |
| | | GTAACATCGTCGACGAAGTCGCCTACCACGAAAAGTACCCAACCATCTACCACTTGAGAAAGAAGTTGGTC |
| | | GACTCTACCGACAAGGCCGACTTGAGATTGATCTACTTGGCCTTGGCCCACATGATCAAGTTCAGAGGTCAC |
| | | TTCTTGATCGAAGGTGACTTGAACCCAGACAACTCTGACGTCGACAAGTTGTTCATCCAATTGGTCCAAACC |
| | | TACAACCAATTGTTCGAAGAAAACCCAATCAACGCCTCTGGTGTCGACGCCAAGGCCATCTTGTCTGCCAGA |
| | | TTGTCTAAGAGCAGAAGATTGGAAAACTTGATCGCCCAATTGCCAGGTGAAAAGAAGAACGGTTTGTTCGGT |
| | | AACTTGATCGCCTTGTCTTTGGGTTTGACCCCAAACTTCAAGTCTAACTTCGACTTGGCCGAAGACGCCAAGT |
| | | TGCAATTGTCTAAGGACACCTACGACGACGACTTGGACAACTTGTTGGCCCAAATCGGTGACCAATACGCCG |
| | | ACTTGTTCTTGGCCGCCAAGAACTTGTCTGACGCCATCTTGTTGTCTGACATCTTGAGAGTCAACACCGAAAT |
| | | CACCAAGGCCCCATTGTCTGCCTCTATGATCAAGAGATACGACGAACACCACCAAGACTTGACCTTGTTGAA |
| | | GGCCTTGGTCAGACAACAATTGCCAGAAAAGTACAAGGAAATCTTCTTCGACCAATCTAAGAACGGTTACGC |
| | | CGGTTACATCGACGGTGGTGCCTCTCAAGAAGAATTCTACAAGTTCATCAAGCCAATCTTGGAAAAGATGGA |
| | | CGGTACCGAAGAATTGTTGGTCAAGTTGAACAGAGAAGACTTGTTGAGAAAGCAAAGAACCTTCGACAACG |
| | | GTTCTATCCCACACCAAATCCACTTGGGTGAATTGCACGCCATCTTGAGAAGACAAGAAGACTTCTACCCAT |
| | | TCTTGAAGGACAACAGAGAAAAGATCGAAAAGATCTTGACCTTCAGAATCCCATACTACGTCGGTCCATTGG |
| | | CCAGAGGTAACAGCAGATTCGCCTGGATGACCAGAAAGTCTGAAGAAACCATCACCCCATGGAACTTCGAA |
| | | GAAGTCGTCGACAAGGGTGCCTCTGCCCAATCTTTCATCGAAAGAATGACCAACTTCGACAAGAACTTGCCA |
| | | AACGAAAAGGTCTTGCCAAAGCACTCTTTGTTGTACGAATACTTCACCGTCTACAACGAATTGACCAAGGTC |
| | | AAGTACGTCACCGAAGGTATGAGAAAGCCAGCCTTCTTGTCTGGTGAACAAAAGAAGGCCATCGTCGACTT |
| | | GTTGTTCAAGACCAACAGAAAGGTCACCGTCAAGCAATTGAAGGAAGACTACTTCAAGAAGATCGAATGCT |
| | | TCGACTCTGTCGAAATCTCTGGTGTCGAAGACAGATTCAACGCCTCTTTGGGTACCTACCACGACTTGTTGAA |
| | | GATCATCAAGGACAAGGACTTCTTGGACAACGAAGAAAACGAAGACATCTTGGAAGACATCGTCTTGACCT |
| | | TGACCTTGTTCGAAGACAGAGAAATGATCGAAGAAAGATTGAAGACCTACGCCCACTTGTTCGACGACAAG |
| | | GTCATGAAGCAATTGAAGAGAAGAAGATACACCGGTTGGGGTAGATTGAGCAGAAAGTTGATCAACGGTAT |
| | | CAGAGACAAGCAATCTGGTAAGACCATCTTGGACTTCTTGAAGTCTGACGGTTTCGCCAACAGAAACTTCAT |
| | | GCAATTGATCCACGACGACTCTTTGACCTTCAAGGAAGACATCCAAAAGGCCCAAGTCTCTGGTCAAGGTGA |
| | | CTCTTTGCACGAACACATCGCCAACTTGGCCGGTTCTCCAGCCATCAAGAAGGGTATCTTGCAAACCGTCAA |
| | | GGTCGTCGACGAATTGGTCAAGGTCATGGGTAGACACAAGCCAGAAAACATCGTCATCGAAATGGCCAGAG |
| | | AAAACCAAACCACCCAAAAGGGTCAAAAGAACAGCAGAGAAAGAATGAAGAGAATCGAAGAAGGTATCAA |
| | | GGAATTGGGTTCTCAAATCTTGAAGGAACACCCAGTCGAAAACACCCAATTGCAAAACGAAAAGTTGTACTT |
| | | GTACTACTTGCAAAACGGTAGAGACATGTACGTCGACCAAGAATTGGACATCAACAGATTGTCTGACTACGA |
| | | CGTCGACCACATCGTCCCACAATCTTTCTTGAAGGACGACTCTATCGACAACAAGGTCTTGACCAGATCTGA |
| | | CAAGAACAGAGGTAAGTCTGACAACGTCCCATCTGAAGAAGTCGTCAAGAAGATGAAGAACTACTGGAGAC |
| | | AATTGTTGAACGCCAAGTTGATCACCCAAAGAAAGTTCGACAACTTGACCAAGGCCGAAAGAGGTGGTTTG |
| | | TCTGAATTGGACAAGGCCGGTTTCATCAAGAGACAATTGGTCGAAACCAGACAAATCACCAAGCACGTCGC |
| | | CCAAATCTTGGACAGCAGAATGAACACCAAGTACGACGAAAACGACAAGTTGATCAGAGAAGTCAAGGTCA |
| | | TCACCTTGAAGTCTAAGTTGGTCTCTGACTTCAGAAAGGACTTCCAATTCTACAAGGTCAGAGAAATCAACA |
| | | ACTACCACCACGCCCACGACGCCTACTTGAACGCCGTCGTCGGTACCGCCTTGATCAAGAAGTACCCAAAGT |
| | | TGGAATCTGAATTCGTCTACGGTGACTACAAGGTCTACGACGTCAGAAAGATGATCGCCAAGTCTGAACAAG |
| | | AAATCGGTAAGGCCACCGCCAAGTACTTCTTCTACTCTAACATCATGAACTTCTTCAAGACCGAAATCACCTT |
| | | GGCCAACGGTGAAATCAGAAAGAGACCATTGATCGAAACCAACGGTGAAACCGGTGAAATCGTCTGGGACA |
| | | AGGGTAGAGACTTCGCCACCGTCAGAAAGGTCTTGTCTATGCCACAAGTCAACATCGTCAAGAAGACCGAA |
| | | GTCCAAACCGGTGGTTTCTCTAAGGAATCTATCTTGCCAAAGAGAAACTCTGACAAGTTGATCGCCAGAAAG |
| | | AAGGACTGGGACCCAAAGAAGTACGGTGGTTTCGACTCTCCAACCGTCGCCTACTCTGTCTTGGTCGTCGCC |
| | | AAGGTCGAAAAGGGTAAGTCTAAGAAGTTGAAGTCTGTCAAGGAATTGTTGGGTATCACCATCATGGAAAG |
| | | ATCTTCTTTCGAAAAGAACCCAATCGACTTCTTGGAAGCCAAGGGTTACAAGGAAGTCAAGAAGGACTTGAT |
| | | CATCAAGTTGCCAAAGTACTCTTTGTTCGAATTGGAAAACGGTAGAAAGAGAATGTTGGCCTCTGCCGGTGA |
| | | ATTGCAAAAGGGTAACGAATTGGCCTTGCCATCTAAGTACGTCAACTTCTTGTACTTGGCCTCTCACTACGAA |
| | | AAGTTGAAGGGTTCTCCAGAAGACAACGAACAAAAGCAATTGTTCGTCGAACAACACAAGCACTACTTGGA |
| | | CGAAATCATCGAACAAATCTCTGAATTCTCTAAGAGAGTCATCTTGGCCGACGCCAACTTGGACAAGGTCTT |
| | | GTCTGCCTACAACAAGCACAGAGACAAGCCAATCAGAGAACAAGCCGAAAACATCATCCACTTGTTCACCT |
| | | TGACCAACTTGGGTGCCCCAGCCGCCTTCAAGTACTTCGACACCACCATCGACAGAAAGAGATACACCTCTA |
| | | CCAAGGAAGTCTTGGACGCCACCTTGATCCACCAATCTATCACCGGTTTGTACGAAACCAGAATCGACTTGT |
| | | CTCAATTGGGTGGTGACGGTGGTGGTTCTCCAAAGAAGAAGAGAAAGGTCTAA |
| 1130 | Cas9 ORF | ATGGACAAGAAGTACTCCATCGGCCTGGACATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCGGCGGCTCCCCCAAGAAGAAGCGGA |
| | | AGGTGTGA |
| 1131 | Cas9 ORF | ATGGACAAGAAGTACAGCATCGGCCTGGACATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCGGCGGCAGC |
| | | CCCAAGAAGAAGCGGAAGGTGTGA |
| 1132 | Cas9 ORF | ATGGACAAGAAGTACTCCATCGGCCTGGACATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCTCCGGCTCCCCCAAGAAGAAGCGGA |
| | | AGGTGGACGGCTCCCCCAAGAAGAAGCGGAAGGTGGACTCCGGCTGA |
| 1133 | Cas9 nickase | ATGGACAAGAAGTACTCCATCGGCCTGGCCATCGGCACCAACTCCGTGGGCTGGGCCGTGATCACCGACGA |
| | ORF | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCGGCGGCTCCCCCAAGAAGAAGCGGA |
| | | AGGTGTGA |
| 1134 | Cas9 nickase ORF | ATGGACAAGAAGTACTCCATCGGCCTGGCCATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACTGA |
| 1135 | Cas9 nickase | ATGGACAAGAAGTACTCCATCGGCCTGGCCATCGGCACCAACTCCGTGGGCTGGGCCGTGATCACCGACGA |
| | ORF | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACCACATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCTCCGGCTCCCCCAAGAAGAAGCGGA |
| | | AGGTGGACGGCTCCCCCAAGAAGAAGCGGAAGGTGGACTCCGGCTGA |
| 1136 | dCas9 ORF | ATGGACAAGAAGTACTCCATCGGCCTGGCCATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACGCCATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCGGCGGCTCCCCCAAGAAGAAGCGGA |
| | | AGGTGTGA |
| 1137 | dCas9 ORF | ATGGACAAGAAGTACTCCATCGGCCTGGCCATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACGCCATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACTGA |
| 1138 | dCas9 ORF | ATGGACAAGAAGTACTCCATCGGCCTGGCCATCGGCACCAACTCCGTGGGCTGGGCCGTGATCACCGACGA |
| | | GTACAAGGTGCCCTCCAAGAAGTTCAAGGTGCTGGGCAACACCGACCGGCACTCCATCAAGAAGAACCTGA |
| | | TCGGCGCCCTGCTGTTCGACTCCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCTCCAACGAGATGGCCAAGGTGGACGA |
| | | CTCCTTCTTCCACCGGCTGGAGGAGTCCTTCCTGGTGGAGGAGGACAAGAAGCACGAGCGGCACCCCATCTT |
| | | CGGCAACATCGTGGACGAGGTGGCCTACCACGAGAAGTACCCCACCATCTACCACCTGCGGAAGAAGCTGG |
| | | TGGACTCCACCGACAAGGCCGACCTGCGGCTGATCTACCTGGCCCTGGCCCACATGATCAAGTTCCGGGGCC |
| | | ACTTCCTGATCGAGGGCGACCTGAACCCCGACAACTCCGACGTGGACAAGCTGTTCATCCAGCTGGTGCAGA |
| | | CCTACAACCAGCTGTTCGAGGAGAACCCCATCAACGCCTCCGGCGTGGACGCCAAGGCCATCCTGTCCGCCC |
| | | GGCTGTCCAAGTCCCGGCGGCTGGAGAACCTGATCGCCCAGCTGCCCGGCGAGAAGAAGAACGGCCTGTTC |
| | | GGCAACCTGATCGCCCTGTCCCTGGGCCTGACCCCCAACTTCAAGTCCAACTTCGACCTGGCCGAGGACGCC |
| | | AAGCTGCAGCTGTCCAAGGACACCTACGACGACGACCTGGACAACCTGCTGGCCCAGATCGGCGACCAGTA |
| | | CGCCGACCTGTTCCTGGCCGCCAAGAACCTGTCCGACGCCATCCTGCTGTCCGACATCCTGCGGGTGAACAC |
| | | CGAGATCACCAAGGCCCCCCTGTCCGCCTCCATGATCAAGCGGTACGACGAGCACCACCAGGACCTGACCCT |
| | | GCTGAAGGCCCTGGTGCGGCAGCAGCTGCCCGAGAAGTACAAGGAGATCTTCTTCGACCAGTCCAAGAACG |
| | | GCTACGCCGGCTACATCGACGGCGGCGCCTCCCAGGAGGAGTTCTACAAGTTCATCAAGCCCATCCTGGAGA |
| | | AGATGGACGGCACCGAGGAGCTGCTGGTGAAGCTGAACCGGGAGGACCTGCTGCGGAAGCAGCGGACCTTC |
| | | GACAACGGCTCCATCCCCCACCAGATCCACCTGGGCGAGCTGCACGCCATCCTGCGGCGGCAGGAGGACTT |
| | | CTACCCCTTCCTGAAGGACAACCGGGAGAAGATCGAGAAGATCCTGACCTTCCGGATCCCCTACTACGTGGG |
| | | CCCCCTGGCCCGGGGCAACTCCCGGTTCGCCTGGATGACCCGGAAGTCCGAGGAGACCATCACCCCCTGGA |
| | | ACTTCGAGGAGGTGGTGGACAAGGGCGCCTCCGCCCAGTCCTTCATCGAGCGGATGACCAACTTCGACAAG |
| | | AACCTGCCCAACGAGAAGGTGCTGCCCAAGCACTCCCTGCTGTACGAGTACTTCACCGTGTACAACGAGCTG |
| | | ACCAAGGTGAAGTACGTGACCGAGGGCATGCGGAAGCCCGCCTTCCTGTCCGGCGAGCAGAAGAAGGCCAT |
| | | CGTGGACCTGCTGTTCAAGACCAACCGGAAGGTGACCGTGAAGCAGCTGAAGGAGGACTACTTCAAGAAGA |
| | | TCGAGTGCTTCGACTCCGTGGAGATCTCCGGCGTGGAGGACCGGTTCAACGCCTCCCTGGGCACCTACCACG |
| | | ACCTGCTGAAGATCATCAAGGACAAGGACTTCCTGGACAACGAGGAGAACGAGGACATCCTGGAGGACATC |
| | | GTGCTGACCCTGACCCTGTTCGAGGACCGGGAGATGATCGAGGAGCGGCTGAAGACCTACGCCCACCTGTTC |
| | | GACGACAAGGTGATGAAGCAGCTGAAGCGGCGGCGGTACACCGGCTGGGGCCGGCTGTCCCGGAAGCTGAT |
| | | CAACGGCATCCGGGACAAGCAGTCCGGCAAGACCATCCTGGACTTCCTGAAGTCCGACGGCTTCGCCAACC |
| | | GGAACTTCATGCAGCTGATCCACGACGACTCCCTGACCTTCAAGGAGGACATCCAGAAGGCCCAGGTGTCC |
| | | GGCCAGGGCGACTCCCTGCACGAGCACATCGCCAACCTGGCCGGCTCCCCCGCCATCAAGAAGGGCATCCT |
| | | GCAGACCGTGAAGGTGGTGGACGAGCTGGTGAAGGTGATGGGCCGGCACAAGCCCGAGAACATCGTGATCG |
| | | AGATGGCCCGGGAGAACCAGACCACCCAGAAGGGCCAGAAGAACTCCCGGGAGCGGATGAAGCGGATCGA |
| | | GGAGGGCATCAAGGAGCTGGGCTCCCAGATCCTGAAGGAGCACCCCGTGGAGAACACCCAGCTGCAGAACG |
| | | AGAAGCTGTACCTGTACTACCTGCAGAACGGCCGGGACATGTACGTGGACCAGGAGCTGGACATCAACCGG |
| | | CTGTCCGACTACGACGTGGACGCCATCGTGCCCCAGTCCTTCCTGAAGGACGACTCCATCGACAACAAGGTG |
| | | CTGACCCGGTCCGACAAGAACCGGGGCAAGTCCGACAACGTGCCCTCCGAGGAGGTGGTGAAGAAGATGAA |
| | | GAACTACTGGCGGCAGCTGCTGAACGCCAAGCTGATCACCCAGCGGAAGTTCGACAACCTGACCAAGGCCG |
| | | AGCGGGGCGGCCTGTCCGAGCTGGACAAGGCCGGCTTCATCAAGCGGCAGCTGGTGGAGACCCGGCAGATC |
| | | ACCAAGCACGTGGCCCAGATCCTGGACTCCCGGATGAACACCAAGTACGACGAGAACGACAAGCTGATCCG |
| | | GGAGGTGAAGGTGATCACCCTGAAGTCCAAGCTGGTGTCCGACTTCCGGAAGGACTTCCAGTTCTACAAGGT |
| | | GCGGGAGATCAACAACTACCACCACGCCCACGACGCCTACCTGAACGCCGTGGTGGGCACCGCCCTGATCA |
| | | AGAAGTACCCCAAGCTGGAGTCCGAGTTCGTGTACGGCGACTACAAGGTGTACGACGTGCGGAAGATGATC |
| | | GCCAAGTCCGAGCAGGAGATCGGCAAGGCCACCGCCAAGTACTTCTTCTACTCCAACATCATGAACTTCTTC |
| | | AAGACCGAGATCACCCTGGCCAACGGCGAGATCCGGAAGCGGCCCCTGATCGAGACCAACGGCGAGACCG |
| | | GCGAGATCGTGTGGGACAAGGGCCGGGACTTCGCCACCGTGCGGAAGGTGCTGTCCATGCCCCAGGTGAAC |
| | | ATCGTGAAGAAGACCGAGGTGCAGACCGGCGGCTTCTCCAAGGAGTCCATCCTGCCCAAGCGGAACTCCGA |
| | | CAAGCTGATCGCCCGGAAGAAGGACTGGGACCCCAAGAAGTACGGCGGCTTCGACTCCCCCACCGTGGCCT |
| | | ACTCCGTGCTGGTGGTGGCCAAGGTGGAGAAGGGCAAGTCCAAGAAGCTGAAGTCCGTGAAGGAGCTGCTG |
| | | GGCATCACCATCATGGAGCGGTCCTCCTTCGAGAAGAACCCCATCGACTTCCTGGAGGCCAAGGGCTACAA |
| | | GGAGGTGAAGAAGGACCTGATCATCAAGCTGCCCAAGTACTCCCTGTTCGAGCTGGAGAACGGCCGGAAGC |
| | | GGATGCTGGCCTCCGCCGGCGAGCTGCAGAAGGGCAACGAGCTGGCCCTGCCCTCCAAGTACGTGAACTTC |
| | | CTGTACCTGGCCTCCCACTACGAGAAGCTGAAGGGCTCCCCCGAGGACAACGAGCAGAAGCAGCTGTTCGT |
| | | GGAGCAGCACAAGCACTACCTGGACGAGATCATCGAGCAGATCTCCGAGTTCTCCAAGCGGGTGATCCTGG |
| | | CCGACGCCAACCTGGACAAGGTGCTGTCCGCCTACAACAAGCACCGGGACAAGCCCATCCGGGAGCAGGCC |
| | | GAGAACATCATCCACCTGTTCACCCTGACCAACCTGGGCGCCCCCGCCGCCTTCAAGTACTTCGACACCACC |
| | | ATCGACCGGAAGCGGTACACCTCCACCAAGGAGGTGCTGGACGCCACCCTGATCCACCAGTCCATCACCGG |
| | | CCTGTACGAGACCCGGATCGACCTGTCCCAGCTGGGCGGCGACGGCTCCGGCTCCCCCAAGAAGAAGCGGA |
| | | AGGTGGACGGCTCCCCCAAGAAGAAGCGGAAGGTGGACTCCGGCTGA |
| 1139 | Cas9 ORF | ATGGACAAGAAGTACAGCATCGGCCTGGACATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCAGCGGCAGC |
| | | CCCAAGAAGAAGCGGAAGGTGGACGGCAGCCCCAAGAAGAAGCGGAAGGTGGACAGCGGCTGA |
| 1140 | Cas9 ORF | ATGGACAAGAAGTACAGCATCGGCCTGGACATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACTGA |
| 1141 | Cas9 nickase | ATGGACAAGAAGTACAGCATCGGCCTGGCCATCGGCACCAACAGCGTGGGCTGGGCCGTGATCACCGACGA |
| | ORF | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCGGCGGCAGC |
| | | CCCAAGAAGAAGCGGAAGGTGTGA |
| 1142 | Cas9 nickase | ATGGACAAGAAGTACAGCATCGGCCTGGCCATCGGCACCAACAGCGTGGGCTGGGCCGTGATCACCGACGA |
| | ORF | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCAGCGGCAGC |
| | | CCCAAGAAGAAGCGGAAGGTGGACGGCAGCCCCAAGAAGAAGCGGAAGGTGGACAGCGGCTGA |
| 1143 | Cas9 nickase | ATGGACAAGAAGTACAGCATCGGCCTGGcCATCGGCACCAACAGCGTGGGCTGGGCCGTGATCACCGACGA |
| | ORF | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACCACATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACTGA |
| 1144 | dCas9 ORF | ATGGACAAGAAGTACAGCATCGGCCTGGcCATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACgcCATCGTGCCCCAGAGCTTCCTGAAGGACGACAGCATCGA |
| | | CAACAAGGTGCTGACCCGGAGCGACAAGAACCGGGGCAAGAGCGACAACGTGCCCAGCGAGGAGGTGGTG |
| | | AAGAAGATGAAGAACTACTGGCGGCAGCTGCTGAACGCCAAGCTGATCACCCAGCGGAAGTTCGACAACCT |
| | | GACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCGGCGGCAGC |
| | | CCCAAGAAGAAGCGGAAGGTGTGA |
| 1145 | dCas9 ORF | ATGGACAAGAAGTACAGCATCGGCCTGGCCATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACGCCATCGTGCCCCAGAGCTTCCTGAAGGACGACAGCATCG |
| | | ACAACAAGGTGCTGACCCGGAGCGACAAGAACCGGGGCAAGAGCGACAACGTGCCCAGCGAGGAGGTGGT |
| | | GAAGAAGATGAAGAACTACTGGCGGCAGCTGCTGAACGCCAAGCTGATCACCCAGCGGAAGTTCGACAACC |
| | | TGACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACGGCAGCGGCAGC |
| | | CCCAAGAAGAAGCGGAAGGTGGACGGCAGCCCCAAGAAGAAGCGGAAGGTGGACAGCGGCTGA |
| 1146 | dCas9 ORF | ATGGACAAGAAGTACAGCATCGGCCTGGcCATCGGCACCAACAGCGTGGGCTGGGCCGTGATCACCGACGA |
| | | GTACAAGGTGCCCAGCAAGAAGTTCAAGGTGCTGGGCAACACCGACCGGCACAGCATCAAGAAGAACCTGA |
| | | TCGGCGCCCTGCTGTTCGACAGCGGCGAGACCGCCGAGGCCACCCGGCTGAAGCGGACCGCCCGGCGGCGG |
| | | TACACCCGGCGGAAGAACCGGATCTGCTACCTGCAGGAGATCTTCAGCAACGAGATGGCCAAGGTGGACGA |
| | | CAGCTTCTTCCACCGGCTGGAGGAGAGCTTCCTGGTGGAGGAGGACAAGAAGCACGAGCGGCACCCCATCT |
| | | TCGGCAACATCGTGGACGAGGTGGCCTACCACGAGAAGTACCCCACCATCTACCACCTGCGGAAGAAGCTG |
| | | GTGGACAGCACCGACAAGGCCGACCTGCGGCTGATCTACCTGGCCCTGGCCCACATGATCAAGTTCCGGGG |
| | | CCACTTCCTGATCGAGGGCGACCTGAACCCCGACAACAGCGACGTGGACAAGCTGTTCATCCAGCTGGTGCA |
| | | GACCTACAACCAGCTGTTCGAGGAGAACCCCATCAACGCCAGCGGCGTGGACGCCAAGGCCATCCTGAGCG |
| | | CCCGGCTGAGCAAGAGCCGGCGGCTGGAGAACCTGATCGCCCAGCTGCCCGGCGAGAAGAAGAACGGCCTG |
| | | TTCGGCAACCTGATCGCCCTGAGCCTGGGCCTGACCCCCAACTTCAAGAGCAACTTCGACCTGGCCGAGGAC |
| | | GCCAAGCTGCAGCTGAGCAAGGACACCTACGACGACGACCTGGACAACCTGCTGGCCCAGATCGGCGACCA |
| | | GTACGCCGACCTGTTCCTGGCCGCCAAGAACCTGAGCGACGCCATCCTGCTGAGCGACATCCTGCGGGTGAA |
| | | CACCGAGATCACCAAGGCCCCCCTGAGCGCCAGCATGATCAAGCGGTACGACGAGCACCACCAGGACCTGA |
| | | CCCTGCTGAAGGCCCTGGTGCGGCAGCAGCTGCCCGAGAAGTACAAGGAGATCTTCTTCGACCAGAGCAAG |
| | | AACGGCTACGCCGGCTACATCGACGGCGGCGCCAGCCAGGAGGAGTTCTACAAGTTCATCAAGCCCATCCT |
| | | GGAGAAGATGGACGGCACCGAGGAGCTGCTGGTGAAGCTGAACCGGGAGGACCTGCTGCGGAAGCAGCGG |
| | | ACCTTCGACAACGGCAGCATCCCCCACCAGATCCACCTGGGCGAGCTGCACGCCATCCTGCGGCGGCAGGA |
| | | GGACTTCTACCCCTTCCTGAAGGACAACCGGGAGAAGATCGAGAAGATCCTGACCTTCCGGATCCCCTACTA |
| | | CGTGGGCCCCCTGGCCCGGGGCAACAGCCGGTTCGCCTGGATGACCCGGAAGAGCGAGGAGACCATCACCC |
| | | CCTGGAACTTCGAGGAGGTGGTGGACAAGGGCGCCAGCGCCCAGAGCTTCATCGAGCGGATGACCAACTTC |
| | | GACAAGAACCTGCCCAACGAGAAGGTGCTGCCCAAGCACAGCCTGCTGTACGAGTACTTCACCGTGTACAA |
| | | CGAGCTGACCAAGGTGAAGTACGTGACCGAGGGCATGCGGAAGCCCGCCTTCCTGAGCGGCGAGCAGAAGA |
| | | AGGCCATCGTGGACCTGCTGTTCAAGACCAACCGGAAGGTGACCGTGAAGCAGCTGAAGGAGGACTACTTC |
| | | AAGAAGATCGAGTGCTTCGACAGCGTGGAGATCAGCGGCGTGGAGGACCGGTTCAACGCCAGCCTGGGCAC |
| | | CTACCACGACCTGCTGAAGATCATCAAGGACAAGGACTTCCTGGACAACGAGGAGAACGAGGACATCCTGG |
| | | AGGACATCGTGCTGACCCTGACCCTGTTCGAGGACCGGGAGATGATCGAGGAGCGGCTGAAGACCTACGCC |
| | | CACCTGTTCGACGACAAGGTGATGAAGCAGCTGAAGCGGCGGCGGTACACCGGCTGGGGCCGGCTGAGCCG |
| | | GAAGCTGATCAACGGCATCCGGGACAAGCAGAGCGGCAAGACCATCCTGGACTTCCTGAAGAGCGACGGCT |
| | | TCGCCAACCGGAACTTCATGCAGCTGATCCACGACGACAGCCTGACCTTCAAGGAGGACATCCAGAAGGCC |
| | | CAGGTGAGCGGCCAGGGCGACAGCCTGCACGAGCACATCGCCAACCTGGCCGGCAGCCCCGCCATCAAGAA |
| | | GGGCATCCTGCAGACCGTGAAGGTGGTGGACGAGCTGGTGAAGGTGATGGGCCGGCACAAGCCCGAGAACA |
| | | TCGTGATCGAGATGGCCCGGGAGAACCAGACCACCCAGAAGGGCCAGAAGAACAGCCGGGAGCGGATGAA |
| | | GCGGATCGAGGAGGGCATCAAGGAGCTGGGCAGCCAGATCCTGAAGGAGCACCCCGTGGAGAACACCCAG |
| | | CTGCAGAACGAGAAGCTGTACCTGTACTACCTGCAGAACGGCCGGGACATGTACGTGGACCAGGAGCTGGA |
| | | CATCAACCGGCTGAGCGACTACGACGTGGACgcCATCGTGCCCCAGAGCTTCCTGAAGGACGACAGCATCGA |
| | | CAACAAGGTGCTGACCCGGAGCGACAAGAACCGGGGCAAGAGCGACAACGTGCCCAGCGAGGAGGTGGTG |
| | | AAGAAGATGAAGAACTACTGGCGGCAGCTGCTGAACGCCAAGCTGATCACCCAGCGGAAGTTCGACAACCT |
| | | GACCAAGGCCGAGCGGGGCGGCCTGAGCGAGCTGGACAAGGCCGGCTTCATCAAGCGGCAGCTGGTGGAG |
| | | ACCCGGCAGATCACCAAGCACGTGGCCCAGATCCTGGACAGCCGGATGAACACCAAGTACGACGAGAACGA |
| | | CAAGCTGATCCGGGAGGTGAAGGTGATCACCCTGAAGAGCAAGCTGGTGAGCGACTTCCGGAAGGACTTCC |
| | | AGTTCTACAAGGTGCGGGAGATCAACAACTACCACCACGCCCACGACGCCTACCTGAACGCCGTGGTGGGC |
| | | ACCGCCCTGATCAAGAAGTACCCCAAGCTGGAGAGCGAGTTCGTGTACGGCGACTACAAGGTGTACGACGT |
| | | GCGGAAGATGATCGCCAAGAGCGAGCAGGAGATCGGCAAGGCCACCGCCAAGTACTTCTTCTACAGCAACA |
| | | TCATGAACTTCTTCAAGACCGAGATCACCCTGGCCAACGGCGAGATCCGGAAGCGGCCCCTGATCGAGACC |
| | | AACGGCGAGACCGGCGAGATCGTGTGGGACAAGGGCCGGGACTTCGCCACCGTGCGGAAGGTGCTGAGCAT |
| | | GCCCCAGGTGAACATCGTGAAGAAGACCGAGGTGCAGACCGGCGGCTTCAGCAAGGAGAGCATCCTGCCCA |
| | | AGCGGAACAGCGACAAGCTGATCGCCCGGAAGAAGGACTGGGACCCCAAGAAGTACGGCGGCTTCGACAG |
| | | CCCCACCGTGGCCTACAGCGTGCTGGTGGTGGCCAAGGTGGAGAAGGGCAAGAGCAAGAAGCTGAAGAGC |
| | | GTGAAGGAGCTGCTGGGCATCACCATCATGGAGCGGAGCAGCTTCGAGAAGAACCCCATCGACTTCCTGGA |
| | | GGCCAAGGGCTACAAGGAGGTGAAGAAGGACCTGATCATCAAGCTGCCCAAGTACAGCCTGTTCGAGCTGG |
| | | AGAACGGCCGGAAGCGGATGCTGGCCAGCGCCGGCGAGCTGCAGAAGGGCAACGAGCTGGCCCTGCCCAG |
| | | CAAGTACGTGAACTTCCTGTACCTGGCCAGCCACTACGAGAAGCTGAAGGGCAGCCCCGAGGACAACGAGC |
| | | AGAAGCAGCTGTTCGTGGAGCAGCACAAGCACTACCTGGACGAGATCATCGAGCAGATCAGCGAGTTCAGC |
| | | AAGCGGGTGATCCTGGCCGACGCCAACCTGGACAAGGTGCTGAGCGCCTACAACAAGCACCGGGACAAGCC |
| | | CATCCGGGAGCAGGCCGAGAACATCATCCACCTGTTCACCCTGACCAACCTGGGCGCCCCCGCCGCCTTCAA |
| | | GTACTTCGACACCACCATCGACCGGAAGCGGTACACCAGCACCAAGGAGGTGCTGGACGCCACCCTGATCC |
| | | ACCAGAGCATCACCGGCCTGTACGAGACCCGGATCGACCTGAGCCAGCTGGGCGGCGACTGA |

sgRNA designations are sometimes provided with one or more leading zeroes immediately following the G. This does not affect the meaning of the designation. Thus, for example, G000282, G0282, G00282, and G282 refer to the same sgRNA. Similarly, crRNA and or trRNA designations are sometimes provided with one or more leading zeroes immediately following the CR or TR, respectively, which does not affect the meaning of the designation. Thus, for example, CR000100, CR00100, CR0100, and CR100 refer to the same crRNA, and TR000200, TR00200, TR0200, and TR200 refer to the same trRNA.

For SEQ ID NOs 201-294 and 301-394, no guide region is shown and the positions corresponding to the remaining regions are each decremented by the length of the guide sequence in SEQ ID NOs: 1-90, 695-698, 101-190, and 795-798, respectively (usually but not always 20) relative to those given for SEQ ID NOs: 1-90 and 101-190. For SEQ ID NOs 401-494 and 501-594, the spacer is the length of 3+x and the positions corresponding to the remaining regions are each decremented by the length of the guide sequence in SEQ ID NOs: 1-90, 695-698, 101-190, and 795-798, respectively (usually but not always 20) and incremented by 3+x relative to those given for SEQ ID NOs: 1-90, 101-190, and 795-798, respectively.

### Definitions

“Editing efficiency” or “editing percentage” or “percent editing” as used herein is the total number of sequence reads with insertions or deletions of nucleotides into the target region of interest over the total number of sequence reads following cleavage by a Cas RNP.

“Regions” as used herein describes conserved groups of nucleic acids. Regions may also be referred to as “modules” or “domains.” Regions of an sgRNA may perform particular functions, e.g., in directing endonuclease activity of the RNP, for example as described in Briner A E et al., Molecular Cell 56:333-339 (2014). Exemplary regions of an sgRNA are described in Table 3.

“Hairpin” as used herein describes a duplex of nucleic acids that is created when a nucleic acid strand folds and forms base pairs with another section of the same strand. A hairpin may form a structure that comprises a loop or a U-shape. In some embodiments, a hairpin may be comprised of an RNA loop. Hairpins can be formed with two complementary sequences in a single nucleic acid molecule bind together, with a folding or wrinkling of the molecule. In some embodiments, hairpins comprise stem or stem loop structures. As used herein, a “hairpin region” refers to hairpin 1 and hairpin 2 and the “n” between hairpin 1 and hairpin 2 of a conserved portion of an sgRNA.

“Ribonucleoprotein” (RNP) or “RNP complex” as used herein describes an sgRNA, for example, together with a nuclease, such as a Cas protein. In some embodiments, the RNP comprises Cas9 and gRNA (e.g., sgRNA, dgRNA, or crRNA).

“Stem loop” as used herein describes a secondary structure of nucleotides that form a base-paired “stem” that ends in a loop of unpaired nucleic acids. A stem may be formed when two regions of the same nucleic acid strand are at least partially complementary in sequence when read in opposite directions. “Loop” as used herein describes a region of nucleotides that do not base pair (i.e., are not complementary) that may cap a stem. A “tetraloop” describes a loop of 4 nucleotides. As used herein, the upper stem of an sgRNA may comprise a tetraloop.

“Substituted” or “Substitution” as used herein with respect to a polynucleotide refers to an alteration of a nucleobase that changes its preferred base for Watson-Crick pairing. When a certain region of a guide RNA is “unsubstituted” as used herein, the sequence of the region can be aligned to that of the corresponding conserved portion of a spyCas9 sgRNA (SEQ ID NO: 400) with gaps and matches only (i.e., no mismatches), where bases are considered to match if they have the same preferred standard partner base (A, C, G, or T/U) for Watson-Crick pairing.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs can include modified RNAs as described herein.

In some embodiments, the gRNA (e.g., sgRNA) comprises a “guide region”, which is sometimes referred to as a “spacer” or “spacer region,” for example, in Briner A E et al., Molecular Cell 56:333-339 (2014) for sgRNA (but applicable herein to all guide RNAs). The guide region or spacer region is also sometimes referred to as a “variable region,” “guide domain” or “targeting domain.” In some embodiments, a “guide region” immediately precedes a “conserved portion of an sgRNA” at its 5′ end, and in some embodiments the sgRNA is shortened. An exemplary “conserved portion of an sgRNA” is shown in Table 2. In some embodiments, a “guide region” comprises a series of nucleotides at the 5′ end of a crRNA. In some embodiments, the guide region comprises one or more YA sites (“guide region YA sites”). In some embodiments, the guide region comprises one or more YA sites located at positions from a given nucleotide relative to the 5′ end to the end of the guide region. Such ranges of positions are referred to as, e.g., “5-end, 6-end, 7-end, 8-end, 9-end, or 10-end from the 5′ end of the 5′ terminus” where the “end” in “5-end”, etc., refers to most 3′ nucleotide in the guide region. (Similarly, expressions such as “nucleotides 21-end of the gRNA” refer to the range from nucleotide 21 from the 5′ end of the 5′ terminus of the gRNA to the final nucleotide at the 3′ end of the gRNA.) Furthermore, a nucleotide that is, for example, 6 nucleotides from the 5′ end of a particular sgRNA segment is the sixth nucleotide of that segment, or “nucleotide 6” from the 5′ end, e.g., where N is the 6^{th} nucleotide from the 5′ end. A range of nucleotides that is located “at or after” 6 nucleotides from the 5′ end begins with the 6^{th} nucleotide and continues down the chain toward the 3′ end. Similarly, a nucleotide that is, for example, 5 nucleotides from the 3′ end of the chain is the 5^{th} nucleotide when counting from the 3′ end of the chain, e.g. NXXXX. A numeric position or range in the guide region refers to the position as determined from the 5′ end unless another point of reference is specified; for example, “nucleotide 5” in a guide region is the 5^{th} nucleotide from the 5′ end.

In some embodiments, a gRNA comprises nucleotides that “match the modification pattern” at corresponding or specified nucleotides of a gRNA described herein. This means that the nucleotides matching the modification pattern have the same modifications (e.g., phosphorothioate, 2′-fluoro, 2′-OMe, etc.) as the nucleotides at the corresponding positions of the gRNA described herein, regardless of whether the nucleobases at those positions match. For example, if in a first gRNA, nucleotides 5 and 6, respectively, have 2′-OMe and phosphorothioate modifications, then this gRNA has the same modification pattern at nucleotides 5 and 6 as a second gRNA that also has 2′-OMe and phosphorothioate modifications at nucleotides 5 and 6, respectively, regardless of whether the nucleobases at positions 5 and 6 are the same or different in the first and second gRNAs. However, a 2′-OMe modification at nucleotide 6 but not nucleotide 7 is not the same modification pattern at nucleotides 6 and 7 as a 2′-OMe modification at nucleotide 7 but not nucleotide 6. Similarly, a modification pattern that matches at least 75% of the modification pattern of a gRNA described herein means that at least 75% of the nucleotides have the same modifications as the corresponding positions of the gRNA described herein. Corresponding positions may be determined by pairwise or structural alignment.

A “conserved region” of a S. pyogenes Cas9 (“spyCas9” (also referred to as “spCas9”)) sgRNA” is shown in Table 2. The first row shows the numbering of the nucleotides; the second row shows the sequence (e.g., SEQ ID NO: 400); and the third row shows the regions.

As used herein, a “shortened” region in a gRNA is a region in a conserved portion of a gRNA that lacks at least 1 nucleotide compared to the corresponding region in the conserved portion shown in Table 2. Similarly, “shortened” with respect to an sgRNA means that its conserved region comprises fewer nucleotides than the sgRNA conserved region shown in Table 2. Under no circumstances does “shortened” imply any particular limitation on a process or manner of production of the gRNA. In some embodiments, a gRNA comprises a shortened hairpin 1 region, wherein (i) the shortened hairpin 1 region lacks 6-8 nucleotides; and (A) one or more of positions H1-1, H1-2, or H1-3 is deleted or substituted relative to SEQ ID NO: 400 and/or (B) one or more of positions H1-6 through H1-10 is substituted relative to SEQ ID NO: 400; or (ii) the shortened hairpin 1 region lacks 9-10 nucleotides including H1-1 and/or H1-12; or (iii) the shortened hairpin 1 region lacks 5-10 nucleotides and one or more of positions N18, H1-12, or N is substituted relative to SEQ ID NO: 400 (see Table 2). In some embodiments, a non-spyCas9 gRNA comprises a shortened hairpin 1 region that lacks 6-8 nucleotides and in which one or more positions corresponding to H1-1, H1-2, or H1-3 in SEQ ID NO: 400 as determined, for example, by pairwise or structural alignment, is deleted or substituted, one or more of positions corresponding to H1-6 through H1-10 in SEQ ID NO: 400 as determined, for example, by pairwise or structural alignment, is substituted. In some embodiments, a non-spyCas9 gRNA comprises a shortened hairpin 1 region that lacks 9-10 nucleotides including nucleotides corresponding to H1-1 and/or H1-12 in SEQ ID NO: 400 as determined, for example, by pairwise or structural alignment. In some embodiments, a non-spyCas9 gRNA comprises a shortened hairpin 1 region that lacks 5-10 nucleotides and one or more positions corresponding to N18, H1-12, or N in SEQ ID NO: 400 as determined, for example, by pairwise or structural alignment, is substituted. In some embodiments, a gRNA comprises a shortened upper stem region, wherein the shortened upper stem region lacks 1-6 nucleotides.

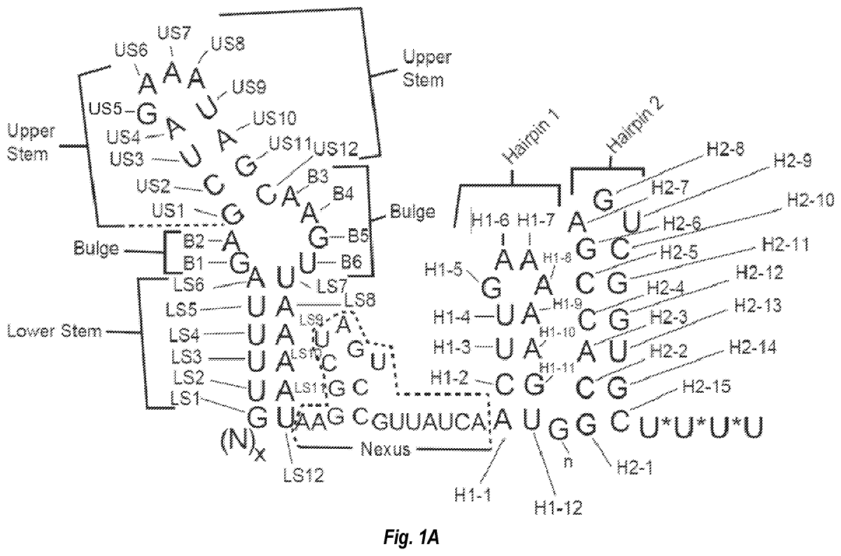

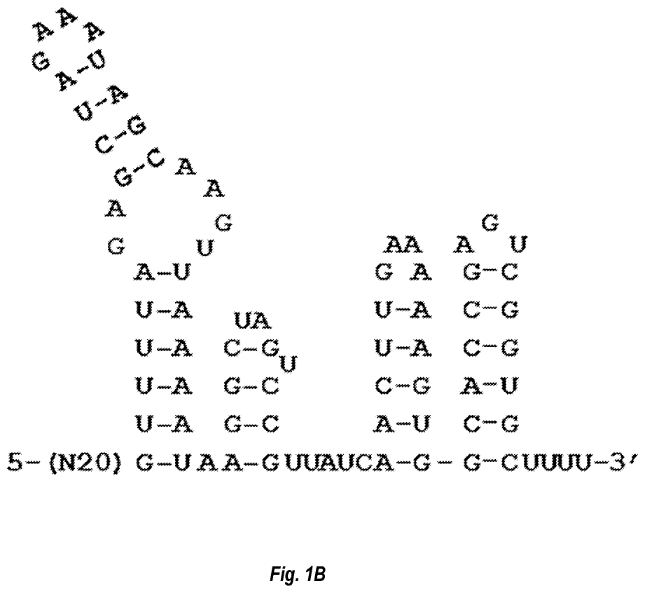

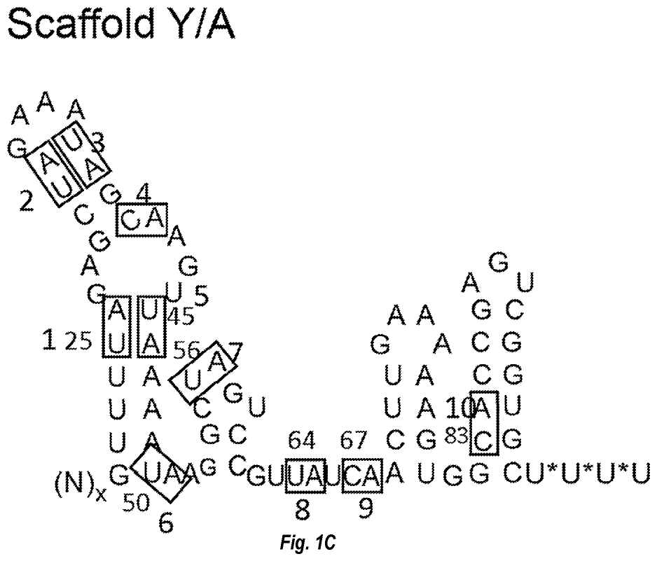

As used herein, a “YA site” refers to a 5′-pyrimidine-adenine-3′ dinucleotide. For clarification, a “YA site” in an original sequence that is altered by modifying a base is still considered a (modified) YA site in the resulting sequence, regardless of the absence of a literal YA dinucleotide. A “conserved region YA site” is present in the conserved region of an sgRNA. A “guide region YA site” is present in the guide region of an sgRNA. An unmodified YA site in an sgRNA may be susceptible to cleavage by RNase-A like endonucleases, e.g., RNase A. In some embodiments, a gRNA comprises about 10 YA sites in its conserved region. In some embodiments, an sgRNA comprises 1, 2, 3, 4, 5, 6, 7, 8, 9, or 10 YA sites in its conserved region. Exemplary conserved region YA sites are indicated in FIG. 1B. Exemplary guide region YA sites are not shown in FIG. 1C, as the guide region may be any sequence, including any number of YA sites. In some embodiments, an sgRNA comprises 1, 2, 3, 4, 5, 6, 7, 8, 9, or 10 of the YA sites indicated in FIG. 1C. In some embodiments, an sgRNA comprises 1, 2, 3, 4, 5, 6, 7, 8, 9, or 10 YA sites at the following positions or a subset thereof: LS5-LS6; US3-US4; US9-US10; US12-B3; LS7-LS8; LS12-N1; N6-N7; N14-N15; N17-N18; and H2-2 to H2-3. In some embodiments, a YA site comprises a substitution, e.g., at any one or more of LS6, LS7, US3, US10, B3, N7, N15, N17, H2-2 and H2-14 relative to SEQ ID NO: 400 (as determined, for example, by pairwise or structural alignment), wherein the substituent nucleotide is neither a pyrimidine that is followed by an adenine, nor an adenine that is preceded by a pyrimidine (thus rendering the substituted position not a part of a YA site). In some embodiments, a YA site comprises a modification, meaning that at least one nucleotide of the YA site is modified. In some embodiments, the pyrimidine (also called the pyrimidine position) of the YA site comprises a modification (which includes a modification altering the internucleoside linkage immediately 3′ of the sugar of the pyrimidine). In some embodiments, the adenine (also called the adenine position) of the YA site comprises a modification (which includes a modification altering the internucleoside linkage immediately 3′ of the sugar of the adenine). In some embodiments, the pyrimidine position and the adenine position of the YA site comprise modifications. In some embodiments, a gRNA guide region or gRNA conserved region described herein comprises one or more YA sites (“guide region YA sites” or “conserved region YA sites”). In some embodiments, a crRNA or a trRNA described herein comprises one or more YA sites.

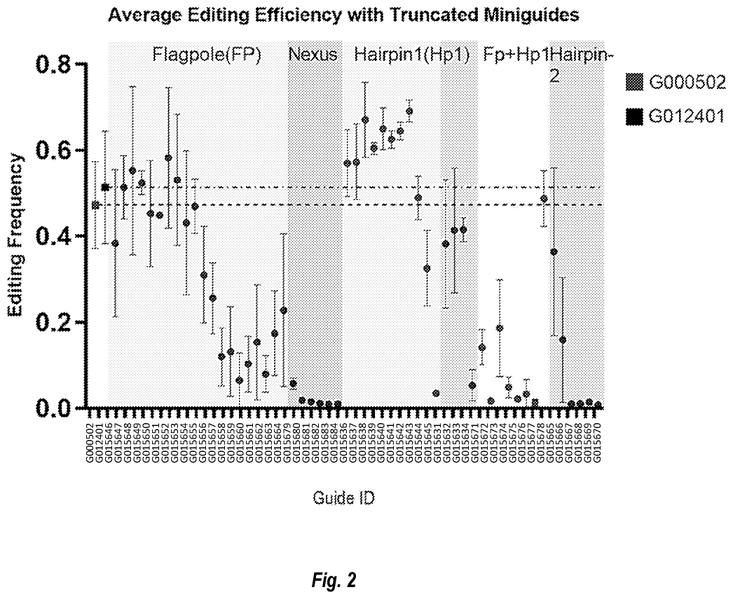

As discussed herein, positions of nucleotides corresponding to those described with respect to spyCas9 gRNA can be identified in another gRNA with sequence and/or structural similarity by pairwise or structural alignment. Structural alignment is useful where molecules share similar structures despite considerable sequence variation. For example, spyCas9 and Staphylococcus aureus Cas9 (“SaCas9”) have divergent sequences, but significant structural alignment. See, e.g., FIG. 2(F) from Nishimasu et al., Cell 162(5): 1113-1126 (2015). Structural alignment can be used to identify nucleotides in a saCas9 or other sgRNA that correspond to particular positions, such as positions H1-1, H1-2, or H1-3, positions H1-6 through H1-10, position H1-12, or positions N18 or N of the conserved portion of a spyCas9 sgRNA (e.g., SEQ ID NO: 400) (see Table 2).

Structural alignment involves identifying corresponding residues across two (or more) sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on distance minimization given position (e.g., nucleobase position 1 or the 1′ carbon of the pentose ring for polynucleotides, or alpha carbons for polypeptides) in the overlaid structures (e.g., what set of paired positions provides a minimized root-mean-square deviation for the alignment). When identifying positions in a non-spyCas9 gRNA corresponding to positions described with respect to spyCas9 gRNA, spyCas9 gRNA can be the “second” sequence. Where a non-spyCas9 gRNA of interest does not have an available known structure, but is more closely related to another non-spyCas9 gRNA that does have a known structure, it may be most effective to model the non-spyCas9 gRNA of interest using the known structure of the closely related non-spyCas9 gRNA, and then compare that model to the spyCas9 gRNA structure to identify the desired corresponding residue in the non-spyCas9 gRNA of interest. There is an extensive literature on structural modeling and alignment for proteins; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein. See also FIG. 2(F) from Nishimasu et al., Cell 162(5): 1113-1126 (2015) for alignment of nucleic acid.

A “target sequence” as used herein refers to a sequence of nucleic acid to which the guide region directs a nuclease for cleavage. In some embodiments, a spyCas9 protein may be directed by a guide region to a target sequence by the nucleotides present in the guide region. In some embodiments, the sgRNA does not comprise a spacer region.

As used herein, the “5′ end” refers to the first nucleotide of the gRNA (including a dgRNA (typically the 5′ end of the crRNA of the dgRNA), sgRNA), in which the 5′ position is not linked to another nucleotide.

As used herein, a “5′ end modification” refers to a gRNA comprising a guide region having modifications in one or more of the one (1) to about seven (7) nucleotides at its 5′ end, optionally wherein the first nucleotide (from the 5′ end) of the gRNA is modified.

As used herein, the “3′ end” refers to the end or terminal nucleotide of a gRNA, in which the 3′ position is not linked to another nucleotide. In some embodiment, the 3′ end is in the 3′ tail. In some embodiments, the 3′ end is in the conserved portion of an gRNA.

As used herein, a “3′ end modification” refers to a gRNA having modifications in one or more of the one (1) to about seven (7) nucleotides at its 3′ end, optionally wherein the last nucleotide (i.e., the 3′ most nucleotide) of the gRNA is modified. If a 3′ tail is present, the 1 to about 7 nucleotides may be within the 3′ tail. If a 3′ tail is not present, the 1 to about 7 nucleotides may be within the conserved portion of a sgRNA.

The “last,” “second to last,” “third to last,” etc., nucleotide refers to the 3′ most, second 3′ most, third 3′ most, etc., nucleotide, respectively in a given sequence. For example, in the sequence 5′-AAACTG-3′, the last, second to last, and third to last nucleotides are G, T, and C, respectively. The phrase “last 3 nucleotides” refers to the last, second to last, and third to last nucleotides; more generally, “last N nucleotides” refers to the last to the Nth to last nucleotides, inclusive. “Third nucleotide from the 3′ end of the 3′ terminus” is equivalent to “third to last nucleotide.” Similarly, “third nucleotide from the 5′ end of the 5′ terminus” is equivalent to “third nucleotide at the 5′ terminus.”

As used herein, a “protective end modification” (such as a protective 5′ end modification or protective 3′ end modification) refers to a modification of one or more nucleotides within seven nucleotides of the end of an sgRNA that reduces degradation of the sgRNA, such as exonucleolytic degradation. In some embodiments, a protective end modification comprises modifications of at least two or at least three nucleotides within seven nucleotides of the end of the sgRNA. In some embodiments, the modifications comprise phosphorothioate linkages, 2′ modifications such as 2′-OMe or 2′-fluoro, 2′-H (DNA), ENA, UNA, or a combination thereof. In some embodiments, the modifications comprise phosphorothioate linkages and 2′-OMe modifications. In some embodiments, at least three terminal nucleotides are modified, e.g., with phosphorothioate linkages or with a combination of phosphorothioate linkages and 2′-OMe modifications. Modifications known to those of skill in the art to reduce exonucleolytic degradation are encompassed.

In some embodiments, a “3′ tail” comprising between 1 and about 20 nucleotides follows the conserved portion of a sgRNA at its 3′ end.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein”, as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases and Class 2 Cas nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, the “minimum uridine content” of a given ORF is the uridine content of an ORF that (a) uses a minimal uridine codon at every position and (b) encodes the same amino acid sequence as the given ORF. The minimal uridine codon(s) for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating minimum uridine content.

As used herein, the “minimum uridine dinucleotide content” of a given ORF is the lowest possible uridine dinucleotide (UU) content of an ORF that (a) uses a minimal uridine codon (as discussed above) at every position and (b) encodes the same amino acid sequence as the given ORF. The uridine dinucleotide (UU) content can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating minimum uridine dinucleotide content.

As used herein, the “minimum adenine content” of a given open reading frame (ORF) is the adenine content of an ORF that (a) uses a minimal adenine codon at every position and (b) encodes the same amino acid sequence as the given ORF. The minimal adenine codon(s) for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating minimum adenine content.

As used herein, the “minimum adenine dinucleotide content” of a given open reading frame (ORF) is the lowest possible adenine dinucleotide (AA) content of an ORF that (a) uses a minimal adenine codon (as discussed above) at every position and (b) encodes the same amino acid sequence as the given ORF. The adenine dinucleotide (AA) content can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating minimum adenine dinucleotide content.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject”.

### Types of Modifications Described Herein

Guide RNAs (e.g., sgRNAs, dgRNAs, and crRNAs) comprising modifications at various positions are disclosed herein. In some embodiments, a position of a gRNA that comprises a modification is modified with any one or more of the following types of modifications.

2′-O-methyl Modifications

Modified sugars are believed to control the puckering of nucleotide sugar rings, a physical property that influences oligonucleotide binding affinity for complementary strands, duplex formation, and interaction with nucleases. Substitutions on sugar rings can therefore alter the conformation and puckering of these sugars. For example, 2′-O-methyl (2′-OMe) modifications can increase binding affinity and nuclease stability of oligonucleotides, though as shown in the Examples, the effect of any modification at a given position in an oligonucleotide needs to be empirically determined.

The terms “mA,” “mC,” “mU,” or “mG” may be used to denote a nucleotide that has been modified with 2′-OMe.

A ribonucleotide and a modified 2′-O-methyl ribonucleotide can be depicted as follows:

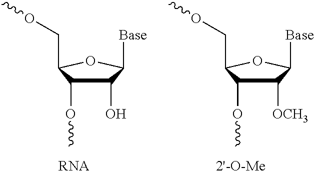

2′-O-(2-methoxyethyl) Modifications

In some embodiments, the modification may be 2′-O-(2-methoxyethyl) (2′-O-moe). A modified 2′-O-moe ribonucleotide can be depicted as follows:

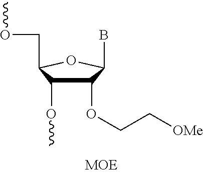

The terms “moeA,” “moeC,” “moeU,” or “moeG” may be used to denote a nucleotide that has been modified with 2′-O-moe.

2′-fluoro Modifications

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

A ribonucleotide without and with a 2′-F substitution can be depicted as follows:

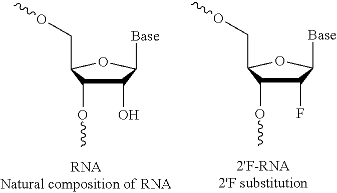

Phosphorothioate Modifications

A phosphorothioate (PS) linkage or bond refers to a bond where a sulfur is substituted for one nonbridging phosphate oxygen in a phosphodiester linkage, for example between nucleotides. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond. Throughout this application, PS modifications are grouped with the nucleotide whose 3′ carbon is bonded to the phosphorothioate; thus, indicating that a PS modification is at position 1 means that the phosphorothioate is bonded to the 3′ carbon of nucleotide 1 and the 5′ carbon of nucleotide 2. Thus, where a YA site is indicated as being “PS modified” or the like, the PS linkage is between the Y and A or between the A and the next nucleotide.

In this application, the terms “mA*,” “mC*,” “mU*,” or “mG*” may be used to denote a nucleotide that has been substituted with 2′-OMe and that is linked to the next (e.g., 3′) nucleotide with a PS linkage, which may sometimes be referred to as a “PS bond.” Similarly, the terms “fA*,” “fC*,” “fU*,” or “fG*” may be used to denote a nucleotide that has been substituted with 2′-F and that is linked to the next (e.g., 3′) nucleotide with a PS linkage. Equivalents of a PS linkage or bond are encompassed by embodiments described herein.

The diagram below shows the substitution of S— for a nonbridging phosphate oxygen, generating a PS linkage in lieu of a phosphodiester linkage:

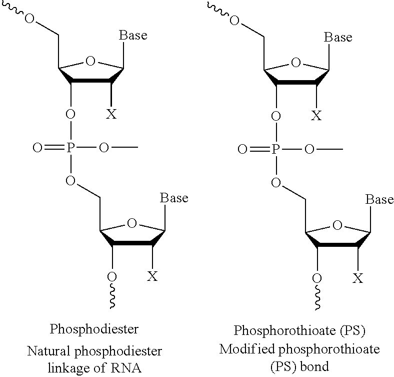

Inverted Abasic Modifications

Abasic nucleotides refer to those which lack nitrogenous bases. The figure below depicts an oligonucleotide with an abasic (in this case, shown as apurinic; an abasic site could also be an apyrimidinic site, wherein the description of the abasic site is typically in reference to Watson-Crick base pairing—e.g., an apurinic site refers to a site that lacks a nitrogenous base and would typically base pair with a pyrimidinic site) site that lacks a base, wherein the base may be substituted by another moiety at the 1′ position of the furan ring (e.g., a hydroxyl group, as shown below, to form a ribose or deoxyribose site, as shown below, or a hydrogen):

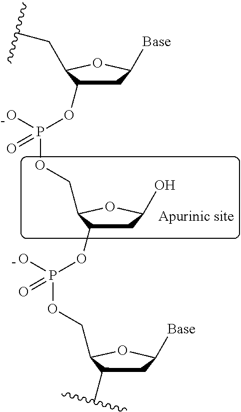

Inverted bases refer to those with linkages that are inverted from the normal 5′ to 3′ linkage (i.e., either a 5′ to 5′ linkage or a 3′ to 3′ linkage). For example:

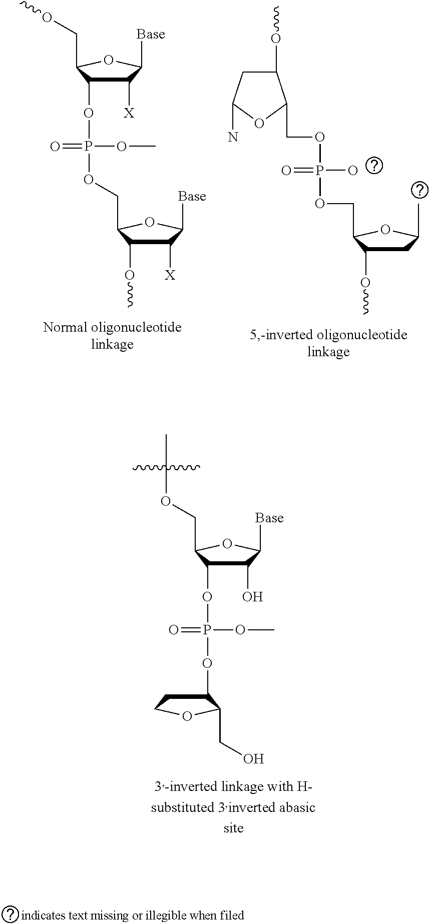

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap. In this application, the terms “invd” indicates an inverted abasic nucleotide linkage.

Deoxyribonucleotides

A deoxyribonucleotide (in which the sugar comprises a 2′-deoxy position) is considered a modification in the context of a gRNA, in that the nucleotide is modified relative to standard RNA by the substitution of a proton for a hydroxyl at the 2′ position. Unless otherwise indicated, a deoxyribonucleotide modification at a position that is U in an unmodified RNA can also comprise replacement of the U nucleobase with a T.

Bicyclic Ribose Analog

Exemplary bicyclic ribose analogs include locked nucleic acid (LNA), ENA, bridged nucleic acid (BNA), or another LNA-like modifications. In some instances, a bicyclic ribose analog has 2′ and 4′ positions connected through a linker. The linker can be of the formula —X—(CH₂)ₙ— where n is 1 or 2; X is O, NR, or S; and R is H or C₁₋₃ alkyl, e.g., methyl. Examples of bicyclic ribose analogs include LNAs comprising a 2′-O—CH₂-4′ bicyclic structure (oxy-LNA) (see WO 98/39352 and WO 99/14226); 2′-NH—CH₂-4′ or 2′-N(CH₃)—CH₂-4′ (amino-LNAs) (Singh et al., J. Org. Chem. 63:10035-10039 (1998); Singh et al., J. Org. Chem. 63:6078-6079 (1998)); and 2′-S—CH₂-4′ (thio-LNA) (Singh et al., J. Org. Chem. 63:6078-6079 (1998); Kumar et al., Biorg. Med. Chem. Lett. 8:2219-2222 (1998)).

### ENA

An ENA modification refers to a nucleotide comprising a 2′-0,4′-C-ethylene modification. An exemplary structure of an ENA nucleotide is shown below, in which wavy lines indicate connections to the adjacent nucleotides (or terminal positions as the case may be, with the understanding that if the 3′ terminal nucleotide is an ENA nucleotide, the 3′ position may comprise a hydroxyl rather than phosphate). For further discussion of ENA nucleotides, see, e.g., Koizumi et al., Nucleic Acids Res. 31: 3267-3273 (2003).

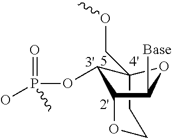

### UNA

A UNA or unlocked nucleic acid modification refers to a nucleotide comprising a 2′,3′-seco-RNA modification, in which the 2′ and 3′ carbons are not bonded directly to each other. An exemplary structure of a UNA nucleotide is shown below, in which wavy lines indicate connections to the adjacent phosphates or modifications replacing phosphates (or terminal positions as the case may be). For further discussion of UNA nucleotides, see, e.g., Snead et al., Molecular Therapy 2: e103, doi:10.1038/mtna.2013.36 (2013).

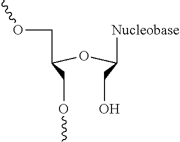

### Base Modifications

A base modification is any modification that alters the structure of a nucleobase or its bond to the backbone, including isomerization (as in pseudouridine). In some embodiments, a base modification includes inosine. In some embodiments, a modification comprises a base modification that reduces RNA endonuclease activity, e.g., by interfering with recognition of a cleavage site by an RNase and/or by stabilizing an RNA structure (e.g., secondary structure) that decreases accessibility of a cleavage site to an RNase. Exemplary base modifications that can stabilize RNA structures are pseudouridine and 5-methylcytosine. See Peacock et al., J Org Chem. 76: 7295-7300 (2011). In some embodiments, a base modification can increase or decrease the melting temperature (Tm) of a nucleic acid, e.g., by increasing the hydrogen bonding in a Watson-Crick base pair, forming non-canonical base pair, or creating a mismatched base pair.

The above modifications and their equivalents are included within the scope of the embodiments described herein.

### YA Modifications

A modification at a YA site (also referred to as a YA modification) can be a modification of the internucleoside linkage, a modification of the base (pyrimidine or adenine), e.g. by chemical modification, substitution, or otherwise, and/or a modification of the sugar (e.g. at the 2′ position, such as 2′-O-alkyl, 2′-F, 2′-moe, 2′-F arabinose, 2′-H (deoxyribose), and the like). In some embodiments, a “YA modification” is any modification that alters the structure of the dinucleotide motif to reduce RNA endonuclease activity, e.g., by interfering with recognition or cleavage of a YA site by an RNase and/or by stabilizing an RNA structure (e.g., secondary structure) that decreases accessibility of a cleavage site to an RNase. See Peacock et al., J Org Chem. 76: 7295-7300 (2011); Behlke, Oligonucleotides 18:305-320 (2008); Ku et al., Adv. Drug Delivery Reviews 104: 16-28 (2016); Ghidini et al., Chem. Commun., 2013, 49, 9036. Peacock et al., Belhke, Ku, and Ghidini provide exemplary modifications suitable as YA modifications. Modifications known to those of skill in the art to reduce endonucleolytic degradation are encompassed. Exemplary 2′ ribose modifications that affect the 2′ hydroxyl group involved in RNase cleavage are 2′-H and 2′-O-alkyl, including 2′-O-Me. Modifications such as bicyclic ribose analogs, UNA, and modified internucleoside linkages of the residues at the YA site can be YA modifications. Exemplary base modifications that can stabilize RNA structures are pseudouridine and 5-methylcytosine. In some embodiments, at least one nucleotide of the YA site is modified. In some embodiments, the pyrimidine (also called “pyrimidine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the adenine (also called “adenine position”) of the YA site comprises a modification (which includes a modification altering the internucleoside linkage immediately 3′ of the sugar of the pyrimidine, a modification of the pyrimidine base, and a modification of the ribose, e.g. at its 2′ position). In some embodiments, the pyrimidine and the adenine of the YA site comprise modifications. In some embodiments, the YA modification reduces RNA endonuclease activity.

The above modifications and their equivalents are included within the scope of the embodiments described herein.

Guide RNAs (gRNAs) Comprising Shortened Regions and/or Substitutions

In some embodiments, a gRNA (e.g., sgRNA, dgRNA, or crRNA) provided herein comprises a conserved portion comprising a hairpin region, wherein the hairpin region lacks 6-8 nucleotides, 9-10 nucleotides, or 5-10 nucleotides. In some embodiments, the gRNA is from S. pyogenes Cas9 (“spyCas9”) or a spyCas9 equivalent. In some embodiments, the gRNA is not from S. pyogenes Cas9 (“non-spyCas9”). In some embodiments, the 6-8 nucleotides, 9-10 nucleotides, or 5-10 nucleotides are consecutive.

In some embodiments, the hairpin regions lack 5, 6, 7, 8, 9, 10, 11, or 12 nucleotides. In some embodiments, the hairpin 1 portion lacks 5, 6, 7, 8, 9, 10, 11, or 12 nucleotides. In some embodiments, the hairpin 2 portion lacks 5, 6, 7, 8, 9, 10, 11, or 12 nucleotides. In some embodiments, the hairpin regions lack 5, 6, 7, 8, 9, 10, 11, or 12 consecutive nucleotides. In some embodiments, the hairpin 1 portion lacks 5, 6, 7, 8, 9, 10, 11, or 12 consecutive nucleotides. In some embodiments, the hairpin 2 portion lacks 5, 6, 7, 8, 9, 10, 11, or 12 consecutive nucleotides. In some embodiments, the 6-8 lacking nucleotides, lacking 9-10 nucleotides, or 5-10 lacking nucleotides are within hairpin 1. In some embodiments, the 6-8 lacking nucleotides, lacking 9-10 nucleotides, or 5-10 lacking nucleotides are within hairpin 2. In some embodiments, the 6-8 lacking nucleotides, lacking 9-10 nucleotides, or 5-10 lacking nucleotides are within hairpin 1 and hairpin 2. In some embodiments, the 6-8 lacking nucleotides, lacking 9-10 nucleotides, or 5-10 lacking nucleotides are within hairpin 1 or hairpin 2. In some embodiments, the 6-8 lacking nucleotides, lacking 9-10 nucleotides, or 5-10 lacking nucleotides are consecutive and include the “N” between hairpin 1 and hairpin 2. In some embodiments, the 5-10 or 6-10 lacking nucleotides include the “N” between hairpin 1 and hairpin 2. In some embodiments, the 5-10 or 6-10 lacking nucleotides are consecutive and span at least a portion of hairpin 1. In some embodiments, the 5-10 or 6-10 lacking nucleotides are consecutive and span at least a portion of hairpin 2. In some embodiments, the 6-8 lacking nucleotides, lacking 9-10 nucleotides, or 5-10 lacking nucleotides are consecutive and span at least a portion of hairpin 1 and a portion of hairpin 2. In some embodiments, the 6-8 lacking nucleotides, lacking 9-10 nucleotides, or 5-10 lacking nucleotides are consecutive and span at least a portion of hairpin 1 and the “N” between hairpin 1 and hairpin 2. In some embodiments, the 5-10 lacking nucleotides comprise or consist of nucleotides 54-58, 54-61, or 53-60 of SEQ ID NO: 400.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 and/or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, and/or H1-4 and H1-9) form a base pair in the gRNA.

In some embodiments, the gRNA further comprises an upper stem region lacking at least 1 nucleotide, e.g., any of the shortened upper stem regions indicated in Table 1C or described elsewhere herein, which may be combined with any of the shortened or substituted hairpin 1 regions described herein, including but not limited to combinations indicated in the numbered embodiments above and represented in the sequences of Table 1A.

TABLE 1C
Exemplary combinations of hairpin 1 regions and upper stem regions

| | May be combined with any one of Upper Stem |
|---|---|
| Hairpin 1 region | regions: |
| shortened hairpin 1 region | Upper stem region lacking 1 nucleotide; or |
| lacking 6-8 nucleotides and one | Upper stem region lacking 2 nucleotides; or |
| or more of positions H1-1, H1-2, | Upper stem region lacking 3 nucleotides; or |
| or H1-3 is deleted or substituted | Upper stem region lacking 4 nucleotides; or |
| relative to SEQ ID NO: 400 | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region | Upper stem region lacking 1 nucleotide; or |
| lacking 6-8 nucleotides and one | Upper stem region lacking 2 nucleotides; or |
| or more of positions H1-6 | Upper stem region lacking 3 nucleotides; or |
| through H1-10 is substituted | Upper stem region lacking 4 nucleotides; or |
| relative to SEQ ID NO: 400 | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region | Upper stem region lacking 1 nucleotide; or |
| lacking 9-10 nucleotides | Upper stem region lacking 2 nucleotides; or |
| including H1-1 and/or H1-12 | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region | Upper stem region lacking 1 nucleotide; or |
| lacking 5-10 nucleotides and one | Upper stem region lacking 2 nucleotides; or |
| or more of positions N18, H1-12, | Upper stem region lacking 3 nucleotides; or |
| or N is substituted relative to | Upper stem region lacking 4 nucleotides; or |
| SEQ ID NO: 400 | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| at least one of the following pairs | Upper stem region lacking 1 nucleotide; or |
| of nucleotides are substituted in | Upper stem region lacking 2 nucleotides; or |
| the substituted and optionally | Upper stem region lacking 3 nucleotides; or |
| shortened hairpin 1 with Watson- | Upper stem region lacking 4 nucleotides; or |
| Crick pairing nucleotides: H1-1 | Upper stem region lacking 5 nucleotides; or |
| and H1-12, H1-2 and H1-11, H1- | Upper stem region lacking 6 nucleotides; or |
| 3 and H1-10, and/or H1-4 and | US3, US4, US9, US10 deleted; or |
| H1-9 | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| at least one of the following pairs | Upper stem region lacking 1 nucleotide; or |
| of nucleotides are substituted in | Upper stem region lacking 2 nucleotides; or |
| the substituted and optionally | Upper stem region lacking 3 nucleotides; or |
| shortened hairpin 1 with Watson- | Upper stem region lacking 4 nucleotides; or |
| Crick pairing nucleotides: H1-1 | Upper stem region lacking 5 nucleotides; or |
| and H1-12, H1-2 and H1-11, H1- | Upper stem region lacking 6 nucleotides; or |
| 3 and H1-10, and/or H1-4 and | US3, US4, US9, US10 deleted; or |
| H1-9, and the hairpin 1 region | US8 deleted; or |
| lacks any one or two of H1-5 | US4 and US9 deleted; |
| through H1-8 | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| at least one of the following pairs | Upper stem region lacking 1 nucleotide; or |
| of nucleotides are substituted in | Upper stem region lacking 2 nucleotides; or |
| the substituted and optionally | Upper stem region lacking 3 nucleotides; or |
| shortened hairpin 1 with Watson- | Upper stem region lacking 4 nucleotides; or |
| Crick pairing nucleotides: H1-1 | Upper stem region lacking 5 nucleotides; or |
| and H1-12, H1-2 and H1-11, H1- | Upper stem region lacking 6 nucleotides; or |
| 3 and H1-10, and/or H1-4 and | US3, US4, US9, US10 deleted; or |
| H1-9, and the hairpin 1 region | US8 deleted; or |
| lacks one, two, or three of the | US4 and US9 deleted; |
| following pairs of nucleotides: | US5 deleted and US3, US4, US9, US10 substituted; or |
| H1-1 and H1-12, H1-2 and H1-11 | US8 deleted and US3, US4, US9, US10 substituted; or |
| , H1-3 and H1-10 and/or H1-4 | US4 and US9 deleted, and US3 and US10 substituted; |
| and H1-9 | US3, US4, US8, US9, US10 deleted; or |
| | US3, US4, US5, US9, US10 deleted |
| at least one of the following pairs | Upper stem region lacking 1 nucleotide; or |
| of nucleotides are substituted in | Upper stem region lacking 2 nucleotides; or |
| the substituted and optionally | Upper stem region lacking 3 nucleotides; or |
| shortened hairpin 1 with Watson- | Upper stem region lacking 4 nucleotides; or |
| Crick pairing nucleotides: H1-1 | Upper stem region lacking 5 nucleotides; or |
| and H1-12, H1-2 and H1-11, H1- | Upper stem region lacking 6 nucleotides; or |
| 3 and H1-10, and/or H1-4 and | US3, US4, US9, US10 deleted; or |
| H1-9, and the hairpin 1 region | US8 deleted; or |
| lacks 1-8 nucleotides of the | US4 and US9 deleted; |
| hairpin 1 region | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-4 or H1-2 | Upper stem region lacking 1 nucleotide; or |
| through H1-5 are deleted, and | Upper stem region lacking 2 nucleotides; or |
| H1-9 through H1-11 are deleted | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-4 or H1-2 | Upper stem region lacking 1 nucleotide; or |
| through H1-5 are deleted, and | Upper stem region lacking 2 nucleotides; or |
| H1-8 through H1-11 are deleted | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1, H1-3 through H1-8, and | Upper stem region lacking 1 nucleotide; or |
| H1-12 are deleted | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-8 are deleted | Upper stem region lacking 1 nucleotide; or |
| | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-3 through H1-9 are deleted | Upper stem region lacking 1 nucleotide; or |
| | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-7 and H1-8 are substituted | Upper stem region lacking 1 nucleotide; or |
| with a G and a C, respectively, | Upper stem region lacking 2 nucleotides; or |
| and positions H1-2 through H1-4 | Upper stem region lacking 3 nucleotides; or |
| and H1-9 through H1-11 are | Upper stem region lacking 4 nucleotides; or |
| deleted | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-6 and H1-7 are substituted | Upper stem region lacking 1 nucleotide; or |
| with a C and a U, respectively, | Upper stem region lacking 2 nucleotides; or |
| and positions H1-2 through H1-4 | Upper stem region lacking 3 nucleotides; or |
| and H1-9 through H1-11 are | Upper stem region lacking 4 nucleotides; or |
| deleted | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1 and H1-12 are substituted | Upper stem region lacking 1 nucleotide; or |
| with a C and a G, respectively, | Upper stem region lacking 2 nucleotides; or |
| and positions H1-2 through H1-4 | Upper stem region lacking 3 nucleotides; or |
| and H1-9 through H1-11 are | Upper stem region lacking 4 nucleotides; or |
| deleted | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 2 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 3 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 4 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 5 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 6 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 7 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 8 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 9 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| shortened hairpin 1 region with a | Upper stem region lacking 1 nucleotide; or |
| length of 10 nucleotides | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1 and H1-12 are substituted | Upper stem region lacking 1 nucleotide; or |
| (optionally with a C and a G, | Upper stem region lacking 2 nucleotides; or |
| respectively), and positions H1-2 | Upper stem region lacking 3 nucleotides; or |
| through H1-4 and H1-9 through | Upper stem region lacking 4 nucleotides; or |
| H1-11 are deleted | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1 through H1-8 and H1-11 | Upper stem region lacking 1 nucleotide; or |
| through H1-12 are deleted | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| N18 is substituted with a C and | Upper stem region lacking 1 nucleotide; or |
| H1-4 through H1-11 are deleted | Upper stem region lacking 2 nucleotides; or |
| | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-12 is substituted with a C, | Upper stem region lacking 1 nucleotide; or |
| position N is substituted with an | Upper stem region lacking 2 nucleotides; or |
| A, and positions H1-4 through | Upper stem region lacking 3 nucleotides; or |
| H1-11 are deleted | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| position H1-12 is substituted with | Upper stem region lacking 1 nucleotide; or |
| an A, position N is substituted | Upper stem region lacking 2 nucleotides; or |
| with an A, and positions H1-4 | Upper stem region lacking 3 nucleotides; or |
| through H1-11 are deleted | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-4 and H1-9 | Upper stem region lacking 1 nucleotide; or |
| through H1-11 deleted; H1-7 and | Upper stem region lacking 2 nucleotides; or |
| H1-8 optionally substituted | Upper stem region lacking 3 nucleotides; or |
| | Upper stem region lacking 4 nucleotides; or |
| | Upper stem region lacking 5 nucleotides; or |
| | Upper stem region lacking 6 nucleotides; or |
| | US3, US4, US9, US10 deleted; or |
| | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-4 and H1-9 | US3, US4, US9, US10 deleted; or |
| through H1-11 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-5 and H1-9 | US3, US4, US9, US10 deleted; or |
| through H1-11 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-8 substituted and H1-2 | US3, US4, US9, US10 deleted; or |
| through 5 and H1-9 through 11 | US8 deleted; or |
| deleted | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-6 and H1-8 substituted; and | US3, US4, US9, US10 deleted; or |
| H1-2 through 5 and H1-9 through | US8 deleted; or |
| 11 deleted | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-2 through H1-5 and H1-8 | US3, US4, US9, US10 deleted; or |
| through 11 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1, H1-3, H1-4, H1-9, H1-10, | US3, US4, US9, US10 deleted; or |
| and H1-12 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1, H1-3, H1-4, H1-5, H1-6, | US3, US4, US9, US10 deleted; or |
| H1-7, H1-8, H1-12 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-1 through H1-9, H1-11, H1- | US3, US4, US9, US10 deleted; or |
| 12 deleted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-6 through H1-10 deleted; H1- | US3, US4, US9, US10 deleted; or |
| 12 and N optionally substituted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |
| H1-6 through H1-10 deleted; N18 | US3, US4, US9, US10 deleted; or |
| substituted | US8 deleted; or |
| | US4 and US9 deleted; |
| | US5 deleted and US3, US4, US9, US10 substituted; or |
| | US8 deleted and US3, US4, US9, US10 substituted; or |
| | US4 and US9 deleted, and US3 and US10 substituted; |
| | US3, US4, US8, US9, US10 deleted; or |
| | US3, US4, US5, US9, US10 deleted |

In Table 1C, where US3, US4, US9, and US10 are substituted, they may be substituted with G-C or C-G base pairs (where US3 pairs to US10 and US4 pairs to US9), e.g., US3, US4, US9, and US10 may be substituted with a G, C, G, and C, respectively. Similarly, where US3 and US10 are substituted, they may be substituted with a G-C or C-G base pair (where US3 pairs to US10), e.g., US3 and US10 may be substituted with a G and C, respectively, or a C and G, respectively.

In some embodiments, the gRNA described herein further comprises a nexus region, wherein the nexus region lacks at least one nucleotide. In some embodiments, the gRNA lacks at least 2, 3, 4, 5, 6, 7, 8, 9, or 10 nucleotides in the nexus region. In some embodiments, the gRNA lacks at least 1-2, 1-3, 1-4 nucleotides, 1-5 nucleotides, 1-6 nucleotides, 1-10 nucleotides, or 1-15 nucleotides in the nexus region. In some embodiments, the gRNA lacks each nucleotide in the nexus region.

In some embodiments, the gRNA further comprises a guide region. In some embodiments, the guide region comprises the first 1-10, 11, 12, 13, 14, 15, 16, 17, 18, 19, or 20 nucleotides at the 5′ end of the gRNA. In some embodiments, the guide region comprises 20 nucleotides. In some embodiments, the guide region comprises 5, 6, 7, 8, 9, 10, 11, 12, 13, 14, 15, 16, 17, 18, 19, 20, 21, 22, 23, 24, or 25 or more nucleotides. In some embodiments, the guide region comprises 17 nucleotides. In some embodiments, the guide region comprises 18 nucleotides. In some embodiments, the guide region comprises 19 nucleotides.

In some embodiments, the selection of the guide region is determined based on target sequences within the gene of interest for editing. For example, in some embodiments, the gRNA comprises a guide region that is complementary to target sequences of a gene of interest.

In some embodiments, the target sequence in the gene of interest may be complementary to the guide region of the gRNA. In some embodiments, the degree of complementarity or identity between a guide region of a gRNA and its corresponding target sequence in the gene of interest may be about 50%, 55%, 60%, 65%, 70%, 75%, 80%, 85%, 90%, 95%, 96%, 97%, 98%, 99%, or 100%. In some embodiments, the guide region of a gRNA and the target region of a gene of interest may be 100% complementary or identical. In other embodiments, the guide region of a gRNA and the target region of a gene of interest may contain at least one mismatch. For example, the guide region of a gRNA and the target sequence of a gene of interest may contain 1, 2, 3, 4, 5, 6, 7, 8, 9, or 10 mismatches, where the total length of the target sequence is at least about 17, 18, 19, 20 or more base pairs. In some embodiments, the guide region of a gRNA and the target region of a gene of interest may contain 1-6 mismatches where the guide sequence comprises at least about 17, 18, 19, 20 or more nucleotides. In some embodiments, the guide region of a gRNA and the target region of a gene of interest may contain 1, 2, 3, 4, 5, or 6 mismatches where the guide sequence comprises about 20 nucleotides. The 5′ terminus may comprise nucleotides that are not considered guide regions (i.e., do not function to direct a Cas9 protein to a target nucleic acid).

In some embodiments, an gRNA comprises a 5′ end modification or a 3′ end modification and a conserved portion of an gRNA comprising a shortened hairpin 1 region, wherein
(i) the shortened hairpin 1 region lacks 6-8 nucleotides; and (A) one or more of positions H1-1, H1-2, or H1-3 is deleted or substituted relative to SEQ ID NO: 400 and/or (B) one or more of positions H1-6 through H1-10 is substituted relative to SEQ ID NO: 400; or
(ii) the shortened hairpin 1 region lacks 9-10 nucleotides including H1-1 and/or H1-12; or
(iii) the shortened hairpin 1 region lacks 5-10 nucleotides and one or more of positions N18, H1-12, or N is substituted relative to SEQ ID NO: 400. N18 and N refer to the nucleotides immediately 5′ and 3′ of hairpin 1, respectively, in Table 2.

In some embodiments, an gRNA comprises a 5′ end modification or a 3′ end modification and a conserved portion of the gRNA comprises a shortened upper stem region, wherein the shortened upper stem region lacks 1-6 nucleotides.

In some embodiments, an gRNA comprises a 5′ end modification or a 3′ end modification and a conserved portion of the gRNA comprises a substitution relative to SEQ ID NO: 400 at any one or more of LS6, LS7, US3, US10, B3, N7, N15, N17, H2-2 and H2-14. The substituent nucleotide is neither a pyrimidine that is followed by an adenine, nor an adenine that is preceded by a pyrimidine.

In some embodiments, an gRNA comprises a 5′ end modification or a 3′ end modification and a conserved portion of the gRNA comprises one or more of (a), (b), and (c) above.

In some embodiments, a conserved portion of an gRNA described herein further comprises one or more deletion or substitution in a nexus region, an lower stem region, or a bulge region.

In some embodiments, an gRNA comprises one or more of the following deletion in the hairpin 1 region (H1-H12).

Shortened Hairpin 1 Region

In some embodiments, a conserved portion of an gRNA described herein comprises (a) a shortened hairpin 1 region lacking 6-8 nucleotides. In some embodiments, a conserved portion of an gRNA described herein comprises (a) a shortened hairpin 1 region lacking 6-8 nucleotides and one or more deletion or substation in a shortened hairpin region. In some embodiments, the hairpin 1 region lacks 6-8 nucleotides, and one or more of positions positions H1-1, H1-2, or H1-3 is deleted or substituted relative to SEQ ID NO: 400. In some embodiments, position H1-1 is deleted. In some embodiments, position H1-1 is substituted. In some embodiments, position position H1-2 is deleted. In some embodiments, position position H1-2 is substituted. In some embodiments, position position H1-3 is deleted. In some embodiments, position H1-3 is substituted.

In some embodiments, the shortened hairpin 1 region lacks 6-8 nucleotides, and one or more of positions H1-6 through H1-10 is substituted relative to SEQ ID NO: 400. In some embodiments, position H1-6 is substituted. In some embodiments, position H1-7 is substituted. In some embodiments, position H1-8 is substituted. In some embodiments, position H1-9 is substituted. In some embodiments, position H1-10 is substituted.

In some embodiments, the shortened hairpin 1 region has a length of 4 nucleotides. In some embodiments, the shortened hairpin 1 region has a length of 5 nucleotides. In some embodiments, the shortened hairpin 1 region has a length of 6 nucleotides. In further embodiments, the 4, 5, or 6 nucleotides of the shortened hairpin 1 region include less than or equal to 2 substitutions. In further embodiments, the 4, 5, or 6 nucleotides of the shortened hairpin 1 region include less than or equal to 1 substitution. In further embodiments, the 4, 5, or 6 nucleotides of the shortened hairpin 1 region are unsubstituted.

In some embodiments, position H1-1 is deleted. In some embodiments, position H1-1 is substituted. In some embodiments, position H1-2 is deleted. In some embodiments, position H1-2 is substituted. In some embodiments, position H1-3 is deleted. In some embodiments, position H1-3 is substituted. In some embodiments, position H1-4 is deleted. In some embodiments, position H1-4 is substituted. In some embodiments, position H1-5 is deleted. In some embodiments, position H1-5 is substituted. In some embodiments, position H1-6 is deleted. In some embodiments, position H1-6 is substituted. In some embodiments, position H1-7 is deleted. In some embodiments, position H1-7 is substituted. In some embodiments, position H1-8 is deleted. In some embodiments, position H1-8 is substituted. In some embodiments, position H1-9 is deleted. In some embodiments, position H1-9 is substituted. In some embodiments, position H1-10 is deleted. In some embodiments, position H1-10 is substituted. In some embodiments, position H1-11 is deleted. In some embodiments, position H1-11 is substituted. In some embodiments, position H1-12 is deleted. In some embodiments, position H1-12 is substituted.

In some embodiments, positions H1-1 through H1-2 are deleted. In some embodiments, positions H1-1 through H1-3 are deleted. In some embodiments, positions H1-1 through H1-4 are deleted. In some embodiments, positions H1-1 through H1-5 are deleted. In some embodiments, positions H1-1 through H1-6 are deleted. In some embodiments, positions H1-1 through H1-7 are deleted. In some embodiments, positions H1-1 through H1-8 are deleted. In some embodiments, positions H1-1 through H1-9 are deleted. In some embodiments, positions H1-1 through H1-10 are deleted.

In some embodiments, positions H1-2 through H1-3 are deleted. In some embodiments, positions H1-2 through H1-4 are deleted. In some embodiments, positions H1-2 through H1-5 are deleted. In some embodiments, positions H1-2 through H1-6 are deleted. In some embodiments, positions H1-2 through H1-7 are deleted. In some embodiments, positions H1-2 through H1-8 are deleted. In some embodiments, positions H1-2 through H1-9 are deleted. In some embodiments, positions H1-2 through H1-10 are deleted. In some embodiments, positions H1-2 through H1-11 are deleted.

In some embodiments, positions H1-3 through H1-4 are deleted. In some embodiments, positions H1-3 through H1-5 are deleted. In some embodiments, positions H1-3 through H1-6 are deleted. In some embodiments, positions H1-3 through H1-7 are deleted. In some embodiments, positions H1-3 through H1-8 are deleted. In some embodiments, positions H1-3 through H1-9 are deleted. In some embodiments, positions H1-3 through H1-10 are deleted. In some embodiments, positions H1-3 through H1-11 are deleted. In some embodiments, positions H1-3 through H1-12 are deleted.

In some embodiments, positions H1-4 through H1-5 are deleted. In some embodiments, positions H1-4 through H1-6 are deleted. In some embodiments, positions H1-4 through H1-7 are deleted. In some embodiments, positions H1-4 through H1-8 are deleted. In some embodiments, positions H1-4 through H1-9 are deleted. In some embodiments, positions H1-4 through H1-10 are deleted. In some embodiments, positions H1-4 through H1-11 are deleted. In some embodiments, positions H1-4 through H1-12 are deleted.

In some embodiments, positions H1-5 through H1-6 are deleted. In some embodiments, positions H1-5 through H1-7 are deleted. In some embodiments, positions H1-5 through H1-8 are deleted. In some embodiments, positions H1-5 through H1-9 are deleted. In some embodiments, positions H1-5 through H1-10 are deleted. In some embodiments, positions H1-5 through H1-11 are deleted. In some embodiments, positions H1-5 through H1-12 are deleted.

In some embodiments, positions H1-6 through H1-7 are deleted. In some embodiments, positions H1-6 through H1-8 are deleted. In some embodiments, positions H1-6 through H1-9 are deleted. In some embodiments, positions H1-6 through H1-10 are deleted. In some embodiments, positions H1-6 through H1-11 are deleted. In some embodiments, positions H1-6 through H1-12 are deleted.

In some embodiments, positions H1-7 through H1-8 are deleted. In some embodiments, positions H1-7 through H1-9 are deleted. In some embodiments, positions H1-7 through H1-10 are deleted. In some embodiments, positions H1-7 through H1-11 are deleted. In some embodiments, positions H1-7 through H1-12 are deleted.

In some embodiments, positions H1-8 through H1-9 are deleted. In some embodiments, positions H1-8 through H1-10 are deleted. In some embodiments, positions H1-8 through H1-11 are deleted. In some embodiments, positions H1-8 through H1-12 are deleted.

In some embodiments, positions H1-9 through H1-10 are deleted. In some embodiments, positions H1-9 through H1-11 are deleted. In some embodiments, positions H1-9 through H1-12 are deleted.

In some embodiments, positions H1-10 through H1-11 are deleted. In some embodiments, positions H1-10 through H1-12 are deleted.

In some embodiments, positions H1-11 through H1-12 are deleted.

In some embodiments, positions H1-2 through H1-4 and H1-9 through H1-11 are deleted. In some embodiments, the shortened hairpin 1 region comprises: (a) the sequence AGAAAU; (b) a sequence having less than or equal to 2 mismatches to the sequence of (a); or (c) a sequence having less than or equal to 1 mismatch to the sequence of (a).

In some embodiments, positions H1-2 through H1-5 and H1-9 through H1-11 are deleted. In further embodiments, each position of the upper stem region is modified. In some embodiments, 1, 2, 3, 4, 5, 6, 7, 8, 9, 10, or 11 positions of the upper stem region are modified. In some embodiments, all but 1, 2, 3, 4, 5, or 6 positions of the upper stem region are modified. In some embodiments, at least 20%, 30%, 40%, 50%, 60%, 70%, 80%, or 90% of the positions of the upper stem region are modified. Optionally, in any of the foregoing embodiments, each modified position of the upper stem region is modified by 2′-O-methylation. In further embodiments, the shortened hairpin 1 region comprises: (a) the sequence AAAAAU; (b) a sequence having less than or equal to 2 mismatches to the sequence of (a); or (c) a sequence having less than or equal to 1 mismatch to the sequence of (a).

In some embodiments, positions H1-2 through H1-5 and H1-8 through H1-11 are deleted. In further embodiments, each position of the upper stem region is modified. Optionally, each position of the upper stem region is modified by 2′-O-methylation. In further embodiments, the shortened hairpin 1 region comprises: (a) the sequence AAAU; (b) a sequence having less than or equal to 2 mismatches to the sequence of (a); or (c) a sequence having less than or equal to 1 mismatch to the sequence of (a).

In some embodiments, positions H1-1, H1-3 through H1-8, and H-12 are deleted. In further embodiments, each position of the upper stem region is modified. Optionally, each position of the upper stem region is modified by 2′-O-methylation. In further embodiments, the shortened hairpin 1 region comprises: (a) the sequence CAAG; (b) a sequence having less than or equal to 2 mismatches to the sequence of (a); or (c) a sequence having less than or equal to 1 mismatch to the sequence of (a).

In some embodiments, positions H1-2 through H1-8 are deleted. In further embodiments, each position of the upper stem region is modified. Optionally, each position of the upper stem region is modified by 2′-O-methylation. In further embodiments, the shortened hairpin 1 region comprises: (a) the sequence AAAGU; (b) a sequence having less than or equal to 2 mismatches to the sequence of (a); or (c) a sequence having less than or equal to 1 mismatch to the sequence of (a).

In some embodiments, positions H1-3 through H1-9 are deleted. In further embodiments, each position of the upper stem region is modified. Optionally, each position of the upper stem region is modified by 2′-O-methylation. In further embodiments, the shortened hairpin 1 region comprises: (a) the sequence ACAGU; (b) a sequence having less than or equal to 2 mismatches to the sequence of (a); or (c) a sequence having less than or equal to 1 mismatch to the sequence of (a).

In some embodiments, position H1-7 is substituted with a G. In some embodiments, position H1-8 is substituted with a C. In some embodiments, positions H1-7 and H1-8 are substituted positions. In some embodiments, H1-7 and H1-8 are substituted with a G and a C, respectively. In some embodiments, positions H1-7 and H1-8 are substituted with a G and a C, respectively, and positions H1-2 through H1-4 and H1-9 through H1-11 are deleted. In further embodiments, the shortened hairpin 1 region comprises: (a) the sequence AGAGCU; (b) a sequence having less than or equal to 2 mismatches to the sequence of (a); or (c) a sequence having less than or equal to 1 mismatch to the sequence of (a).

In some embodiments, position H1-6 is substituted with a G. In some embodiments, position H1-7 is substituted with a U. In some embodiments, positions H1-6 and H1-7 are substituted positions. In some embodiments, positions H1-6 and H1-7 are substituted with a G and a U, respectively. In some embodiments, positions H1-6 and H1-7 are substituted with a G and a C, respectively, and positions H1-2 through H1-4 and H1-9 through H1-11 are deleted. In further embodiments, the shortened hairpin 1 region comprises: (a) the sequence AGCUAU; (b) a sequence having less than or equal to 2 mismatches to the sequence of (a); or (c) a sequence having less than or equal to 1 mismatch to the sequence of (a).

In some embodiments, position H1-1 is substituted with a C. In some embodiments, position H1-12 is substituted with a G. In some embodiments, positions H1-1 and H1-12 are substituted positions. In some embodiments, positions H1-1 and H1-12 are substituted with a C and a G, respectively. In some embodiments, positions H1-1 and H1-12 are substituted with a C and a G, respectively, and positions H1-2 through H1-4 and H1-9 through H1-11 are deleted. In further embodiments, each position of the upper stem region is modified. Optionally, each position of the upper stem region is modified by 2′-O-methylation. In further embodiments, the shortened hairpin 1 region comprises: (a) the sequence CGAAAG; (b) a sequence having less than or equal to 2 mismatches to the sequence of (a); or (c) a sequence having less than or equal to 1 mismatch to the sequence of (a).

In some embodiments, the shortened hairpin 1 region lacks 9-10 nucleotides. In some embodiments, the shortened hairpin 1 region has a length of 2 nucleotides. In some embodiments, the shortened hairpin 1 region has a length of 3 nucleotides. In further embodiments, the 2 or 3 nucleotides of the shortened hairpin 1 region are unsubstituted.

In some embodiments, position H1-1 is deleted. In some embodiments, position H1-12 is deleted. In some embodiments, positions H1-11 through H1-12 are deleted. In further embodiments, positions H1-1 through H1-8 and H1-11 through H1-12 are deleted. In further embodiments, the shortened hairpin 1 region comprises: (a) the sequence AA; (b) a sequence having less than or equal to 1 mismatch to the sequence of (a).

In some embodiments, positions H1-11 through H1-9 are deleted. In further embodiments, the shortened hairpin 1 region comprises: (a) the sequence AG; (b) a sequence having less than or equal to 1 mismatch to the sequence of (a).

In some embodiments, the shortened hairpin 1 region lacks 5-10 nucleotides. In some embodiments, the shortened hairpin 1 region lacks 5-10 nucleotides and one or more of positions N18, H1-12, or N is substituted relative to SEQ ID NO: 400. In some embodiments, the shortened hairpin 1 region has a length of 2 nucleotides. In some embodiments, the shortened hairpin 1 region has a length of 3 nucleotides. In some embodiments, the shortened hairpin 1 region has a length of 4 nucleotides. In some embodiments, the shortened hairpin 1 region has a length of 5 nucleotides. In some embodiments, the shortened hairpin 1 region has a length of 6 nucleotides. In some embodiments, the shortened hairpin 1 region has a length of 7 nucleotides. In further embodiments, positions H1-4 through H1-11 are deleted.

In some embodiments, a conserved portion of an gRNA described herein further comprises a shortened hairpin 1 region lacking 5-10 nucleotides wherein one or more nucleotide is deleted.

In some embodiments, position N18 is substituted. In further embodiments, position N18 is substituted with a C. In further embodiments, position N18 is substituted with a C and positions H1-4 through H1-11 are deleted. In further embodiments, the gRNA comprises a segment containing position N18, the shortened hairpin 1 region, and position N, and the segment comprises: (a) the sequence CACUUG; (b) a sequence having less than or equal to 2 mismatches to the sequence of (a); or (c) a sequence having less than or equal to 1 mismatch to the sequence of (a).

In some embodiments, position H1-12 is substituted. In some embodiments, position H1-12 is substituted with a C. In further embodiments, position H1-12 is substituted with an A. In some embodiments, position N is substituted. In further embodiments, position N is substituted with an A. In further embodiments, position H1-12 is substituted with a C and position N is substituted with an A. In further embodiments, position H1-12 is substituted with a C, position N is substituted with an A, and positions H1-4 through H1-11 are deleted. In further embodiments, the gRNA comprises a segment containing position N18, the shortened hairpin 1 region, and position N, and the segment comprises: (a) the sequence AACUCA; (b) a sequence having less than or equal to 2 mismatches to the sequence of (a); or (c) a sequence having less than or equal to 1 mismatch to the sequence of (a).

In some embodiments, position H1-12 is substituted with an A and position N is substituted with an A. In further embodiments, position H1-12 is substituted with an A, position N is substituted with an A, and positions H1-4 through H1-11 are deleted. In further embodiments, the gRNA comprises a segment containing position N18, the shortened hairpin 1 region, and position N, and the segment comprises: (a) the sequence AACUAA; (b) a sequence having less than or equal to 2 mismatches to the sequence of (a); or (c) a sequence having less than or equal to 1 mismatch to the sequence of (a).

Shortened Upper Stem Region

In some embodiments, a conserved portion of an gRNA described herein a shortened upper stem region. In some embodiments, the shortened upper stem region lacks 1-6 nucleotides. In some embodiments, the upper stem region may comprise a loop (e.g., a tetraloop), and the length of the upper stem region includes nucleotides in the loop. In some embodiments, the shortened upper stem region has a length of 6 nucleotides. In some embodiments, the shortened upper stem region has a length of 7 nucleotides. In some embodiments, the shortened upper stem region has a length of 8 nucleotides. In some embodiments, the shortened upper stem region has a length of 9 nucleotides. In some embodiments, the shortened upper stem region has a length of 10 nucleotides. In some embodiments, the shortened upper stem region has a length of 11 nucleotides. In further embodiments, the 6, 7, 8, 9, 10, or 11 nucleotides of the shortened upper stem region include less than or equal to 4 substitutions, less than or equal to 2 substitutions, or one substitution. In further embodiments, the 6, 7, 8, 9, 10, or 11 nucleotides of the shortened upper stem region are unsubstituted.

In some embodiments, one or more of positions US3, US4, US5, US8, US9, or US10 is deleted. In some embodiments, position US3 is deleted. In some embodiments, position US4 is deleted. In some embodiments, position US5 is deleted. In some embodiments, position US8 is deleted. In some embodiments, position US9 is deleted. In some embodiments, position US10 is deleted. It should be noted that in sequences such as SEQ ID NO: 400, where US6, US7, and US8 are each A residues, deletions of any one of US 6, US7, and US8 are equivalent.

In some embodiments, positions US4 and US9 are deleted. In further embodiments, positions H1-2 through H1-5 and H1-8 through H1-11 are deleted. In further embodiments, the shortened upper stem region comprises: (a) the sequence GCUGAAAGGC (SEQ ID NO: 1004); (b) a sequence having less than or equal to 2 mismatches to the sequence of (a); or (c) a sequence having less than or equal to 1 mismatch to the sequence of (a).

In some embodiments, positions US3 and US4 are deleted. In some embodiments, positions US9 and US10 are deleted. In some embodiments, positions US3, US4, US9, and US10 are deleted.

In some embodiments, a conserved portion of an gRNA having a shortened upper stem region described herein further comprises a shortened hairpin region 1. In further embodiments, positions H1-2 through H1-5 and H1-8 through H1-11 are deleted. In further embodiments, positions H1-2 through H1-5 and H-12 are deleted. In further embodiments, the shortened upper stem region comprises: (a) the sequence GCGAAAGC; (b) a sequence having less than or equal to 2 mismatches to the sequence of (a); or (c) a sequence having less than or equal to 1 mismatch to the sequence of (a). In further embodiments, positions H1 and H1-4 through H1-12 are deleted.

In some embodiments, positions US3, US4, US8, US9, and US10 are deleted. In further embodiments, the shortened upper stem region comprises: (a) the sequence GCGAAGC; (b) a sequence having less than or equal to 2 mismatches to the sequence of (a); or (c) a sequence having less than or equal to 1 mismatch to the sequence of (a).

In some embodiments, positions US3, US4, US5, US9, and US10 are deleted. In further embodiments, the shortened upper stem region comprises: (a) the sequence GCAAAGC (SEQ ID NO: 1005); (b) a sequence having less than or equal to 2 mismatches to the sequence of (a); or (c) a sequence having less than or equal to 1 mismatch to the sequence of (a).

In some embodiments, position US3 is substituted, optionally with a G. In some embodiments, position US4 is substituted, optionally with a C. In some embodiments, position US9 is substituted, optionally with a G. In some embodiments, position US10 is substituted, optionally with a C.

In some embodiments, positions US3 and US10 are substituted, optionally with a G and a C, respectively. In some embodiments, positions US4 and US9 are substituted, optionally with a C and a G, respectively.

In some embodiments, positions US3 and US10 are substituted with a G and a C, respectively, and positions US4 and US9 are substituted with a C and a G, respectively. In some embodiments, position US5 is deleted. In some embodiments, positions US3 and US10 are substituted with a G and a C, respectively, and positions US4 and US9 are substituted with a C and a G, respectively, and position US8 is deleted. Optionally, instead of deletion of position US8, one of position US6 or US7 may be deleted as US6, US7, US8 each comprise an A. In some embodiments, positions H1-2 through H1-5 and H1-8 through H1-11 are deleted. In further embodiments, shortened upper stem region comprises: (a) the sequence GCGCGAAGCGC; (b) a sequence having less than or equal to 2 mismatches to the sequence of (a); or (c) a sequence having less than or equal to 1 mismatch to the sequence of (a).

In some embodiments, positions US3 and US10 are substituted with a C and a G, respectively. In some embodiments, positions US3 and US10 are substituted with a C and a G, respectively, and positions US4 and US9 are deleted. In some embodiments, the shortened upper stem region comprises: (a) the sequence GCGGAAACGC (SEQ ID NO: 1006); (b) a sequence having less than or equal to 2 mismatches to the sequence of (a); or (c) a sequence having less than or equal to 1 mismatch to the sequence of (a).

In some embodiments, positions US3 and US10 are substituted with a G and a C, respectively, and positions US4 and US9 are deleted. In some embodiments, the shortened upper stem region comprises: (a) the sequence GCCGAAAGGC (SEQ ID NO: 1007); (b) a sequence having less than or equal to 2 mismatches to the sequence of (a); or (c) a sequence having less than or equal to 1 mismatch to the sequence of (a).

YA Site Substitutions and Modifications

In some embodiments, a conserved portion of an gRNA described herein has a substitution relative to SEQ ID NO: 400 at any one or more of LS6, LS7, US3, US10, B3, N7, N15, N17, H2-2 and H2-14. The substituent nucleotide is neither a pyrimidine that is followed by an adenine, nor an adenine that is preceded by a pyrimidine.

In some embodiments, one of positions LS6, LS7, US3, US10, B3, N7, N15, N17, H2-2 and H2-14 is substituted. In some embodiments, position LS6 is substituted. In some embodiments, position LS7 is substituted. In some embodiments, position US3 is substituted. In some embodiments, position US10 is substituted. In some embodiments, position B3 is substituted, optionally with a G. In some embodiments, position N7 is substituted, optionally with a C or with a U. In some embodiments, position N15 is substituted, optionally with a C or with a U. In some embodiments, position N17 is substituted, optionally with a G. In some embodiments, position H2-2 is substituted. In some embodiments, position H-14 is substituted.

In some embodiments, two of positions LS6, LS7, US3, US10, B3, N7, N15, N17, H2-2 and H2-14 is substituted. In some embodiments, positions LS 6 and LS7 are substituted, optionally with a U and an A, respectively. In some embodiments, positions US3 and US10 are substituted, optionally with a G and a C, respectively. In some embodiments, positions H2-2 and H2-14 are substituted, optionally with an A and a U, respectively. In some embodiments, positions H2-2 and H2-14 are substituted, optionally with a G and a C, respectively.

In some embodiments, at least 2, 3, 4, 5, 6, 7, or 8 of positions US3, US10, LS6, LS7, B3, N15, N17, H2-2, and H2-14 are substituted. In some embodiments, positions US3, US10, LS6, LS7, B3, N15, N17, H2-2, and H2-14 are substituted.

In some embodiments, at least 2, 3, 4, 5, or all of the following are true:

(a) positions US3 and US10 are substituted with a G and a C, respectively;
(b) positions LS 6 and LS7 are substituted with a U and an A, respectively;
(c) position B3 is substituted with a G;
(d) position N15 is substituted with a C;
(e) position N17 is substituted with a G; and/or
(f) positions H2-2 and H2-14 are substituted with an A and a U, respectively.

In some embodiments, a conserved portion of an gRNA described herein has both a shortened upper stem region described herein and a shortened hairpin 1 region.

In some embodiments, positions H1-4 through H1-11 are deleted. In further embodiments, the shortened hairpin 1 region comprises: (a) the sequence ACUU; (b) a sequence having less than or equal to 2 mismatches to the sequence of (a); or (c) a sequence having less than or equal to 1 mismatch to the sequence of (a).

In some embodiments, position N2 is substituted with a C. In some embodiments, positions US1-US4 and US9-US12 are deleted. Optionally, positions H1-2 through H1-11 are deleted. Optionally, positions H1-4 through H1-11 are deleted.

In some embodiments, positions US2-US4 and US9-US11 are deleted. In further embodiments, positions H1-2 to H1-11 are deleted or positions H1-1 and H1-4 through H1-12 are deleted.

In some embodiments, a conserved portion of an gRNA described herein comprises both a shortened upper stem region described herein and a hairpin 1 region truncation (i.e., positions H1-1 through H1-12 are deleted).

In some embodiment a conserved portion of an gRNA described herein further comprises one or more of the following deletions in the upper stem region. In some embodiments, positions US3-US5 and US8-US10 are deleted. In some embodiments, positions US3-US4 and US7-US10 are deleted. In further embodiments, positions US3-US10 are deleted. In further embodiments, positions US2-US5 and US8-US11 are deleted. In further embodiments, positions US2-US6 and US8-US11 are deleted. In further embodiments, positions US2-US11 are deleted. In further embodiments, positions US1-US5 and US8-US12 are deleted. In further embodiments, positions US1-US5 and US7-US12 are deleted.

In some embodiments, a conserved portion of an gRNA described herein further comprises a shortened hairpin 2 region. In some embodiments, position H2-15 is deleted. In some embodiments, positions H2-14 and H2-15 are deleted.

In some embodiments, a conserved portion of an gRNA described herein further comprises one or more deletion or substitution in a nexus region, a lower stem region, or a bulge region. In some embodiments, position N6 is deleted, optionally wherein positions H1-4 through H1-11 are deleted. In some embodiments, position LS6 is substituted, optionally with a C. In some embodiments, position B3 is substituted, optionally with a C. In some embodiments, position N1 is substituted, optionally with a C. In some embodiments, N7 is substituted, optionally with a G. In some embodiments, position N15 is substituted, optionally with a G. In some embodiments, position N17 is substituted with a non-pyrimidine, optionally with a G.

Modified Guide RNA (gRNA)

In some embodiments, a gRNA described herein is modified. The term “modified” or “modification” in the context of a gRNA described herein includes the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein.

Exemplary patterns of modifications are shown in Table 1. Additional exemplary patterns are discussed below.

Modifications of Guide Regions and/or YA Sites

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

Guide Region Modifications, Including YA Site Modifications

In some embodiments, the guide region comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. In some embodiments, two or more YA sites located at 5-end, 6-end, 7-end, 8-end, 9-end, or 10-end from the 5′ end of the 5′ terminus comprise YA modifications. In some embodiments, three or more YA sites located at 5-end, 6-end, 7-end, 8-end, 9-end, or 10-end from the 5′ end of the 5′ terminus comprise YA modifications. In some embodiments, four or more YA sites located at 5-end, 6-end, 7-end, 8-end, 9-end, or 10-end from the 5′ end of the 5′ terminus comprise YA modifications. In some embodiments, five or more YA sites located at 5-end, 6-end, 7-end, 8-end, 9-end, or 10-end from the 5′ end of the 5′ terminus comprise YA modifications. A modified guide region YA site comprises a YA modification.

In some embodiments, a modified guide region YA site is within 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a YA modification is located within a YA site 20, 19, 18, 17, 16, 15, 14, 13, 12, 11, 10, 9, 8, 7, 6, 5, 4, 3, 2, or 1 nucleotides from the 3′ terminal nucleotide of the guide region. In some embodiments, a YA modification is located 20, 19, 18, 17, 16, 15, 14, 13, 12, 11, 10, 9, 8, 7, 6, 5, 4, 3, 2, or 1 nucleotides from the 3′ terminal nucleotide of the guide region.

In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a gRNA can comprise a 5′ end modification as described herein and further comprise a modified guide region YA site. Alternatively, a gRNA can comprise an unmodified 5′ end and a modified guide region YA site. Alternatively, a gRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above.

Additional embodiments of guide region modifications, including guide region YA site modifications, are set forth elsewhere herein, including in the summary above and in the discussion of gRNAs comprising modifications, including modifications at YA sites above, and elsewhere herein. The guide region of a gRNA may be modified according to any embodiment comprising a modified guide region set forth herein. Any embodiments set forth elsewhere in this disclosure may be combined to the extent feasible with any of the foregoing embodiments.

Conserved Region YA Site Modifications

Conserved region YA sites 1-10 are illustrated in FIG. 1C. In some embodiments, 1, 2, 3, 4, 5, 6, 7, 8, 9, or 10 conserved region YA sites comprise modifications.

In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, 1, 2, 3, or 4 of conserved region YA sites 2, 3, 4, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above.

Additional embodiments of conserved region YA site modifications are set forth in the summary above. Any embodiments set forth elsewhere in this disclosure may be combined to the extent feasible with any of the foregoing embodiments.

Modifications to Terminal Nucleotides

In some embodiments, the 5′ and/or 3′ terminus regions of a gRNA are modified.

3′ Terminus Region Modifications

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, at least one of the terminal (i.e., last) 1, 2, 3, 4, 5, 6, or 7 nucleotides in the 3′ terminus region are modified. In some embodiments, at least two of the terminal (i.e., last) 1, 2, 3, 4, 5, 6, or 7 nucleotides in the 3′ terminus region are modified. In some embodiments, at least three of the terminal (i.e., last) 1, 2, 3, 4, 5, 6, or 7 nucleotides in the 3′ terminus region are modified. In some embodiments, the modification comprises a PS linkage. In some embodiments, the modification to the 3′ terminus region is a 3′ protective end modification. In some embodiments, the 3′ end modification comprises a 3′ protective end modification.

In some embodiments, the 3′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 3′ end modification comprises or further comprises a 2′-O-methyl (2′-O-Me) modified nucleotide.

In some embodiments, the 3′ end modification comprises or further comprises a 2′-fluoro (2′-F) modified nucleotide.

In some embodiments, the 3′ end modification comprises or further comprises a phosphorothioate (PS) linkage between nucleotides.

In some embodiments, the 3′ end modification comprises or further comprises an inverted abasic modified nucleotide.

In some embodiments, the 3′ end modification comprises or further comprises a modification of any one or more of the last 7, 6, 5, 4, 3, 2, or 1 nucleotides. In some embodiments, the 3′ end modification comprises or further comprises one modified nucleotide. In some embodiments, the 3′ end modification comprises or further comprises two modified nucleotides. In some embodiments, the 3′ end modification comprises or further comprises three modified nucleotides. In some embodiments, the 3′ end modification comprises or further comprises four modified nucleotides. In some embodiments, the 3′ end modification comprises or further comprises five modified nucleotides. In some embodiments, the 3′ end modification comprises or further comprises six modified nucleotides. In some embodiments, the 3′ end modification comprises or further comprises seven modified nucleotides.

In some embodiments, the 3′ end modification comprises or further comprises a modification of between 1 and 7 or between 1 and 5 nucleotides.

In some embodiments, the 3′ end modification comprises or further comprises modifications of 1, 2, 3, 4, 5, 6, or 7 nucleotides at the 3′ end of the gRNA.

In some embodiments, the 3′ end modification comprises or further comprises modifications of about 1-3, 1-5, 1-6, or 1-7 nucleotides at the 3′ end of the gRNA.

In some embodiments, the 3′ end modification comprises or further comprises any one or more of the following: a phosphorothioate (PS) linkage between nucleotides, a 2′-0-Me modified nucleotide, a 2′-O-moe modified nucleotide, a 2′-F modified nucleotide, an inverted abasic modified nucleotide, and a combination thereof.

In some embodiments, the 3′ end modification comprises or further comprises 1, 2, 3, 4, 5, 6, or 7 PS linkages between nucleotides.

In some embodiments, the 3′ end modification comprises or further comprises at least one 2′-O-Me, 2′-O-moe, inverted abasic, or 2′-F modified nucleotide. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides.

In some embodiments, the 3′ end modification comprises or further comprises PS linkages between any one or more of the last four nucleotides. In some embodiments, the 3′ end modification comprises or further comprises PS linkages between any one or more of the last five nucleotides. In some embodiments, the 3′ end modification comprises or further comprises PS linkages between any one or more of the last 2, 3, 4, 5, 6, or 7 nucleotides.

In some embodiments, the 3′ end modification comprises or further comprises a modification of one or more of the last 1-7 nucleotides, wherein the modification is a PS linkage, inverted abasic nucleotide, 2′-OMe, 2′-O-moe, 2′-F, or combinations thereof.

In some embodiments, the 3′ end modification comprises or further comprises a modification to the last nucleotide with 2′-OMe, 2′-O-moe, 2′-F, or combinations thereof, and an optionally one or two PS linkages to the next nucleotide and/or the first nucleotide of the 3′ tail.

In some embodiments, the 3′ end modification comprises or further comprises a modification to the last and/or second to last nucleotide with 2′-OMe, 2′-O-moe, 2′-F, or combinations thereof, and optionally one or more PS linkages.

In some embodiments, the 3′ end modification comprises or further comprises a modification to the last, second to last, and/or third to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, and/or fourth to last nucleotides with 2′-OMe, 2′-O-moe, 2′-F, or combinations thereof, and optionally one or more PS linkages.

In some embodiments, the 3′ end modification comprises or further comprises a modification to the last, second to last, third to last, fourth to last, and/or fifth to last nucleotides with 2′-OMe, 2′-O-moe, 2′-F, or combinations thereof, and optionally one or more PS linkages.

In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified.

In some embodiments, a gRNA is provided comprising a 3′ end modification, wherein the 3′ end modification comprises the 3′ end modification as shown in any one of SEQ ID NOs: 101-190 or 795-798. In some embodiments, a gRNA is provided comprising a 3′ protective end modification.

In some embodiments, a gRNA is provided comprising a 3′ end modification, wherein the 3′ end modification comprises (i) a 2′-OMe modified nucleotide at the last nucleotide of the conserved region of an gRNA (ii) three consecutive 2′O-moe modified nucleotides immediately 5′ to the 2′-OMe modified nucleotide, and (iii) three consecutive PS linkages between the last three nucleotides.

In some embodiments, a gRNA is provided comprising a 3′ end modification, wherein the 3′ end modification comprises (i) five consecutive 2′-OMe modified nucleotides from the last nucleotide of the conserved region of an sgRNA or the conserved region of a gRNA, and (ii) three PS linkages between the last three nucleotides.

In some embodiments, a gRNA is provided comprising a 3′ end modification, wherein the 3′ end modification comprises an inverted abasic modified nucleotide at the last nucleotide of the conserved region of an sgRNA or the conserved region of a gRNA.

In some embodiments, a gRNA is provided comprising a 3′ end modification, wherein the 3′ end modification comprises (i) an inverted abasic modified nucleotide at the last nucleotide of the conserved region of a gRNA, and (ii) three consecutive 2′-OMe modified nucleotides at the last three nucleotides of the conserved region of a gRNA or the conserved region of a gRNA.

In some embodiments, a gRNA is provided comprising (i) 15 consecutive 2′-OMe modified nucleotides from the last nucleotide of the conserved region of a gRNA, (ii) five consecutive 2′-F modified nucleotides immediately 5′ to the 2′-OMe modified nucleotides, and (iii) three PS linkages between the last three nucleotides.

In some embodiments, a gRNA is provided comprising (i) alternating 2′-OMe modified nucleotides and 2′-F modified nucleotides at the last 20 nucleotides of the conserved region of a gRNA, and (ii) three PS linkages between the last three nucleotides.

In some embodiments, a gRNA is provided comprising a 3′ end modification, wherein the 3′ end modification comprises (i) two or three consecutive 2′-OMe modified nucleotides, and (ii) three PS linkages between the last three nucleotides.

In some embodiments, a gRNA is provided comprising a 3′ end modification, wherein the 3′ end modification comprises one PS linkage between the last and next to last nucleotides.

In some embodiments, a gRNA is provided comprising (i) 15 or 20 consecutive 2′-OMe modified nucleotides, and (ii) three PS linkages between the last three nucleotides.

In some embodiments, the gRNA comprises a 5′ end modification and a 3′ end modification.

### 3′ Tail

In some embodiments, the gRNA comprises a 3′ terminus comprising a 3′ tail, which follows and is 3′ of the conserved portion of a gRNA. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the 3′ tail comprises about 1, 2, 3, 4, 5, 6, 7, 8, 9, or 10 nucleotides. In some embodiments, the 3′ tail comprises 1, 2, 3, 4, 5, 6, 7, 8, 9, or 10 nucleotides. In some embodiments, the 3′ tail comprises 1 nucleotide. In some embodiments, the 3′ tail comprises 2 nucleotides. In some embodiments, the 3′ tail comprises 3 nucleotides. In some embodiments, the 3′ tail comprises 4 nucleotides. In some embodiments, the 3′ tail comprises about 1-2, 1-3, 1-4, 1-5, 1-7, 1-10, at least 1-5, at least 1-3, at least 1-4, at least 1-5, at least 1-5, at least 1-7, or at least 1-10 nucleotides.

In some embodiments, the 3′ tail comprising between 1 and 20 nucleotides and follows the 3′ end of the conserved portion of a gRNA.

In some embodiments, the 3′ tail comprises or further comprises one or more of a protective end modification, a phosphorothioate (PS) linkage between nucleotides, a 2′-OMe modified nucleotide, a 2′-O-moe modified nucleotide, a 2′-F modified nucleotide, an inverted abasic modified nucleotide, and a combination thereof.

In some embodiments, the 3′ tail comprises or further comprises one or more phosphorothioate (PS) linkages between nucleotides. In some embodiments, the 3′ tail comprises or further comprises one or more 2′-OMe modified nucleotides. In some embodiments, the 3′ tail comprises or further comprises one or more 2′-O-moe modified nucleotides. In some embodiments, the 3′ tail comprises or further comprises one or more 2′-F modified nucleotide. In some embodiments, the 3′ tail comprises or further comprises one or more an inverted abasic modified nucleotides. In some embodiments, the 3′ tail comprises or further comprises one or more protective end modifications. In some embodiments, the 3′ tail comprises or further comprises a combination of one or more of a phosphorothioate (PS) linkage between nucleotides, a 2′-OMe modified nucleotide, a 2′-O-moe modified nucleotide, a 2′-F modified nucleotide, and an inverted abasic modified nucleotide.

In some embodiments, the gRNA does not comprise a 3′ tail.

5′ Terminus Region Modifications

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, at least two of the terminal 1, 2, 3, 4, 5, 6, or 7 nucleotides at the 5′ terminus region are modified. In some embodiments, at least three of the terminal 1, 2, 3, 4, 5, 6, or 7 nucleotides at the 5′ terminus region are modified. In some embodiments, the 5′ end modification is a 5′ protective end modification.

In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified.

In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds.

In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, the gRNA comprises one or more phosphorothioate (PS) linkages between the first one, two, three, four, five, six, or seven nucleotides at the 5′ terminus. In some embodiments, the gRNA comprises one or more PS linkages between the last one, two, three, four, five, six, or seven nucleotides at the 3′ terminus. In some embodiments, the gRNA comprises one or more PS linkages between both the last one, two, three, four, five, six, or seven nucleotides at the 3′ terminus and the first one, two, three, four, five, six, or seven nucleotides from the 5′ end of the 5′ terminus. In some embodiments, in addition to PS linkages, the 5′ and 3′ terminal nucleotides may comprise 2′-O-Me, 2′-O-moe, or 2′-F modified nucleotides.

In some embodiments, the gRNA comprises a 5′ end modification, e.g., wherein the first nucleotide of the guide region is modified. In some embodiments, the gRNA comprises a 5′ end modification, wherein the first nucleotide of the guide region comprises a 5′ protective end modification.

In some embodiments, the 5′ end modification comprises a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof.

In some embodiments, the 5′ end modification comprises or further comprises a 2′-O-methyl (2′-O-Me) modified nucleotide.

In some embodiments, the 5′ end modification comprises or further comprises a 2′-fluoro (2′-F) modified nucleotide.

In some embodiments, the 5′ end modification comprises or further comprises a phosphorothioate (PS) linkage between nucleotides.

In some embodiments, the 5′ end modification comprises or further comprises an inverted abasic modified nucleotide.

In some embodiments, the 5′ end modification comprises or further comprises a modification of any one or more of nucleotides 1-7 of the guide region of a gRNA. In some embodiments, the 5′ end modification comprises or further comprises one modified nucleotide. In some embodiments, the 5′ end modification comprises or further comprises two modified nucleotides. In some embodiments, the 5′ end modification comprises or further comprises three modified nucleotides. In some embodiments, the 5′ end modification comprises or further comprises four modified nucleotides. In some embodiments, the 5′ end modification comprises or further comprises five modified nucleotides. In some embodiments, the 5′ end modification comprises or further comprises six modified nucleotides. In some embodiments, the 5′ end modification comprises or further comprises seven modified nucleotides.

In some embodiments, the 5′ end modification comprises or further comprises a modification of between 1 and 7, between 1 and 5, between 1 and 4, between 1 and 3, or between 1 and 2 nucleotides.

In some embodiments, the 5′ end modification comprises or further comprises modifications of 1, 2, 3, 4, 5, 6, or 7 nucleotides from the 5′ end. In some embodiments, the 5′ end modification comprises or further comprises modifications of about 1-3, 1-4, 1-5, 1-6, or 1-7 nucleotides from the 5′ end.

In some embodiments, the 5′ end modification comprises or further comprises modifications at the first nucleotide at the 5′ end of the gRNA. In some embodiments, the 5′ end modification comprises or further comprises modifications at the first and second nucleotide from the 5′ end of the gRNA. In some embodiments, the 5′ end modification comprises or further comprises modifications at the first, second, and third nucleotide from the 5′ end of the gRNA. In some embodiments, the 5′ end modification comprises or further comprises modifications at the first, second, third, and fourth nucleotide from the 5′ end of the gRNA. In some embodiments, the 5′ end modification comprises or further comprises modifications at the first, second, third, fourth, and fifth nucleotide from the 5′ end of the gRNA. In some embodiments, the 5′ end modification comprises or further comprises modifications at the first, second, third, fourth, fifth, and sixth nucleotide from the 5′ end of the gRNA. In some embodiments, the 5′ end modification comprises or further comprises modifications at the first, second, third, fourth, fifth, sixth, and seventh nucleotide from the 5′ end of the gRNA.

In some embodiments, the 5′ end modification comprises or further comprises a phosphorothioate (PS) linkage between nucleotides, and/or a 2′-O-Me modified nucleotide, and/or a 2′-O-moe modified nucleotide, and/or a 2′-F modified nucleotide, and/or an inverted abasic modified nucleotide, and/or combinations thereof.

In some embodiments, the 5′ end modification comprises or further comprises 1, 2, 3, 4, 5, 6, and/or 7 PS linkages between nucleotides. In some embodiments, the 5′ end modification comprises or further comprises about 1-2, 1-3, 1-4, 1-5, 1-6, or 1-7 PS linkages between nucleotides.

In some embodiments, the 5′ end modification comprises or further comprises at least one PS linkage, wherein if there is one PS linkage, the linkage is between nucleotides 1 and 2 of the guide region.

In some embodiments, the 5′ end modification comprises or further comprises at least two PS linkages, and the linkages are between nucleotides 1 and 2, and 2 and 3 of the guide region.

In some embodiments, the 5′ end modification comprises or further comprises PS linkages between any one or more of nucleotides 1 and 2, 2 and 3, and 3 and 4 of the guide region.

In some embodiments, the 5′ end modification comprises or further comprises PS linkages between any one or more of nucleotides 1 and 2, 2 and 3, 3 and 4, and 4 and 5 of the guide region.

In some embodiments, the 5′ end modification comprises or further comprises PS linkages between any one or more of nucleotides 1 and 2, 2 and 3, 3 and 4, 4 and 5, and 5 and 6 of the guide region.

In some embodiments, the 5′ end modification comprises or further comprises PS linkages between any one or more of nucleotides 1 and 2, 2 and 3, 3 and 4, 4 and 5, 5 and 6, and 7 and 8 of the guide region.

In some embodiments, the 5′ end modification comprises or further comprises a modification of one or more of nucleotides 1-7 of the guide region, wherein the modification is a PS linkage, inverted abasic nucleotide, 2′-O-Me, 2′-O-moe, 2′-F, and/or combinations thereof.

In some embodiments, the 5′ end modification comprises or further comprises a modification to the first nucleotide of the guide region with 2′-O-Me, 2′-O-moe, 2′-F, or combinations thereof, and an optional PS linkage to the next nucleotide;

In some embodiments, the 5′ end modification comprises or further comprises a modification to the first and/or second nucleotide of the guide region with 2′-O-Me, 2′-O-moe, 2′-F, or combinations thereof, and optionally one or more PS linkages between the first and second nucleotide and/or between the second and third nucleotide.

In some embodiments, the 5′ end modification comprises or further comprises a modification to the first, second, and/or third nucleotides of the variable region with 2′-O-Me, 2′-O-moe, 2′-F, or combinations thereof, and optionally one or more PS linkages between the first and second nucleotide, between the second and third nucleotide, and/or between the third and the fourth nucleotide.

In some embodiments, the 5′ end modification comprises or further comprises a modification to the first, second, third, and/or fourth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, and/or between the fourth and the fifth nucleotide.

In some embodiments, the 5′ end modification comprises or further comprises a modification to the first, second, third, fourth, and/or fifth nucleotides of the variable region with 2′-O-Me, 2′-O-moe, 2′-F, or combinations thereof, and optionally one or more PS linkages between the first and second nucleotide, between the second and third nucleotide, between the third and the fourth nucleotide, between the fourth and the fifth nucleotide, and/or between the fifth and the sixth nucleotide.

In some embodiments, a gRNA is provided comprising a 5′ end modification, wherein the 5′ end modification comprises a 5′ end modification as shown in any one of SEQ ID NOs: 101-190 or 795-798.

In some embodiments, the sgRNA comprises a 5′ end modification comprising a 5′ protective end modification. In some embodiments, a gRNA is provided comprising a 5′ end modification, wherein the 5′ end modification comprises 2′-OMe modified nucleotides at nucleotides 1, 2, and 3 of the guide region.

In some embodiments, a gRNA is provided comprising a 5′ end modification, wherein the 5′ end modification comprises 2′-OMe modified nucleotides at nucleotides 1, 2, and 3 of the guide region and PS linkages between nucleotides 1 and 2, 2 and 3, and 3 and 4 of the guide region.

In some embodiments, a gRNA is provided comprising a 5′ end modification, wherein the 5′ end modification comprises 2′-OMe modified nucleotides at nucleotides 1, 2, 3, 4, and 5 of the guide region.

In some embodiments, a gRNA is provided comprising a 5′ end modification, wherein the 5′ end modification comprises 2′-OMe modified nucleotides at nucleotides 1, 2, 3, 4, and 5 of the guide region and PS linkages between nucleotides 1 and 2, 2 and 3, 3 and 4, 4 and 5, and 5 and 6 of the guide region.

In some embodiments, a gRNA is provided comprising a 5′ end modification, wherein the 5′ end modification comprises 2′O-moe modified nucleotides at nucleotides 1, 2, and 3 of the guide region.

In some embodiments, a gRNA is provided comprising a 5′ end modification, wherein the 5′ end modification comprises 2′O-moe modified nucleotides at nucleotides 1, 2, and 3 of the guide region and PS linkages between nucleotides 1 and 2, 2 and 3, and 3 and 4 of the guide region.

In some embodiments, a gRNA is provided comprising a 5′ end modification, wherein the 5′ end modification comprises an inverted abasic modified nucleotide at nucleotide 1 of the guide region.

In some embodiments, a gRNA is provided comprising a 5′ end modification, wherein the 5′ end modification comprises an inverted abasic modified nucleotide at nucleotide 1 of the guide region and 2′-OMe modified nucleotides at nucleotides 1, 2, and 3 of the guide region.

In some embodiments, a gRNA is provided comprising a 5′ end modification, wherein the 5′ end modification comprises an inverted abasic modified nucleotide at nucleotide 1 of the guide region, 2′-OMe modified nucleotides at nucleotides 1, 2, and 3 of the guide region, and PS linkages between nucleotides 1 and 2, 2 and 3, 3 and 4, 4 and 5, and 5 and 6 of the guide region.

In some embodiments, a gRNA is provided comprising a 5′ end modification and a 3′ end modification. In some embodiments, the gRNA comprises modified nucleotides at the 5′ and 3′ terminus, and modified nucleotides in one or more other regions described in Table 3.

In some embodiments, the sgRNA comprises modified nucleotides that are not at the 5′ or 3′ ends. Exemplary patterns of modifications are described below and in Table 1.

Upper Stem Modifications

In some embodiments, a gRNA is provided comprising an upper stem modification, wherein the upper stem modification comprises a modification to any one or more of US1-US12 in the upper stem region.

In some embodiments, a gRNA is provided comprising an upper stem modification, wherein the upper stem modification comprises a modification of at least 1, 2, 3, 4, 5, 6, 7, 8, 9, 10, 11, or all 12 nucleotides in the upper stem region.

In some embodiments, a gRNA is provided comprising an upper stem modification, wherein the upper stem modification comprises a modification of about 1-2, 1-3, 1-4, 1-5, 1-6, 1-7, 1-8, 1-9, 1-10, or 1-12 nucleotides in the upper stem region.

In some embodiments, a gRNA is provided comprising an upper stem modification, wherein the upper stem modification comprises 1, 2, 3, 4, or 5 YA modifications in a YA site. In some embodiments, an sgRNA is provided comprising an upper stem modification, wherein the upper stem modification comprises at least 1, 2, 3, 4, or 5 YA modifications. In some embodiments, an sgRNA is provided comprising an upper stem modification, wherein the upper stem modification comprises one YA modification. In some embodiments, an sgRNA is provided comprising an upper stem modification, wherein the upper stem modification comprises 2 YA modifications. In some embodiments, the upper stem modification comprises 3 YA modifications. In some embodiments, one or more YA modifications are in a YA site. In some embodiments, one or more YA modifications are distal to a YA site.

In some embodiments, a gRNA is provided comprising an upper stem modification, wherein the upper stem modification comprises a 2′-OMe modified nucleotide. In some embodiments, a gRNA is provided comprising an upper stem modification, wherein the upper stem modification comprises a 2′-O-moe modified nucleotide. In some embodiments, a gRNA is provided comprising an upper stem modification, wherein the upper stem modification comprises a 2′-F modified nucleotide.

In some embodiments, a gRNA is provided comprising an upper stem modification, wherein the upper stem modification comprises a 2′-OMe modified nucleotide, a 2′-O-moe modified nucleotide, a 2′-F modified nucleotide, and/or combinations thereof.

In some embodiments, the sgRNA comprises an upper stem modification as shown in any one of the sequences in Table 1. In some embodiments, such an upper stem modification is combined with a 5′ protective end modification, e.g. as shown for the corresponding sequence in Table 1. In some embodiments, such an upper stem modification is combined with a 3′ protective end modification, e.g. as shown for the corresponding sequence in Table 1. In some embodiments, such an upper stem modification is combined with 5′ and 3′ end modifications as shown for the corresponding sequence in Table 1.

In some embodiments, the gRNA comprises a 5′ end modification and an upper stem modification. In some embodiments, the gRNA comprises a 3′ end modification and an upper stem modification. In some embodiments, the gRNA comprises a 5′ end modification, a 3′ end modification and an upper stem modification.

Hairpin Modifications

In some embodiments, the gRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof.

In some embodiments, the hairpin region modification is in the hairpin 1 region. In some embodiments, the hairpin region modification is in the hairpin 2 region. In some embodiments, modifications are within the hairpin 1 and hairpin 2 regions, optionally wherein the “n” between hairpin 1 and 2 is also modified.

In some embodiments, a gRNA is provided comprising a hairpin modification, wherein the hairpin modification comprises 1, 2, or 3 YA modifications in a YA site. In some embodiments, a gRNA is provided comprising a hairpin modification, wherein the hairpin modification comprises at least 1, 2, 3, 4, 5, or 6 YA modifications. In some embodiments, a gRNA is provided comprising a hairpin modification, wherein the hairpin modification comprises one YA modification. In some embodiments, a gRNA is provided comprising a hairpin modification, wherein hairpin modification comprises 2 YA modifications. In some embodiments, the hairpin modification comprises 3 YA modifications. In some embodiments, one or more YA modifications are in a YA site. In some embodiments, one or more YA modifications are distal to a YA site.

In some embodiments, the hairpin modification comprises or further comprises a 2′-O-methyl (2′-OMe) modified nucleotide.

In some embodiments, the hairpin modification comprises or further comprises a 2′-fluoro (2′-F) modified nucleotide.

In some embodiments, the hairpin region modification comprises at least one modified nucleotide selected from a 2′H modified nucleotide (DNA), PS modified nucleotide, a YA modification, a 2′-O-methyl (2′-O-Me) modified nucleotide, a 2′-fluoro (2′-F) modified nucleotide, and/or combinations thereof.

In some embodiments, the gRNA comprises a 3′ end modification, and a modification in the hairpin region.

In some embodiments, the gRNA comprises a 5′ end modification, and a modification in the hairpin region.

In some embodiments, the gRNA comprises an upper stem modification, and a modification in the hairpin region.

In some embodiments, the gRNA comprises a hairpin modification as shown in any one of the sequences in Table 1A. In some embodiments, such a hairpin modification is combined with a 5′ end modification as shown for the corresponding sequence in Table 1A. In some embodiments, such a hairpin modification is combined with a 3′ end modification as shown for the corresponding sequence in Table 1A. In some embodiments, such a hairpin modification is combined with 5′ and 3′ end modifications as shown for the corresponding sequence in Table 1A.

In some embodiments, the gRNA comprises a 3′ end modification, a modification in the hairpin region, an upper stem modification, and a 5′ end modification.

Exemplary Modified gRNAs

In some embodiments, the gRNAs described herein comprise or consist of any of the sequences shown in Table 1A. Further, gRNAs are encompassed that comprise the modifications of any of the sequences shown in Table 1A, and identified therein by SEQ ID No. That is, the nucleotides may be the same or different, but the modification pattern shown may be the same or similar to a modification pattern of a guide sequence of Table 1A. A modification pattern includes the relative position and identity of modifications of the gRNA (e.g. 5′ terminus region, lower stem region, bulge region, upper stem region, nexus region, hairpin 1 region, hairpin 2 region, 3′ tail region).

In some embodiments, the modification pattern contains at least 50%, 55%, 60%, 70%, 75%, 80%, 85%, 90%, 95%, 96%, 97%, 98%, and 99% of the modifications of any one of the sequences shown in the sequence column of Table 1A, or over one or more regions of the sequence. In some embodiments, the modification pattern is at least 50%, 55%, 60%, 70%, 75%, 80%, 85%, 90%, 95%, 96%, 97%, 98%, and 99% identical to the modification pattern of any one of the sequences shown in the sequence column of Table 1A. In some embodiments, the modification pattern is at least 50%, 55%, 60%, 70%, 75%, 80%, 85%, 90%, 95%, 96%, 97%, 98%, and 99% identical over one or more (e.g., 1, 2, 3, 4, 5, 6, 7, or 8) regions of the sequence shown in Table 1A, e.g., a 5′ terminus region, lower stem region, bulge region, upper stem region, nexus region, hairpin 1 region, hairpin 2 region, and/or 3′ terminus region.

For example, in some embodiments, a gRNA is encompassed wherein the modification pattern is least 50%, 55%, 60%, 70%, 75%, 80%, 85%, 90%, 95%, 96%, 97%, 98%, and 99% identical to the modification pattern of a sequence over the 5′ terminus region. In some embodiments, a gRNA is encompassed wherein the modification pattern is least 50%, 55%, 60%, 70%, 75%, 80%, 85%, 90%, 95%, 96%, 97%, 98%, and 99% identical over the lower stem. In some embodiments, a gRNA is encompassed wherein the modification pattern is least 50%, 55%, 60%, 70%, 75%, 80%, 85%, 90%, 95%, 96%, 97%, 98%, and 99% identical over the bulge. In some embodiments, a gRNA is encompassed wherein the modification pattern is least 50%, 55%, 60%, 70%, 75%, 80%, 85%, 90%, 95%, 96%, 97%, 98%, and 99% identical over the upper stem. In some embodiments, a gRNA is encompassed wherein the modification pattern is least 50%, 55%, 60%, 70%, 75%, 80%, 85%, 90%, 95%, 96%, 97%, 98%, and 99% identical over the nexus. In some embodiments, a gRNA is encompassed wherein the modification pattern is least 50%, 55%, 60%, 70%, 75%, 80%, 85%, 90%, 95%, 96%, 97%, 98%, and 99% identical over the hairpin 1. In some embodiments, a gRNA is encompassed wherein the modification pattern is least 50%, 55%, 60%, 70%, 75%, 80%, 85%, 90%, 95%, 96%, 97%, 98%, and 99% identical over the hairpin 2. In some embodiments, a gRNA is encompassed wherein the modification pattern is least 50%, 55%, 60%, 70%, 75%, 80%, 85%, 90%, 95%, 96%, 97%, 98%, and 99% identical over the 3′ terminus. In some embodiments, the modification pattern differs from the modification pattern of a sequence of Table 1A, or a region (e.g. 5′ terminus, lower stem, bulge, upper stem, nexus, hairpin 1, hairpin 2, 3′ terminus) of such a sequence, at 0, 1, 2, 3, 4, 5, or 6 nucleotides. In some embodiments, the gRNA comprises modifications that differ from the modifications of a sequence of Table 1A, at 0, 1, 2, 3, 4, 5, or 6 nucleotides. In some embodiments, the gRNA comprises modifications that differ from modifications of a region (e.g. 5′ terminus, lower stem, bulge, upper stem, nexus, hairpin 1, hairpin 2, 3′ terminus) of a sequence of Table 1A, at 0, 1, 2, 3, 4, 5, or 6 nucleotides.

In some embodiments, the gRNA comprises a 2′-O-methyl (2′-O-Me) modified nucleotide. In some embodiments, the gRNA comprises a 2′-O-(2-methoxyethyl) (2′-O-moe) modified nucleotide. In some embodiments, the gRNA comprises a 2′-fluoro (2′-F) modified nucleotide. In some embodiments, the gRNA comprises a phosphorothioate (PS) bond between nucleotides. In some embodiments, the sgRNA comprises a YA modification.

In some embodiments, the gRNA comprises a 5′ end modification, a 3′ end modification, or 5′ and 3′ end modification, such as a protective end modification. In some embodiments, the 5′ end modification comprises a phosphorothioate (PS) bond between nucleotides. In some embodiments, the 5′ end modification comprises a 2′-O-methyl (2′-O-Me), 2′-O-(2-methoxyethyl) (2′-O-moe), and/or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-moe), and/or 2′-fluoro (2′-F) modified nucleotide. The end modification may comprise a phosphorothioate (PS), 2′-O-methyl (2′-O-Me), 2′-O-(2-methoxyethyl) (2′-O-moe), and/or 2′-fluoro (2′-F) modification. Equivalent end modifications are also encompassed by embodiments described herein. In some embodiments, the gRNA comprises an end modification in combination with a modification of one or more regions of the gRNA.

Modified gRNAs comprising combinations of 5′ end modifications, 3′ end modifications, upper stem modifications, hairpin modifications, and 3′ terminus modifications, as described above, are encompassed. Exemplary modified gRNAs are described below.

In some embodiments, a gRNA is provided comprising or consisting of any one of the sequences described in SEQ ID NOs: 1-98, 201-294, 401-494, 601-698, 801-875. In some embodiments, a gRNA is provided compromising or consisting of any one of the sequences described in SEQ ID NOs: 101-198, 301-394, 501-594, 701-798, or 901-975 including the modifications shown in Table 1A.

In some embodiments, a gRNA is provided comprising any one of the sequences of SEQ ID NOs: 201-294, wherein the gRNA further comprises a guide region that is complementary to a target sequence, and directs a Cas9 to its target for cleavage. In some embodiments, a gRNA is provided comprising any one of the modified sequences of SEQ ID NOs: 301-394, wherein the gRNA further comprises a guide region that is complementary to a target sequence, and directs a Cas9 to its target for cleavage. In some instances, the invention comprises gRNA is provided comprising nucleic acids having at least 99, 98, 97, 96, 95, 94, 93, 92, 91, 90, 85, 80, 75, or 70% identity to the nucleic acids of any one of SEQ ID NOs: 1-98, 201-294, 401-494, 601-698, or 801-875. In some embodiments, a gRNA is provided comprising nucleic acids having at least 99, 98, 97, 96, 95, 94, 93, 92, 91, 90, 85, 80, 75, or 70% identity to the nucleic acids of any one of SEQ ID NOs: 101-198, 301-394, 501-594, 701-798, or 901-975, wherein the modification pattern is identical to the modification pattern shown in the reference sequence identifier in Table 1A. Any of the foregoing the gRNAs may further comprises three phosphorothioate (PS) bonds linking the first four nucleotides at the 5′ terminus and three PS bonds linking the last four nucleotides at the 3′ terminus.

In some embodiments, the gRNA comprises modifications at 1, 2, 3, or 4 of the first 4 nucleotides at its 5′ end. In some embodiments, the first three or four nucleotides at the 5′ terminus, and the last three or four nucleotides at the 3′ terminus are modified. In some embodiments, the first four nucleotides at the 5′ end, and the last four nucleotides at the 3′ terminus are linked with phosphorothioate (PS) bonds. In some embodiments, the modification comprises 2′-O-Me. In some embodiments, the modification comprises 2′-F. In some embodiments, the modification comprises 2′-O-moe.

In some embodiments, the gRNA comprises, if the nucleotide mentioned is present in the gRNA, modifications at 1, 2, 3, or 4 of the first 4 nucleotides at the 5′ end. In some embodiments, the gRNA comprises modifications at 1, 2, 3, or 4 of the last 4 nucleotides at the 3′ end (3′ tail or conserved portion of an sgRNA). In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-O-Me or 2′-O-moe modifications.

In some embodiments, the first four nucleotides at the 5′ terminus and the last four nucleotides at the 3′ terminus are linked with a PS bond, and the first three nucleotides at the 5′ terminus and the last three nucleotides at the 3′ terminus comprise 2′-F modifications.

In some embodiments, a gRNA is provided, if the nucleotide mentioned is present in the gRNA, wherein LS1, LS6, LS7, LS8, LS11, and LS12 are modified with 2′-O-Me. In some embodiments, each of the nucleotides in the bulge region of the gRNA are modified with 2′-O-Me. In some embodiments, each of the nucleotides in the upper stem region of the gRNA are modified with 2′-O-Me. In some embodiments, N16, N17, and N18 in the nexus region of the gRNA are modified with 2′-O-Me. In some embodiments, each of the nucleotides remaining in the hairpin 1 region of the gRNA are modified with 2′-O-Me. In some embodiments, each of the nucleotides remaining in the hairpin 2 region of the gRNA are modified with 2′-O-Me.

In some embodiments, a gRNA comprising a 5′ end modification and one or more modifications in one or more of: the upper stem region; the hairpin 1 region; and the hairpin 2 region is provided, wherein the 5′ end modification comprises at least two phosphorothioate linkages within the first seven nucleotides of the 5′ terminus.

In some embodiments, a gRNA comprising a 5′ end modification and one or more modifications in one or more of: the upper stem region; the hairpin 1 region; and the hairpin 2 region is provided, wherein the 5′ end modification comprises one or more phosphorothioate linkages at the 5′ end. In some embodiments, one or more phorphorothioate bonds link the 5′ terminal nucleotides.

In some embodiments, a gRNA comprising a 5′ end modification and one or more modifications in one or more of: the upper stem region; the hairpin 1 region; and the hairpin 2 region is provided, wherein the 5′ end modification comprises one or more phosphorothioate linkages within the first seven nucleotides of the 5′ terminus.

In some embodiments, the invention comprises a gRNA comprising any one of the modified sequences of SEQ ID NOs: 201-294 or 301-394, wherein the gRNA further comprises a 5′ guide region that is at least partially complementary to a target sequence, and optionally directs a Cas9 to its target for cleavage.

In some embodiments, the invention comprises a gRNA comprising nucleotides having at least 99, 98, 97, 96, 95, 94, 93, 92, 91, 90, 85, 80, 75, or 70% identity to the nucleotides of any one of SEQ ID NOs: 1-98, 201-294, 401-494, 601-698, or 801-875 wherein the modification pattern is identical to the modification pattern shown in the reference sequence identifier. That is, the nucleotides A, U, C, and G may differ by 99, 98, 97, 96, 95, 94, 93, 92, 91, 90, 85, 80, 75, or 70% compared to what is shown in in the sequences, but the modification remains unchanged.

In some embodiments, a gRNA is provided comprising, if the nucleotide mentioned is present in the guide, 2′-O-Me modified nucleotides at: the first three nucleotides in the 5′ terminus; LS1, LS6, LS7, LS8, LS11, and LS12 in the lower stem; B1 and B2 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides at the 3′ terminus.

In some embodiments, a gRNA is provided comprising, if the nucleotide mentioned is present in the guide, 2′-O-Me modified nucleotides at: the first three nucleotides in the 5′ terminus; LS1, LS6, LS7, LS8, LS11, and LS12 in the lower stem; B1-B6 in the bulge region; each of the nucleotides in the upper stem region; N16, N17, and N18 in the nexus region; each of the nucleotides in the hairpin 1 region; one nucleotide between hairpin 1 and hairpin 2; each of the nucleotides in the hairpin 2 region; and the last four nucleotides at the 3′ terminus. In some embodiments, the sgRNA further comprises three PS bonds between the first four nucleotides at the 5′ terminus and three PS bonds between the last four nucleotides at the 3′ terminus.

In some embodiments, a gRNA is provided comprising 2′-F modified nucleotides at: LS9 and LS10 in the lower stem; 15-N18 in the nexus region; H2-9-HS-15 in the hairpin 2 region; and the second to last, third to last, and fourth to last nucleotide in the 3′ terminus region.

In some embodiments, a gRNA is provided comprising 2′-F modified nucleotides at: each nucleotide in the lower stem; 15-N18 in the nexus region; H2-9-HS-15 in the hairpin 2 region; and the second to last, third to last, and fourth to last nucleotide in the 3′ terminus region.

In some embodiments, a gRNA is provided comprising, if the nucleotide mentioned is present in the guide, 2′-OMe modified nucleotides at LS8, LS10, LS12, H1-2, H1-4, H1-6, H1-8, H1-10, H1-12, H2-1, H2-3, H2-5, H2-7, H2-9, H2-11, H2-13, H2- 15, and the last and third to last nucleotides at the 3′ terminus; and 2′-F modifications at LS7, LS9, LS11; H1-1, H1-3, H1-5, H1-7, H1-9, H1-11, H1-13, H2-2, H2-4, H2-6, H2-8, H2-10, H2-12, H2-14, and the second to last and fourth to last nucleotide at the 3′ terminus.

Any of the foregoing modification patterns can be combined with a modification pattern set forth in the embodiments described above, e.g., in the summary section or Table 1, to the extent that they are non-overlapping. In the event that combining a foregoing modification pattern with a modification pattern set forth in the summary section or Table 1A would result in incompatible modifications (e.g., the same position would be both 2′-OMe and 2′-fluoro), the modification set forth in the summary section or Table 1A controls.

sgRNAs; Domains/Regions Thereof.

In some embodiments, a gRNA provided herein is an sgRNA. Briner A E et al., Molecular Cell 56:333-339 (2014) describes functional domains of sgRNAs, referred to herein as “domains”, including the “spacer” domain responsible for targeting, the “lower stem”, the “bulge”, “upper stem” (which may include a tetraloop), the “nexus”, and the “hairpin 1” and “hairpin 2” domains. See Briner et al. at page 334, FIG. 1A. As described in detail elsewhere herein, one or more domains (e.g., hairpin 1 and/or the upper stem) may be shortened in an sgRNA described herein.

Table 3 provides a schematic of the domains of an sgRNA as used herein. In Table 3, the “n” between regions represents a variable number of nucleotides, for example, from 0 to 1, 2, 3, 4, 5, 6, 7, 8, 9, 10, 11, 12, 13, 14, 15, 16, 17, 18, 19, 20, or more. In some embodiments, n equals 0. In some embodiments, n equals 1.

5′ Terminus Region

In some embodiments, the sgRNA comprises nucleotides at the 5′ terminus as shown in Table 3. In some embodiments, the 5′ terminus of the sgRNA comprises a spacer or guide region that functions to direct a Cas protein, e.g., a Cas9 protein, to a target nucleotide sequence. In some embodiments, the 5′ terminus does not comprise a guide region. In some embodiments, the 5′ terminus comprises a spacer and additional nucleotides that do not function to direct a Cas protein to a target nucleotide region.

Lower Stem

In some embodiments, the sgRNA comprises a lower stem (LS) region that when viewed linearly, is separated by a bulge and upper stem regions. See Table 3.

In some embodiments, the lower stem regions comprise 1-12 nucleotides, e.g. in one embodiment the lower stem regions comprise LS1-LS12. In some embodiments, the lower stem region comprises fewer nucleotides than shown in Table 3. In some embodiments, the lower stem region comprises more nucleotides than shown in Table 3. When the lower stem region comprises fewer or more nucleotides than shown in the schematic of Table 3, the modification pattern, as will be apparent to the skilled artisan, should be maintained.

In some embodiments, the lower stem region has nucleotides that are complementary in nucleic acid sequence when read in opposite directions. In some embodiments, the complementarity in nucleic acid sequence of lower stem leads to a secondary structure of a stem in the sgRNA (e.g., the regions may base pair with one another). In some embodiments, the lower stem regions may not be perfectly complimentary to each other when read in opposite directions.

Bulge

In some embodiments, the sgRNA comprises a bulge region comprising six nucleotides, B1-B6. When viewed linearly, the bulge region is separated into two regions. See Table 3. In some embodiments, the bulge region comprises six nucleotides, wherein the first two nucleotides are followed by an upper stem region, followed by the last four nucleotides of the bulge. In some embodiments, the bulge region comprises fewer nucleotides than shown in Table 3. In some embodiments, the bulge region comprises more nucleotides than shown in Table 3. When the bulge region comprises fewer or more nucleotides than shown in the schematic of Table 3, the modification pattern, as will be apparent to the skilled artisan, should be maintained.

In some embodiments, the presence of a bulge results in a directional kink between the upper and lower stem modules in an sgRNA.

Upper Stem

In some embodiments, the upper stem region is a shortened upper stem region, such as any of the shortened upper stem regions described elsewhere herein.

In other embodiments, the sgRNA comprises an upper stem region comprising 12 nucleotides. In some embodiments, the upper stem region comprises a loop sequence. In some instances, the loop is a tetraloop (loop consisting of four nucleotides). In some embodiments, the upper stem region comprises more nucleotides than shown in Table 3.

When the upper stem region comprises fewer or more nucleotides than shown in the schematic of Table 3, the modification pattern, as will be apparent to the skilled artisan, should be maintained.

In some embodiments, the upper stem region has nucleotides that are complementary in nucleic acid sequence when read in opposite directions. In some embodiments, the complementarity in nucleic acid sequence of upper stem leads to a secondary structure of a stem in the sgRNA (e.g., the regions may base pair with one another). In some embodiments, the upper stem regions may not be perfectly complimentary to each other when read in opposite directions.

Nexus

In some embodiments, the sgRNA comprises a nexus region that is located between the lower stem region and the hairpin 1 region. In some embodiments, the nexus comprises 18 nucleotides. In some embodiments, the nexus region comprises nucleotides N1 through N18 as shown in Table 3. In some embodiments, the nexus region comprises a substitution (e.g., at position N18) or lacks a nucleotide, such as any of the nexus regions with a substitution or lacking a nucleotide described in detail elsewhere herein.

In some embodiments, the nexus region comprises fewer nucleotides than shown in Table 3. In some embodiments, the nexus region comprises more nucleotides than shown in Table 3. When the nexus region comprises fewer or more nucleotides than shown in the schematic of Table 3, the modification pattern, as will be apparent to the skilled artisan, should be maintained.

In some embodiments, the nexus region has nucleotides that are complementary in nucleic acid sequence when read in opposite directions. In some embodiments, the complementarity in nucleic acid sequence leads to a secondary structure of a stem and/or stem loop in the sgRNA (e.g., certain nucleotides in the nexus region may base pair with one another). In some embodiments, the nexus regions may not be perfectly complimentary to each other when read in opposite directions.

Hairpin

In some embodiments, the sgRNA comprises one or more hairpin regions. In some embodiments, the hairpin region is downstream of (e.g., 3′ to) the nexus region. In some embodiments, the region of nucleotides immediately downstream of the nexus region is termed “hairpin 1” or “H1”. In some embodiments, the region of nucleotides 3′ to hairpin 1 is termed “hairpin 2” or “H2”. In some embodiments, the hairpin region comprises both hairpin 1 and hairpin 2. In some embodiments, the sgRNA comprises hairpin 1 or hairpin 2.

In some embodiments, the hairpin 1 region is a shortened hairpin 1 region, such as any of the shortened hairpin 1 regions described elsewhere herein.

In other embodiments, the hairpin 1 region comprises 12 nucleotides immediately downstream of the nexus region. In some embodiments, the hairpin 1 region comprises nucleotides H1-1 through H1-12 as shown in Table 3.

In some embodiments, the hairpin 2 region comprises 15 nucleotides downstream of the hairpin 1 region. In some embodiments, the hairpin 2 region comprises nucleotides H2-1 through H2-15 as shown in Table 3.

In some embodiments, one or more nucleotides is present between the hairpin 1 and the hairpin 2 regions. The one or more nucleotides between the hairpin 1 and hairpin 2 region may be modified or unmodified. In some embodiments, hairpin 1 and hairpin 2 are separated by one nucleotide. In some embodiments, the hairpin regions comprise fewer nucleotides than shown in Table 3. In some embodiments, the hairpin regions comprise more nucleotides than shown in Table 3. When a hairpin region comprises fewer or more nucleotides than shown in the schematic of Table 3, the modification pattern, as will be apparent to the skilled artisan, should be maintained.

In some embodiments, a hairpin region has nucleotides that are complementary in nucleic acid sequence when read in opposite directions. In some embodiments, the hairpin regions may not be perfectly complimentary to each other when read in opposite directions (e.g., the top or loop of the hairpin comprises unpaired nucleotides).

In some embodiments, the sgRNA comprises replacement of hairpin 1 with nucleotides “n”, wherein “n” is an integer between 1 and 50, 40, 30, 20, 15, 10, 5, 4, 3, and 2. In some embodiments, the hairpin 1 region of an sgRNA is replaced by 2 nucleotides.

3′ Terminus

The sgRNA has a 3′ end, which is the last nucleotide of the sgRNA. The 3′ terminus region includes the last 1-7 nucleotides from the 3′ end. In some embodiments, the 3′ end is the end of hairpin 2. In some embodiments, the sgRNA comprises nucleotides after the hairpin region(s). In some embodiments, the sgRNA includes a 3′ tail region, in which case the last nucleotide of the 3′ tail is the 3′ terminus. In some embodiments, the 3′ tail comprises 1, 2, 3, 4, 5, 6, 7, 8, 9, 10, 15 or 20 or more nucleotides, e.g. that are not associated with the secondary structure of a hairpin. In some embodiments, the 3′ tail region comprises 1, 2, 3, or 4 nucleotides that are not associated with the secondary structure of a hairpin. In some embodiments, the 3′ tail region comprises 4 nucleotides that are not associated with the secondary structure of a hairpin. In some embodiments, the 3′ tail region comprises 1, 2, or 3 nucleotides that are not associated with the secondary structure of a hairpin.

TABLE 2
(Conserved Portion of a spyCas9 sgRNA; SEQ ID NO: 400)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 |
|---|---|---|---|---|---|---|---|---|---|---|---|---|---|---|---|---|---|---|---|---|---|---|---|---|---|---|---|---|
| G | U | U | U | U | A | G | A | G | C | u | A | G | A | A | A | U | A | G | C | A | A | G | U | U | A | A | A | A |

| | | | | |
|---|---|---|---|---|
| LS1-LS6 | B1-B2 | US1-US12 | B2-B6 | LS7-LS12 |

| 30 | 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 |
|---|---|---|---|---|---|---|---|---|---|---|---|---|---|---|---|---|---|---|---|---|---|---|---|---|---|---|---|---|---|
| U | A | A | G | G | C | U | A | G | U | C | C | G | U | U | A | U | C | A | A | C | U | U | G | A | A | A | A | A | G |

| | | |
|---|---|---|
| LS7- | Nexus | H1-1 through H1-12 |
| LS12 | | |

| 60 | 61 | 62 | 63 | 64 | 65 | 66 | 67 | 68 | 69 | 70 | 71 | 72 | 73 | 74 | 75 | 76 |
|---|---|---|---|---|---|---|---|---|---|---|---|---|---|---|---|---|
| U | G | G | C | A | C | C | G | A | G | U | C | G | G | U | G | C |

| | | |
|---|---|---|
| H1-1 | N | H2-1 through H2-15 |
| through | | |
| H1-12 | | |

TABLE 3
(Regions of sgRNA (linear view, 5′ to 3′)

| | LSI-6 | | B1-2 | | US1-12 | | B3-6 | |
|---|---|---|---|---|---|---|---|---|
| 5′ terminus (n) | lower stem | n | bulge | n | upper stem | n | bulge | n |

| LS7-12 | | N1-18 | | H1-1 thru H1-12 | | H2-1 thru H2-15 | |
|---|---|---|---|---|---|---|---|
| lower stem | n | nexus | n | hairpin 1 | n | hairpin 2 | 3′ terminus |

### Compositions and Kits

Compositions comprising any of the gRNAs (e.g., sgRNAs, dgRNAs, or crRNAs) described herein and a carrier, excipient, diluent, or the like are encompassed. In some instances, the excipient or diluent is inert. In some instances, the excipient or diluent is not inert. In some embodiments, a pharmaceutical formulation is provided comprising any of the gRNAs (e.g., sgRNAs, dgRNAs, or crRNAs) described herein and a pharmaceutically acceptable carrier, excipient, diluent, or the like. In some embodiments, the pharmaceutical formulation further comprises an LNP. In some embodiments, the pharmaceutical formulation further comprises a Cas9 protein or an mRNA encoding a Cas9 protein. In some embodiments, the pharmaceutical formulation comprises any one or more of the gRNAs (e.g., sgRNAs, dgRNAs, or crRNAs), an LNP, and a Cas9 protein or mRNA encoding a Cas9 protein.

Also provided are kits comprising one or more gRNAs (e.g., sgRNAs, dgRNAs, or crRNAs), compositions, or pharmaceutical formulations described herein. In some embodiments, a kit further comprises one or more of a solvent, solution, buffer, each separate from the composition or pharmaceutical formulation, instructions, or desiccant.

Compositions Comprising an RNA-Guided DNA Binding Agent or mRNA Encoding RNA-Guided DNA Binding Agent

In some embodiments, compositions or pharmaceutical formulations are provided comprising at least one gRNA (e.g., sgRNA, dgRNA, or crRNA) described herein and an RNA-guided DNA binding agent or a nucleic acid (e.g., an mRNA) encoding an RNA-guided DNA binding agent. In some embodiments, the RNA-guided DNA binding agent is a Cas protein. In some embodiments, the gRNA together with a Cas protein or nucleic acid (e.g., mRNA) encoding Cas protein is called a Cas RNP. In some embodiments, the RNA-guided DNA binding agent is one that functions with the gRNA to direct a RNA-guided DNA binding agent to a target nucleic acid sequence. In some embodiments, the RNA-guided DNA binding agent is a Cas protein from the Type-II CRISPR/Cas system. In some embodiments, the Cas protein is Cas9. In some embodiments, the Cas9 protein is a wild type Cas9. In some embodiments, the Cas9 protein is derived from the Streptococcus pyogenes Cas9 protein, e.g., a S. pyogenes Cas9 (sypCas9). In some embodiments, compositions are provided comprising at least one gRNA and a nuclease or an mRNA encoding a spyCas9. In some embodiments, the Cas9 protein is not derived from S. pyogenes, but functions in the same way as S. pyogenes Cas9 such that gRNA that is specific to S. pyogenes Cas9 will direct the non-S. pyogenes Cas9 to its target site. In some embodiments, the Cas9 protein is derived from the Staphylococcus aureus Cas9 protein, e.g., a SaCas9. In some embodiments, compositions are provided comprising at least one gRNA and a nuclease or an mRNA encoding a saCas9. In some embodiments, the Cas induces a double strand break in target DNA. Equivalents of spyCas9 and saCas9 protein are encompassed by the embodiments described herein.

RNA-guided DNA binding agents, including Cas9, encompass modified and variants thereof. Modified versions having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase RNA-guided DNA binding agent, such as a nickase Cas9, that induces a nick rather than a double strand break in the target DNA.

In some embodiments, the nuclease, e.g. the RNA-guided DNA binding agent, may be modified to contain only one functional nuclease domain. For example, the RNA-guided DNA binding agent may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within an RNA-guided DNA binding agent nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include D10A (based on the S. pyogenes Cas9 protein). In some embodiments, the Cas protein may comprise an amino acid substitution in the HNH or HNH-like nuclease domain. Exemplary amino acid substitutions in the HNH or HNH-like nuclease domain include E762A, H840A, N863A, H983A, and D986A (based on the spyCas9 protein).

In some embodiments, the RNP complex described herein comprises a nickase or an mRNA encoding a nickase and a pair of gRNAs (one or both of which may be sgRNAs) that are complementary to the sense and antisense strands of the target sequence, respectively. In this embodiment, the gRNAs (e.g., sgRNAs) direct the nickase to a target sequence and introduce a double stranded break (DSB) by generating a nick on opposite strands of the target sequence (i.e., double nicking). In some embodiments, use of double nicking may improve specificity and reduce off-target effects. In some embodiments, a nickase RNA-guided DNA binding agent is used together with two separate gRNAs (e.g., sgRNAs) that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fok1. In some embodiments, a Cas protein may be a modified nuclease.

In some embodiments, the Cas protein comprises a fusion protein comprising a catalytically inactive Cas nuclease (e.g., Cas9) linked to a heterologous functional domain (see, e.g., WO2014152432). In some embodiments, the catalytically inactive Cas9 is from S. pyogenes. In some embodiments, the catalytically inactive Cas comprises mutations that inactivate the Cas. In some embodiments, the heterologous functional domain is a domain that modifies gene expression, histones, or DNA. In some embodiments, the heterologous functional domain is a transcriptional activation domain or a transcriptional repressor domain. In some embodiments, the nuclease is a catalytically inactive Cas nuclease, such as dCas9.

In some embodiments, the target sequence may be adjacent to a PAM. In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Cas9 protein or Cas9 ortholog, including those disclosed in FIG. 1 of Ran et al., Nature 520:186-191 (2015). In some embodiments, the PAM may comprise 2, 3, 4, 5, 6, 7, 8, 9, or 10 nucleotides in length. Non-limiting exemplary PAM sequences include NGG, NAG, NGA, NGAG, NGCG, NNGRRT, TTN, NGGNG, NG, NAAAAN, NNAAAAW, NNNNACA, GNNNCNNA, and NNNNGATT (wherein N is defined as any nucleotide, and W is defined as either A or T, and R is defined as either A or G). In some embodiments, the PAM sequence may be NGG. In some embodiments, the PAM sequence may be NGGNG. In some embodiments, the PAM sequence may be NNAAAAW.

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) at the carboxy terminus. In some embodiments, the RNA-guided DNA-binding agent may be fused with two NLSs, one at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 1001) or PKKKRRV (SEQ ID NO: 1002). In some embodiments, the NLS may be a bipartite sequence, such as the NLS of nucleoplasmin, KRPAATKKAGQAKKKK (SEQ ID NO: 1003). In a specific embodiment, a single PKKKRKV (SEQ ID NO: 1001) NLS may be fused at the C-terminus of the RNA-guided DNA-binding agent. One or more linkers are optionally included at the fusion site.

In some embodiments, an nucleic acid (e.g., mRNA) comprising an ORF encoding an RNA-guided DNA binding agent is used which has one or more of the following features. In some embodiments, the ORF encoding the RNA-guided DNA-binding agent, e.g. a Cas9 nuclease such as an S. pyogenes Cas9, has an adenine content ranging from its minimum adenine content to about 150% of its minimum adenine content. In some embodiments, the adenine content of the ORF is less than or equal to about 145%, 140%, 135%, 130%, 125%, 120%, 115%, 110%, 105%, 104%, 103%, 102%, or 101% of its minimum adenine content. In some embodiments, the ORF has an adenine content equal to its minimum adenine content. In some embodiments, the ORF has an adenine content less than or equal to about 150% of its minimum adenine content. In some embodiments, the ORF has an adenine content less than or equal to about 145% of its minimum adenine content. In some embodiments, the ORF has an adenine content less than or equal to about 140% of its minimum adenine content. In some embodiments, the ORF has an adenine content less than or equal to about 135% of its minimum adenine content. In some embodiments, the ORF has an adenine content less than or equal to about 130% of its minimum adenine content. In some embodiments, the ORF has an adenine content less than or equal to about 125% of its minimum adenine content. In some embodiments, the ORF has an adenine content less than or equal to about 120% of its minimum adenine content. In some embodiments, the ORF has an adenine content less than or equal to about 115% of its minimum adenine content. In some embodiments, the ORF has an adenine content less than or equal to about 110% of its minimum adenine content. In some embodiments, the ORF has an adenine content less than or equal to about 105% of its minimum adenine content. In some embodiments, the ORF has an adenine content less than or equal to about 104% of its minimum adenine content. In some embodiments, the ORF has an adenine content less than or equal to about 103% of its minimum adenine content. In some embodiments, the ORF has an adenine content less than or equal to about 102% of its minimum adenine content. In some embodiments, the ORF has an adenine content less than or equal to about 101% of its minimum adenine content.

In some embodiments, the ORF has an adenine dinucleotide content ranging from its minimum adenine dinucleotide content to 200% of its minimum adenine dinucleotide content. In some embodiments, the adenine dinucleotide content of the ORF is less than or equal to about 195%, 190%, 185%, 180%, 175%, 170%, 165%, 160%, 155%, 150%, 145%, 140%, 135%, 130%, 125%, 120%, 115%, 110%, 105%, 104%, 103%, 102%, or 101% of its minimum adenine dinucleotide content. In some embodiments, the ORF has an adenine dinucleotide content equal to its minimum adenine dinucleotide content. In some embodiments, the ORF has an adenine dinucleotide content less than or equal to about 200% of its minimum adenine dinucleotide content. In some embodiments, the ORF has an adenine dinucleotide content less than or equal to about 195% of its minimum adenine dinucleotide content. In some embodiments, the ORF has an adenine dinucleotide content less than or equal to about 190% of its minimum adenine dinucleotide content. In some embodiments, the ORF has an adenine dinucleotide content less than or equal to about 185% of its minimum adenine dinucleotide content. In some embodiments, the ORF has an adenine dinucleotide content less than or equal to about 180% of its minimum adenine dinucleotide content. In some embodiments, the ORF has an adenine dinucleotide content less than or equal to about 175% of its minimum adenine dinucleotide content. In some embodiments, the ORF has an adenine dinucleotide content less than or equal to about 170% of its minimum adenine dinucleotide content. In some embodiments, the ORF has an adenine dinucleotide content less than or equal to about 165% of its minimum adenine dinucleotide content. In some embodiments, the ORF has an adenine dinucleotide content less than or equal to about 160% of its minimum adenine dinucleotide content. In some embodiments, the ORF has an adenine dinucleotide content less than or equal to about 155% of its minimum adenine dinucleotide content. In some embodiments, the ORF has an adenine dinucleotide content equal to its minimum adenine dinucleotide content. In some embodiments, the ORF has an adenine dinucleotide content less than or equal to about 150% of its minimum adenine dinucleotide content. In some embodiments, the ORF has an adenine dinucleotide content less than or equal to about 145% of its minimum adenine dinucleotide content. In some embodiments, the ORF has an adenine dinucleotide content less than or equal to about 140% of its minimum adenine dinucleotide content. In some embodiments, the ORF has an adenine dinucleotide content less than or equal to about 135% of its minimum adenine dinucleotide content. In some embodiments, the ORF has an adenine dinucleotide content less than or equal to about 130% of its minimum adenine dinucleotide content. In some embodiments, the ORF has an adenine dinucleotide content less than or equal to about 125% of its minimum adenine dinucleotide content. In some embodiments, the ORF has an adenine dinucleotide content less than or equal to about 120% of its minimum adenine dinucleotide content. In some embodiments, the ORF has an adenine dinucleotide content less than or equal to about 115% of its minimum adenine dinucleotide content. In some embodiments, the ORF has an adenine dinucleotide content less than or equal to about 110% of its minimum adenine dinucleotide content. In some embodiments, the ORF has an adenine dinucleotide content less than or equal to about 105% of its minimum adenine dinucleotide content. In some embodiments, the ORF has an adenine dinucleotide content less than or equal to about 104% of its minimum adenine dinucleotide content. In some embodiments, the ORF has an adenine dinucleotide content less than or equal to about 103% of its minimum adenine dinucleotide content. In some embodiments, the ORF has an adenine dinucleotide content less than or equal to about 102% of its minimum adenine dinucleotide content. In some embodiments, the ORF has an adenine dinucleotide content less than or equal to about 101% of its minimum adenine dinucleotide content.

In some embodiments, the ORF has an adenine dinucleotide content ranging from its minimum adenine dinucleotide content to the adenine dinucleotide content that is 90% or lower of the maximum adenine dinucleotide content of a reference sequence that encodes the same protein as the mRNA in question. In some embodiments, the adenine dinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine dinucleotide content of a reference sequence that encodes the same protein as the mRNA in question.

In some embodiments, the ORF has an adenine trinucleotide content ranging from 0 adenine trinucleotides to 1, 2, 3, 4, 5, 6, 7, 8, 9, 10, 20, 30, 40, or 50 adenine trinucleotides (where a longer run of adenines counts as the number of unique three-adenine segments within it, e.g., an adenine tetranucleotide contains two adenine trinucleotides, an adenine pentanucleotide contains three adenine trinucleotides, etc.). In some embodiments, the ORF has an adenine trinucleotide content ranging from 0% adenine trinucleotides to 0.1%, 0.2%, 0.3%, 0.4%, 0.5%, 0.6%, 0.7%, 0.8%, 0.9%, 1%, 1.5%, or 2% adenine trinucleotides, where the percentage content of adenine trinucleotides is calculated as the percentage of positions in a sequence that are occupied by adenines that form part of an adenine trinucleotide (or longer run of adenines), such that the sequences UUUAAA and UUUUAAAA would each have an adenine trinucleotide content of 50%. For example, in some embodiments, the ORF has an adenine trinucleotide content less than or equal to 2%. For example, in some embodiments, the ORF has an adenine trinucleotide content less than or equal to 1.5%. In some embodiments, the ORF has an adenine trinucleotide content less than or equal to 1%. In some embodiments, the ORF has an adenine trinucleotide content less than or equal to 0.9%. In some embodiments, the ORF has an adenine trinucleotide content less than or equal to 0.8%. In some embodiments, the ORF has an adenine trinucleotide content less than or equal to 0.7%. In some embodiments, the ORF has an adenine trinucleotide content less than or equal to 0.6%. In some embodiments, the ORF has an adenine trinucleotide content less than or equal to 0.5%. In some embodiments, the ORF has an adenine trinucleotide content less than or equal to 0.4%. In some embodiments, the ORF has an adenine trinucleotide content less than or equal to 0.3%. In some embodiments, the ORF has an adenine trinucleotide content less than or equal to 0.2%. In some embodiments, the ORF has an adenine trinucleotide content less than or equal to 0.1%. In some embodiments, a nucleic acid is provided that encodes an RNA-guided DNA-binding agent comprising an ORF containing no adenine trinucleotides.

In some embodiments, the ORF has an adenine trinucleotide content ranging from its minimum adenine trinucleotide content to the adenine trinucleotide content that is 90% or lower of the maximum adenine trinucleotide content of a reference sequence that encodes the same protein as the mRNA in question. In some embodiments, the adenine trinucleotide content of the ORF is less than or equal to about 85%, 80%, 75%, 70%, 65%, 60%, 55%, 50%, 45%, 40%, 35%, 30%, 25%, 20%, 15%, 10%, or 5% of the maximum adenine trinucleotide content of a reference sequence that encodes the same protein as the mRNA in question.

A given ORF can be reduced in adenine content or adenine dinucleotide content or adenine trinucleotide content, for example, by using minimal adenine codons in a sufficient fraction of the ORF. For example, an amino acid sequence for an RNA-guided DNA-binding agent can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 4.

TABLE 4
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, a nucleic acid is provided that encodes an RNA-guided DNA-binding agent, e.g. a Cas9 nuclease such as an S. pyogenes Cas9, comprising an ORF consisting of a set of codons of which at least about 75%, 80%, 85%, 90%, 95%, 98%, 99%, or 100% of the codons are codons listed in Table 4. In some embodiments, the ORF has minimal nucleotide homopolymers, e.g., repetitive strings of the same nucleotides. For example, in some embodiments, when selecting a minimal uridine codon from the codons listed in Table 4, a nucleic acid is constructed by selecting the minimal adenine codons that reduce the number and length of nucleotide homopolymers, e.g., selecting GCG instead of GCC for alanine or selecting GGC instead of GGG for glycine.

In any of the foregoing embodiments, the nucleic acid may be an mRNA.

In some embodiments, at least 75%, 80%, 85%, 90%, 95%, 96%, 97%, 98%, 99%, or 100% of the codons in an ORF are codons from a codon set shown in Table 5 (e.g., the low U, low A, or low A/U codon set). The codons in the low U, low A, and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U codon set shown in Table 5. In some embodiments, at least 75%, 80%, 85%, 90%, 95%, 96%, 97%, 98%, 99%, or 100% of the codons in an ORF are codons from the low A codon set shown in Table 5. In some embodiments, at least 75%, 80%, 85%, 90%, 95%, 96%, 97%, 98%, 99%, or 100% of the codons in an ORF are codons from the low A/U codon set shown in Table 5.

TABLE 5
Exemplary Codon Sets

| Amino | | | | Long Half |
|---|---|---|---|---|
| Acid | Low U | Low A | Low A/U | Life |
| Gly | GGC | GGC | GGC | GGT |
| Glu | GAG | GAG | GAG | GAA |
| Asp | GAC | GAC | GAC | GAC |
| Val | GTG | GTG | GTG | GTC |
| Ala | GCC | GCC | GCC | GCC |
| Arg | AGA | CGG | CGG | AGA |
| Ser | AGC | TCC | AGC | TCT |
| Lys | AAG | AAG | AAG | AAG |
| Asn | AAC | AAC | AAC | AAC |
| Met | ATG | ATG | ATG | ATG |
| Ile | ATC | ATC | ATC | ATC |
| Thr | ACC | ACC | ACC | ACC |
| Trp | TGG | TGG | TGG | TGG |
| Cys | TGC | TGC | TGC | TGC |
| Tyr | TAC | TAC | TAC | TAC |
| Leu | CTG | CTG | CTG | TTG |
| Phe | TTC | TTC | TTC | TTC |
| Gln | CAG | CAG | CAG | CAA |
| His | CAC | CAC | CAC | CAC |

Exemplary Sequences

In some embodiments, the ORF encoding the RNA-guided DNA binding agent comprises a sequence with at least 90%, 93%, 95%, 96%, 97%, 98%, 99%, 99.5%, or 100% identity to any one of SEQ ID NOs: 1102-1122, 1125, 1126, or 1129-1146; and/or the ORF has at least 90%, 93%, 95%, 96%, 97%, 98%, 99%, 99.5%, or 100% identity to any one of SEQ ID NOs: 1102-1122, 1125, 1126, or 1129-1146 over at least its first 50, 200, 250, or 300 nucleotides, or at least 95% identity to any one of SEQ ID NOs: 1102-1122, 1125, 1126, or 1129-1146 over at least its first 30, 50, 70, 100, 150, 200, 250, or 300 nucleotides; and/or the ORF consists of a set of codons of which at least 95%, 96%, 97%, 98%, 99%, 99.5%, or 100% of the codons are codons listed in Table 4 or 5; and/or the ORF has an adenine content ranging from its minimum adenine content to 123% of the minimum adenine content; and/or the ORF has an adenine dinucleotide content ranging from its minimum adenine dinucleotide content to 150% of the minimum adenine dinucleotide content. In some embodiments, the polynucleotide encoding the RNA-guided DNA binding agent comprises a sequence with at least 95%, 96%, 97%, 98%, 99%, 99.5%, or 100% identity to any one of SEQ ID NOs: 1102-1122, 1125, 1126, or 1129-1146.

In some embodiments, the mRNA comprises a sequence with at least 90% identity to any one of SEQ ID NOs: 1101, 1123, 1124, or 1127, wherein the sequence comprises an ORF encoding an RNA-guided DNA binding agent. In some embodiments, the mRNA comprises a sequence with at least 90% identity to any one of SEQ ID NOs: 1101, 1123, 1124, or 1127, wherein the sequence comprises an ORF encoding an RNA-guided DNA binding agent, wherein the first three nucleotides of SEQ ID NOs: 1101, 1123, 1124, or 1127 are omitted. In some embodiments, the mRNA comprises a sequence with at least 90% identity to any one of SEQ ID NOs: 1101, 1123, 1124, or 1127, wherein the sequence comprises an ORF encoding an RNA-guided DNA binding agent, wherein the first three nucleotides of SEQ ID NOs: 1101, 1123, 1124, or 1127 are omitted and/or the ORF coding sequence contained within SEQ ID NO: 1101, 1123, 1124, or 1127 is substituted with the coding sequence of any one of SEQ ID NOs: 1102-1122, 1125, 1126, or 1129-1146. In some embodiments, any of the foregoing levels of identity is at least 95%, at least 98%, at least 99%, or 100%.

### Methods of Gene Modulation

In some embodiments, any one or more of the gRNAs (e.g., sgRNAs, dgRNAs, or crRNAs), compositions, or pharmaceutical formulations described herein is for use in preparing a medicament for treating or preventing a disease or disorder in a subject.

In some embodiments, the invention comprises a method of treating or preventing a disease or disorder in subject comprising administering any one or more of the gRNAs (e.g., sgRNAs, dgRNAs, or crRNAs), compositions, or pharmaceutical formulations described herein.

In some embodiments, the invention comprises a method or use of modifying a target DNA comprising, administering or delivering any one or more of the gRNAs (e.g., sgRNAs, dgRNAs, or crRNAs), compositions, or pharmaceutical formulations described herein.

In some embodiments, the invention comprises a method or use for modulation of a target gene comprising, administering or delivering any one or more of the gRNAs (e.g., sgRNAs, dgRNAs, or crRNAs), compositions, or pharmaceutical formulations described herein. In some embodiments, the modulation is editing of the target gene. In some embodiments, the modulation is a change in expression of the protein encoded by the target gene.

In some embodiments, the method or use results in gene editing. In some embodiments, the method or use results in a double-stranded break within the target gene. In some embodiments, the method or use results in formation of indel mutations during non-homologous end joining of the DSB. In some embodiments, the method or use results in an insertion or deletion of nucleotides in a target gene. In some embodiments, the insertion or deletion of nucleotides in a target gene leads to a frameshift mutation or premature stop codon that results in a non-functional protein. In some embodiments, the insertion or deletion of nucleotides in a target gene leads to a knockdown or elimination of target gene expression. In some embodiments, the method or use comprises homology directed repair of a DSB. In some embodiments, the method or use further comprises delivering to the cell a template, wherein at least a part of the template incorporates into a target DNA at or near a double strand break site induced by the nuclease.

In some embodiments, the method or use results in gene modulation. In some embodiments, the gene modulation is an increase or decrease in gene expression, a change in methylation state of DNA, or modification of a histone subunit. In some embodiments, the method or use results in increased or decreased expression of the protein encoded by the target gene.

The efficacy of gRNAs (e.g., sgRNAs, dgRNAs, or crRNAs) can be tested in vitro and in vivo. In some embodiments, the invention comprises one or more of the gRNAs (e.g., sgRNAs, dgRNAs, or crRNAs), compositions, or pharmaceutical formulations described herein, wherein the gRNA results in gene modulation when provided to a cell together with Cas9 or mRNA encoding Cas9. In some embodiments, the efficacy of gRNA can be measured in vitro or in vivo.

In some embodiments, the activity of a Cas RNP comprising a gRNA is compared to the activity of a Cas RNP comprising an unmodified sgRNA or a reference sgRNA lacking modifications present in the sgRNA, such as one or more shortened regions and/or YA site substitutions.

In some embodiments, the efficiency of a gRNA in increasing or decreasing target protein expression is determined by measuring the amount of target protein.

In some embodiments, the efficiency of editing with specific gRNAs is determined by the editing present at the target location in the genome following delivery of Cas9 and the gRNA. In some embodiments, the efficiency of editing with specific gRNAs is measured by next-generation sequencing. In some embodiments, the editing percentage of the target region of interest is determined. In some embodiments, the total number of sequence reads with insertions or deletions of nucleotides into the target region of interest over the total number of sequence reads is measured following delivery of a gRNA and Cas9.

In some embodiments, the efficiency of editing with specific gRNAs is measured by the presence of insertions or deletions of nucleotides introduced by successful gene editing. In some embodiments, activity of a Cas9 and gRNAs is tested in biochemical assays. In some embodiments, activity of a Cas9 and gRNAs is tested in a cell-free cleavage assay. In some embodiments, activity of a Cas9 and gRNAs is tested in Neuro2A cells.

In some embodiments, the activity of modified gRNAs is measured after in vivo dosing of LNPs comprising modified gRNAs and Cas protein or mRNA encoding Cas protein.

In some embodiments, in vivo efficacy of a gRNA or composition provided herein is determined by editing efficacy measured in DNA extracted from tissue (e.g., liver tissue) after administration of gRNA and Cas9.

In some embodiments, activation of the subject's immune response is measured by serum concentrations of cytokine(s) following in vivo dosing of sgRNA together with Cas9 mRNA or protein (e.g., formulated in a LNP). In some embodiments, the cytokine is interferon-alpha (IFN-alpha), interleukin 6 (IL-6), monocyte chemotactic protein 1 (MCP-1), and/or tumor necrosis factor alpha (TNF-alpha).

In some embodiments, administration of Cas RNP or Cas9 mRNA together with the modified gRNA (e.g., sgRNA, or dgRNA) produces lower serum concentration(s) of immune cytokines compared to administration of unmodified sgRNA. In some embodiments, the invention comprises a method of reducing a subject's serum concentration of immune cytokines comprising, administering any one of the gRNAs disclosed herein, wherein the gRNA produces a lower concentration of immune cytokines in a subject's serum as compared to a control gRNA that is not similarly modified.

LNP Delivery of gRNA

Lipid nanoparticles (LNPs) are a well-known means for delivery of nucleotide and protein cargo, and may be used for delivery of the gRNAs (e.g., sgRNAs, dgRNAs, or crRNAs), compositions, or pharmaceutical formulations disclosed herein. In some embodiments, the LNPs deliver nucleic acid, protein, or nucleic acid together with protein.

In some embodiments, the invention comprises a method for delivering any one of the gRNAs (e.g., sgRNAs, dgRNAs, or crRNAs) disclosed herein to a subject, wherein the gRNA is associated with an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). In some embodiments, the LNPs comprise molar ratios of a cationic lipid amine to RNA phosphate (N:P) of about 4.5.

In some embodiments, LNPs associated with the gRNAs disclosed herein are for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—In Vitro Editing in Primary Mouse Hepatocytes (PMH)

Short sgRNAs targeting the mouse and cyno TTR gene were designed as shown in Table 1A and lipofected, as provided below, into primary mouse hepatocytes (PMH). PMH (Gibco, Lot #793) were thawed and resuspended in hepatocyte thawing medium with supplements (Gibco, Cat. CM7500) followed by centrifugation. The supernatant was discarded and the pelleted cells resuspended in hepatocyte plating medium plus supplement pack (Invitrogen, Cat. A1217601 and CM3000). Cells were counted and plated on Bio-coat collagen I coated 96-well plates (ThermoFisher, Cat. 877272) at a density of 15,000 cells/well for PMH. Plated cells were allowed to settle and adhere for 5 hours in a tissue culture incubator at 37° C. and 5% CO2 atmosphere. After incubation cells were checked for monolayer formation and were washed once with hepatocyte culture medium (Takara, Cat. Y20020 and/or Invitrogen, Cat. A1217601 and CM4000). Lipofection of Cas9 mRNA and gRNAs used pre-mixed lipid formulations. The lipofection reagent contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-(((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. This mixture was reconstituted in 100% ethanol then mixed with RNA cargos (e.g., Cas9 mRNA and gRNA) at a lipid amine to RNA phosphate (N:P) molar ratio of about 6.0. Guide RNA was chemically synthesized by commercial vendors or using standard in vitro synthesis techniques with modified nucleotides. A Cas9 ORF of Table 1B was produced by in vitro transcription (IVT) as described in WO2019/067910, see e.g. ¶354, using a 2 hour IVT reaction time and purifying the mRNA by LiCl precipitation followed by tangential flow filtration.

Lipofections were performed with 6% cynomolgus monkey serum and a ratio of gRNA to mRNA of 1:1 by weight.

Genomic DNA Isolation

PMH cells were harvested post-transfection at 72 hours. The gDNA was extracted from each well of a 96-well plate using 50 μL/well QuickExtract DNA Extraction solution (Epicentre, Cat. QE09050) according to manufacturer's protocol.

Next-Generation Sequencing (“NGS”) and Analysis for Editing Efficiency

To quantitatively determine the efficiency of editing at the target location in the genome, sequencing was utilized to identify the presence of insertions and deletions introduced by gene editing. PCR primers were designed around the target site within the gene of interest (e.g. TTR), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

Dose response of editing efficiency to guide concentration was performed in duplicate samples. Table 6 show mean and standard deviation (SD) editing at each guide concentration and a calculated EC₅₀ value.

TABLE 6
Editing in primary mouse hepatocytes

| | | | Guide | | |
|---|---|---|---|---|---|
| | EC₅₀ | | concentration | Mean | |
| Guide | (nM) | SEM | (nM) | % Edit | SD |
| G013772 | 38.0 | n/d | 150 | 3% | 0% |
| | | | 75 | 5% | 2% |
| | | | 38 | 2% | 1% |
| | | | 19 | 1% | 0% |
| | | | 9 | 0% | 0% |
| | | | 5 | 0% | 0% |
| | | | 2 | 0% | 0% |
| G013773 | 12.7 | 1.4 | 150 | 96% | 2% |
| | | | 75 | 96% | 4% |
| | | | 38 | 90% | 1% |
| | | | 19 | 72% | 5% |
| | | | 9 | 42% | 7% |
| | | | 5 | 28% | 7% |
| | | | 2 | 20% | 10% |
| G013774 | n/d | n/d | 150 | 2% | 0% |
| | | | 75 | 3% | 0% |
| | | | 38 | 1% | 0% |
| | | | 19 | 1% | 0% |
| | | | 9 | 0% | 0% |
| | | | 5 | 0% | 0% |
| | | | 2 | 0% | 0% |
| G013775 | n/d | n/d | 150 | 0% | 0% |
| | | | 75 | 0% | 0% |
| | | | 38 | 0% | 0% |
| | | | 19 | 0% | 0% |
| | | | 9 | 0% | 0% |
| | | | 5 | 0% | 0% |
| | | | 2 | 0% | 0% |
| G013776 | 10.1 | 1.9 | 150 | 96% | 0% |
| | | | 75 | 94% | 0% |
| | | | 38 | 89% | 6% |
| | | | 19 | 71% | 11% |
| | | | 9 | 52% | 16% |
| | | | 5 | 25% | 9% |
| | | | 2 | 15% | 11% |
| G010039 | 13.4 | 2.0 | 150 | 83% | 0% |
| | | | 75 | 81% | 3% |
| | | | 38 | 72% | 0% |
| | | | 19 | 53% | 22% |
| | | | 9 | 32% | 10% |
| | | | 5 | 12% | 6% |
| | | | 2 | 6% | 1% |
| G012402 | 7.9 | 0.8 | 150 | 86% | 0% |
| | | | 75 | 86% | 1% |
| | | | 38 | 81% | 7% |
| | | | 19 | 73% | 6% |
| | | | 9 | 60% | 6% |
| | | | 5 | 33% | 6% |
| | | | 2 | 23% | 4% |
| G012401 | 8.5 | 1.1 | 150 | 97% | 0% |
| | | | 75 | 94% | 1% |
| | | | 38 | 89% | 2% |
| | | | 19 | 82% | 2% |
| | | | 9 | 61% | 14% |
| | | | 5 | 33% | 12% |
| | | | 2 | 23% | 7% |

### Example 2—In Vitro Editing of Deletion Series in Primary Cynomologus Hepatocytes (PCH)

Additional sgRNAs targeting mouse and cynomolgus monkey TTR genes were designed as shown in Table 1A and lipofected into primary cynomolgus monkey hepatocytes (PCH). Cells were prepared, treated and analyzed as described above unless otherwise noted. Specifically, PCH cells from In Vitro ADMET Laboratories, Inc. Lot #10281011 were used and plated at a density of 50,000 cells/well. Duplicate samples were included in the assay except for G015651 which was assayed singly. Mean editing results with standard deviation are shown in Table 7 and FIG. 2.

TABLE 7
In vitro editing in PCH

| Guide | Mean % Edit | SD |
|---|---|---|
| G015631 | 38.3% | 14.9% |
| G015632 | 41.4% | 14.5% |
| G015633 | 41.5% | 2.8% |
| G015634 | 5.4% | 3.6% |
| G015635 | 57.0% | 7.8% |
| G015636 | 57.3% | 8.8% |
| G015637 | 67.1% | 8.7% |
| G015638 | 60.5% | 1.3% |
| G015639 | 65.0% | 4.8% |
| G015640 | 62.5% | 2.0% |
| G015641 | 64.5% | 2.1% |
| G015642 | 69.1% | 2.5% |
| G015643 | 49.0% | 5.0% |
| G015644 | 32.6% | 8.8% |
| G015645 | 3.5% | 0.4% |
| G015646 | 38.4% | 17.1% |
| G015647 | 51.4% | 7.4% |
| G015648 | 55.3% | 19.6% |
| G015649 | 52.5% | 2.8% |
| G015650 | 45.3% | 12.3% |
| G015651 | 44.9% | n/a |
| G015652 | 58.3% | 16.3% |
| G015653 | 53.1% | 15.3% |
| G015654 | 43.2% | 16.8% |
| G015655 | 47.0% | 6.3% |
| G015656 | 31.1% | 11.2% |
| G015656 | 25.6% | 8.3% |
| G015657 | 12.0% | 6.7% |
| G015658 | 13.2% | 10.4% |
| G015659 | 6.5% | 6.4% |
| G015660 | 10.3% | 6.4% |
| G015661 | 15.4% | 13.4% |
| G015662 | 8.0% | 4.2% |
| G015663 | 17.5% | 9.8% |
| G015664 | 22.8% | 17.7% |
| G015665 | 36.4% | 19.5% |
| G015666 | 15.9% | 14.6% |
| G015667 | 1.1% | 0.4% |
| G015668 | 1.1% | 0.1% |
| G015669 | 1.5% | 0.4% |
| G015670 | 0.9% | 0.1% |
| G015671 | 14.2% | 4.1% |
| G015672 | 1.7% | 0.1% |
| G015673 | 18.7% | 11.2% |
| G015674 | 4.9% | 2.4% |
| G015675 | 2.2% | 0.1% |
| G015676 | 3.3% | 3.4% |
| G015677 | 1.4% | 0.8% |
| G015678 | 48.8% | 6.5% |
| G015679 | 5.8% | 1.3% |
| G015680 | 1.9% | 0.4% |
| G015681 | 1.5% | 0.1% |
| G015682 | 1.2% | 0.1% |
| G015683 | 1.0% | 0.1% |
| G015684 | 1.1% | 0.4% |
| G015685 | 37.5% | 8.9% |
| G015686 | 42.7% | 10.3% |
| G015687 | 38.8% | 21.6% |
| G015688 | 40.5% | 12.0% |
| G015689 | 25.7% | 17.5% |
| G015690 | 8.2% | 6.8% |
| G015691 | 16.9% | 12.9% |
| G015692 | 50.7% | 17.9% |
| G015693 | 41.9% | 8.0% |
| G015694 | 32.6% | 17.2% |
| G015695 | 43.6% | 7.6% |
| G015696 | 38.7% | 12.0% |
| G015697 | 23.3% | 15.8% |
| G015698 | 44.0% | 13.7% |
| G015699 | 38.4% | 19.4% |
| G015700 | 0.8% | 0.1% |
| G015701 | 40.0% | 21.2% |
| G015702 | 0.8% | 0.1% |
| G000502 | 47.3% | 10.1% |
| G012401 | 51.4% | 13.1% |

### Example 3—In Vitro Editing of Additional Guides in PCH Using Lipofection

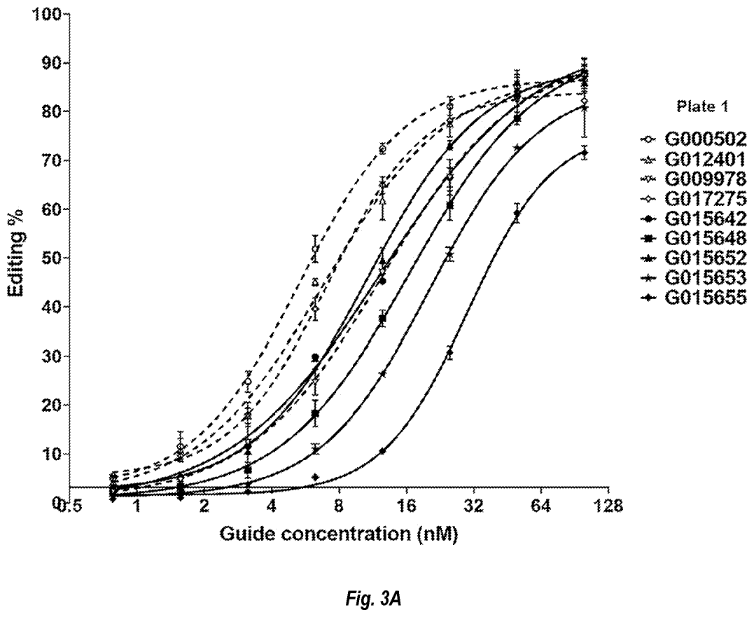

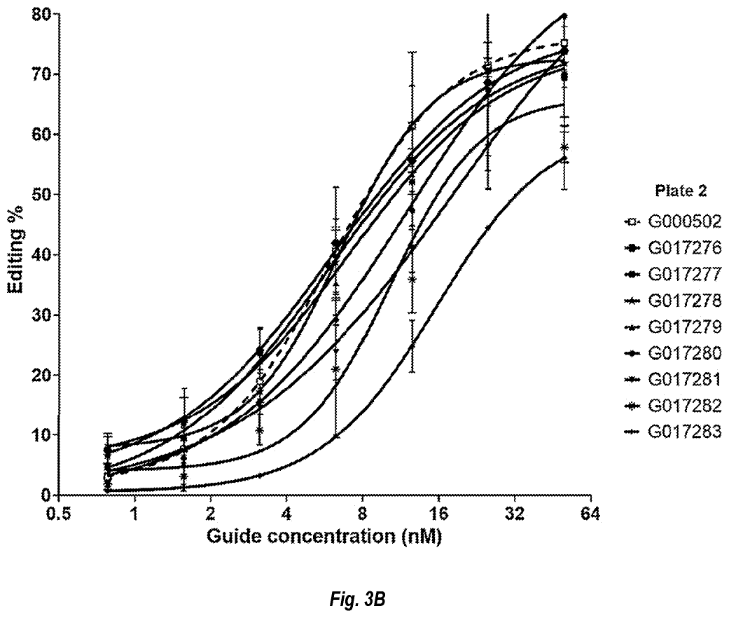

sgRNAs targeting cynomolgus monkey TTR genes were designed as shown in Table 1A that combined deletions or chemical modifications in different regions of the sgRNA constant region. Guides and Cas9 mRNA were lipofected into primary cynomolgus monkeyhepatocytes (PCH). Cells were prepared, treated and analyzed as described above unless otherwise noted. Guides were assayed in a 7 point 2-fold dose response curve starting at 50 nM guide concentration as shown in Tables 8-9. Duplicate samples were included in the assay except for G000502 which was assayed in quadruplicate. EC₅₀ values and mean editing results are shown in Tables 8-9. Dose response curves are plotted in FIG. 3A and FIG. 3B.

TABLE 8
Editing efficiency in PCH using lipofection

| | | | Guide | | |
|---|---|---|---|---|---|
| | | EC₅₀ | concentration | Mean % | |
| Guide | EC₅₀ | SEM | (nM) | Edit | SD |
| G000502 | 5.3 | 0.2 | 100 | 87.6 | 2.1 |
| | | | 50 | 85.1 | 2.4 |
| | | | 25 | 81.1 | 1.9 |
| | | | 12.5 | 72.4 | 1.0 |
| | | | 6.25 | 51.8 | 2.7 |
| | | | 3.125 | 24.8 | 2.1 |
| | | | 1.563 | 11.5 | 3.1 |
| | | | 0.781 | 5 | 0.4 |
| G012401 | 6.8 | 0.4 | 100 | 86.9 | 2.7 |
| | | | 50 | 84.1 | 2.9 |
| | | | 25 | 77.4 | 0.2 |
| | | | 12.5 | 61.7 | 3.8 |
| | | | 6.25 | 45.1 | 0.7 |
| | | | 3.125 | 17.8 | 1.5 |
| | | | 1.563 | 10.8 | 2.4 |
| | | | 0.781 | 4.2 | 1.4 |
| G009978 | 12.5 | 0.6 | 100 | 87.2 | 3.4 |
| | | | 50 | 82.3 | 3.8 |
| | | | 25 | 66.5 | 3.6 |
| | | | 12.5 | 47.0 | 0.4 |
| | | | 6.25 | 24.7 | 2.6 |
| | | | 3.125 | 12.4 | 0.4 |
| | | | 1.563 | 4.6 | 0 |
| | | | 0.781 | 2.3 | 0.2 |
| G017275 | 7.0 | 0.3 | 100 | 82.2 | 7.3 |
| | | | 50 | 84.2 | n/a |
| | | | 25 | 78.1 | 3.2 |
| | | | 12.5 | 64.8 | 1.7 |
| | | | 6.25 | 39.5 | 2.3 |
| | | | 3.125 | 18.0 | 2.4 |
| | | | 1.563 | 9.6 | 0.6 |
| | | | 0.781 | 5.6 | 0.4 |
| G015642 | 12.5 | 0.8 | 100 | 87.6 | 0.9 |
| | | | 50 | 83.2 | 0.4 |
| | | | 25 | 66.5 | 1.9 |
| | | | 12.5 | 45.3 | 0.2 |
| | | | 6.25 | 29.9 | 0.1 |
| | | | 3.125 | 11.6 | 4.0 |
| | | | 1.563 | 5.2 | 0.4 |
| | | | 0.781 | 2.5 | 0.7 |
| G015648 | 16.5 | 0.7 | 100 | 87.8 | 3.1 |
| | | | 50 | 78.6 | 1.2 |
| | | | 25 | 60.8 | 2.9 |
| | | | 12.5 | 37.6 | 1.6 |
| | | | 6.25 | 18.3 | 2.6 |
| | | | 3.125 | 6.6 | 1.6 |
| | | | 1.563 | 3.2 | 1.0 |
| | | | 0.781 | 2.2 | 0.7 |
| G015652 | 10.7 | 0.5 | 100 | 85.9 | 3.3 |
| | | | 50 | 86 | 2.5 |
| | | | 25 | 73.1 | 0.9 |
| | | | 12.5 | 49.5 | 2.6 |
| | | | 6.25 | 29.5 | 0.2 |
| | | | 3.125 | 10.5 | 0.4 |
| | | | 1.563 | 5.6 | 0.5 |
| | | | 0.781 | 3.4 | 1.5 |
| G015653 | 20.2 | 0.4 | 100 | 80.7 | 0.7 |
| | | | 50 | 72.6 | 0.1 |
| | | | 25 | 50.7 | 1.4 |
| | | | 12.5 | 26.3 | 0.7 |
| | | | 6.25 | 11.0 | 1.0 |
| | | | 3.125 | 3.7 | 0.1 |
| | | | 1.563 | 2.1 | 0.6 |
| | | | 0.781 | 1.2 | 0 |
| G015655 | 30.0 | 0.8 | 100 | 71.6 | 1.4 |
| | | | 50 | 59.2 | 1.9 |
| | | | 25 | 30.7 | 1.3 |
| | | | 12.5 | 10.5 | 0.3 |
| | | | 6.25 | 5.2 | 0.2 |
| | | | 3.125 | 2.3 | 0.1 |
| | | | 1.563 | 1.0 | 0.3 |
| | | | 0.781 | 0.7 | 0.2 |

TABLE 9
Editing efficiency in PCH using lipofection

| | | | Guide | | |
|---|---|---|---|---|---|
| | | EC₅₀ | concentration | Mean % | |
| Guide | EC₅₀ | SEM | (nM) | Edit | SD |
| G000502 | 5.9 | 0.5 | 50 | 75.2 | 4.4 |
| | | | 25 | 71.5 | 3.7 |
| | | | 12.5 | 61.4 | 6.6 |
| | | | 6.25 | 40.6 | 10.6 |
| | | | 3.125 | 19 | 2.9 |
| | | | 1.563 | 7.6 | 0.3 |
| | | | 0.781 | 3 | 0.6 |
| G017276 | 6.2 | 1.0 | 50 | 73.9 | 12.4 |
| | | | 25 | 68.6 | 2.3 |
| | | | 12.5 | 55.7 | 2.0 |
| | | | 6.25 | 42.0 | 2.2 |
| | | | 3.125 | 24.1 | 0.7 |
| | | | 1.563 | 12.3 | 0.7 |
| | | | 0.781 | 7.5 | 2.3 |
| G017277 | 5.9 | 1.2 | 50 | 69.7 | 0.7 |
| | | | 25 | 70.5 | 14.0 |
| | | | 12.5 | 52.2 | 0.1 |
| | | | 6.25 | 39.9 | 6.1 |
| | | | 3.125 | 23.7 | 4.3 |
| | | | 1.563 | 9.5 | 1.7 |
| | | | 0.781 | 4.6 | 1.8 |
| G017278 | 6.5 | 0.8 | 50 | 71.8 | 8.9 |
| | | | 25 | 70.8 | 4.5 |
| | | | 12.5 | 61.9 | 11.8 |
| | | | 6.25 | 38.8 | 5.9 |
| | | | 3.125 | 17.3 | 1.8 |
| | | | 1.563 | 11.9 | 4.4 |
| | | | 0.781 | 6.6 | 3.7 |
| G017279 | 7.0 | 1.5 | 50 | 69.8 | 1.9 |
| | | | 25 | 67.0 | 16.1 |
| | | | 12.5 | 53.4 | 0.2 |
| | | | 6.25 | 35.4 | 1.9 |
| | | | 3.125 | 24.0 | 3.7 |
| | | | 1.563 | 11.9 | 5.9 |
| | | | 0.781 | 7.9 | 0.5 |
| G017280 | 11.8 | 2.9 | 50 | 79.7 | 5.0 |
| | | | 25 | 67.3 | 13.3 |
| | | | 12.5 | 47.4 | 3.2 |
| | | | 6.25 | 29.2 | 0.8 |
| | | | 3.125 | 15.7 | 2.2 |
| | | | 1.563 | 6.1 | 1.1 |
| | | | 0.781 | 2.3 | 0.9 |
| G017281 | 18.1 | 8.6 | 50 | 74.4 | 3.6 |
| | | | 25 | 57.9 | 6.8 |
| | | | 12.5 | 41.0 | 3.9 |
| | | | 6.25 | 23.9 | 4.6 |
| | | | 3.125 | 14.7 | 6.3 |
| | | | 1.563 | 5.1 | 1.6 |
| | | | 0.781 | 3.3 | 1.8 |
| G017282 | 10.3 | 1.9 | 50 | 57.9 | 2.5 |
| | | | 25 | 71.1 | 1.6 |
| | | | 12.5 | 36.0 | 5.6 |
| | | | 6.25 | 21.0 | 11.5 |
| | | | 3.125 | 10.8 | 0.5 |
| | | | 1.563 | 3.1 | 1.7 |
| | | | 0.781 | 1.9 | 0.9 |
| G017283 | 15.6 | 1.4 | 50 | 56.1 | 5.2 |
| | | | 25 | 44.5 | 0.1 |
| | | | 12.5 | 24.9 | 4.3 |
| | | | 6.25 | 9.9 | 0.3 |
| | | | 3.125 | 3.2 | 0.0 |
| | | | 1.563 | 1.3 | 0.5 |
| | | | 0.781 | 0.8 | 0.2 |

### Example 4—In Vitro Editing of Additional Guides in PMH and PCH Using Lipid Nanoparticles

sgRNAs targeting mouse and cynomolgus monkey TTR genes were designed as shown in Table 1A that combined deletions or chemical modifications in different regions of the sgRNA constant region. These guides were tested for efficacy in primary mouse and primary cynomolgus monkey hepatocytes using lipid nanoparticles to deliver Cas9 mRNA and sgRNA.

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used in Examples 5-6 contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The LNPs were formulated with a lipid amine to RNA phosphate (N:P) molar ratio of about 6, and a ratio of gRNA to mRNA of 1:2 by weight.

The LNPs were prepared using a cross-flow technique utilizing impinging jet mixing of the lipid in ethanol with two volumes of RNA solutions and one volume of water. The lipid mixture in ethanol was mixed through a mixing cross with the two volumes of RNA solution. A fourth stream of water was mixed with the outlet stream of the cross through an inline tee (See WO2016010840 FIG. 2). The resulting LNPs were held for 1 hour at room temperature, and further diluted with water (approximately 1:1 v/v). Diluted LNPs were buffer exchanged using PD-10 desalting columns (GE) into 50 mM Tris, 45 mM NaCl, 5% (w/v) sucrose, pH 7.5 (TSS). The resulting mixture was then filtered using a 0.2 μm sterile filter and optionally further diluted. The final LNP was stored at 4° C. or −80° C. until further use.

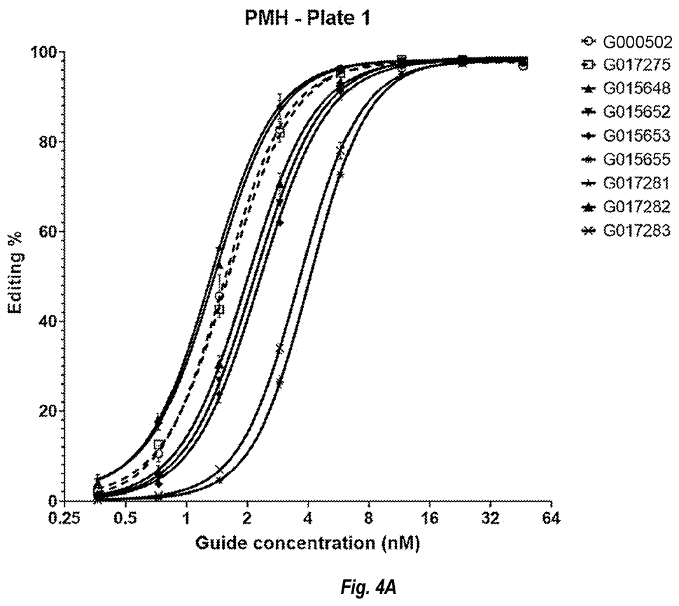

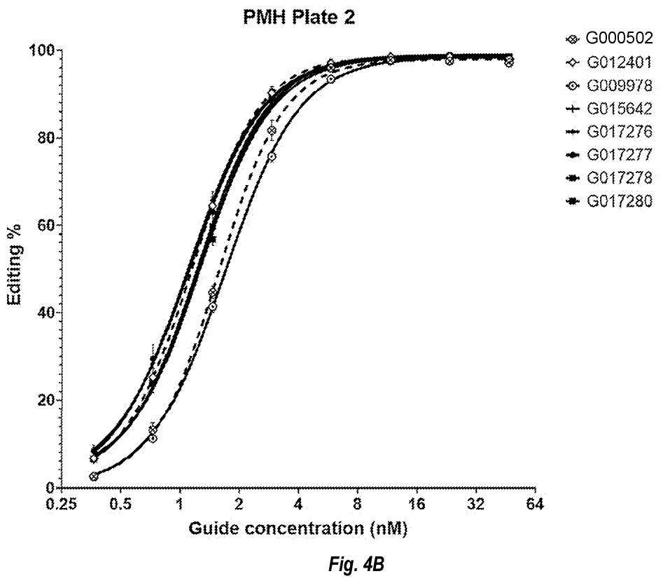

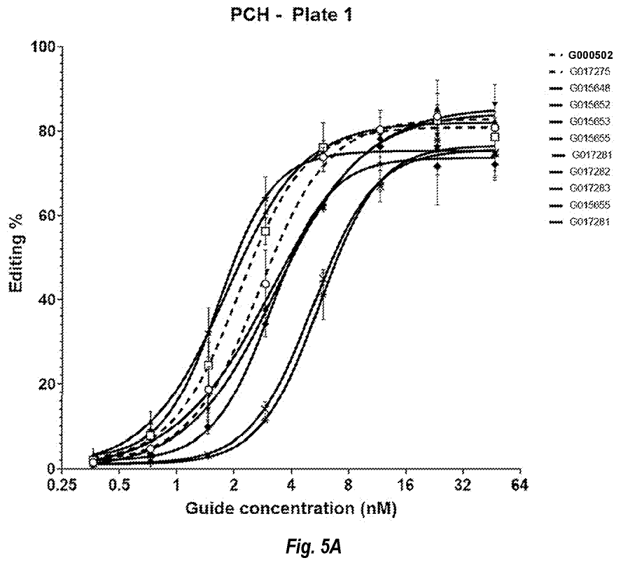

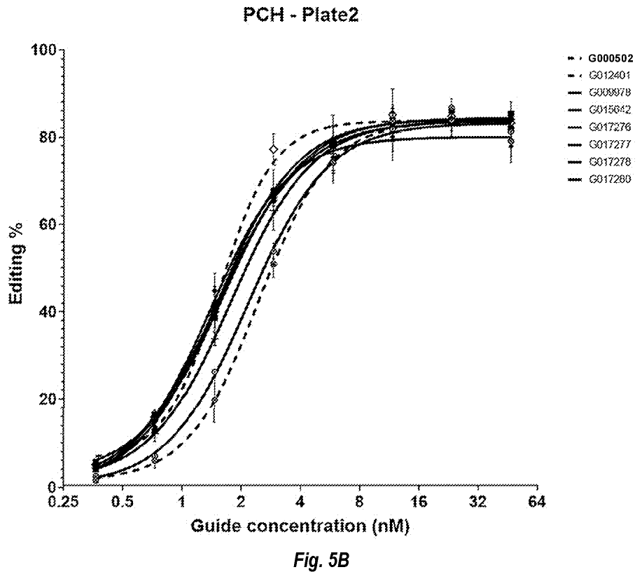

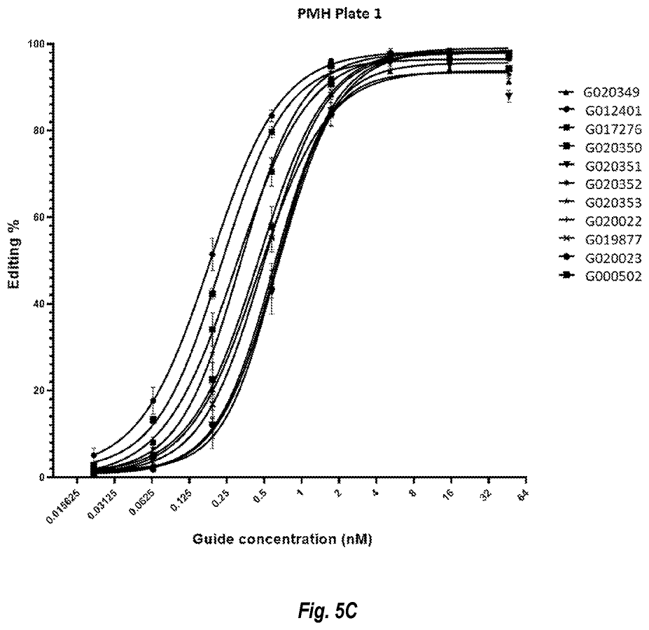

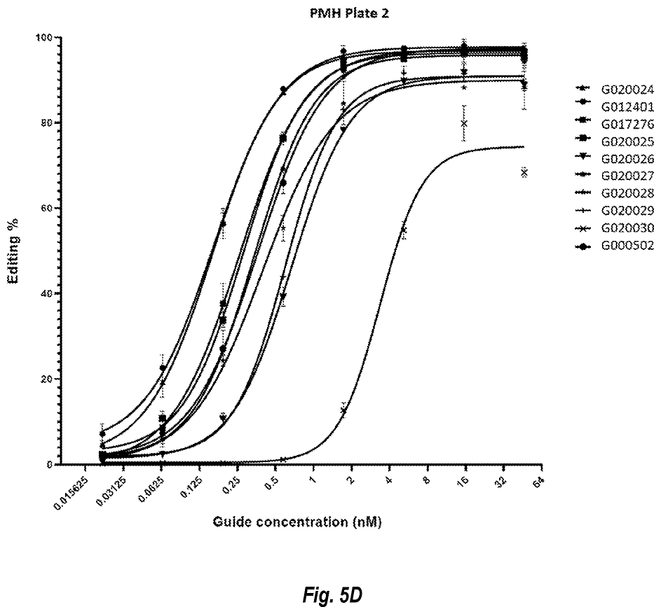

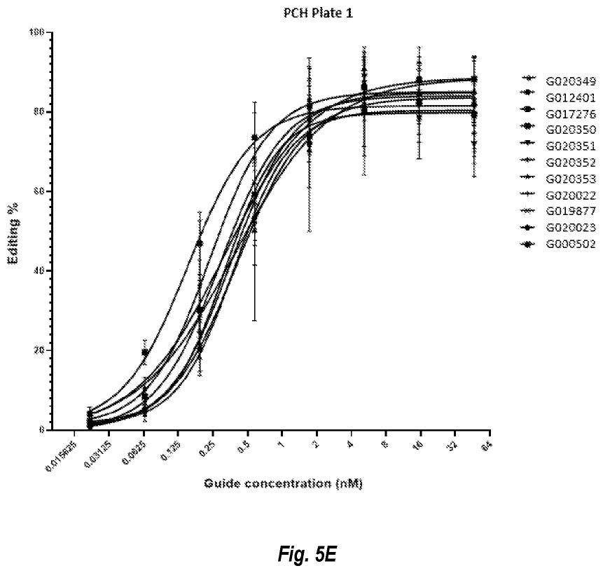

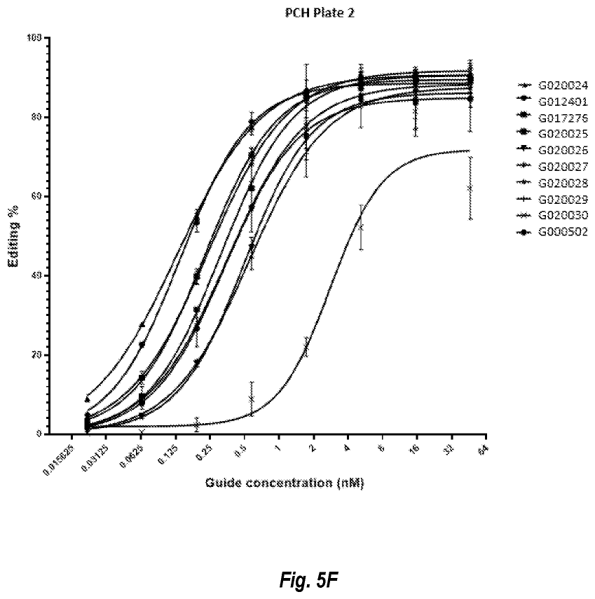

Lipid nanoparticle (LNP) formulations of sgRNAs were tested on PMH and PCH in a dose response assays. PMH and PCH were prepared as in Example 1 and Example 2, respectively. In addition, cells were incubated at 37° C., 5% CO₂ for 24 hours prior to treatment with LNPs. LNPs were incubated in media containing 6% fetal bovine serum (FBS) at 37° C. for 10 minutes. Post-incubation the LNPs were added to the mouse or cynomolgus hepatocytes in an 8 point 3-fold dose response curve starting at 300 ng Cas9 mRNA. The cells were lysed 96 hours post-treatment for NGS analysis as described in Example 1. Duplicate samples were included in the assay. EC₅₀ values and mean editing results for PMH are shown in Table 10 and plotted as dose response curves in FIGS. 4A and 4B. EC₅₀ values and mean editing results for PCH are shown in Table 11A and plotted as dose response curves in FIGS. 5A and 5B.

The precise formulation of guides in LNPs achieve lower EC50s and higher percent editing as compared to the above experiments that used transfection-based delivery methods termed lipofection.

TABLE 10
Table 10 - In vitro editing in PMH using lipid nanoparticles

| | | | Guide | | |
|---|---|---|---|---|---|
| | | EC₅₀ | concentration | Mean % | |
| Guide | EC₅₀ | SEM | (nM) | Edit | SD |
| G000502 | 1.54 | 0.03 | 46.56 | 97.0 | 0.1 |
| | | | 23.28 | 97.7 | 1.0 |
| | | | 11.64 | 97.8 | 0.1 |
| | | | 5.82 | 96.2 | 0.1 |
| | | | 2.91 | 82.7 | 1.8 |
| | | | 1.46 | 45.6 | 4.8 |
| | | | 0.73 | 10.5 | 1.8 |
| | | | 0.36 | 2.1 | 0.2 |
| G017275 | 1.61 | 0.02 | 46.56 | 98.1 | 0.2 |
| | | | 23.28 | 98.1 | 0.1 |
| | | | 11.64 | 98.3 | 0.6 |
| | | | 5.82 | 95.3 | 0.3 |
| | | | 2.91 | 82.1 | 2.1 |
| | | | 1.46 | 42.7 | 0.7 |
| | | | 0.73 | 12.6 | 0.7 |
| | | | 0.36 | 2.1 | 0.6 |
| G015648 | 1.38 | 0.02 | 46.56 | 97.8 | 0.1 |
| | | | 23.28 | 98.1 | 0.1 |
| | | | 11.64 | 97.9 | 0.3 |
| | | | 5.82 | 96.4 | 0.4 |
| | | | 2.91 | 88.1 | 1.2 |
| | | | 1.46 | 52.7 | 0.5 |
| | | | 0.73 | 18.2 | 0.4 |
| | | | 0.36 | 4.1 | 0.3 |
| G015652 | 2.17 | 0.03 | 46.56 | 97.9 | 0.3 |
| | | | 23.28 | 98.4 | 0.0 |
| | | | 11.64 | 98.0 | 0.4 |
| | | | 5.82 | 92.4 | 1.5 |
| | | | 2.91 | 66.4 | 0.6 |
| | | | 1.46 | 26.8 | 0.1 |
| | | | 0.73 | 5.1 | 1.2 |
| | | | 0.36 | 1.1 | 0.1 |
| G015653 | 2.31 | 0.04 | 46.56 | 97.7 | 0.0 |
| | | | 23.28 | 98.0 | 0.6 |
| | | | 11.64 | 96.9 | 1.2 |
| | | | 5.82 | 91.3 | 2.0 |
| | | | 2.91 | 62.1 | 0.4 |
| | | | 1.46 | 23.8 | 1.9 |
| | | | 0.73 | 3.8 | 0.1 |
| | | | 0.36 | 0.8 | 0.1 |
| G015655 | 4.09 | 0.03 | 46.56 | 97.7 | 1.0 |
| | | | 23.28 | 97.9 | 0.6 |
| | | | 11.64 | 95.2 | 0.1 |
| | | | 5.82 | 72.8 | 0.5 |
| | | | 2.91 | 26.6 | 1.3 |
| | | | 1.46 | 4.6 | 0.4 |
| | | | 0.73 | 0.9 | 0.1 |
| | | | 0.36 | 0.4 | 0.1 |
| G017281 | 1.32 | 0.02 | 46.56 | 98.1 | 0.4 |
| | | | 23.28 | 98.4 | 0.2 |
| | | | 11.64 | 98.4 | 0.9 |
| | | | 5.82 | 96.4 | 0.2 |
| | | | 2.91 | 87.9 | 2.8 |
| | | | 1.46 | 56.3 | 0.2 |
| | | | 0.73 | 17.6 | 1.8 |
| | | | 0.36 | 4.7 | 1.1 |
| G017282 | 2.00 | 0.03 | 46.56 | 97.9 | 0.4 |
| | | | 23.28 | 98.2 | 0.2 |
| | | | 11.64 | 97.4 | 0.6 |
| | | | 5.82 | 93.3 | 0.0 |
| | | | 2.91 | 70.7 | 2.3 |
| | | | 1.46 | 30.5 | 1.8 |
| | | | 0.73 | 6.8 | 1.1 |
| | | | 0.36 | 1.4 | 0.2 |
| G017283 | 3.63 | 0.03 | 46.56 | 97.7 | 0.4 |
| | | | 23.28 | 97.6 | 0.4 |
| | | | 11.64 | 95.4 | 0.8 |
| | | | 5.82 | 78.1 | 1.8 |
| | | | 2.91 | 34.0 | 1.1 |
| | | | 1.46 | 7.1 | 0.8 |
| | | | 0.73 | 1.2 | 0.1 |
| | | | 0.36 | 0.4 | 0.2 |
| G000502 | 1.56 | 0.02 | 46.56 | 97.2 | 0.1 |
| | | | 23.28 | 97.5 | 0.7 |
| | | | 11.64 | 97.8 | 0.3 |
| | | | 5.82 | 96.0 | 0.0 |
| | | | 2.91 | 81.7 | 2.3 |
| | | | 1.46 | 44.7 | 1.4 |
| | | | 0.73 | 13.2 | 1.7 |
| | | | 0.36 | 2.6 | 0.2 |
| G012401 | 1.14 | 0.02 | 46.56 | 98.1 | 0.2 |
| | | | 23.28 | 98.7 | 0.2 |
| | | | 11.64 | 98.6 | 0.1 |
| | | | 5.82 | 97.1 | 0.4 |
| | | | 2.91 | 90.3 | 1.4 |
| | | | 1.46 | 64.5 | 1.9 |
| | | | 0.73 | 25.3 | 0.8 |
| | | | 0.36 | 6.7 | 0.0 |
| G009978 | 1.68 | 0.02 | 46.56 | 97.9 | 0.5 |
| | | | 23.28 | 98.6 | 0.1 |
| | | | 11.64 | 97.6 | 0.4 |
| | | | 5.82 | 93.5 | 0.4 |
| | | | 2.91 | 75.8 | 1.2 |
| | | | 1.46 | 41.4 | 1.3 |
| | | | 0.73 | 11.3 | 0.4 |
| | | | 0.36 | 2.5 | 0.1 |
| G015642 | 1.24 | 0.03 | 46.56 | 98.2 | 0.2 |
| | | | 23.28 | 98.3 | 0.4 |
| | | | 11.64 | 97.8 | 0.5 |
| | | | 5.82 | 96.0 | 0.3 |
| | | | 2.91 | 88.4 | 0.1 |
| | | | 1.46 | 57.4 | 0.5 |
| | | | 0.73 | 24.5 | 2.8 |
| | | | 0.36 | 6.2 | 0.8 |
| G017276 | 1.09 | 0.02 | 46.56 | 97.7 | 0.9 |
| | | | 23.28 | 98.7 | 0.4 |
| | | | 11.64 | 98.5 | 0.1 |
| | | | 5.82 | 96.8 | 1.2 |
| | | | 2.91 | 89.0 | 0.6 |
| | | | 1.46 | 65.8 | 1.9 |
| | | | 0.73 | 28.5 | 1.1 |
| | | | 0.36 | 8.2 | 1.0 |
| G017277 | 1.12 | 0.03 | 46.56 | 98.5 | 0.3 |
| | | | 23.28 | 98.4 | 0.0 |
| | | | 11.64 | 98.2 | 0.6 |
| | | | 5.82 | 96.9 | 0.1 |
| | | | 2.91 | 90.7 | 0.6 |
| | | | 1.46 | 63.1 | 0.6 |
| | | | 0.73 | 29.6 | 3.2 |
| | | | 0.36 | 8.4 | 1.3 |
| G017278 | 1.22 | 0.02 | 46.56 | 97.8 | 0.8 |
| | | | 23.28 | 98.7 | 0.2 |
| | | | 11.64 | 97.7 | 0.1 |
| | | | 5.82 | 96.8 | 0.1 |
| | | | 2.91 | 88.6 | 0.3 |
| | | | 1.46 | 59.8 | 2.8 |
| | | | 0.73 | 23.8 | 0.7 |
| | | | 0.36 | 6.6 | 0.7 |
| G017280 | 1.27 | 0.03 | 46.56 | 98.6 | 0.0 |
| | | | 23.28 | 98.6 | 0.1 |
| | | | 11.64 | 98.3 | 0.5 |
| | | | 5.82 | 96.4 | 0.6 |
| | | | 2.91 | 89.8 | 0.8 |
| | | | 1.46 | 56.8 | 1.3 |
| | | | 0.73 | 24.5 | 0.6 |
| | | | 0.36 | 6.6 | 0.8 |

TABLE 11A
In vitro editing in PCH using lipid nanoparticles

| | | | Guide | | |
|---|---|---|---|---|---|
| | EC₅₀ | EC₅₀ | concentration | Mean % | |
| Guide | (nM) | SEM | (nM) | Edit | SD |
| | | | 46.56 | 80.8 | 0.84853 |
| | | | 23.28 | 83.475 | 5.48008 |
| | | | 11.64 | 80.3 | 4.59619 |
| G000502 | 2.70 | 0.17 | 5.82 | 73.825 | 2.22739 |
| | | | 2.91 | 43.75 | 7.99031 |
| | | | 1.46 | 18.65 | 5.44472 |
| | | | 0.73 | 4.8 | 1.48492 |
| | | | 0.36 | 1.525 | 0.45962 |
| | | | 46.56 | 78.6 | 4.52548 |
| | | | 23.28 | 82.55 | 3.74767 |
| | | | 11.64 | n/d | n/d |
| G017275 | 2.09 | 0.11 | 5.82 | 76.1 | 1.69706 |
| | | | 2.91 | 56.3 | 3.25269 |
| | | | 1.46 | 24.45 | 5.44472 |
| | | | 0.73 | 7.8 | 1.13137 |
| | | | 0.36 | 2.1 | 0.70711 |
| | | | 46.56 | 80.85 | 10.2531 |
| | | | 23.28 | 83.85 | 8.27315 |
| G015648 | 1.80 | 0.15 | 11.64 | 79.7 | 0.42426 |
| | | | 5.82 | 75.45 | 2.19203 |
| | | | 2.91 | 60.95 | 1.62635 |
| | | | 1.46 | 32.2 | 3.25269 |
| | | | 0.73 | 11.05 | 2.47487 |
| | | | 0.36 | 3.4 | 1.41421 |
| G015652 | 3.30 | 0.19 | 46.56 | 86.3 | 1.69706 |
| | | | 23.28 | 79.05 | 4.17193 |
| | | | 11.64 | 77.6 | 6.78823 |
| | | | 5.82 | 62.6 | 0 |
| | | | 2.91 | 37.35 | 0.91924 |
| | | | 1.46 | 13.9 | 1.69706 |
| | | | 0.73 | 3.5 | 1.41421 |
| | | | 0.36 | 1.45 | 0.3535 |
| G015653 | 3.11 | 0.18 | 46.56 | 72.05 | 3.04056 |
| | | | 23.28 | 71.6 | 9.05097 |
| | | | 11.64 | 76.35 | 3.88909 |
| | | | 5.82 | 62.55 | 0.91924 |
| | | | 2.91 | 34.2 | 2.96985 |
| | | | 1.46 | 9.85 | 1.62635 |
| | | | 0.73 | 3.55 | 0.49498 |
| | | | 0.36 | 1.1 | 0.56569 |
| G015655 | 5.59 | 0.24 | 46.56 | 74.45 | 6.15183 |
| | | | 23.28 | 77.85 | 2.33345 |
| | | | 11.64 | 67.35 | 1.3435 |
| | | | 5.82 | 41.2 | 5.9397 |
| | | | 2.91 | 11.45 | 0.07071 |
| | | | 1.46 | 3.15 | 0.3535 |
| | | | 0.73 | 1.55 | 1.06066 |
| | | | 0.36 | 0.7 | 0.28284 |
| G017281 | 1.64 | 0.11 | 46.56 | 75.05 | 5.58614 |
| | | | 23.28 | 76.35 | 5.02046 |
| | | | 11.64 | 72.3 | 6.78823 |
| | | | 5.82 | 76.2 | 5.79828 |
| | | | 2.91 | 64 | 5.09117 |
| | | | 1.46 | 32.3 | 5.79828 |
| | | | 0.73 | 9.05 | 0.49498 |
| | | | 0.36 | 2.85 | 0.49498 |
| G017282 | 3.18 | 0.24 | 46.56 | 82.2 | n/a |
| | | | 23.28 | 85.2 | n/a |
| | | | 11.64 | 78.6 | 4.66691 |
| | | | 5.82 | 61.95 | 1.06066 |
| | | | 2.91 | 37.95 | 0.6364 |
| | | | 1.46 | 19.4 | 8.34386 |
| | | | 0.73 | 3.4 | 0.42426 |
| | | | 0.36 | 1.4 | 0.14142 |
| G017283 | 5.16 | 0.23 | 46.56 | 74.8 | 5.79828 |
| | | | 23.28 | 75.75 | 6.15183 |
| | | | 11.64 | 66.9 | 3.67696 |
| | | | 5.82 | 45 | 1.41421 |
| | | | 2.91 | 14.05 | 1.76777 |
| | | | 1.46 | 3.3 | 0.42426 |
| | | | 0.73 | 2 | 0.56569 |
| | | | 0.36 | 1.3 | 0.56569 |
| G000502 | 2.44 | 0.14 | 46.56 | 79.1 | 3.53553 |
| | | | 23.28 | 86.725 | 0.67175 |
| | | | 11.64 | 83.7 | 3.67696 |
| | | | 5.82 | 74.225 | 4.77297 |
| | | | 2.91 | 50.975 | 3.14663 |
| | | | 1.46 | 19.775 | 5.05581 |
| | | | 0.73 | 5.875 | 1.6617 |
| | | | 0.36 | 1.6 | 0.49498 |
| G012401 | 1.53 | 0.08 | 46.56 | 83.25 | 1.90919 |
| | | | 23.28 | 84.8 | 0.70711 |
| | | | 11.64 | 85.05 | 6.01041 |
| | | | 5.82 | 78.4 | 6.6468 |
| | | | 2.91 | 77.2 | 3.53553 |
| | | | 1.46 | 40 | 1.13137 |
| | | | 0.73 | 15.9 | 1.55564 |
| | | | 0.36 | 5.05 | 0.6364 |
| G009978 | 2.15 | 0.08 | 46.56 | 81.25 | 1.3435 |
| | | | 23.28 | 83.85 | 0.77782 |
| | | | 11.64 | 81.85 | 2.47487 |
| | | | 5.82 | 74.55 | 4.59619 |
| | | | 2.91 | 53.9 | 1.83848 |
| | | | 1.46 | 26.25 | 0.07071 |
| | | | 0.73 | 7 | 0.84853 |
| | | | 0.36 | 2.35 | 0.07071 |
| G015642 | 1.77 | 0.07 | 46.56 | 84.1 | 1.69706 |
| | | | 23.28 | 82.6 | 2.68701 |
| | | | 11.64 | 83.35 | 3.18198 |
| | | | 5.82 | 76.2 | 3.81838 |
| | | | 2.91 | 63.2 | 0.98995 |
| | | | 1.46 | 33.8 | 1.55564 |
| | | | 0.73 | 12.1 | 0.98995 |
| | | | 0.36 | 4.2 | 0 |
| G017276 | 1.37 | 0.11 | 46.56 | 77.9 | 3.67696 |
| | | | 23.28 | 83.1 | 1.69706 |
| | | | 11.64 | 80.1 | 5.37401 |
| | | | 5.82 | 75.4 | 3.67696 |
| | | | 2.91 | 65.6 | 6.92965 |
| | | | 1.46 | 44.8 | 0.98995 |
| | | | 0.73 | 13.7 | 1.13137 |
| | | | 0.36 | 4.45 | 0.91924 |
| G017277 | 1.50 | 0.09 | 46.56 | 84.1 | 3.9598 |
| | | | 23.28 | 83.2 | 2.96985 |
| | | | 11.64 | 81.8 | 5.09117 |
| | | | 5.82 | 78.6 | 2.40416 |
| | | | 2.91 | 65.45 | 1.20208 |
| | | | 1.46 | 40.8 | 2.68701 |
| | | | 0.73 | 15.1 | 1.69706 |
| | | | 0.36 | 4.45 | 0.07071 |
| G017278 | 1.50 | 0.08 | 46.56 | 85.35 | 0.21213 |
| | | | 23.28 | 83.65 | 1.06066 |
| | | | 11.64 | 83.15 | 0.21213 |
| | | | 5.82 | 78.05 | 2.33345 |
| | | | 2.91 | 67.7 | 0.70711 |
| | | | 1.46 | 41.35 | 7.42462 |
| | | | 0.73 | 12.65 | 2.33345 |
| | | | 0.36 | 3.95 | 0.91924 |
| G017280 | 1.60 | 0.07 | 46.56 | 81.85 | 0.07071 |
| | | | 23.28 | 86.15 | 2.6163 |
| | | | 11.64 | 83.75 | 1.20208 |
| | | | 5.82 | 78.7 | 3.39411 |
| | | | 2.91 | 66.65 | 0.6364 |
| | | | 1.46 | 38.65 | 2.33345 |
| | | | 0.73 | 16.45 | 1.20208 |
| | | | 0.36 | 5.6 | 0.14142 |

Lipid nanoparticle (LNP) formulations of sgRNAs were tested on PMH and PCH in dose response assays as described in Example 1 and Example 2 with some protocol modifications. Specifically, PMH (Gibco, Lot #839) and PCH (InVitro ADMET Laboratories, Lot #10361011) were prepared using hepatocyte medium with supplements (Invitrogen, William's E (Gibco, Cat. A1217601), plating supplements (Cocktail A and Dexamethasone) (Gibco, Cat. A15563), plating Supplements (FBS) (Gibco, Cat. A13450), maintenance supplement (Gibco, Cat. A15564), respectively. PCH were counted and plated at a density of 30,000 cells/well. PMH and PCH were incubated at 37° C., 5% CO2 for 24 hours prior to treatment with LNPs. LNPs were prepared using the process as described above with an alternative mixing technology. Post-incubation the LNPs were added to the mouse or cynomolgus hepatocytes in an 8 point 3-fold dose response curve starting at 300 ng Cas9 mRNA. The cells were lysed 72 hours post-treatment for NGS analysis as described in Example 1. Samples included in the assay were run in duplicate or triplicate and the control with 6 replicates. G000502 was used as a benchmark in this study. EC₅₀ values and mean editing results for PMH are shown in Table 11B and plotted as dose response curves in FIGS. 5C and 5D. EC₅₀ values and mean editing results for PCH are shown in Table 11C and plotted as dose response curves in FIGS. 5E and 5F.

TABLE 11B
In vitro editing in PMH using lipid nanoparticles

| | | | Guide | | |
|---|---|---|---|---|---|
| | | EC₅₀ | concentration | Mean % | |
| Guide | EC₅₀ | SEM | (nM) | Edit | SD |
| G012401 | 0.18 | 0.01 | 46.56 | 97.5 | 1.1 |
| | | | 15.52 | 98.3 | 0.5 |
| | | | 5.17 | 98.0 | 1.0 |
| | | | 1.72 | 96.1 | 0.2 |
| | | | 0.57 | 83.4 | 1.4 |
| | | | 0.19 | 51.5 | 3.8 |
| | | | 0.06 | 17.6 | 3.1 |
| | | | 0.02 | 5.1 | 1.7 |
| G019877 | 0.638 | 0.012 | 46.56 | 97.2 | 0.2 |
| | | | 15.52 | 98.2 | 0.3 |
| | | | 5.17 | 97.1 | 0.5 |
| | | | 1.72 | 85.2 | 2.4 |
| | | | 0.57 | 44.6 | 0.5 |
| | | | 0.19 | 11.5 | 2.5 |
| | | | 0.06 | 2.9 | 1.0 |
| | | | 0.02 | 0.5 | 0.1 |
| G020350 | 0.225 | 0.006 | 46.56 | 94.3 | 0.6 |
| | | | 15.52 | 96.6 | 0.4 |
| | | | 5.17 | 96.7 | 1.1 |
| | | | 1.72 | 94.9 | 0.4 |
| | | | 0.57 | 79.7 | 1.3 |
| | | | 0.19 | 42.3 | 1.2 |
| | | | 0.06 | 13.3 | 0.9 |
| | | | 0.02 | 2.8 | 0.3 |
| G020024 | 0.158 | 0.006 | 46.56 | 96.0 | 2.0 |
| | | | 15.52 | 96.6 | 0.1 |
| | | | 5.17 | 97.1 | 0.7 |
| | | | 1.72 | 95.7 | 2.5 |
| | | | 0.57 | 87.2 | 0.4 |
| | | | 0.19 | 56.6 | 2.3 |
| | | | 0.06 | 19.3 | 3.5 |
| | | | 0.02 | 4.8 | 0.9 |
| G020025 | 0.278 | 0.009 | 46.56 | 95.4 | 0.4 |
| | | | 15.52 | 96.8 | 1.1 |
| | | | 5.17 | 95.0 | 0.3 |
| | | | 1.72 | 94.7 | 2.2 |
| | | | 0.57 | 76.4 | 1.0 |
| | | | 0.19 | 33.9 | 1.8 |
| | | | 0.06 | 10.9 | 1.6 |
| | | | 0.02 | 2.4 | 0.7 |
| G017276 | 0.297 | 0.011 | 46.56 | 96.9 | 0.9 |
| | | | 15.52 | 98.1 | 0.6 |
| | | | 5.17 | 97.2 | 0.7 |
| | | | 1.72 | 91.9 | 1.4 |
| | | | 0.57 | 70.5 | 3.3 |
| | | | 0.19 | 34.1 | 3.8 |
| | | | 0.06 | 8.1 | 1.2 |
| | | | 0.02 | 2.1 | 1.3 |
| G020353 | 0.322 | 0.008 | 46.56 | 96.9 | 0.9 |
| | | | 15.52 | 98.1 | 0.3 |
| | | | 5.17 | 97.8 | 0.4 |
| | | | 1.72 | 93.6 | 1.6 |
| | | | 0.57 | 72.0 | 1.6 |
| | | | 0.19 | 28.7 | 3.9 |
| | | | 0.06 | 6.8 | 0.3 |
| | | | 0.02 | 1.4 | 0.0 |
| G020027 | 0.338 | 0.009 | 46.56 | 94.5 | 2.5 |
| | | | 15.52 | 96.1 | 0.6 |
| | | | 5.17 | 95.7 | 1.3 |
| | | | 1.72 | 92.2 | 1.8 |
| | | | 0.57 | 69.3 | 0.5 |
| | | | 0.19 | 26.7 | 0.8 |
| | | | 0.06 | 6.0 | 1.8 |
| | | | 0.02 | 2.0 | 0.7 |
| G000502 | 0.448 | 0.013 | 46.56 | 97.0 | 1.1 |
| | | | 15.52 | 96.9 | 2.7 |
| | | | 5.17 | 95.7 | 1.2 |
| | | | 1.72 | 92.6 | 1.9 |
| | | | 0.57 | 66.0 | 2.5 |
| | | | 0.19 | 27.2 | 4.2 |
| | | | 0.06 | 7.4 | 1.7 |
| | | | 0.02 | 1.9 | 1.0 |
| G020028 | 0.394 | 0.025 | 46.56 | 88.4 | 0.4 |
| | | | 15.52 | 88.3 | 0.4 |
| | | | 5.17 | 89.8 | 1.7 |
| | | | 1.72 | 84.6 | 5.1 |
| | | | 0.57 | 55.4 | 3.0 |
| | | | 0.19 | 24.4 | 1.2 |
| | | | 0.06 | 6.1 | 1.3 |
| | | | 0.02 | 0.7 | 0.1 |
| G020349 | 0.449 | 0.015 | 46.56 | 91.4 | 0.3 |
| | | | 15.52 | 94.3 | 0.8 |
| | | | 5.17 | 93.8 | 0.3 |
| | | | 1.72 | 83.8 | 2.6 |
| | | | 0.57 | 55.7 | 3.1 |
| | | | 0.19 | 20.3 | 1.2 |
| | | | 0.06 | 4.7 | 0.4 |
| | | | 0.02 | 1.0 | 0.3 |
| G020022 | 0.497 | 0.011 | 46.56 | 97.4 | 0.4 |
| | | | 15.52 | 97.9 | 0.5 |
| | | | 5.17 | 97.0 | 1.1 |
| | | | 1.72 | 88.3 | 1.1 |
| | | | 0.57 | 55.3 | 3.3 |
| | | | 0.19 | 16.9 | 1.3 |
| | | | 0.06 | 4.5 | 1.2 |
| | | | 0.02 | 0.7 | 0.3 |
| G020352 | 0.596 | 0.014 | 46.56 | 93.0 | 0.9 |
| | | | 15.52 | 96.2 | 0.6 |
| | | | 5.17 | 95.9 | 0.9 |
| | | | 1.72 | 84.5 | 1.1 |
| | | | 0.57 | 46.2 | 1.6 |
| | | | 0.19 | 12.3 | 1.3 |
| | | | 0.06 | 2.8 | 1.0 |
| | | | 0.02 | 0.5 | 0.2 |
| G020029 | 0.597 | 0.023 | 46.56 | 88.1 | 4.9 |
| | | | 15.52 | 91.6 | 2.1 |
| | | | 5.17 | 91.7 | 1.6 |
| | | | 1.72 | 83.2 | 1.3 |
| | | | 0.57 | 44.0 | 0.5 |
| | | | 0.19 | 10.6 | 1.4 |
| | | | 0.06 | 2.6 | 0.1 |
| | | | 0.02 | 1.2 | 0.4 |
| G020351 | 0.619 | 0.026 | 46.56 | 87.9 | 1.4 |
| | | | 15.52 | 95.0 | 0.4 |
| | | | 5.17 | 95.7 | 1.3 |
| | | | 1.72 | 84.6 | 1.8 |
| | | | 0.57 | 42.7 | 1.3 |
| | | | 0.19 | 12.1 | 1.7 |
| | | | 0.06 | 2.0 | 0.4 |
| | | | 0.02 | 0.5 | 0.4 |
| G020023 | 0.652 | 0.027 | 46.56 | 97.0 | 0.6 |
| | | | 15.52 | 98.4 | 0.5 |
| | | | 5.17 | 97.1 | 0.8 |
| | | | 1.72 | 83.8 | 2.8 |
| | | | 0.57 | 43.5 | 5.8 |
| | | | 0.19 | 12.0 | 5.4 |
| | | | 0.06 | 1.8 | 0.6 |
| | | | 0.02 | 0.6 | 0.5 |
| G020026 | 0.669 | 0.019 | 46.56 | 89.0 | 1.3 |
| | | | 15.52 | 91.9 | 2.2 |
| | | | 5.17 | 89.8 | 0.1 |
| | | | 1.72 | 78.3 | 0.4 |
| | | | 0.57 | 39.3 | 2.2 |
| | | | 0.19 | 10.7 | 0.1 |
| | | | 0.06 | 2.4 | 0.1 |
| | | | 0.02 | 1.0 | 0.4 |
| G020030 | 3.359 | 0.27 | 46.56 | 68.4 | 1.1 |
| | | | 15.52 | 79.9 | 4.1 |
| | | | 5.17 | 54.9 | 2.1 |
| | | | 1.72 | 12.7 | 1.8 |
| | | | 0.57 | 1.2 | 0.1 |
| | | | 0.19 | 0.4 | 0.1 |
| | | | 0.06 | 0.4 | 0.1 |
| | | | 0.02 | 0.3 | n/d |
| G012401 | 0.1 | 0.02 | 46.6 | 90.0 | 3.7 |
| | | | 15.5 | 84.5 | 7.3 |
| | | | 5.2 | 90.4 | 3.0 |
| | | | 1.7 | 86.7 | 6.7 |
| | | | 0.6 | 78.9 | 2.4 |
| | | | 0.2 | 53.4 | 2.4 |
| | | | 0.1 | 22.7 | 0.6 |
| | | | 0.0 | 4.9 | 0.2 |
| G019877 | 0.4 | 0.05 | 46.6 | 80.0 | 12.9 |
| | | | 15.5 | 85.7 | 3.0 |
| | | | 5.2 | 87.9 | 4.0 |
| | | | 1.7 | 78.4 | 8.2 |
| | | | 0.6 | 56.8 | 10.4 |
| | | | 0.2 | 23.4 | 5.2 |
| | | | 0.1 | 5.6 | 0.7 |
| | | | 0.0 | 1.0 | 0.2 |
| G020350 | 0.2 | 0.03 | 46.6 | 79.5 | 3.0 |
| | | | 15.5 | 82.5 | 2.3 |
| | | | 5.2 | 80.3 | 16.1 |
| | | | 1.7 | 81.4 | 12.2 |
| | | | 0.6 | 73.7 | 6.2 |
| | | | 0.2 | 47.0 | 7.8 |
| | | | 0.1 | 19.6 | 3.1 |
| | | | 0.0 | 4.1 | 1.7 |
| G020024 | 0.1 | 0.01 | 46.6 | 91.3 | 0.3 |
| | | | 15.5 | 88.4 | 0.2 |
| | | | 5.2 | 90.7 | 1.6 |
| | | | 1.7 | 87.1 | 0.6 |
| | | | 0.6 | 77.6 | 1.9 |
| | | | 0.2 | 54.9 | 1.9 |
| | | | 0.1 | 27.8 | 0.4 |
| | | | 0.0 | 8.9 | 0.5 |
| G020025 | 0.2 | 0.01 | 46.6 | 89.9 | 0.1 |
| | | | 15.5 | 89.2 | 3.5 |
| | | | 5.2 | 87.4 | 0.6 |
| | | | 1.7 | 86.0 | 3.5 |
| | | | 0.6 | 70.5 | 1.6 |
| | | | 0.2 | 39.9 | 1.8 |
| | | | 0.1 | 14.3 | 1.6 |
| | | | 0.0 | 3.3 | 0.8 |
| G017276 | 0.3 | 0.01 | 46.6 | 89.3 | 1.9 |
| | | | 15.5 | 90.5 | 1.5 |
| | | | 5.2 | 88.9 | 1.0 |
| | | | 1.7 | 84.8 | 1.3 |
| | | | 0.6 | 62.1 | 0.3 |
| | | | 0.2 | 31.5 | 0.6 |
| | | | 0.1 | 2.4 | 2.0 |
| | | | 0.0 | 1.9 | 0.4 |
| G020353 | 0.2 | 0.05 | 46.6 | 83.2 | 5.7 |
| | | | 15.5 | 83.1 | 10.7 |
| | | | 5.2 | 87.6 | 6.2 |
| | | | 1.7 | 83.1 | 8.3 |
| | | | 0.6 | 68.6 | 13.9 |
| | | | 0.2 | 36.3 | 16.5 |
| | | | 0.1 | 10.3 | n/d |
| | | | 0.0 | 2.6 | 0.4 |
| G020027 | 0.2 | 0.02 | 46.6 | 92.3 | 2.1 |
| | | | 15.5 | 89.3 | 2.1 |
| | | | 5.2 | 91.4 | 1.2 |
| | | | 1.7 | 84.9 | 0.9 |
| | | | 0.6 | 68.2 | 4.3 |
| | | | 0.2 | 39.5 | 1.6 |
| | | | 0.1 | 14.4 | 0.3 |
| | | | 0.0 | 3.8 | 0.1 |
| G000502 | 0.3 | 0.03 | 46.6 | 84.8 | 8.3 |
| | | | 15.5 | 83.7 | 8.4 |
| | | | 5.2 | 84.5 | 7.1 |
| | | | 1.7 | 75.5 | 6.2 |
| | | | 0.6 | 57.5 | 6.3 |
| | | | 0.2 | 26.7 | 4.7 |
| | | | 0.1 | 8.0 | 1.7 |
| | | | 0.0 | 2.0 | 0.5 |
| G020028 | 0.4 | 0.02 | 46.6 | 87.5 | 1.5 |
| | | | 15.5 | 88.2 | 3.6 |
| | | | 5.2 | 85.8 | 3.4 |
| | | | 1.7 | 78.2 | 0.9 |
| | | | 0.6 | 56.8 | 0.1 |
| | | | 0.2 | 27.8 | 1.8 |
| | | | 0.1 | 7.8 | 0.1 |
| | | | 0.0 | 1.9 | 0.1 |
| G020349 | 0.4 | 0.16 | 46.6 | 85.3 | 8.6 |
| | | | 15.5 | 84.8 | 7.7 |
| | | | 5.2 | 91.1 | 2.1 |
| | | | 1.7 | 70.5 | 20.5 |
| | | | 0.6 | 50.4 | 22.8 |
| | | | 0.2 | 30.3 | 12.4 |
| | | | 0.1 | 8.5 | 2.2 |
| | | | 0.0 | 2.0 | 1.1 |
| G020022 | 0.3 | 0.05 | 46.6 | 77.9 | 9.1 |
| | | | 15.5 | 91.3 | 1.0 |
| | | | 5.2 | 81.9 | 10.6 |
| | | | 1.7 | 79.3 | 9.1 |
| | | | 0.6 | 62.4 | 8.6 |
| | | | 0.2 | 29.2 | 8.5 |
| | | | 0.1 | 7.0 | 2.1 |
| | | | 0.0 | 1.7 | 1.0 |
| G020352 | 0.3 | 0.05 | 46.6 | 74.7 | 3.9 |
| | | | 15.5 | 78.2 | 3.8 |
| | | | 5.2 | 81.9 | 12.9 |
| | | | 1.7 | 82.7 | 5.1 |
| | | | 0.6 | 57.1 | 9.3 |
| | | | 0.2 | 24.7 | 4.5 |
| | | | 0.1 | 9.6 | 1.8 |
| | | | 0.0 | 1.7 | 0.2 |
| G020029 | 0.5 | 0.05 | 46.6 | 88.3 | 4.1 |
| | | | 15.5 | 84.0 | 7.1 |
| | | | 5.2 | 84.9 | 0.6 |
| | | | 1.7 | 70.9 | 5.9 |
| | | | 0.6 | 44.8 | 3.2 |
| | | | 0.2 | 18.5 | 0.2 |
| | | | 0.1 | 4.6 | 0.7 |
| | | | 0.0 | 1.4 | 0.8 |
| G020351 | 0.4 | 0.07 | 46.6 | 71.9 | 8.2 |
| | | | 15.5 | 78.6 | 10.4 |
| | | | 5.2 | 89.0 | 4.2 |
| | | | 1.7 | 77.8 | 6.4 |
| | | | 0.6 | 51.8 | 10.3 |
| | | | 0.2 | 21.3 | 6.5 |
| | | | 0.1 | 4.1 | 1.8 |
| | | | 0.0 | 1.3 | 0.0 |
| G020023 | 0.4 | 0.08 | 46.6 | 81.8 | 11.8 |
| | | | 15.5 | 82.3 | 14.1 |
| | | | 5.2 | 86.6 | 8.7 |
| | | | 1.7 | 72.2 | 11.1 |
| | | | 0.6 | 53.4 | 11.9 |
| | | | 0.2 | 20.5 | 6.7 |
| | | | 0.1 | 5.2 | 0.1 |
| | | | 0.0 | 1.1 | 0.6 |
| G020026 | 0.5 | 0.02 | 46.6 | 84.5 | 1.9 |
| | | | 15.5 | 86.3 | 1.1 |
| | | | 5.2 | 84.7 | 1.1 |
| | | | 1.7 | 74.6 | 2.5 |
| | | | 0.6 | 47.1 | 2.7 |
| | | | 0.2 | 17.8 | 0.8 |
| | | | 0.1 | 4.6 | 0.5 |
| | | | 0.0 | 0.9 | 0.1 |
| G020030 | 2.8 | 0.52 | 46.6 | 62.1 | 7.8 |
| | | | 15.5 | 81.6 | 1.8 |
| | | | 5.2 | 52.2 | 5.7 |
| | | | 1.7 | 22.1 | 2.3 |
| | | | 0.6 | 8.9 | 4.3 |
| | | | 0.2 | 2.4 | 1.8 |
| | | | 0.1 | 0.5 | 0.3 |
| | | | 0.0 | 0.3 | 0.0 |

### Example 5—In Vivo Editing in Mouse Liver

Selected guide designs were tested for editing efficiency in vivo. CD-1 female mice, ranging 6-10 weeks of age were used in each study involving mice. Animals were weighed pre-dose for dosing calculations. LNPs were dosed via the lateral tail vein in a volume of 0.2 mL per animal (approximately 10 mL per kilogram body weight). The animals were observed at approximately 6 hours post dose for adverse effects. Body weight was measured at twenty-four hours post-administration, and animals were euthanized at various time points by exsanguination under isoflurane anesthesia. Blood was collected via cardiac puncture into serum separator tubes or into tubes containing buffered sodium citrate for plasma as described herein. For studies involving in vivo editing, liver tissue was collected from the left median lobe from each animal for DNA extraction and analysis.

For the in vivo studies, genomic DNA was extracted from 10 mg of tissue using a bead-based extraction kit, e.g. the Zymo Quick-DNA 96 kit (Zymo Research, Cat. #D3010) according to the manufacturer's protocol, which includes homogenizing the tissue in lysis buffer (approximately 400 μL/10 mg tissue). All DNA samples were normalized to 100 ng/μL concentration for PCR and subsequent NGS analysis, as described in Example 1.

LNPs used in all in mouse studies were generated as described in Example 4. Deviations from the protocol are noted in the respective Example.

Transthyretin (TTR) ELISA Analysis Used in Animal Studies

Blood was collected, and the serum was isolated as indicated. The total TTR serum levels were determined using a Mouse Prealbumin (Transthyretin) ELISA Kit (Aviva Systems Biology, Cat. OKIA00111); rat TTR serum levels were measured using a rat specific ELISA kit (Aviva Systems Biology catalog number OKIA00159). Kit reagents and standards were prepared according to the manufacturer's protocol. Mouse or rat serum was diluted to a final dilution of 10,000-fold with 1× assay diluent. This was done by carrying out two sequential 50-fold dilutions resulting in a 2500-fold dilution. A final 4-fold dilution step was carried out for a total sample dilution of 10,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum TTR levels were calculated by SoftMax Pro software ver. 6.4.2 or Mars software ver. 3.31 using a four parameter logistic curve fit off the standard curve. Final serum values were adjusted for the assay dilution. Percent knockdown (% KD) values were determined relative to controls, which generally were animals sham-treated with vehicle (transport and storage solution or TSS) unless otherwise indicated.

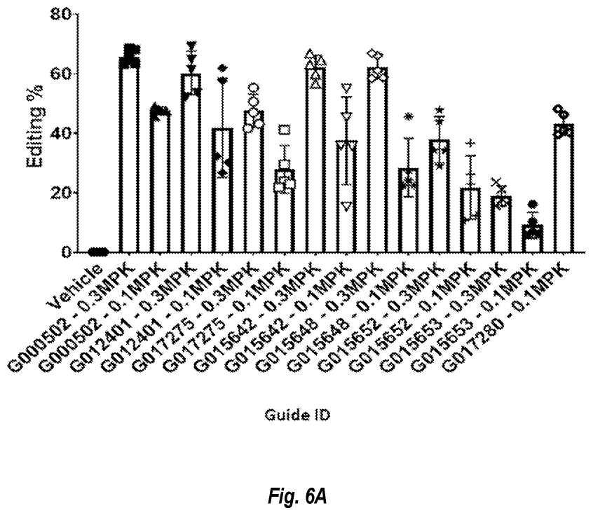

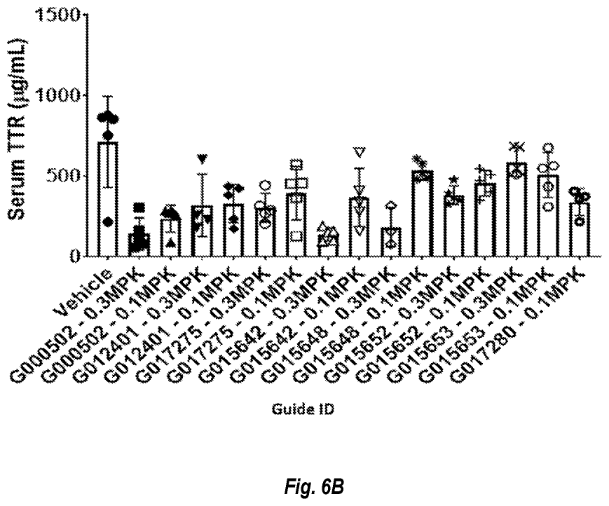

Table 12 shows the editing efficiency and TTR protein levels, respectively, for LNPs containing the sgRNAs indicated (See Table 1A for sgRNA nucleotide sequences) which all target the same sequence in the TTR gene. Guide G000502 and guide G012401, for example, served as controls. The LNPs were made as described in Example 4. The data shown in FIGS. 6A-B and Table 12 are from female CD-1 mice (n=3, 4, or 5) administered 0.1 mg/kg and 0.3 mg/kg of total RNA.

TABLE 12
Liver Editing and Serum TTR

| | Dose | | | | Serum TTR | | |
|---|---|---|---|---|---|---|---|
| Guide | (mg/kg) | % Editing | SD | n | (μg/ml) | SD | n |
| Vehicle | TSS | 0.1 | 0.0 | 4 | 713 | 282.8 | 5 |
| G000502 | 0.3 | 65.8 | 2.5 | 5 | 141 | 99.3 | 5 |
| G000502 | 0.1 | 47.6 | 1.3 | 5 | 236 | 83.2 | 5 |
| G012401 | 0.3 | 60.3 | 7.3 | 5 | 319 | 193.1 | 4 |
| G012401 | 0.1 | 41.8 | 16.7 | 5 | 330 | 119.3 | 5 |
| G017275 | 0.3 | 47.6 | 5.6 | 5 | 304 | 87.9 | 5 |
| G017275 | 0.1 | 27.9 | 8.0 | 5 | 394 | 165.7 | 5 |
| G015642 | 0.3 | 62.3 | 4.0 | 5 | 138 | 40.4 | 5 |
| G015642 | 0.1 | 37.5 | 14.8 | 5 | 367 | 181.2 | 5 |
| G015648 | 0.3 | 62.3 | 4.0 | 5 | 183 | 121.2 | 3 |
| G015648 | 0.1 | 28.5 | 9.8 | 5 | 534 | 55.5 | 5 |
| G015652 | 0.3 | 37.9 | 7.8 | 5 | 383 | 58.3 | 5 |
| G015652 | 0.1 | 21.9 | 10.9 | 5 | 456 | 79.0 | 5 |
| G015653 | 0.3 | 19.0 | 3.3 | 5 | 586 | 90.1 | 5 |
| G015653 | 0.1 | 9.1 | 4.3 | 5 | 507 | 139.4 | 5 |
| G017280 | 0.1 | 43.1 | 3.9 | 5 | 339 | 83.8 | 4 |

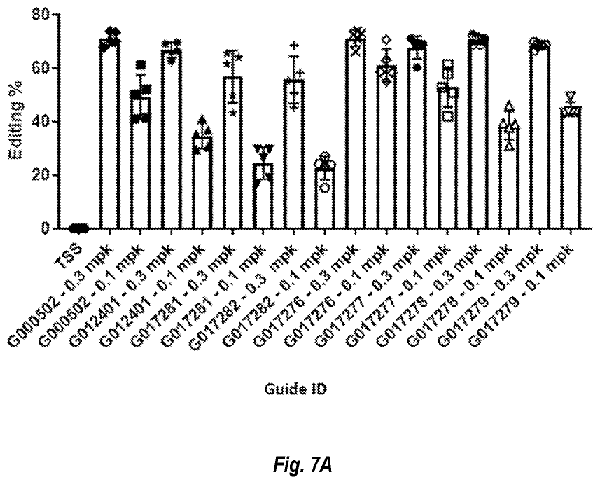

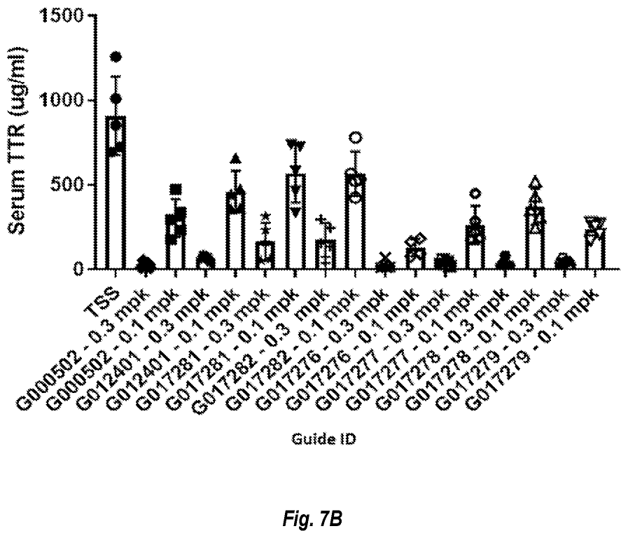

Table 13 shows the editing efficiency and TTR protein levels, respectively, for LNPs containing the sgRNAs indicated (See Table 1A for sgRNA nucleotide sequences) which all target the same sequence in the TTR gene. Guide G000502 and guide G012401, for example, served as controls. The LNPs were made as described in Example 4. The data shown in FIGS. 7A-B and Table 13 are from CD-1 female mice (n=5) administered 0.1 mg/kg and 0.3 mg/kg of total RNA.

TABLE 13
Liver Editing and Serum TTR

| | Dose | | | | Serum TTR | | |
|---|---|---|---|---|---|---|---|
| Guide | (mpk) | % Editing | SD | n | μg/ml | SD | n |
| TSS | TSS | 0.1 | 0.1 | 5 | 908 | 231.6 | 5 |
| G000502 | 0.3 | 71.1 | 2.5 | 5 | 29 | 18.6 | 5 |
| G000502 | 0.1 | 49.2 | 8.4 | 5 | 303 | 112.9 | 5 |
| G012401 | 0.3 | 66.7 | 2.9 | 5 | 68 | 17.3 | 5 |
| G012401 | 0.1 | 34.7 | 4.7 | 5 | 461 | 122.3 | 5 |
| G017281 | 0.3 | 56.9 | 9.8 | 5 | 165 | 112.4 | 5 |
| G017281 | 0.1 | 24.4 | 6.0 | 5 | 570 | 172.6 | 5 |
| G017282 | 0.3 | 55.7 | 8.8 | 5 | 175 | 100.2 | 5 |
| G017282 | 0.1 | 22.7 | 4.4 | 5 | 566 | 131.2 | 5 |
| G017276 | 0.3 | 71.3 | 3.1 | 5 | 26 | 25.8 | 5 |
| G017276 | 0.1 | 61.1 | 6.1 | 5 | 130 | 52.0 | 4 |
| G017277 | 0.3 | 67.8 | 4.3 | 5 | 38 | 15.2 | 5 |
| G017277 | 0.1 | 53.0 | 7.4 | 5 | 264 | 112.3 | 5 |
| G017278 | 0.3 | 70.6 | 1.7 | 5 | 41 | 26.7 | 5 |
| G017278 | 0.1 | 38.7 | 5.3 | 5 | 372 | 106.7 | 5 |
| G017279 | 0.3 | 68.6 | 1.4 | 5 | 45 | 14.6 | 5 |
| G017279 | 0.1 | 44.8 | 2.6 | 5 | 238 | 45.1 | 5 |

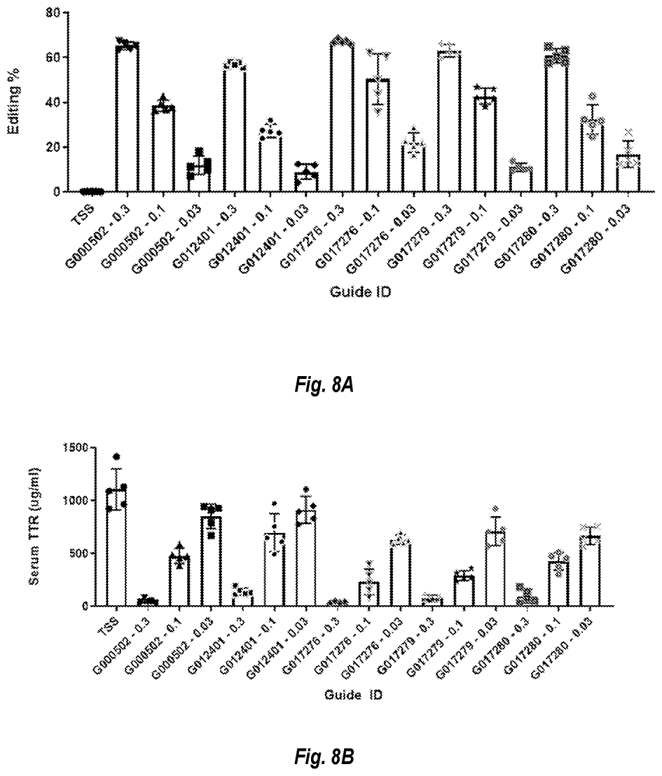

Table 14 shows the editing efficiency and TTR protein levels, respectively, for LNPs containing the sgRNAs indicated (See Table 1A for sgRNA nucleotide sequences) which all target the same sequence in the TTR gene. LNPs without guide, noted as TSS, were included in the experiment as a relative control. The data shown in FIGS. 8A-B and Table 14 are from CD-1 female mice (n=5) administered 0.03 mg/kg, 0.1 mg/kg, and 0.3 mg/kg of total RNA. Liver editing and serum protein levels were measured in the mice as described above.

TABLE 14
Liver Editing and Serum TTR

| | Dose | | | | Serum TTR | | |
|---|---|---|---|---|---|---|---|
| Guide | (mpk) | % Editing | SD | n | (ug/ml) | SD | n |
| TSS | TSS | 0.2 | 0.1 | 5 | 1106.4 | 17.5 | 5 |
| G000502 | 0.03 | 12.0 | 4.1 | 5 | 851.1 | 10.4 | 5 |
| G012401 | 0.03 | 9.1 | 2.5 | 5 | 913.5 | 6.5 | 5 |
| G017276 | 0.03 | 22.1 | 1.7 | 5 | 632.0 | 1.7 | 5 |
| G017279 | 0.03 | 11.0 | 3.4 | 5 | 711.5 | 11.6 | 5 |
| G017280 | 0.03 | 16.9 | 2.9 | 5 | 667.3 | 16.3 | 5 |
| G000502 | 0.1 | 38.6 | 1.1 | 5 | 478.9 | 3.0 | 5 |
| G012401 | 0.1 | 27.4 | 4.4 | 5 | 697.7 | 3.9 | 5 |
| G017276 | 0.1 | 50.4 | 11.3 | 5 | 233.9 | 10.9 | 5 |
| G017279 | 0.1 | 42.9 | 1.0 | 5 | 294.4 | 0.7 | 5 |
| G017280 | 0.1 | 32.3 | 1.8 | 5 | 427.0 | 12.2 | 5 |
| G000502 | 0.3 | 65.3 | 3.6 | 5 | 51.7 | 4.2 | 5 |
| G012401 | 0.3 | 56.9 | 2.8 | 5 | 143.8 | 2.8 | 5 |
| G017276 | 0.3 | 67.1 | 5.9 | 5 | 40.1 | 7.5 | 5 |
| G017279 | 0.3 | 63.1 | 6.6 | 5 | 77.3 | 7.5 | 5 |
| G017280 | 0.3 | 60.8 | 3.2 | 5 | 98.4 | 5.8 | 5 |

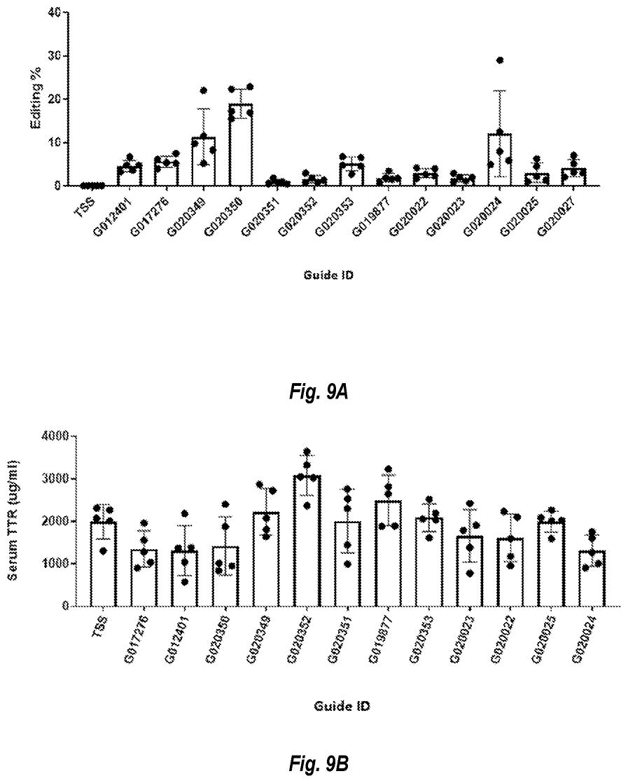

Table 15 shows the editing efficiency and TTR protein levels, respectively, for LNPs containing the sgRNAs indicated (See Table 1A for sgRNA nucleotide sequences) which all target the same sequence in the TTR gene. LNPs without guide, noted as TSS, were included in the experiment as a relative control. The data shown in Table 15 and FIGS. 9A-B are from CD-1 female mice (n=5) administered 0.03 mg/kg of total RNA. Liver editing and serum protein levels were measured in the mice as described above except animals were observed 24 hours post dose.

TABLE 15
Liver Editing and Serum TTR

| | | | | Serum TTR | | |
|---|---|---|---|---|---|---|
| Guide | % Editing | SD | n | (μg/ml) | SD | n |
| TSS | 0.1 | 0.0 | 5 | 1050.1 | 192.5 | 5 |
| G012401 | 4.7 | 1.3 | 5 | 1201.4 | 180.2 | 5 |
| G017276 | 5.7 | 1.3 | 5 | 1267.2 | 110.3 | 5 |
| G020349 | 11.4 | 6.4 | 5 | 1616.9 | 398.1 | 5 |
| G020350 | 19.0 | 3.4 | 5 | 1333.5 | 422.7 | 5 |
| G020351 | 1.0 | 0.5 | 5 | 1475.8 | 230.7 | 5 |
| G020352 | 1.6 | 0.9 | 5 | 1324.0 | 422.0 | 5 |
| G020353 | 5.1 | 1.6 | 5 | 990.9 | 153.1 | 5 |
| G019877 | 1.9 | 1.0 | 5 | 1168.1 | 236.7 | 5 |
| G020022 | 3.0 | 1.0 | 5 | 1147.2 | 304.4 | 5 |
| G020023 | 1.8 | 0.8 | 5 | 1021.9 | 139.7 | 5 |
| G020024 | 12.1 | 9.9 | 5 | 947.5 | 177.5 | 5 |
| G020025 | 3.1 | 2.3 | 5 | 998.4 | 247.0 | 5 |
| G020027 | 4.1 | 2.0 | 5 | 1193.2 | 226.6 | 5 |

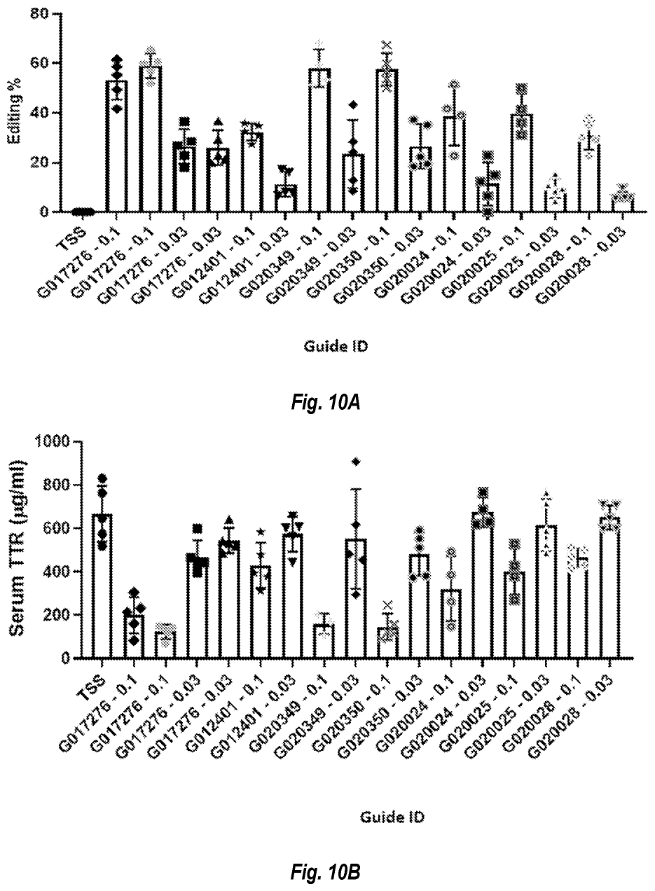

Table 16 shows the editing efficiency and TTR protein levels, respectively, for LNPs containing the sgRNAs indicated (See Table 1A for sgRNA nucleotide sequences) which all target the same sequence in the TTR gene. LNPs without guide, noted as TSS, were included in the experiment as a relative control and G000502 as a benchmark in this study. The data shown in FIGS. 10A-B and Table 16 are from CD-1 female mice (n=3, 4, or 5) administered 0.03 mg/kg and 0.1 mg/kg of total RNA. Liver editing and serum protein levels were measured in the mice as described above except animals were observed 24 hours post dose.

TABLE 16
Liver Editing and Serum TTR

| Guide | Dose | | | | Serum TTR | | |
|---|---|---|---|---|---|---|---|
| ID | (mpk) | % Editing | SD | n | (μg/ml) | SD | n |
| TSS | N/A | 0.1 | 0.04 | 5 | 667.5 | 115.6 | 5 |
| G017276 | 0.03 | 26.6 | 6.2 | 5 | 468.1 | 69.3 | 5 |
| G017276 | 0.03 | 26.0 | 6.3 | 5 | 544.8 | 52.4 | 5 |
| G012401 | 0.03 | 11.4 | 4.5 | 5 | 574.9 | 72.2 | 5 |
| G020349 | 0.03 | 23.5 | 12.3 | 5 | 552.11 | 205.3 | 5 |
| G020350 | 0.03 | 26.6 | 8.0 | 5 | 482.4 | 89.8 | 5 |
| G020024 | 0.03 | 11.4 | 7.7 | 5 | 633.3 | 99.4 | 5 |
| G020025 | 0.03 | 9.7 | 3.5 | 5 | 616.9 | 99.4 | 5 |
| G020028 | 0.03 | 6.5 | 1.6 | 5 | 651.1 | 49.8 | 5 |
| G017276 | 0.1 | 53.4 | 7.1 | 5 | 199.4 | 74.4 | 5 |
| G017276 | 0.1 | 59.1 | 7.1 | 5 | 123.6 | 29.2 | 5 |
| G012401 | 0.1 | 32.3 | 3.0 | 5 | 429.8 | 93.5 | 5 |
| G020349 | 0.1 | 58.2 | 6.6 | 4 | 159.2 | 38.8 | 3 |
| G020350 | 0.1 | 57.6 | 5.9 | 5 | 147.0 | 54.0 | 5 |
| G020024 | 0.1 | 38.8 | 10.4 | 4 | 382.0 | 165.8 | 4 |
| G020025 | 0.1 | 39.7 | 7 | 4 | 474.8 | 167.3 | 4 |
| G020028 | 0.1 | 31.1 | 5.3 | 5 | 465.9 | 37.5 | 5 |

### Example 6. In Vivo Editing in Rat Liver

Selected guide designs were further tested in rats. Sprague Dawley female rats from Charles River, ranging 6-8 weeks of age, were used in each study involving rats. LNPs were dosed via the lateral tail vein injection in a volume of 0.3-0.4 mL per animal (approximately 10 mL per kilogram body weight) or 0.35 mL per animal. The animals were observed post dose for adverse effects. Body weight was measured at twenty-four hours post-administration and animals euthanized post dose via exsanguination under isoflurane anesthesia or CO₂ asphyxiation. Blood was collected via cardiac puncture into serum separator tubes (Geriner Bio One, Catalog #450472). For studies involving in vivo editing, liver tissue was collected from the left lateral lobe from each animal. Genomic DNA was isolated and processed as described in Example 5. All DNA samples were prepared for PCR and subsequent NGS analysis as described in Example 1.

Editing efficiency in the liver and protein serum TTR levels were evaluated for each rat sample as described in Example 6. The results shown in each of the following study tables denote the sgRNA contained within each LNP (See Table 1A for sgRNA nucleotide sequences) which all target the same sequence in the TTR gene. The LNPs were made as described in Example 5. Deviations from the protocol are noted in the respective Example below.

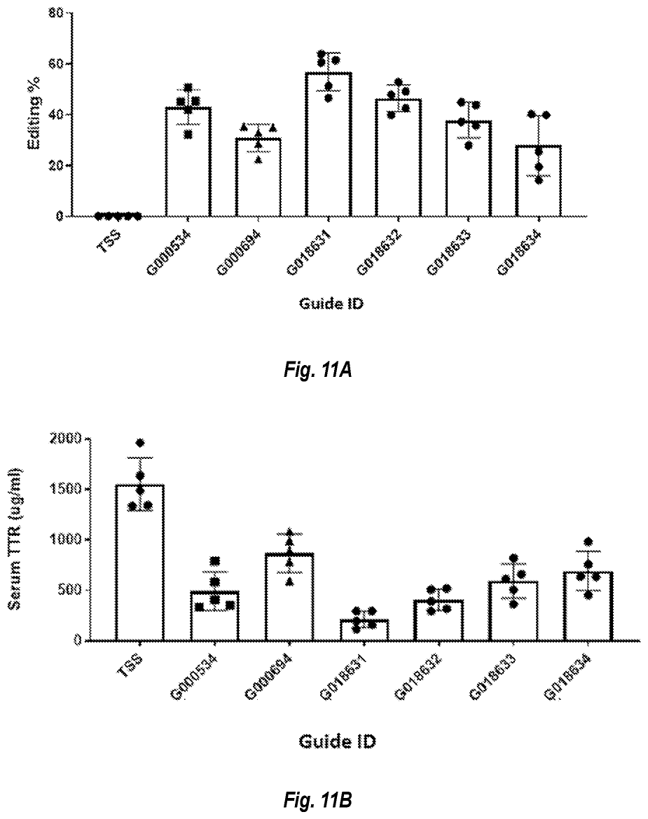

The data shown in FIGS. 11A-11B and Table 17 are from Sprague Dawley female rats (n=5 per group) administered 0.1 mg/kg of total RNA and euthanized at 7 days post dose. Samples were processed as described above.

TABLE 17
Liver Editing and Serum TTR

| | | | | Serum TTR | | | |
|---|---|---|---|---|---|---|---|
| Guide | % Editing | SD | n | (μg/ml) | SD | N | % TTR |
| TSS | 0.1 | 0 | 5 | 1553.3 | 259.4 | 5 | 100 |
| G000534 | 43.1 | 6.8 | 5 | 493.4 | 191.9 | 5 | 31.7 |
| G000694 | 30.9 | 5.4 | 5 | 867.8 | 190.2 | 5 | 55.9 |
| G018631 | 56.8 | 7.4 | 5 | 212.4 | 80.7 | 5 | 13.7 |
| G018632 | 46.5 | 5.2 | 5 | 406.6 | 104.8 | 5 | 26.2 |
| G018633 | 37.9 | 6.9 | 5 | 593 | 170.3 | 5 | 38.2 |
| G018634 | 27.9 | 11.8 | 5 | 693.2 | 194.5 | 5 | 44.6 |

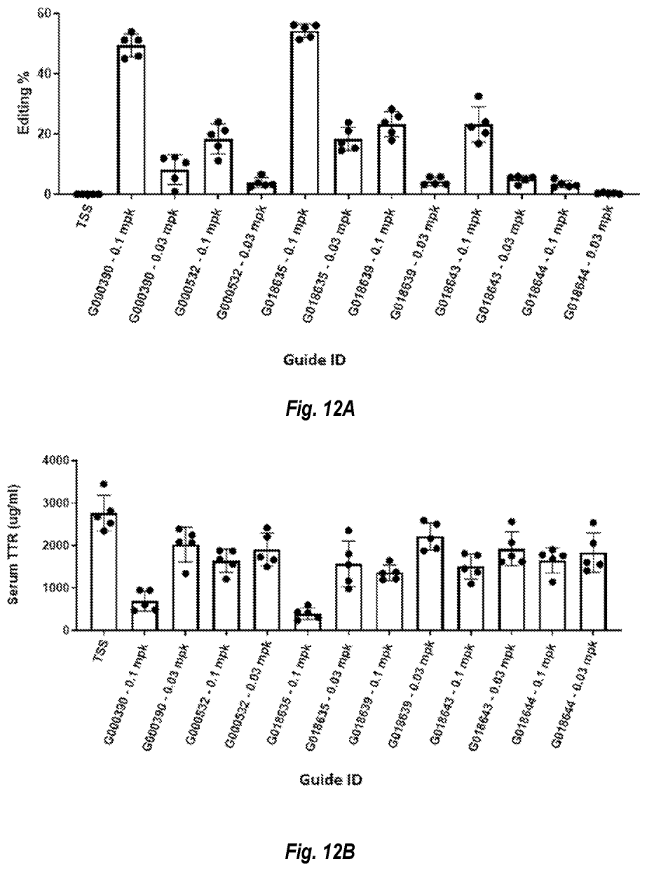

The data shown in FIGS. 12A-12B and Table 18 are from Sprague Dawley female rats (n=5 per group) administered 0.03 mg/kg and 0.1 mg/kg of total RNA and euthanized at 7 days post dose. Samples were processed as described above.

TABLE 18
Liver Editing and Serum TTR

| | Dose | | | | Serum TTR | | |
|---|---|---|---|---|---|---|---|
| Guide | (mpk) | % Editing | SD | n | (μg/ml) | SD | n |
| TSS | NA | 0.1 | 0 | 5 | 2764.5 | 420.1 | 5 |
| G000390 | 0.03 | 8.3 | 3.8 | 5 | 2027.3 | 238.1 | 5 |
| G000532 | 0.03 | 3.9 | 4.9 | 5 | 1909.8 | 405.1 | 5 |
| G018635 | 0.03 | 18.44 | 5.0 | 5 | 1575 | 272.8 | 5 |
| G018639 | 0.03 | 4.4 | 1.7 | 5 | 2216.4 | 386.8 | 5 |
| G018643 | 0.03 | 5.1 | 2.3 | 5 | 1929.3 | 136.1 | 5 |
| G018644 | 0.03 | 0.4 | 3.9 | 5 | 1836.1 | 542.2 | 5 |
| G000390 | 0.1 | 49.4 | 4.1 | 5 | 698.6 | 182.0 | 5 |
| G000532 | 0.1 | 18.5 | 1.4 | 5 | 1644.1 | 324.4 | 5 |
| G018635 | 0.1 | 54.2 | 5.8 | 5 | 403.4 | 297.6 | 5 |
| G018639 | 0.1 | 23.3 | 1.2 | 5 | 1359.9 | 400.7 | 5 |
| G018643 | 0.1 | 23.3 | 1.2 | 5 | 1508.1 | 297.0 | 5 |
| G018644 | 0.1 | 3.4 | 0.2 | 5 | 1657.5 | 464.7 | 5 |

We claim:

1. A guide RNA (gRNA) comprising a 5′ end modification or a 3′ end modification and a conserved portion of an gRNA comprising one or more of:
(a) a shortened hairpin 1 region or a substituted and optionally shortened hairpin 1 region, wherein
(i) at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9, and the hairpin 1 region optionally lacks
(aa) any one or two of H1-5 through H1-8,
(bb) one, two, or three of the following pairs of nucleotides: H1-1 and H1-12, H1-2 and H1-11, H1-3 and H1-10 and/or H1-4 and H1-9, and/or
(cc) 1-8 nucleotides of the hairpin 1 region; or
(ii) the shortened hairpin 1 region lacks 6-8 nucleotides, preferably 6 nucleotides; and
(A) one or more of positions H1-1, H1-2, or H1-3 is deleted or substituted relative to SEQ ID NO: 400 and/or
(B) one or more of positions H1-6 through H1-10 is substituted relative to SEQ ID NO: 400; or
(iii) the shortened hairpin 1 region lacks 5-10 nucleotides, preferably 5-6 nucleotides, and one or more of positions N18, H1-12, or n is substituted relative to SEQ ID NO: 400; and/or
(b) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 400; and/or
(c) a substitution relative to SEQ ID NO: 400 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; and/or
(d) an upper stem region, wherein the upper stem modification comprises a modification to any one or more of US1-US12 in the upper stem region.

2. The gRNA of claim 1, wherein position H1-1 is deleted.

3. The gRNA of claim 1, wherein position H1-1 is substituted.

4. The gRNA of any one of claims 1-3, wherein position H1-2 is deleted.

5. The gRNA of any one of claims 1-3, wherein position H1-2 is substituted.

6. The gRNA of any one of claims 1-5, wherein position H1-3 is deleted.

7. The gRNA of any one of claims 1-5, wherein position H1-3 is substituted.

8. The gRNA of any one of claims 1-7, wherein position H1-4 is deleted.

9. The gRNA of any one of claims 1-7, wherein position H1-5 is deleted.

10. The gRNA of any one of claims 1-9, wherein position H1-6 is deleted.

11. The gRNA of any one of claims 1-9, wherein position H1-6 is substituted.

12. The gRNA of any one of claims 1-11, wherein position H1-7 is deleted.

13. The gRNA of any one of claims 1-11, wherein position H1-7 is substituted.

14. The gRNA of any one of claims 1-13, wherein position H1-8 is deleted.

15. The gRNA of any one of claims 1-13, wherein position H1-8 is substituted.

16. The gRNA of any one of claims 1-15, wherein position H1-9 is deleted.

17. The gRNA of any one of claims 1-15, wherein position H1-9 is substituted.

18. The gRNA of any one of claims 1-17, wherein position H1-10 is deleted.

19. The gRNA of any one of claims 1-17, wherein position H1-10 is substituted.

20. The gRNA of any one of claims 1-19, wherein position H1-11 is deleted.

21. The gRNA of any one of claims 1-20, wherein position H1-12 is deleted.

22. The gRNA of any one of claims 1-21, wherein positions H1-11 and H1-12 are deleted.

23. The gRNA of any one of claims 1-22, wherein positions H1-7 is substituted with a G and/or H1-8 is substituted with a C.

24. The gRNA of any one of claims 1-23, wherein positions H1-6 and/or H1-7 are substituted.

25. The gRNA of any one of claims 1-24, wherein position H1-6 is substituted with a C and/or position H1-7 is substituted with a U.

26. The gRNA of any one of claims 1-25, wherein positions H1-1 and/or H1-12 are substituted.

27. The gRNA of any one of claims 1-26, wherein position H1-1 is substituted with a C and/or position H1-12 is substituted with a G.

28. The gRNA of any one of claims 1-27, wherein position N18 is substituted.

29. The gRNA of claim 28, wherein position N18 is substituted with a C.

30. The gRNA of any one of claims 1-29, wherein position H1-12 is substituted.

31. The gRNA of claim 30, wherein position H1-12 is substituted with a C or an A.

32. The gRNA of any one of claims 1-31, wherein position n is substituted.

33. The gRNA of claim 32, wherein position n is substituted with an A.

34. The gRNA of any one of claims 1-33, comprising a shortened upper stem region, wherein the shortened upper stem region lacks 1-6 nucleotides.

35. The gRNA of any one of claims 1-34, wherein the gRNA is an sgRNA.

36. The gRNA of any one of claims 1-35, wherein the gRNA comprises a 5′ end modification.

37. The gRNA of any one of claims 1-36, wherein the gRNA comprises a 3′ end modification.

38. The gRNA of any one of claims 1-37, wherein the gRNA comprises a 5′ end modification and a 3′ end modification.

39. The gRNA of any one of claims 1-38, wherein the gRNA comprises a 3′ tail.

40. The gRNA of claim 39, wherein the 3′ tail comprises 1-2, 1-3, 1-4, 1-5, 1-7, 1-10 nucleotides or 1, 2, 3, 4, 5, 6, 7, 8, 9, or 10 nucleotides.

41. The gRNA of any one of claims 1-38, wherein the gRNA does not comprise a 3′ tail.

42. The gRNA of any one of claims 1-41, comprising a modification in the hairpin region.

43. The gRNA of claim 42, further comprising a 3′ end modification.

44. The gRNA of claim 42, further comprising a 3′ end modification and a 5′ end modification.

45. The gRNA of claim 42, further comprising a 5′ end modification.

46. The gRNA of any one of claims 1-45, further comprising a guide region.

47. The gRNA of claim 46, wherein the guide region is 17, 18, 19, or 20 nucleotides in length.

48. The gRNA of any one of claims 1-47, wherein the 3′ and/or 5′ end modification comprises a protective end modification, optionally a modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof.

49. The gRNA of any one of claims 1-48, comprising a modification in the hairpin region, wherein the modification in the hairpin region comprises a modified nucleotide selected from a 2′-O-methyl (2′-Ome) modified nucleotide, a 2′-fluoro (2′-F) modified nucleotide, a phosphorothioate (PS) linkage between nucleotides, or a combination thereof.

50. The gRNA of any one of claims 1-49, wherein the 3′ and/or 5′ end modification comprises or further comprises a 2′-O-methyl (2′-Ome) modified nucleotide.

51. The gRNA of any one of claims 1-50, wherein the 3′ and/or 5′ end modification comprises or further comprises a 2′-fluoro (2′-F) modified nucleotide.

52. The gRNA of any one of claims 1-51, wherein the 3′ and/or 5′ end modification comprises or further comprises a phosphorothioate (PS) linkage between nucleotides.

53. The gRNA of any one of claims 1-52, wherein the 3′ and/or 5′ end modification comprises or further comprises an inverted abasic modified nucleotide.

54. The gRNA of any one of claims 1-53, comprising a modification in the hairpin region, wherein the modification in the hairpin region comprises or further comprises a 2′-O-methyl (2′-Ome) modified nucleotide.

55. The gRNA of any one of claims 1-54, comprising a modification in the hairpin region, wherein the modification in the hairpin region comprises or further comprises a 2′-fluoro (2′-F) modified nucleotide.

56. The gRNA of any one of claims 1-55, wherein the sgRNA comprise a 3′ tail, wherein the 3′ tail comprises a modification of any one or more of the nucleotides present in the 3′ tail.

57. The gRNA of claim 56, wherein the 3′ tail is fully modified.

58. The gRNA of any one of claims 1-57, wherein the upper stem region comprises at least one modification.

59. The gRNA of claim 58, wherein the upper stem modification comprises any one or more of:
i. a modification of any one or more of US1-US12 in the upper stem region; and
ii. a modification of at least 1, 2, 3, 4, 5, 6, 7, 8, 9, 10, 11, or all 12 nucleotides in the upper stem region.

60. The gRNA of claim 59, wherein the upper stem modification comprises one or more of:
i. a 2′-OMe modified nucleotide;
ii. a 2′-O-moe modified nucleotide;
iii. a 2′-F modified nucleotide; and
iv. combinations of one or more of (i.)-(iii.).

61. The gRNA of any one of claims 1-60, comprising a nucleotide sequence having at least 99, 98, 97, 96, 95, 94, 93, 92, 91, 90, 85, 80, 75, or 70% identity to the nucleotide sequence of any one of SEQ ID NOs: 1-98, 201-294, 401-494, 601-698, or 801-875.

62. The gRNA of any one of claims 1-61, comprising a nucleotide sequence having at least 99, 98, 97, 96, 95, 94, 93, 92, 91, 90, 85, 80, 75, or 70% identity to the nucleotide sequence of any one of SEQ ID Nos: 101-198, 301-394, 501-594, 701-798, or 901-975, wherein the modification at each nucleotide of the gRNA that corresponds to a nucleotide of the reference sequence identifier in Table 1A is identical to or equivalent to the modification shown in the reference sequence identifier in Table 1A.

63. A guide RNA comprising any of SEQ ID NOs: 1-98, 201-294, 401-494, 601-698, or 801-875.

64. A guide RNA comprising any of SEQ ID NOs: 101-198, 301-394, 501-594, 701-798, or 901-975, including the modifications of Table 1A.

65. The gRNA of any one of claims 1-64, comprising a YA modification of one or more guide region YA sites.

66. The gRNA of any one of claims 1-65, comprising a YA modification wherein the modification comprises 2′-fluoro, 2′-H, 2′-OMe, ENA, UNA, inosine, or PS modification.

67. The gRNA of any one of claims 1-66, comprising a YA modification of one or more conserved region YA sites.

68. The gRNA of any one of claims 1-67, wherein at least one modified YA site comprises
(i) a 2′-OMe modification, optionally of the pyrimidine of the YA site;
(ii) a 2′-fluoro modification, optionally of the pyrimidine of the YA site; and/or
(iii) a PS modification, optionally of the pyrimidine of the YA site.

69. An LNP composition comprising a gRNA of any one of claims 1-68.

70. A composition comprising a gRNA of any one of claims 1-68 associated with a lipid nanoparticle (LNP).

71. A composition comprising the gRNA of any one of claims 1-68, or the composition of claim 69 or 70, further comprising a nuclease or an mRNA which encodes the nuclease.

72. The composition of claim 71, wherein the nuclease is a Cas protein.

73. The composition of claim 72, wherein the Cas protein is a Cas9.

74. The composition of claim 73, wherein the Cas9 is an S. pyogenes Cas9 or an S. aureus Cas9.

75. The composition of any one of claims 71-74, wherein the nuclease is a nickase or a dCas.

76. The composition of any one of claims 71-75, wherein the nuclease is modified.

77. The composition of claim 76, wherein the modified nuclease comprises a nuclear localization signal (NLS).

78. The composition of any one of claims 71-77, comprising an mRNA which encodes the nuclease.

79. The composition of claim 78, wherein the mRNA comprises the sequence of any one of SEQ ID NOs: 1099-1127 or 1129-1146.

80. A pharmaceutical formulation comprising the gRNA of any one of claims 1-68 or the composition of any one of claims 69-79 and a pharmaceutically acceptable carrier.

81. A method of modifying a target DNA comprising, delivering a Cas protein or a nucleic acid encoding a Cas protein, and any one or more of the following to a cell:
i. the gRNA of any one of claims 1-68;
ii. the composition of any one of claims 69-79; and
iii. the pharmaceutical formulation of claim 80.

82. The method of claim 81, wherein the method results in an insertion or deletion in a gene.

83. The method of claim 81 or 82, further comprising delivering to the cell a template, wherein at least a part of the template incorporates into a target DNA at or near a double strand break site induced by the Cas protein.

84. The gRNA of any one of claims 1-68, the composition of claims 69-79, or the pharmaceutical formulation of claim 80 for use in preparing a medicament for treating a disease or disorder.

85. Use of the gRNA of any one of claims 1-68, the composition of claims 69-79, or the pharmaceutical formulation of claim 80 in the manufacture of a medicament for treating a disease or disorder.
